SEQUENCE LISTING (108 sequences)

SEQ ID NO: 1 - DNA, 957 bp, Homo sapiens
aggagtccgggggttcgcccgcggaggccggggagcagccgaccatggagccccagaacggagtcttgctctgtcaccaggctggagtgcagtagtggtgcaatctcagctcactgcaacctccgcctcctgacttcaagtgattctcctgcctcagcctcccaagtagcagggactacaggtcacaaggcccttgggtgagaggtgccctctctgtccctggaggaaggaacatccttctctctgaagataacgctgaatcgaatctgaatgatacgtcctgctgttcctcccagtttactacgcttagctcctacccctttttgtcctctcacaattttctgcttctctccacttttcatcaaactgttaaaaattatgggaggccattcttttgggctgagctcctgcactagccctcaacagatcagaccaaaccaaaatggagttacttatgctaaatgctgtgtcatcaaactgaaactttaaggaagcagatagatccccaaacagaccagtttttcctgaaaacatgagattccagtctacttgaatcagcggaagaaggaagtcccctctgctttaactattacaaaaaagtaaccgaagtagcttgatgttaaccaatcaggtttttctattctgtttccttgttgctacctcataaaacctgtggttctgctattgcccagtgggagctctcattctgttttgtagagtggaaggtgcccagattcatgaatcatgaataaaagccaattaaatctataaatttgttgtagtcctctgataatataaaacctaatttgtaatttagtcttttgacgaacctaatatataaagacataggtttaactgtttcttcaggtctttatttccttacaaaggttcctagttagatcaagttacttccagaaaagtcaatttcttctgtgtcatataaaacttatattaagt

SEQ ID NO: 2 - DNA, 1136 bp, Homo sapiens
atacactgggtttgagatttaaatttctgagtggaccaacttttaaaaactgaaagtgattgtgaaattgtggaatcattccaaaaggtcattacattaagggataataaagggggaaaacaaaaattgggggaaaaagtgttaagacttgattggaaaactagttacatatatctatcccacttactcccttgagactactctttattttatatcaatctgtatcttttattataaccatatgcgtgtattatttttatatgttaaaatagtgttatttcttgatgtagatgcttaccaaagtatttctcttttcttctgggtcctcagattggctaatttctcaggtccctggaagtgagggtgaggctattgaaatgtgggcagaagtgatacatgctacttctaggcctaaattcatgagatccttcataaatgcatttctcttcccctaaacacccccctctctctcgtttcttgtcagctatctgatttcagaggatccaaccaggactcaggcttaggggagagcggggcctctgaatataaggatcctgagtctactacttaaagtgacagccacctaggaaaatccattaagcaaaaaattctgcatcagacttaagcctctgagctttagatttgtttatttctgcagctccagcctgggtgacagaacgagactgtctccaaaaaaaaaaaaaaaaaagagtgaatctggaaacatgaaatttgaaaaattatattaatagaggattattatggctttttcaggtattaaatggtattgtgattaggttttaaaagagtccttatcttttagagatacataaggattttccacaataaggaagaaaaagagagaccaagagaagaggtacagatatacagacagagaataaagaagtaaaagctgcagatacagacaactatttttgagaaattttgctgtaaaaaggagacaagtaatgaagaggtggagggagacaaatggcccaagaaattttattttcacatgggaaatattaccatgtatttgtgcctgactttgtttccctattacaactcttaagaaagaaatgaaaagaagaaagagaaagaaaagagagagagagaaagaaagaaagaaaagaatg

SEQ ID NO: 3 - DNA, 846 bp, Homo sapiens
agcatggagtgttgggtggaggaaaggccagggcaaatgagagacaaagctggaaaaggaggctgggacctggctgggaaggtctgagcccagagatctgagctatgtttgttaagtctgggtggggaagaagtgggaatgtttatttacttagtgttttgaatctgttaactcatttcttaaacctttacatcactttatcccacaaattgtcattataccatcaacttctccctccacaagccactggactatttgagaatatccctcaagtcttcatgcgtgcatgcctcagccccaagattagagacagctattccactaaggctgtgttttcagactcttttaacagtatctcttagtaagaaatccattttcttatgtgtgcatgtatgcatatagatccatgaagcaggcgtttcacagtacaatccttttggtttgatgcatgttgatacttgcctctctattctattttattgccaatggtgactaaattgattttacaactcactaacagatcttgacctacagtttgaaaaacagagtactgaagagtcttgtttcctttatttagaacagtgcaaggatgagtatctgggtagagttgtgcgttgtacataacctttgatagtcatatagttgaaccaaaaacaatttctgtagtgacattgaaaaacattttgacttgatttatcctgacagcatacataatttttggctggctaatacattatccgattgttaattacagcaaaatggataagggaaccttataagaaaacctaccaaataccaatactgtaacattaaacttatttaaatataacaaaaaaaaaaaaactg

SEQ ID NO: 4 - DNA, 857 bp, Homo sapiens
tgctctggtttctcttcaaatcgtataaatctttcgccttttactaaagatttccgtggagagaaaccgttttgagtttcaagcaaattttttgaagccctatgttggtggggttcatcaacggtgtttaaaaatcagatagaaagttgtgtgtttgttgcgaggtgtgagacaacattttgtttgaacatttattttgggctgttgtaatggtgtgagtgtgcgttttttatttttttggaagtggggttttagactagactgtagtgagcaatctttgcctgccggcttcaagggattttcctgcttcagtctctcaagtagctggggctacaggtggcaacaccattccctgttagtttttgtattttaatagagatggggatttcccacattggccaggctgatctgcccgccttggcctcccaaagtgctgggattacaagcatgagccaccgtgcgaaaatttggttttgttttgtggttttgttttgttttgtttttaattaatttattctagaaacggtctcactgtgtcactcagtctggagtgcagtggcacgattatagctcactgcggccttaaactcctggcctcaagtgatccacccacctcaacctcccgtgagccaccgcttcccgcttggccagtttagtttttaaagggttttttttttttttaaattatgatcactttagcccagtggttcctagatttttggatttcccaattattaaaatttccgaaaaagttctggggtactatttttttaccattatacaaaagaaaacatttcaagactaaaaactaaaaccaaacaatgaccaaaaataaaaagaaaaaaaagactaaaaac

SEQ ID NO: 5 - DNA, 1298 bp, Homo sapiens
gaggtcccggctgagagcgccggctgccggctggggacaggcatctaactaccatacaagttcagaccaatggaagaaaagagcagcaaaaattgaactgaatgaatcccaaaagcaagaaattaaagacgcctttaatttatttgatattgatgggtctggaaccatcgatgtaaaagaaggaaatgcttgatgaagctgatcatgatagagatggagaaataaacgaggaagatgttttgagagtgatgaaaaagactactcgttattaatagcgttgttttagtccttgtggaaaattaacaaatttgtatttgttatgcagttttataatttaatatctgaatgtattcattttcagtttttagtttatatgtacacattggcttcttgatgtctaatccatgtaagaagttacacatctctaccaatatgatgttaaatctacagcatcaagaaacaaaacataatgacttctttgagccattaatccaagaacagtattgattctagctgctcttttgagaaccttaggtcacaaagacttagaattttaaagtttgatgaagttggcttcacatccaaatgaatttggcaaacaaaacttatattttccttttgtgcaacccaaaaaaacctgatgaacatttgctttttagtcagttactcaaatacataaagtattaaatagaactgtctggaaacattatttgctaattttcttcttttagttataaagaaaattcaaaatagcacttacatcacccatatttccctgtaagtttgactctatagtgagcacttaatacctctgtgctaggacaggtggtgtagagttttatttttaaaaaattttatttatatattttttgagacaaaatctccctctgtcacccaggctggagtgcagtggtgcgatctcggctcaatgcaacctctgcctcctgggctcaagagattctcctgcctcagcctcccgagtagctgggactacaggtacctgccaccatgcctagctaattttttgcatttttagtagagacggagtttcactgtgttagccatgatggtctcaatctcctgacctcatgatctgcctacctcagcctcccaaactgctgggattacaggggtgagccaccgtgcctggccgagattttatgtgttaacctcagtaaacatattacaagttggaagctgattgtattagctccattttacatacaagaaaactaaagttcagagaagttttaaaaattccccagttgcccattgataagaaataaaacatggattcc

SEQ ID NO: 6 - DNA, 839 bp, Homo sapiens
cagggatcccgcggctcctcgcggcttggcctgaccggcttcctccacatacgcccctctcctacacaagtccggccctcggcgctcctgccgaagcagcaggcgcctcggactctgcgcggctcccgccggccactcacctatgatggtccgcgccgccgagacgatgaccacaggatctgagcctgcattcatcttgcttctcctgccgccgtctgccctgcgctgcctgcaagctaggcaaagctcctcctcgcagcccacaggcctgccgcgggcgggacctgagctcaggactggaatcccacgggcgagaatctcgagtgcgagcccggcccgcggcgggtctcagcacagctctgatgggtccttttgctctcggcggcttcgggaggtcctatgcgtttctcctggcgccagtcattcgctcgcgtgaggggcttgctaggagcgtctcggtgcgagccccctgccttctaagatcaactctggctgtgaacgggtgaggacgccgggagcgtcttagcgtcacaaaagttcctttggttgacgtttggacagtgaaaggagcatgtaacaagtggggtcagttttgtttaaaaccctttctgacttcccagcctcttactgggcaaagttcataatttttctgagctttctagggtttccaggcaagcttatgaggggccaccgttacagcaacttgttgggaacacctggatagtggaacaagaaagctttgtcacaaagtcatgaaggggaaaactgttgacggacctggagaatcttcctgaagaactttaaagttgattgaaagaatcattaaatcctggaaacct

SEQ ID NO: 7 - DNA, 1155 bp, Homo sapiens
gagagcggcggtgctgggaggcccggccagctcgatcgcaggcttccacctggcggccagtaagtagccggtccggttaagtagtaggtgcgctaagagctccccgcgcctcttagcgcgccgtgtacgcgcctggaggcagggaccggggacgcggagctgggcgggagactcgcgggtcagggacgcggggtgaggctgggggtgagagacaaggctggagtccgtggcgcgcttcggagggctgagataatgggggcgagcgcgcgggcagtcccttccccccgtgaaaggtagaggtggcttccagctctcttctctagctcggctaaatgcaaaggcgccttacctggagggcttgtcagagccaggactgaaggtgggatctgctgcttgtaacctgcaggtccccggatgcgcaccccaacggcgcctgcgcctacgggggcctcagccttgcaaaatgtcactcagcagagcctgctgctgaaacgtgggagttgaaaaaggtacagcacctgcctagcaatgaccagctcctttcgtcacaaggaagatgacatttttagaaaacatgtcatgggttttcactagatctccatgtttatttcttggtggttatgtattaacatcttatctctactattaggcttgtaggctagctggcgtcagtagtcattctgtgtctcataaattaccgttttattagcgccagatacattgtcatgtaatttcatttgatcacaagtatcctatgagataggcgaagcagacacattgctagtttactgaaaattaagatccagacaagttaacccagagtcactctgtgtggaagtggcaaagttggaaaccaaacttaatttttttaaattaatatttatgagcatttaccctatgctaggtcctatactagttacacagtcatgtatggtatatgccatcacttctgatctttgtgtcatttgtgaccttgtgtgtatcagccaggcgcagttcactggtgaacacctaaacttgaaacgtgcggtctgggtgcgatggttcatgcctgtaatcccagcaccttgggaggccaaggcgggcggatcacgaggtcaggagatcgagaccatcctggctaacacggtgaaaccccgtctctactaaaaatac

SEQ ID NO: 8 - DNA, 1389 bp, Homo sapiens
aagggagaggccggggcttcctgcttgggctggttccgttgagaaagctctagccacctggtatctggcacggggactaatgtggaaaaggataactggccttccaggccgagagcctaaaggaaagcttcaggagaagatttgaatgcaggaaagaaaccagacagcctcaaaaaggacaatctcagtgttagccctgtcggtgcaggagcagactggtacctgccacctctggagaggtgttttctttggagacggctaaggcgccagcttatgcttgcctgtggacccactggagccgagtgaaatccctgtagatgcaacccatgctcacagcacctgggtcctcaagggctccagtaagagggtaactcccccaagcctgcctgtggctgctcatagcaggcggagcagggcaggccgtgagcagtggcccggaattaggaggtccagctgcctgggctgcccagaccacctgtccagcggttagagctcaggacccatgtgacaggctctgaagctgcagccccacaacggatggctacctctgcacagggaaagacctggatgctctattcattcaacagaatacagaaggcactgggaatagagtgaacaatgacaaccaatgcacagctgcccacaaggtgggctcctgggggagggtcatccctctgagaagagggcggcaccaagacccacacacctgaaaaatgtggtacttcatgtcgctgatctcgatggtcttgctgctgtccccatcctgttctgatttattggtcattagtgtcttgaacctggagcaaaggagacaaagcaaggtgggttttgaaccttttacttcaccactgtgtggcgatggcaccatctgtcacctgaccggctaccacaagacggaacattttaaaaattactgctgtgctcctaaaataattttcagcaagtgccattttacaccatcttaggaagacatctgagctgagcccaattctgtccccaccacccaccctacaagcgacctgacgcctgtggccagaatgctgactcttcattccaggatatttatgttttctaataataaaagcaataactaggccagaaagaacaccacctcagagcccccctttcctgctgccctgggtccaccccgtctcatcccgctgtggggcgagtggggctctgctgcaatgtgactgcagtctgaggggcagaggctgcagggtacagccccagcgagtcactctctgtcacctggaatctgaaacaaggtgcttctgtgcccctgggctgggagtttgttatctgaggctgcctacctgttagaagctgtcaccagcaggactttatgtgcataaaacagctttccttccaccaggaaggtcacatctg

SEQ ID NO: 9 - DNA, 1146 bp, Homo sapiens
gtctgctccggttcagctgcaggaggtttcttgggagttcagcccagcgcgcagccccgccgcgacgccgcgagccgagaactctcctggggcgaagggagcgctgagaagtcgctactcttactaaaaggatgcgttggatgctgggccatacccttggcgatgcacactggagacccgctcggatacggactccattaacatcaaacggagcgtgttagaggatccgagtttactgagcactttctaggaagcgtgcactgagctacgtgctttctaggccttagttcaatacttaaccatattcattataatccggtgaaatcgtaattatttagccttattttacagatgaggacactgcgtaccaacaagtaacctcactaagaatttcgtcatggaggagaagggaacctgtattcaaattaggaaggatcctgaagaaagggcacctttaggtggaattttatctttggttctgctgcagagtacttgctgctttttagtgctccctccacccccctcattcttccttgtagattagtctgttgtttgtatgctgagttccttttttaagctccttgaaagttggaacccactgctgttgcctgttcaggatgctacacattgaatcaacatggatttattaagcgctgaacgagggctagggatcgtgctgggtgctgaggattgagtggcgaacacttgggtcagacaagtaaaagggcaattagaataactgttaaatgcttagatggggttagttctatggaagcacccatgggggtgggatacagatcagagagaaaagggtggtctgcaaatagtttgggatggctacactggagagtgatcaagagtgagtgtagggcaggggttggccgactttttccataaagtgctctgaaagttgcagattccaggagaagtaactattgtcagacccagacaaattggagcctagagaccagaaaggaaggaaactcatgcttgtatgtctgagataagaactgtctcaaggccagacgcggtggctcacacctgtaatcctagtactttgggaggccgaggcgggcggatcacgaggtcaggagtttgagaccagcctggccaacgtggtgaaattcccgtctctactaaaaatac

SEQ ID NO: 10 - DNA, 1440 bp, Homo sapiens
ggcagaaaggtaatgcttaatgcagataactcttctaatcagtgtccatggcaatatgaacgcttgaagaaaactcagtgacatatcttgctcagtagtttcttattcctgaagaaccacaagcataaagtgaggcctcagtgctggtgctcttggagtatggggaatgtgcaaatatttaactgttttgtatgctgcacattgcaggtctgctcatgtgcattctccctttgtcttcctttgtcatatgtgtttttgcttttttgaaagtgcagtctttattgtaccctcctccagcttgtagcaaattagaatgcttagcatttatgttcattcattattgtatttgccatgtaaaatttttattaccttagacaagcttataagctgttactacataacttatcttactgtaactcttttatttcccccgacgttgtaatttgtttgtgatgtatattgtgaaattgtattctatgttaatttaatcagcacaattcactgacatgctggactgacatgctggctgctgtttcaaagtgtaaagtttgtgtagggctgttgggacaactgcaactctgttgtcaaggtactgtgctttggttcctatagcaacactgggtgtggcccttgaattgctaagggcatttaatacatcctggagcaaattttaactgcagattttctttgtagaaattctatgtataatgcaggtacctacttggcccatggctggtaactatttgggcaattagaaaaaaagaaaaaaacataaaaactagtgtctattgctgctttgaatatgtttgaaagtctgaaaatgtaaatagtttatcaaaaaaaaatcttgtacagtccagtgtaaagtttttaaatgaccttaagggttgccatcacatctttctcacactctcctcttgataataataataataaaaagtttgctaaggattaaagaaatgggaaaaataaaaaaaatctcttcaaatttacaggaatgaatcattgttcttagctttgttgcatacacaaacttcttggattttgttgtgcagtattgacgtgagataaagctcaacattgaataatctttcagtggtacttttcaaagtcttcccctcctctgcctcataattaagggaaaagacaaaattgaaagacacactgtctttatctatcctggtgtatgttggcaccttagctactttttttttttcctttttgcacaaggtgctttcctgatatgttcaacatgccatctttgggtgataatgtatatgccgtgatggggctcaggccccttaggggagtgtctataagaactgcctatttatgctcatttacctcaagactgtcctctctaccctaatctagttgtcatcactccatcttttgtactgctgttgacacttacaaattaaagataaattttgttttatg

SEQ ID NO: 11 - DNA, 1285 bp, Homo sapiens
aggtaggcagagaaaaaggaagaataactgggatttagaacagaaacttatataagccttcaaacagataaaacccgactttacagagttaatgaaaagcatattcccctacatgcagttgtatcttctgccaactctctttatcctttttaggtcaatgacagatataattctagtacctgttttgtgcgggcacctaacatgtttattatttaattctcacaacttccaaggtacatattattttctccatataaaagatgatgaaactgaagcaagaaaaaaaaaaattttatgaaattgtaggccaggtgcagtggctcatgcctgtaatcccagctactcgggatggagactaaggcaggaggattgcttgagcccataagttcaaggttacaatgagctatgctcatcccactgcactccagcctgggtgacagagcaagaccctgtctcaaataaaaaaaagaaattgcagataaaacattttataaatggcagagccaaagtatagtctttgatacttgactagagaatgaaattaaagaaaatcatagcatggacatcaaaaaacgtaatgttacattaagtatgagtctggttattatacaaaggttattttgggaggcaagaaaaaaactaaacatggaaattcttaaaaatgtctttctcaaattcctttatgaagtacaggaagtaaaagctgctaactcagctcagcagtaaagcgattactgtacctgtctacacaggtagtgttcattgtgtccaaacgcatgtatagcatttcagttgcctgtgcaagctgtgaatttaggataaggaaaactattctccacttttatcccaaataagttgtaatagacaagaatagaaataaaacatttcaagtctttataatgattaataatgctgaacatcttttcacacaatcaatcttgtttttgttttttcttacagctttgaaatatcagtgatgtataatgaatctgacctgcatacactcgtgaaatcaagataatattttcatcacctcccaaatttccccatgcccctccatacgcacaggcatacattgtttcattgtgcttagcagaaattgcttcttttttttttttttaactgaaggtttgtggcaaccctgcaacaagcaagtctactggcaccatttttccaatggcatctgcttgcttcttgtctctgtgtcacatactggtaatttttacaataaaacaagcttttccattattaattgctgcaatcccataataaaactttaaaaaat

SEQ ID NO: 12 - DNA, 561 bp, Homo sapiens
aaaggggggccagggggaaaagtgcacccgctcccgataaagtacaaatttttacttctcatctctggaaaaaagtccacaccggccgtctacacccgcgcctgggggagaaagcagggaagaaacggggggtgcatgagaaacgttttcatttgctccagggggaaaaatgtttctgcatcttctgatggaaagaaatctttacaagacacaggttttccggtggttattgttttttattttctgtttttaatttttttcatgttagtgacagtgatattttaatatttttttaagccagtaataatttttctccattacagggctaagttctgtggctggtggtcagtttgtaaatttatacttaaagagacttaatagtaacttcatttatttgtctggttatgttattgtatatataaatatttatgttttcatatattgcatataaaaatttgtgttacatgttactctcagaacagattgctgtaagacaattgtaaaaaaacatgtctttcgtctgtttctcaaagcaatgtaaataaaacctatggactgtcc

SEQ ID NO: 13 - DNA, 921 bp, Homo sapiens
gtcggaaggtgacttgagatgtgaagaaatgaaggtgaaatgtccagctgttaagtgaattggtatggtatggtggctagttccaaatgaaatggctaagcttcacccctttgaacactcaacttctcagtgttgctgggttagggtctccccgaccaagctggtctcggccagtggcgtccatttttgggggctcaaatccaggtcgaagggtcactggagcgacggttggagaatgtggaactagctggaagacacccgagtactcttaaagcaatccctgtgatgggcctagcaatggtaaagcttcttattctggatcaaaaagcaaagttttccagatgccctatacttcagctcaaaaattggagcttgtagctgtaattgagatgtggatcctgactcctgtgagaagtagctcaccgtgacaaagctgcctttgcttttattgatttgcaaaccaaagaagggggacatgttgggaacaagccccccctccccaaaaatctggccataaactggccccaaaactggccataaacaaaatctctgcagtactgtgacatgttcatgatggccctaatgcccatgctggaaagttgtggctttaccaaaatgagggcaaggaatacctggcccacccatggtggaaaaccacttaaaggcattcttaagccacaaacaatagcatgagcgatctgtaccttaaggaaatgctcctgctgcagttaactagcccaacctattcctttaattcagcccatcccttcttttcccataagggatacttttaattaatttaagatctatagaaacaatgctaatgactggcttgctgttaataaatacgtgggtaaatctctgtttggggctttcagctctgaaggctgtgagacccctgatttcccactttatgcctct

SEQ ID NO: 14 - DNA, 1125 bp, Homo sapiens
gaagccctgcgaaccccaaatgtgtgcgctgagccgaggcctctaagcggtaggagagaatctgacctccgacattcttttcaagacacgcgccggtggaccgggtcctgggattggcttactcccgggctggaccttggggtttaattcggaaagaagagactgcagaggagagggaagaccgtgagtctgctttcacctaggtttaaagcgaatcaaagacggctgtaaaaagggaaaccgggaccaaaaacagatttggagcctcgaaactctccattgaaggtttggaagatacgttgctgcagacttgaaaccggctccaaatccggcccaaacatgggctgaaaatatggcgcgctaatctgctgcttcccctcctcctcaagttctttctgactcttcccacctttcccagtttcccttccagacgtctgcggctccccactccacccccaggatagctactctacatcccgacaattcccagcttctacacccaggaggaggaattctggaaccacccagagcgaactcgtgccggggcggggtggggaccggaggaaggtcctgctccgaattctctccagagcagaaaagaattctagaggctacatgtgggctgctttccccccttcttccttttttccctcgcaaaccaacaaccaaaaagtgtttggtgatggaaagaacaccagtggaaaacggcaaataacagtatttccaactccgagtcaggtcctctttgcactgctttttcctgtttcattgcaatttattgattttattgtggttttttgtttatttggggcaagaacggagatgtgaaccaggacgagccagcagacccccgagccagtatctcctccctgggcagggaacaccgcctgcaccggtgcatactaggagaccctggcggagaatgtaaacaaaccgggcgccgagcccttgatctttatttaaatagaggcttgtttatcggtgtcatcctaggaggattaattagatacatctcttcacaatttggtgtctgacactccatcttaggaatgccttatcataaggtgttaattggggccactcagccacttacgtttctattaaacactgtgagggacttttcac

SEQ ID NO: 15 - DNA, 1069 bp, Homo sapiens
agtcccccttgaacgcacctcaggatggcccgtactttggaaccactagcaaagaagatctttaaaggagttttggtagccgaacttgtaggcgtttttggagcatattttttgtttagcaagatgcacacaagccaagatttcaggcaaacaatgagcaagaaatatcccttcatcttggaagtttattacaaatccactgagaagtctggaatgtatggaatcagagagctagatcaaaaaacatggttgaacagcaaaaattagatccagtcatcacgttcagcctcccatctaagctgtttgagacctttgagagaagaagaaaagatgagtgtactaccacactgtagactcttggtggtcccacagaacatgctgctgagtcacaggaacttctagcctgccttggcctgtggtttcccacccactatacaaacccactgcttgtttgttgcttttcttctcatatttattgtcaaagataaatgtttcaaaaagaaatgactaaggaaggaaaagaaacaaatgctctaaagattttctctccccaagcacttttactggtgaaataaaaaccagtaacaatcaatatgtaaaaacggcccacttccctaaaaaaaagtaatttttgtagtctgcaaggttttttttttttttgctttagtctaaatacttgttaatcttacatgttctcctgagagaagaaaaagccattcctttcaggttgtaaagtaccatgaaaaggtctttcaaaaatattcctatcagccaggcatggtggctcacaccagtaatctcagcactttgggaggccgaggcaggcgggtcacttgaggtcaggagttcgagaccagcctggccaacatggtgaaaccctgtttctactaaaaatacaaaaattagctgggcgtggtggtgcatgcctgtaatcccagctacttgggaggctaaggtaggagaattacttgaacatgggagatggaggttgcagtgagccaagatcatgccactgcattccaacctgggcaagggagtgagacgctgtctcaaaaaaaaaaaaaaaaaa

SEQ ID NO: 16 - DNA, 1159 bp, Homo sapiens
gaggctgcgtccgcacgccggcggggcgaggcggcccggccctgcgcgtcaggcctgagacctgggaggaagctggagaaaagatgccctctgaatctttgtgtttggctgcccgggctcgcctcgactccaaatggttgaaaacagatatacagatggagtcttgctctgttgcccaggctggagtgcagtggtgccatcttggctcactgcaagctctgcctcctgggttcatgccattgtcctgcctcagcctcctgagtagctggaactatagacgtccaccatcatacctggtgaatttttgtatttttagtagagacggggttttaccatgttagccaagatggtctccatcttctgacctcgtgatccacccacctcggcctcttagagtgcttgggattacaggcgtaagccaccactcccggccgatatattgctttatgaaaattatactggatctgttacagatgatagtgttgaaccaagtggaacaaagaaagaagatctggatgacagagagaaaaaagatgaaactcctgcacctgtatatggggccaagtcaattctggagagctgggtatggagtaagcaaccagggatgttcacggaataccaggcactaaagggccagaatcatccccctacaggaccagcacttggccctggccatcctgctggagctggctgtgcagagaggcatgctgaggtgagggctggtgcagaccgggaatgctttggggaagcgcctctgtatccaaatacctgttgcattgtgtgcgtttcactgaatcgtgtgactgcagcaggtgtggtgctctacagagaaccatgtcccagggctctctcttttccttttcttcacttcctgttttatgctcagttttctagcctgggaactgttcttctttttttttctttcagttttcctcatttaattatttttattccatgaatttaagaccctagatcttcatgtaaatgtgctctttgagcttcttaactggtctttcctatcagcagaaggcgatgtcttgtgctaaaatctcagtgtcaattcagtgatttaactaccacggctttactttcgtttcctttcatatcccaagtatttcttcacttctatctagctgtttgcttttatttttgatcaacc

SEQ ID NO: 17 - DNA, 1094 bp, Homo sapiens
acagcctcagccgcagcggccgtgctacctaggtgatagcggagcggctgggtaggaagcaattgttctcaaacttcactagccccgtcggcgcggacgcttgtcgagaatgcagattcctgggtactgccagatacgaattgagcataccacaaaaaagttctcattttgtgtcctcccatcccattctcctcactaaccaaaggctaggaattatctgtgaatgtaggaccactggatttgcagtcttcatctgacactgtggagagtttctaggaatgaaacagatatatggccttgggtcccctttttttttcttttttttttttttaatagagacgagcatctcactatgttgcctagggtagtcttgaactcctggcctcaagcaatccccacccgactccgcctctcgaagtgatgggattacaggcataaaccaccacgcctggccagaaggtgctttaacaccaaatctgaaaattgttcagaagagaaacattgagcatgaacaccatctgtgcgagtcatttacttattgcccctcacctctaaatctaccttctgtactcttcttccctgtaatgatggggctagttgtcctcaaactgtttctcagacttctttttaagcttgcttcctgttcagttctgccaataggggtcactagagagagactgggaggcagaaggagagaatatgcttcctgttttttctgttcttgttaatgttgcttacaggaccagcaatgcttcttcacctagagacacttctcccagcagtggcagtgccacttcagcttctttcagcactactggaatcagcctcagtgattccccctgtacccgctcagagattatccacagcagccagatggttctaccttccacaaagattgtggttgcaattctgggcttctaagttctggttacttcatatttttccttttgttcctccagccctagaggtggtagctgctttctgaagttattatttctagatgacttttggtttttcagcctttgtattttgcttttcagccctctaatgcctgtataaccaatttccctgtaataaataaatttcctccattg

SEQ ID NO: 18 - DNA, 801 bp, Homo sapiens
aaaggacaacctcagaatagcatacaaagaacttcagatctatccttctcagcctgctcctgtgtttatgctgtagagtccatttgaaaccagcacatctgagagagcaaaacattctatactataaaaacacagaagaaaaatggattaagttgaggataaaagtgaacagagaaaataacaatgggctttgttgccacgctggaggaatagcttgtcaggtcagctttgcagagaggcatattcccaaaccagtcatcattaacagactcacttccacaggaaaaaaaaaaaaaaaaaaaagacagagtctgaaaactccggacaaaagaaatccctgcagacaaagtaaaaacaaattaaaagacccattataaaacacagatgctacaaggaaaataaactttctaagatttactgggctttgacaaacttggacatttgtggaaaagacatcagcagcaactcccagatgagaatgtcaagcttcccagctgcagttccaggactttcaccctcctttatgaccttttctcaggcatgttccagtataatctgcaatctactcaagtcggaaaaggaaacagcagcaccctggtgagaaacagtgccttctgacccacataccaaactgggcggaagcaatgcttgttcacaggagccaaggagagtagaacacacactaaaagaaaatgaattttatgtgtgtgtgtgtgtgtgtgtgtcgacacacagacacacatttttaaagactataaacacaacaaacagtttgggtgacaaataaagacagaaaatact

SEQ ID NO: 19 - DNA, 816 bp, Homo sapiens
agcttcgtgacttccttctcaggatggaaccagcttggtggcttgagggaaaggggttctgagttgcaaatcagaaacttgaagatgaaatggctactacgctgggtattgcctttatcttgaatgccagcctgatggtccaaggtgaacaagccttcctgtgttttctgaatgtgcagttcccccgcactttgcctgccaccatgtaagatgtgtctttgcgcctcctttgccttccaccatgattgtgcggcctccccagccatgtggaacgacacaactcttcaccagtgcacaaaacctgatgagaaggatacagactggattttggtccagcttgctgcttaactaggttcccttgcacaacgcacaacctgtgcaatgattatgcagtgaaattgatggccattacctaacgtggcttcagtgaccatgttttcaaaactgggagtcagagtgcatggcttgacatcaaggctttgtgtcacaaagcagaagatcattaatctgggagttgacaacattggaagcacctaaacaagttgattattcatacaagatttttttcaagtattcatttaaaactatgcagttaaaggtggtgcatgtcctgttctaattggtaggctggaaatgatggtatggagatgttgcaccacgtggtcccttcactagaggaagagtggtatgaatcgtgtccgtcactgtgacaccatgcagtcttcctggcagtacacaagttgtgagagtttgctaccatttttacatttttgtgatttaaagtgttgaataacaattaataatgtcatacatact

SEQ ID NO: 20 - DNA, 760 bp, Homo sapiens
tccgccagtcttggcggaagcctgagacgcaatagatggagactctccttttcgccttggcgtactctttttttttttttcctccctggagctggtcttgtggggcagccctaaaatgtatctccaagtgaccctgcactcatcacttgctgccttggacttagtttcctcattcgtagaatggagacaccacactttctgaagtgcttatgagacttgcaggaggaagttcttgctgcatcctgtgaactgctggaacttgctgattgcacttcatagacaattccccaaagcctgcatttgcaaagctggccttttccatttggaacactcctagcagctaccagtgccaccctccccacagcctctctggcaaagtatgctggggaacttggggatttttgctaagctgattagttagatattgtgcatctcagtgtagtttcaattactatttttcatattatgagtgagtttgtgcattttttcatatgtttaagggttattcacactggtttttctatgagcggtctatatcctttgcccgcttctttttctaagtttcttgtttttttatccattttggggagctcttttatattaggaaatccatcctttgtcttgaatataactgaacatatttttccttggtgtttatattgaaatcactatatcttttgacaattgatacttgccttttctttgtcctgtgaatttgtcatatgctgagagatgaattaaagtctctttctaccgctgg

SEQ ID NO: 21 - DNA, 1227 bp, Homo sapiens
gtctgggaagtgaggagcgtctctgcctggccgtccatcgtctgggatgtgaggagcccctctgcccggctgcccagtctgggaagtgaggagcgcctcttcccggccgccatcccatctaggaagtgaggagcgtctctgcccggccgcccatcgtctgagatgtggggagcgtctctgccccgctgccccgtctgggatgtgaggagcacctctgcccggccgccaccccatctgggagtctacaggtgtaaccagcagctccgaagagacagtaaccatcaagaacaggccataatgaagatggcggttttgtcaaaagaaaagggggaagtgtgaggaaaagaaaaagagatcagattgttactgtgtctatctaggaaaaggaagacaaaagaaactccattttgatctgtactaagaaaaattgttctgctttgagatgctgttaatctgtaactcttgtcccaaccctgtgctcgcaaaaacatgtgctgtattgactcaaggtttaagggagggctgtgcaggatgtgctttgttaaaaatgtgtttgcaggcagtatattggtgaaagtcatcgccattctccattctctgttaaccagggacacaatgcactgtggaagtctgcagggacccctgcccaagaaagcctgggtattgtccaggtttcccccgactgagacagcctgagatatggcctcatgggaagggaaagaacttacagccccccagccctacacccgtaaagggtctgtgctaaggaggattagtgaaagaggaaggcctctatgcggttgagataagagcgtggcatctgtctcctgcacgtccctgggattggatgtctcagcataaaaccgaccatatattctattctgagacaggagaaaaccaccttatggctggaggtgagacatcatggcggcaatactgctctgttactctttactgcactgagttgtttatgtaagcttaaacataaatctagcgattgtgcacatccaggcacagcaccttttcttaaacttatttatgacagagtctttgctcacatgttcctctgctgaccctctccccaccttcaccctatagccccgccacactcccctcgcagagatagtaaagatagtgatcaataaatatggagggaaccagagaccagtgccagtgcaggtcctcacttgctgagtgccggtcccctgggcccacttttcttcctct

SEQ ID NO: 22 - DNA, 981 bp, Homo sapiens
acagaccacggtgaggacactgaccaccggcagccacctgtggcctccagacccaggtccctcctggctctttccagccagccctctcctccctgcgcagatgctgtggctgctattcctgaccctcccctgcctgggggggctccatgtccaagaccccaggaaggacaccgacccgtccatctaccggatccacgctggggacgtgtatctctacgggggccgggggctgctgaacgtcagccggatcatcgtccaccccaactatgtcactgcggggctgggtgcggatgtggccctgctccagctgagtcgctgccgccgccctaccgcctgcagcaggcgagtgtgcaggtgctggagaacgccgtctgtgagcagccctaccgcaacgcctcagggcacactggcgaccggcagctcatcctggatgacatgctgtgtgccggcagcgagggccgagactcctgctacggtgactccggcggccctctggtctgcaggctgcgggggtcctggcgcctggtgggggtggtcagctggggctacggctgtaccctgcgggactttcccggcgtctacacccacgtccagatctacgtgctctggatcctgcagcaagtcggggagttgccctgagcaggctgggctgggctcccacctgggtcggctgaggagggaccaggaccttcctcctcccagcgatctccgcttcggcctccgctgcaggccaccgtcttgagcccggcttctctggctcctcagcgcccaggacctccctgatgccggggtggggaaggggccggggaagggagggtgggggcctcgctgcgtctctgtctgattaaagagcaagagcagagtgtgtggcgtctctgtgggatggatttgcattccaagctgcagccaggtgcggtttgctcagccacctcctgttggaggcctccacattttggctatggtaataaagatgctgagaac

SEQ ID NO: 23 - DNA, 1265 bp, Homo sapiens
  modified_base (252): a, c, g, t, unknown, or other
gggccgccctgtgccctgcagggccgcggggccgcgtagctctctcggtgcggcgggtaagtgctgcccggcgtcggggcgtcccgcgccgtcggtcccagcgtgcccggccgctgcctcccggggcaccccgcgctgcgcgcatccctcgggctcggcgcccgccccgggcccctccagccgcgggcgctgcctcggcgcccgggggacgcgcctccgctgcgggagctgccggtaggtgccccgctcccnggacccgctgcgagccacatttggcctagcccagatagcgtttgtacctggaaggaatgagggcgtaaaaggcctgaggggtggtggctacaaacccattagtgtatgaaagcgggcattctttcattcattcaccaaacatttattgcgcgcttactccgtgccagaaattgagagtatggtagtaaataagatggttagagagcctgctcagctggaattggcagtctagggggaaataaaattttttcgggtattaaaatacaagatatttcaatcttttttaaaaagcagatttatttttctcttttgatatacatgtgggtttctagttttgcccattgttttagctcctgtaaacattcctgtaagtttgcgtggatagttttcctttatcttgggtaactagggagtggagttgttcgatcatatcagtgtatgtttaacttataagaaactgccaaacgatttttttctaaatggtttttccattctagatttccaaaatcagtgtatggtggttccagttattccatggcatggccaacgttggatattgtcagtctttagcgttggccatttaatgggtgttgtgggcacatcattgcaatttgatttgcatttgtctggagatcaatgatattgaccatgtttcatgtgctgattggccatttgtatgtcttcttttataaagtgcctattcagtcttttgctattttttatttatttttatttttatttttatgagacggagtctcgctctttttttttctttttttgagatggagtcttgctgtgttgcctccaccctgggcgagagagtgagactctgtcccccaaaaaaaagatctttgacgtgattaagtgaaatgagacaacatgtaaaagtctctagcatttacctgacacagcataattaatcaattacaatcatcttcataacaggggacaggaactatcagaccatttaatgagaatctaacatttccctgaataataaagtttcttattct

SEQ ID NO: 24 - DNA, 1369 bp, Homo sapiens
aaagtccaggagaaagtctgacaaccacggggagagggggagggagagaccctgtggaagagagacggagactgcggcaaaaagaggaaaggagggttacgggcaccgagggggagctggagtcagccgcaaggagagagggagcgggggaggaaaagaggcgggagagagaccctaaaaagcaagctagactctccagccggccgctctggtgtggggagggagcgtcatctcaaggacactgaaaaagctgtttgccgttttgctgtttgcctccttgtcacgacctcagcgacggcagatggagccagtggagggagagagacagacggcaatgcttgggtagccagggctccctgccggccccactcctcctgcagacacgagcacgcacacacacacgcgcgcaaaacacacacgctgcccttcgccacacgtagccaagtaaaatacacaaaaagcaactgagataccattcgcacatgaccacatggaaacacacaaatgaacaaacgcattcaggcatagccacatgccagtcacacagatggatagacaagagcctggggtacaaagccacatggcacaaagccacctggatgtatatgcccctacacacgcacacagaaaaaaacatgcaaacttaagtacacacctatccagacaagtgtgagtgcacacacacacacactcacacactctacctgacacgttagtgcatccccattgggccccagtttctctcagcaaattcagatcacccagcctgacttcaaaacctgcccccttccattccccttcatgctggttcctgaatccagctcgttgctccaggctagcaaagctgtccacagcctcatttgtggtttgctcacctgcctcagcaccatcagccagagggggcgaggaacgcggctctgcggaggagcggaatcttctcagcagcgatctgcgaagggctcagcggccacaggagggcggcagagaacagcgctccctacagatccagacgtcggccctggatgctgtctggaatccaggcaaggccaggctgggctgccctaggccggggacgtctttagctcgagcaaaaacccatgaatttcagaattcaggaatccttgcatcccttctcaagctctctagtccctgaaatgccatcgccctcctcagcctcccttccatttctcgttctcctagcactggggctgctggcacagaaggaccctgtggcttttccttcatggtatagcagatgacacccacgctcctccgtgacattccctagggtcccagctgataactggagctagaactagaacccacgacttcttggttctctgcgcaggggtcttcccattgtgtcagc

SEQ ID NO: 25 - DNA, 1368 bp, Homo sapiens
agggttatgagatcagtctggattcttgttgtaccagaaattttaaaaatgctcaaagtatggattggggcatgtcaaatggacacaagagctagctcaaagaggcactgactggccagatcttggacaatattttttggtaacagctttattgagatataattcatgtatcatagaattaattccattaacgtgtacaattcaatggtttttagtatatttgcagagttatgcaactaccacctcactcagttttagaatattttcaccaatctaaaacaaaaccccatttagcagttactgcccaccccgctcctcccaatgcctggcaaccactagtctactttctgtctctatggatttgcctactctggacatttcatatacatggagttacataaaacatggcattttgtatctggcttctttcacttaatgttttcaaggttcattcaggctggagcgcataatgatactttattcctttctatggttgaataatattccattgtatgaatagaccatattttgtctatccattcatcagttgatggacatctgggttatttctatttttggctatcgtgaataatgctgccatggacattcacgtataagtttttgtgtggatatatgttttcatttctttggagtagagttgctgggtcatggggtaaccctaggtttaagcttttgaggcctaccagatttccaaagtgactgcatcattttgcattcccatcaacagtatatgaaggttctaacttctctacatcttcaccaatatttgttattgtctgtcttcttgacaaaagttctcctagtgggtgtgaactggtatcattttgtggttttgatttgcatttcctggatggttatgaatgttgattttactttcatgtgcttattggccattgtatatctttgggaaaatagctattttcccaaacttttgcccagtttaaaattgagttttctttttattactgagttggaagtgttctttatatattctggatactagaccagcagcagatatatggcagatattttttcccattctgtgggttgcctttcactttcttggtggtgttctttgaagccccaaagtttttaattttgatgatatgcaatttatctatttttccttttgttgcatgtgcttttggtgtcatatctaagaaatcattgcataatggtttccaaggtaatctgggactattagaccatcaaaatagatgatagtaacagattatacattgaatggaataggaattcatgagcccataataataataaattgacaaaaagatacatagtgtggagctgaattggaaacctcttcctgacaataaaaggttgactgat

SEQ ID NO: 26 - DNA, 941 bp, Homo sapiens
aagaaaatgttgccttctctgggtttcctcaacattatatttccattgaaaaatgtgctatggtctgaatatgcctcccccaatttcatatgttgacacataatcccaatgcaacattactaaagagatggctccttttaggaagtgaataagtcaggagggctctgtccttgctaaaataataacatcaagagatgcaagggagccatttctccccttttgcctttttgcccttctgccatataaggatacagagggggcattatctgtaaggaacaggcccacatcagacactgaacttgctgacacatcgatcttggaactcccagccttcagaactgtgagaaataaatttccattatttatcaattgcctagtcttggatattttgttatggcagaagaaacagacagcatggatcacctagaataataattactgatttatttatttatttattagtgtgcgatatggttgggctctgtgtccttactcaaatctcatgtcaaactgtaattcccaatgttgggggagatacctggtgagaggtgattgaatcatgggggataatttccccctttctgttctcatgatagtgagtgagttctcatgagatctggttgtttaaaggtatgtagcctttcctcattgttctctctcttctgctctgccatggaaagatgtgcttgcttccgctctgcctcccaccatgattgcaagtttcttgaggtctcccagccatgcttcctgtacagcctgtggatctagatcaacatactgaaaggaaacagaggaaatggactcttcttgattacaggagatggaaagtaacaggatactgaaactatgtgcttatttcttgactggagagtgaattgcaagaaaaaagtgattaaaatattattttaaagttgggaaattttaaataaaagctgtatagt

SEQ ID NO: 27 - DNA, 1028 bp, Homo sapiens
agcccgctcggagcgtcctaggcccggggctgcgctgtgaaagacccagattctcatcccagaggcccagcagtcctgaaaggcctcctctccgaccctgagccgggtccgccgaacaaagttcggaagctcgggctagctgggccagcgccattttctcgcacttgtggctggatctggttgtcccggcgactgcgccccggcgcggtctcttttcctctacctcggatccccagcactgactcgccctcagacgccggggaaggtgtggtgagctcccggccccggccgaggggtccctggagaggagctgggtggcggtggccaggccgagcgcggttgctggcccgcgcctccctccccgaggcaccattgttccgggatcgctgtgaccgccacaaagtgaatcctttcggtgcggacagtcgccttcaaagccaggccccggattcaggtcagggagaatctcagctcctgagaaatttgctcctgtttgccgcttctgctactcgaggaaatgaaacgccattaatatttgaaaaggcaattattttcctgttgaatcgagaactgtcttcatgaataatttgtagtgaggtctattgccatttgagaaatattttttttccctctctctctctctctctctctctcactctctctctcttctttttatgactaggagggggatttgagggaaatgcccgagcaggccagactctgtagttcgttgttggtcgtgtttgtttgtttgtttgtttttgtttgttgtttcccccaccccccaccgctaatgaatcaggaccgcgaaccgaaagaacgcacaaaattctgtcctgaaaacacgaaaacctgagccagccgtggcgcactgaacttgcttctctgctgtaggtcgactgtgctaagaatttaaccattttctgcttcacagaattcaaacagtgggccggcaaagagctgtaaaagtttgtttgtttaaaaaaaaaaaaaaaaaaaccctgtcattaaagatgagttccttctcc

SEQ ID NO: 28 - DNA, 1409 bp, Homo sapiens
agtcgcctcagccgcggtgtgagggagcgggagtcttccttagcttctccgccatgggtgtcgcttcgtagccgggctgctccgggaaaggcctcgtacaggaaaactagacaatccaccagcccaggaggggacaagcaggcttattcctcctcctcgtcatctctggctccagccccaccctggcccttgctggcattcttcctcttcacgtggctggggttggccaccccaatagggaagcagagggagaagtcaatgtgcttctgggaatccaggcggacagtgaagaacaggatatttaccacctgctcacggacacggatatgacactgttgaatcagcacatgagcgtggcggatggacttagccaagccaagcttgaagacctgggtctgtaggaatctctaagaaatcattgatcttcaggcccaggatgtaatccagcttcatcttgccctcatccagcaccccagtgcggaccagccactgcagcagggcaatgccttcgaacagacgccgtgggtccttctcatcaagcgtcagcagctcccgggcggccttatggatcttgaccagggtaaatttgaccctccagacctcacatttgttctggagcccatactcaccaatcagctttagcttttggtcgagatgaggtttctcgaagggtctctgcagggtcacataagtttggcaacaaacccagctctgggccgctggcatgttggctccacttgcccgtctatgcctaagcacaggccctggtcactgagaaagagctcaaattttttatatatttcatctaaggtgttgattctattgatacccttatccttttaacatgcccttatccttttaatgttggtaatgatgctacctgtcatttctgagactggtttgtgtctttcgtgtttttttctgatctgtctggctagaggtttaccaattgatttgattttcctcaaacttctggttacaatgattctctctctctctcttttcctgattatattaatttctgttctgatctttgtttcctttcttctgcttactggattttatttgctctttttctggtttcttactgtaaaggcagaagtcattgatttgcattgatttgagacttcttttctaatgtaggcagtaagtggtgtaaattcccttttagtatgactttagtggcatctcacattgatatgttttcaatttcattcagtttatttttatttattttttaaattatttatttatttatttattttgagttgtgtagccctttattagcaactaaaatagaaggtatctgttgtacaacatgggtagtaattgactataactggagatttattatattctaatacagatcatttataaatgcaatttctttattaaaaagcttccacctt

SEQ ID NO: 29 - DNA, 1322 bp, Homo sapiens
cgttttgaaaacggtgttgatacagtggaaggcttgtggtgttgctggcccttgatcgctggaaggattccgaggtgtagttttcgaagcgggagttttgttcgcattgggcgcttagcgtctgttactgtcgctaacgggagattgtcaacgtgtttgcattgggccatttgcatcagttgggaccgttgttactgacctcctaggttgtgtgttgcgtttacacgcttaaatccgggtttcacggtctcttagtactcgcgtatttaagctcatcgcgcgtaccttacttctacgcgttcacgtcggttcgtccgggcctatcagcgccgctgcttttaaattggccgtgtgcgctttacgtttgcacctttacgtcggatcactggggtccatcagcgccgtagtttttgagctggccgggcgtgctttcgagcgcttagttgctttcggtttcacttggttgcggcgtgacgatcattgagttaaaggttactgcactcacttcagtcgccatcggtctccgtaatctgactgccctttgctattacttgcttcactcacatacgatatgcggacggtcctatttaagttgagcgcccatacttcactatcccaagtgggactgctttttcatttacgtgtctctatccgaggcagtataaacatctttccattcaagtttaccgcatgtgtttcactatcgagagtcggactgcttttggatttacgtggctttgtgggatgcggtatgaagatcgttgcattcaagtttaccgcctctacttcactgcagaatctgactgccgtcagatctgtatgacagattgttgcattcgttttctgcgggtacgtcactatccagtctactttttccattgacgtggctgagtcatatgtagtatgactgtcagagacgttggaacctgaagcgaccccattttgagtgagggctagaaaaatgaggccgggacttacgggcctgcattctcagaaggatattcctagctttcagatacttacggttaagggaacaaattaatgtttactgaagagacccgagtgtccagatagctggatatctggagaacaaaggcgttcctaattttgctttaaaggtagtaatagggattcttgcaaaatgtaataattaaagttaattatcacaaacccttgtaacagaacacctctccccatgtgtacaagcattgtacctagggtggatacgttccttctcttagtttcaggaacgcccttctctgtctgtggagtagctgttctttcaccactttactttcttaataaacttgcttttattttgc

SEQ ID NO: 30 - DNA, 1383 bp, Homo sapiens
agtctaggtggagagtcatggtgacttggacccatccaacagagacgaagacaactaaatggattcaggatatattctagagataacgagtcctaactctttgccaaggatatggcaaagactctgatggaaaaggcagtaatgtgtacacaaaggtgaacataaataaatgtgtgtatgataaacgctgggaatggacatagaaaaacgggtatgcaattggacagagacgacgggagacagaaggaaagatacttgctcagctgtatctaagtaagttcataggagtttcatggtctgaaaaatctgtgacagtggaatttctactaagaagcttaaaaaagtttcaccttctccttggacacttcagaataaggccttcttagaatataatgcattatatcacaaatatacttttatataaaatatttgggaccaaaacaagagtaaagaaccaggatccgaaagggtgaatgcctactaaatatctttgattatctaaagctgtttaaaagctattagattggcacctggaaccccaaaatatccacttttcactgggaaggagtggctgtgcctcttcaatcatcccctaaatgcaaagaagtttgacccccacgcaatggataaaagtcacccaaaaaatgaacaaaagaggtgtttggaaaaaccagaacaccattcatcccagctaacgacttgttcattatgagtgatgatggataaagacttctcatgctgagatgaaatcaagatttatatgcattgtttcctctcccaacttgtgggaagatcatcatcatttaattctatctctgcggccagtgatgggcatccagagagctggttatcaaacggcgcagtcagctggcaaaagccgaaacgtgcagttggcacatctggacggagccgccacagccgagggtgaacaatgactcctggcaagcatccaaattgcttgcagacgctagcaggtctcatctacaaccgcccctaccagcatctctctctctctctcttttttatgagaatcagattttcattgctccattcaattgctctcctctgccattttttacagctcttatcgttcccctggaatatggccttgagacagctcatccgtgtccttcactgtgttatctcttctaacttttattaaaacttcagctttcatctgaagatagctaattaaatctggaacagattatatgcctccccttaaaaagagcacaaaactattttcttcaaccccagcaatgctttttttcctttccaaaaggtaaaatcaaaggtgtttaagaagtctctttatttacagagctaagatattcaacacgtcatatatgagttgatttatattaaagttgtctgtgatatgac

SEQ ID NO: 31 - DNA, 1378 bp, Homo sapiens
gtcaatcatgtatttgggtacataaacagaaaagcagcacaaacaaatgtgtaaaaacgaatctcatatcagttataataagtaaaaattcactaaattttgcaattaaaagataaagagtacaagtaaataataaacaaagaagatatgtcttcaatttattatttataatagactcactttaagtgttaaaagaaatgaatactataaaataaactattggattatatatgcattcactgatgtggattaatatcaaaatatataatattgggtaaaaagtcatagatgaaagcatacagaatgattcccatttataaaacctcaaaagttttaaaagtaaataatacatcttccaggaatatgcagaatcaataaaagggatcaacatctgaatgaagacaaacaagatacaccttcaaaactagaacttcattaaatcaatcttgttaaaattgaccacttaaaatgcagtgctaaatcatataatgcaggtgtgcaaaaatatactcaaaaggcatataaggttgtcacagtttaatacagagcaaaggagtagaactgtctgtgacaaatataaaccattgtaaagatatatgccttggcggaggatagttgattcatcagaattttattacattaaaacaccctagtttcttcttctgattataagtccataaaacaaaattctaatctaacattaaaaagaagccctaaaaattagggtggagttggtcaaaggtattaaatttcagttacacaaggggaataagttcaagagatctattgtacaaagtgtgactattgttactaacaatgtattctataattgaaagttggtagaataaaatttcaagtgttctcaccacaaataagtatgtgaggtaatatatatgttaattagtccaatttaggctttccacaatgtatacacatatttcagaacatcatgttgtacactataaatacatacaattttgaggtcaatttaaaaatttaaaaagaggctttgtcacaccagaatattgtcctgtatccatgtttaaattattcatgatgtatgccagctgtttttgtttctttgtatgttttttcttttctttttcagcaacctcagtccctccttttagaatgatttttacactctgcatatttcagtatttctgagctctgaaaatattttgctactcggattgagcctggacactagaagaatgctgctcaacaaatgttaaacaatagtggggtttatcatctactctcctccttgatcttccacatctgctcttgtgctgctcggtttaactttatagactattctgaaaactaatgattcctaaaggttaaggtttttcaataaatataaagtcatgaatat

SEQ ID NO: 32 - DNA, 1470 bp, Homo sapiens
gcagtcctccaacgccccgcggcgagtctgcaccccggaacggcgcggcggggcctcgcagccggcgagcgcagcccgcggcggtgctcctgtcagcggcggctcgggggccagctctcgcccctcggctcggctcggcggcggcgggcgcctcgctccgcctagcgcgcggcacagccgggagaggcatgctcactctctgtcacccaggctggagtgcagtggtgcaagatcataactcattgcagcctcgaactcctggactcaggcaatcctcctgcctcagcttcctgtgtgcatgagtcttcaatcacctgcccagaagagacatgtatcacttccgctcacattgcattgactaaagacgacaaacttggaaactacatttccctgatcttgccaacagtattcagcccggactccaccaatgagaggcacttgcatgagatttggttgggaagaaaaggagaagccattgttttcttgaggcagcagcagatggctgacatggaattttgcctaaaacttttgggtgttctcctgaaaatactccaactggctctacaggcagctgagagcaatggcagtggcttccttgtgattcctgcactaccagatttcctgaaagaggtgtcccgatcactatcactcttgcagctttcctagagttatttaagcctctaattccctgtataagcctccttctatctgagatacctagaggggtatctgtttttctgactgtaccatagcagataaaaggacctagtctgcaagcttccactggaaattctaggagagtgaaacttgaggtagaggtcccctaagtcccttagagcagttgcaatggtacctttggaattgatggttcttgactttagggattgtgagctttttccaagtagtcacccaaacctgtgcttgttggatctaagaaaatagagttcatgtatgaaatgaaaacaattaacattgaatctataaagctgtataatctactgagacgaaagaaatgggatagaaaaagtttaatgttttactacacacatgtaactgcataaaatgtatctgaagagcacacaagaaacagacaacagaacagagaactaagtggtggttatggtttgaatgatggtgttccctccaaatttcatgttgaaacttgatccttatcgtggtgactttaaaggcatggggacttttaggaggtaatcaggtcatgagggttcttccctcatgaatgggattaaggtctttataaaagaggcttcacacagagcttgaccctttcttgccctctgtctctgtctcacacgaggacacactgttcctcctctccagaggatgcagcaacaaggcaccagcttggaagcagagatcaaacccttatcagatgccaaacctgcttatgccttgatctgggacttcccagcctccagaaccatgagaaataaatttctattgtttgtg

SEQ ID NO: 33 - DNA, 1204 bp, Homo sapiens
agatttctgttgtttcaagcccccgagcctgtggtattttgtcccagcagcctgagcaggctgaaatgggaggaaactgcagccgagtcagctcctctgactctaggccgaccgttcttccctgtatgacatgtgactcaccagataaagccacacacgtcattcttttaaaattctctactctgtcccaagttcttgtcccacatccagaagaataaggttatgcagacaaccagagagtgagcagggcagaaaaaaacttctattgcatgatggaacagctcccagcgttgaggggacctgagagtgggcagccactacctgaaggtgagtagtctctacccaaaggtggtagtccctgctgtgtggctgagtccagggtttttatgggctcagaaagggggagtgtgtgctgattggcccatgggcgggcctagaaaaaaatcaccattcgattggctaaaaggtcgtggacttaacctggaactggcagcttggttttccagcttcaggctatctttggcttgaaggtcgggcttcactggggacctgccccagtctgcctaggaatctgtcttctgctactatcagagggaccacgcttcgcttatccagtcgtcattcacggaccctgcattgtctctgcctcttgggctttgtgaagaatgctgctgtgaacaggggagtgcccctgcctgtttgggtccctgctttccatttctttgggaggtacgtggagaggagattgcggggtcatgtggtcattctgtttggctcttggaggagcaggcatcctgttttccacagcagctctgccatcaaatggcactttcttagtgacacctcccgtatctctccccataaatgtatctctccccataaacattgcaactcctggctcaccttccttctcctgtaacttgtcactgtggaactctcaggtgtgcctgagccgactcgtagcagcccaggagggccagcagtgaggccccttccccactccgtggtcagtgccttcacacagggagtttggaggtggctgtagtgggaggactcacaccgcagaaatcagccagtgctgcaggtcaggggccgccatcccccagagctgctgtgagacatcgaccagcacatcagtgggctgctttccgctggttttatttgcatgtattttggtctgtttccctacctagaaaataaactc

SEQ ID NO: 34 - DNA, 1043 bp, Homo sapiens
gaggggggcggacggaatgtttttgaagtgtgtgtttgcctaaggtgtgtgtagaaaggtagctacttttcagtctctgcctgcctttaatccattacaatgtcctaattaaaaacatttaaaaagctacattggaaaatgatccctgaggctagaatgctgttggcacagtgaaaaagacgcaatcaataagtaaacaggcaatgtcagcccggagagctttcagaacaacatgcctactcgggggaaaaaaaattacaagtcgctttagcctatttaaatattttccaagatctttgttaaatttcaggtaataatatttcttcctttgaacaagcgttgtatgtttgcctttgactgagaatggcctaggctagagaccatttggtattcaacttgtgactattttagtatggggctggaccccaaaaattcatgctcaatgaaatgattttcacttactgagatgaatttactttttgcttccttcagcaattcatcaatctaatggaagaaagagtagctatcatcataatgagaatagacttctacgagtatgagtcaatgtcaatactttatttaataatgcaaattctattactatatatttggtctcaatccaaatttgctttaaattgagtttccttgccattgcacactcctatctttctgaacacacaccaccccacacacacaccatacacacttgtttcattgcgcttcattttattgcacttctcgaagaatgtgttttttacaaattgaagatttgtggcaacttttcatcgagtgagtctattggcatcatttttgcaagagcatatgctcacttcctgcctctgtgacagcattttttagcaataacatatttttaatgaaggtatgtacattgttttttagacataatgctattgcacacttgactaaaatgtagcctaagtataatttttatatgccatgggaaactaaaaaaagtgacttactttattgtaatatttgctttattactttggtctggaactgcctcacactatctctgaagt

SEQ ID NO: 35 - DNA, 833 bp, Homo sapiens
aaagggaacaaagatgtgtaactataacggtcctaaggtagcgagtcgaggtcgagctctatttaggtgacactatagaaccagatttttattgaactacctcacactaattttctatgctttcccaagtaagctgttgccctgttagatctttactgagtgaattataaatgtgtgttaaatactttctagccaatgttgacacaataccagtaagtatgtaaagtatataccttacatcagtaagagacacgtgtaaaatctttgactgtatgtcttgcaaaattgtgctcgttgacattattactgtttttgtaagtagaaacctgctcgtgatatcggtccatttacattttacaaaaggagtaaatcttagtaaaaattttacgaagaaataaattacttttgtaggcccaatatttggtatatttttgagaagctgttaatcttttagctgaataatgaagttagactgaattacgtgtctccctggactgtgacatctattttctcattacagtttatcctggtcagcagggtgtcacacctggaaacctgagtatgatagctgacatttgcttttctccctctgcgatgtcattcctcctccattcctctccttccctgtgttccgttccctctcctttcctctagacaaaacaaaatggggcactttttagggaatgctgagatcattattgtggtttttcatcattcatgccctagtcattaaacatgcaccactggaatgtaaacaatgttatctagtatgtcaattggttataatattttaaataaaaaagaaaaaagtggtatgaaaaaaaaaaaaaaaa

SEQ ID NO: 36 - DNA, 1002 bp, Homo sapiens
agattcccttttttccttagatgtgagtgcctgtgggcctgagaatctggaagactgcaggggtctggtggtttaagccaaagtgaattcttctctgagcactgataggattgcccataagaagaaatcatctcttcagccttctcatcctccacaggaacagaaggacacgtcccaagtggcgagctgtgcaaggccacagacacagtccctgatcaactggcgaagacactatcacttccaatgaggccatatagagccttacttcaggccacttatacttccactcaacgtggacaaccagatgtctcacctgcttgtagtgcttctcttcattgctcttagaggatcacccccaagaggggatacagtgcagcagaagttcactttcagtttctgctcacatatcaaacaaacccatccatgcccatgatgaatctctttctcctgtgtcaggtagttgttgtccagagccgccgtgtaagaagtacctttcaccttccaccatgattgtgaggcctccccagccacgtggaaatggcaggtgcagaggatccagcatgttattctgaaaccccagaagacgctgcagctaccaaatagaaggagcctggatccctgaatgtcttcatggaacagaacattctcctcactccagtctgtttgaaccagaatatgacctgagcaagaaataagccttaattatatttgaccatagaattctggtgatatttctatggctgtaagtccatgttgattaatacaaagggattctgtaaagcctcttctattaaaaaaagttgcccaaggagtgaaaggagagatattttctgtaattaagattttcataatacatagaacaggctatagcacctggcaatcaatcatatggggtacaacagaaagaggaaacattcctgctattaaattaaccaaactggagttcttgttatataatatagtaaaatttcctcaatctttcagccaaaaaaaaaaaaaaaa

SEQ ID NO: 37 - DNA, 960 bp, Homo sapiens
  modified_base (723): a, c, g, t, unknown, or other
acaattgctctacagctcagaacagcaactgctgaggctgccttgggaagaggatgatcctaaacaaagctctgatgctgggggccctcgccctgaccaccgtgatgagcccttgtggaggtgaagacattgtggctgaccatgttgcctcttacggtgtaaacttgtaccagtcttatggtccctctgggcagtacagccatgaatttgatggagacgaggagttctatgtggacctggagaggaaggagactgtctggcagttgcctctgttccgcagatttagaagatttgacccgcaatttgcactgacaaacatcgctgtgctaaaacataacttgaacatcgtgattaaacgctccaactctaccgctgctaccaatgaggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggatgagcctcttctgaaacactgggagcctgagattccaacacctatgtcagagctcacagagactgtggtctgcgccctgnggttgtctgtgggcctcgtgngcattgtggtggggaccgtcttgatcatccgaggcctgcgttcagttggtgcttccagacaccaagggcccttgtgaatcccatcctgaaaaggaaggtgttacctactaagagatgcctggggtaagccgcccagctacctaattcctcagtaacatcgatctaaaatctccatggaagcaataaattccctttaagagaaaaaaaaaaaaaaaa

SEQ ID NO: 38 - DNA, 1405 bp, Homo sapiens
gaggaggcgggcaggctggtgggctgggcgggcggcgagcggccgggagcgcgcggtggactcggccgcggcgagtagttagttagttgttgttagtcagtgtcagttgctcggcggcggcggccgtggtcaccaggaaggggacgggacggacggtgatggtggtcgccgcggcggcgtgtgcgcccctcaggtatctgcccaaactggaacttatgctgtagacatttctttgtggtttatctaaaaaccaaaggaaaagaatacttgagatccatttgtaaggaaagaaaaggaagcaacaacataatgcccagagttggagccaattaaaccaggtttgtactttttctttccgtaatgtaacatgtttattctggtcttaaatctcttttattaatctcctgtcttatttcagcaaaaatctaataattttaaaccttgccatagtagtatggaggagataaaggtgaataagacatgctcttttcaaactatttgcaactaatgcaaaagaacagaaatcataacagtctctgagaccacagtgcaatcaaattagaactcaggattaagaaactcactcaaaaccgcacaaccaaatggaaactgaacagcctgctcctgaatgactactgggtaaataacgaaatgaaggcagaaataaagatgttctttgaaaccaaatggtctttgagaacaaagatacaacataccagaatctctgggacacatttaaagcagtgtatagagggaaatttatagcactagatgcccacaagagaaagcaggaaagatctaaaatcaacactctaacatcgaaattaaaagaactagagaagcaataacaaacaaattcaaaagctagcagaagacaagaaataactaagatcagagcagaactgaaggaggcagagacacgaaaaacccttcaaaaaatcaatgaatgcaggagctgttttttttgaaaagatcagcaaaatagaccgctagccagactaatgaagaaaagagagaagaatcaaatagatgcaataaaaaaaaatgacataggggatatcaaatgattagcaacttaactatgaatatgaaagacaaaattataaacagctagatagcaatctcattttaacatggaaagttttgtgaggtttggtaaattgcttgaggtcagaagctcttaaagagttaagttgggattcagactcatatctgctgactccaaaccgtatttgccttccattatgtcacaatgttccctattttatttaggtttagttgttgtgcaactgctgattattgaagtagagggagaggagaaagaaaaaagaaaagaaaagaaaggaaaggaaaagaaaagaaaagaaaagaaaagaaaagaaaagaaaagaaaaaaaaaaaaaaa

SEQ ID NO: 39 - DNA, 746 bp, Homo sapiens
  modified_base (615): a, c, g, t, unknown, or other
ctctatttaggtgacactatagaaccacagaattaggcctctaaaaagcctcatactgctaatctctgggaatgaatggtgttctttgggataatgggatatgaagctcagtctgatttttctgttctgctggtagcttagggccccctttcttctgttgggttttttgggagaagggaagttgtgattaagaatgagaattctttttttttttttttgtctcaagagcctgggcaacagagtgagaccctgtctcaaaaacaacaacaacaaaatcttatgtaccccataaatatatacacctactgtgtatccacaaaagttaaaaattagaaaaggcaaattgcagagatttccatatgctatgataccgtttatatgaagttttacatatgtcataaaaatacagataacctttaggggaatgatcattaccaaacttttggataacggtttctggggatgggcagagagggctatacagtcatgaagaggtgtataggggctttcaactctttgtagtgttttatttcttcagtcccatggtggttatatgattcttcactcccctttttttgtgtggaatatttttcttataaaaagtgtgtcttttanttatttatttatctttttcacaggagaatggcgtgaacccaggaggcggagcttgcaatgagctgagatcatgccactgtacttcagcctggacgacagagcaagactccgtctcaaaaaaaaaaaaaaaa

SEQ ID NO: 40 - DNA, 775 bp, Homo sapiens
gaattgcgcgcaattaaccctcactaaagggaacaaagatgtgtaactataacggtcctaaggtagcgagtcgaggtcgagctctatttaggtgacactatagaaccatagtttgcaagaatggagtgcagacagtgttgcctcatgaaagacaggaactccatggactgaggaagatatactgagaaaataaagaagaacaagaatttctgcccttgcctaaatgagagatatgttatcaagataattgagtaaattctcctgaaaatggataaaaccaaagtggtaggagataagcacttctagcaaaagatgttacctcctccttgcagattcaagaacatgaagaattttactaaaatgaggggaagatgggtgcagaggaaggggaaagtcagtgattgggaacctgtgttacgactattgggaaagagtctaagttggtgaagggtctgagattaccacactttcaagatgacaagtcggcctgccacacattcaagtatgctggcagaaggctcaagagtcctggatcctggatgaatgagctatgacgatgtggatggctggatgtcaggagaagatgatgtcagtgtttggggatcctcaatagttgaaggtttttgttttgttttgttttgtttttgccaaaaacttttggaagagcattgtaatagaatgttattgtctctttctttttaactcattaaagtgttgccacagatgttgtaaaaaaataaaaaaaaaaaaaaagcacgtccaaggatcctgccat

SEQ ID NO: 41 - DNA, 819 bp, Homo sapiens
  modified_base (628): a, c, g, t, unknown, or other
aagggggatgtgctgcaaggcgattaagttgggtaacgccagggttttcccagtcacgacgttgtaaaacgacggccagtgaattgcgcgcaattaaccctcactaaagggaacaaagatgtgtaactataacggtcctaaggtagcgagtcgaggtcgagctctatttaggtgacactatagaaccagagtgagaccgcgcggcaacagcttgcggctgcggggagctcccgtgggcgctccgctggctgtgcaggcggccatggattccttgcggaaaatgctgatctcagtcgcaatgctgggcgcaggggctggcgtgggctacgcgctcctcgttatcgtgaccccgggagagcggcggaagcaggaaatgctaaaggagatgccactgcaggacccaaggagcagggaggaggcggccaggacccagcagctattgctggccactctgcaggaggcagcgaccacgcaggagaacgtggcctggaggaagaactggatggttggcggcgaaggcggcgccagcgggaggtcaccgtgagaccggacttgcctccgtgggcgccggaccttggcttgggcgcaggaatccgaggcagcctttctccttcgtgggcccagcggnagagtccngaccgagataccatgccaggactctccggggtcctgtgagctgccgtcgggtgagcacgtttcccccaaaccctggactgactgctttaaggtccgcaaggcgggccagggccgagacgcgagtcggatgtggtgaactgaaagaaccaataaaatcatgttcctccaaaaaaaaaaaaaaaa

SEQ ID NO: 42 - DNA, 757 bp, Homo sapiens
  modified_base (716): a, c, g, t, unknown, or other
cctcttcgctattacgccagctggcgaaagggggatgtgctgcaaggcgattaagttgggtaacgccagggttttcccagtcacgacgttgtaaaacgacggccagtgaattgcgcgcaattaaccctcactaaagggaacaaagatgtgtaactataacggtcctaaggtagcgagtcgaggtcgagctctatttaggtgacactatagaaccagacacagcacgcatgctagaccgtggtagaagaatgagctgtcattgctgtgcagtcctttttgtggcacaggccctggggctgatcatggttagtccagcatgtcattgtcaatcctgctgtttcatagtcatgagacacaaagcacagacaactaacagcacactggctcaggcgcttcttatggtctagagaatctgtgtgcttttgtacaggagtccagcctctttctttgtgtgcccttttgaaaaactgaaatgttcttgaagtctggagacaaacttctgggtgaaccgcatctgaacttttaaaattcctttttttcccctcttggttaaatctcagtggagccagagacgatcatcattttcatgaacacaagctaaaggtttctgttgcctgttgagcaaagtagctgcttcctattgcaaaagagctgattcctattcgcactgcgttggcgttttcattaaaattcccattatcctcttccttacaatncagnnaaaaaaaaaaaaaaaaaacacctcaaaagatactgc

SEQ ID NO: 43 - DNA, 801 bp, Homo sapiens
  modified_base (14)..(15): a, c, g, t, unknown, or other
agcctaggatcccnnacagttttgcanaacactgaaatctatggactctaaaatggacttcatttaaagaaacccacggaccattaatggacaaaaacatgagtcaatatgtattgtggcattcaaatcctagcactctgggagaggaacatatgggaaagaaatcccctcggaaaccaaagcccagccatcggagattttaagatttttcaggctttccttataatcttccttacatttgtctctttaaacacatttaagtcgacctttgagaagttgctgataagcagttatcaaacaagagttagagtaacaaaccctcccacctcgctgtgttggttggtagcttccaaggccactgtaaatgtgtatcagtgtgaacctgatccagaaacagccaggaagggagcaaagtttagtttagtctgtgaggaaactggcggctgctggtagttccatgctgtctgttcagacttcagcttggtgtaagtagtttttttaaaaaaaaactattatgacattttcatataaaaaggattgtaagaataatttccatcaaagattaaatcaagtttctacctggctctaaatcttagggattagaactactgaaaagaaagtttcagcactcagcagtagttttattttttttaatggaaaagaaagccgtgaggtgtatcagcaagtgtgctgctaaaacaggtcccgcgtgcacgaaatgatttctaatgtcttatgttgagtgcaagtgtttacagttagaaaataaaagtgagtatgtacctaaaaaaaaaaaaaaaa

SEQ ID NO: 44 - DNA, 715 bp, Homo sapiens
  modified_base (278): a, c, g, t, unknown, or other
ctattacgccagctggcgaaagggggatgtgctgcaaggcgattaagttgggtaacgccagggttttcccagtcacgacgttgtaaaacgacggccagtgaattgcgcgcaattaaccctcactaaagggaacaaagatgtgtaactataacggtcctaaggtagcgagtcgaggtcgagctctatttaggtgacactatagaaccaaattggattttttccattatgttcatcacccttatatcatgtacctcagatctctctctctctcctctctntcagttatatagtttcttgtcttggacttttttttttcttttctttttcttttttttttgctttaaaacaagtgtgatgccatatcaagtccatgttattntctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcagctgccttttaagaggtnttatctgttcagtgttaagtgatttaaaaaaataataacctgttttctgactagtttaaagatggatttgaaaatggttttgaatgcaattaggttntgctatttggacaataaactcaccttgccctaaaaaaaaaaaaaaaaaaaaaagaaaaaaaaaaaaaaaa

SEQ ID NO: 45 - DNA, 1255 bp, Homo sapiens
ctgaggccgtggtgggctgtgtctctgctgctgaggccgtggtgggctgtgtctctgctgctgaggctgtggtgggctgtgtctctgtgctgctgaggccgtggcgggctgtgtgtctctgtgttgccgaggccgtggcgggctgtgtgtctgtgctgctgcctgagctgcttgttatgtgcatattacttcatcgatcagaaaagcagcacaaccaccagagaacaccgtatcctcccagtgcagtcctgtggctcatggtaccactgtcatggaaaaatggaaaatcagacatttacaacttgagccacttgaaaagggaggattagccaaaaatcgttaattgcaagcaaaacccctgaaatggaagatactcccaaatgaccagaataaacagggtttaactgttaattgttacattatgctggaaaccacttaaacacgacactaaggtggtcactgtataagtagccctgggaaagaaggcccaggggacattgtaattaatatttttttgtaatagacttttgccctaacctgggcaacaaaagcgaaactctgtctcaaaaaagaaaaaaaagaaaaaaaagcacgtgaccttatgaggctctcgctgtattgttattttaaggactataaagagtttgatttaaaattatgcagggcccctatgtgggattttttaaaaagcaaactggtgtgtattctcatgtggtttgcacagcccagcctcacagcactattgtaaaccctgctctttctgtctcgctagacagattttttttgtttgttttcttttttctggttgttttttgttgttgttgttgttgttttacagctgaaaccaaccagcaagcccttgatgaccaagaggcgtttctttcaaagctatagggcacaaacaattgaccatagatgactccgtttgcattcttctgcagaattatttccttcagggacagattttccaacctaagaaactacctaccgtgtgtattctcttgacggggagagatgaacccttcagctgctaagatccaagaaaacgcctcactgccttaaccttaactgttcttcctggcgctaaaaagagctgtattttttaaagtgctggggcaaacaaagcaaccccaaaagagttgatgtgtgttttaaaagaaaaaacccaatgaggaacaattggagatttttatgcagaaactaaataatccttaataaataaatctctattttggaatcacaaaaaaaaaaaaaa

SEQ ID NO: 46 - DNA, 836 bp, Homo sapiens
  modified_base (377): a, c, g, t, unknown, or other
ggcgaaagggggatgtgctgcaaggcgattaagttgggtaacgccagggttttcccagtcacgacgttgtaaaacgacggccagtgaattgcgcgcaattaaccctcactaaagggaacaaagatgtgtaactataacggtcctaaggtagcgagtcgaggtcgagctctatttaggtgacactatagaaccagctgctcgccgtccgctccgtccgcccttagacctgttgcccagcatccctgcagttcgcggtacagtctctagtagagcgcgtgtatagaggcagagaggagtgaagtccacagttcctctcctccaagagcctgccgaccatgcccgcgggcgtgcccatgtccacctacctgaaaatgttngcagccagtctcctggccatgtgcgcaggggcagaagtggtgcacaggtactaccgaccggacctgacaatacctgaaattccaccaaagcgtggagaactcaaaacggagcttttgggactgaaagaaagaaaacacaaacctcaagtttctcaacaggaggaacttaaataactatgccaagaattctgtgaataatataagtcttaaatatgtatttcttaatttattgcatcaaactacttgtccttaagcacttagtctaatgctaactgcaagaggaggtgctcagtggatgtttagccgatacgttgaaatttaattacggtttgattgatatttcttgaaaactgccaaagcacatatcatcaaaccatttcatgaatatggtttggaagatgtttagtcttgaatataacgcgaaatagaatatttgtaagtctaaaaaaaaaaaaaaaa

SEQ ID NO: 47 - DNA, 819 bp, Homo sapiens
cctcactaaagggaacaaagatgtgtaactataacggtcctaaggtagcgagtcgaggtcgagctctatttaggtgacactatagaaccaggtctgtaccggtgctgcaacgggagggtcgggcggcgacgctctcactgccgggccagaaggaggcctcccggctccctcacgctagaagggagctggttaccagaggttttcagagacgccaaagttttgccagaatttgttgcaagcacatgaaagattttgtggctgaagtaatgtaaaaccattgatcaaaccggaaattgagacacagccagcgtcacacaacaggccatctgaaaggctggaatagcatcgagggagtctccctggaaccaagcagccaaggcaggctaatggtgaaggcccaggcacagtaatggtcccctggagatggagtcctgctctgtcatccatgctggagtacagtggcgtgatctcggctcgctgcggcctccgcctcctgggttcaagtgattctcctgcctcagcctcccgagtagctgggattgcaagtgtgcaccatcacaccctgctgatttttgtgtttttggtggagacggggtttcaccatgttggccaggctgttcttgaactcctggcctagagcgatccacccgccttggcctcccaaagtcctgggattacaggcgtgagccaccgtacctgacccctgtccctgaacatttgtcacaatatattatgagtgaatgagtaagaaaaaaaaaaaaaaaaggtcgtttggggatcctgccatttcattacctctttctccgcacccgacataga

SEQ ID NO: 48 - DNA, 1354 bp, Homo sapiens
ctcacaattgctctacagctcagaacagcaactgctgaggctgccttgggaagaggatgatcctaaacaaagctctgatgctgggggccctcgccctgaccaccgtgatgagcccttgtggaggtgaagacattgtggctgaccatgttgcctcttacggtgtaaacttgtaccagtcttatggtccctctgggcagtacagccatgaatttgatggagacgaggagttctatgtggacctggagaggaaggagactgtctggcagttgcctctgttccgcagatttagaagatttgacccgcaatttgcactgacaaacatcgctgtgctaaaacataacttgaacatcgtgattaaacgctccaactctaccgctgctaccaatgaggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggatgagcctcttctgaaacactgggagcctgagattccaacacctatgtcagagctcacagagactgtggtctgcgccctggggttgtctgtgggcctcgtgggcattgtggtggggaccgtcttgatcatccgaggcctgcgttcagttggtgcttccagacaccaagggcccttgtgaatcccatcctgaaaaggaaggtgttacctactaagagatgcctggggtaagccgcccagctacctaattcctcagtaacatcgatctaaaatctccatggaagcaataaattccctttaagagatctatgtcaaatttttctatctttcatccggggctgactgaacctatggctaagaattgtgacactctcatgtttcaagccaatttcatctcatttcccagatcatatttcatatccagtaacacagaagcaaccaagtacaatatagcctgataatatgttgatttcttagctgacattaatatttctttcttctttgtgttctcacccttggcactgccgcccatccctcaattcaggcaacaatgaagttaatggatactctctgccctttgctcagaattgttatagcaaaaattttaaaaccaaaaaataagtttgtactaatttcaatatggcttttaaaagtatgatggagaaataaattaggataaaggaactttgaatcacaaaaaaaaaaaaaaaa

SEQ ID NO: 49 - DNA, 927 bp, Homo sapiens
acaggggaagattttcttttttagaggtacagattcctcttagtcaagtcctgattaaaactccagctaagacattagtaagccttggttagtgaagtggcatcaggaagtgcctacattttcatggcctggtagcgttcagtgaaaatgttcattaacagacacaggccattcagtcccgaatcccaagacactgaagactctgtttgaatcagactcatgggttccttcctagccactctcagggacaggaatgcttctggtgaagaagttttcggtggtggttcatggagcttccctacaccaacttggaaatggcattcattttattggcttttgttatcttttccttatttaccctggcttccatctacactactccggatgacagtaatgaagagtctcactctgtcagccaggctggaatgcagtggcgtgatcttggctcactgaaacatcggtctcccaggttcaagcgattctcctgcctcagcctccggagtggctggaactacagaggaagaacatgaaaaaaagggaagggaaaagaaaaggaaaaagtctgaaaagaagaaaaattgctcagaggaagagcacagaattgaagctgttgagctatgatctcatagccaccgatatttctcgctaagaagacagaggaagcaatccatgggaactacttatccacagttaaacaagaggaggggataatgaagaaagttaaaatcacttactgattaaacacgatgataataacctttaatgaactcaatactcgggaaaggcttcacatttctgggactcagcattatccaaaatatctattaagagccatacaccattctagctgcaattgattatacaaaaaaaaaaagaccaaagtggttacaataataaaatagaacacagagaaaaaaaaaaaaaaaa

SEQ ID NO: 50 - DNA, 776 bp, Homo sapiens
  modified_base (122): a, c, g, t, unknown, or other
ctgacagcctgggcgacagagcaagactgtctcaaaaaaaaaaaaaaaaaaaaaaaaaggcagggatatctgagacttaagttcctcttggagagctggagggtcaggagagcgaagctttntatcttgctttgtacctgagatctccttgaatcagggtgggcagggagagagaagggatctgttcagacatttccttttggggtcaaatgagaggaaggagtccttgccccttggagaattactgcaaggatctggttgagttgagaatttgtttcccccaccagtattgtttttggtgtttttttgttttgttttatttgttttgttttgttttaacatctctgtttccttcccctttctgttgttgctccttcctcctcccactccaactaccacacaaatcctgacaggagcctttctgcccctctagggaaggggcccgtgtgaggaactcttactggacgcccccttccctgttgctgtgctgatcttacacatcagtttccacggatcccatgtgaatcagttgtcttcctcatttactctgagcaagggtggcagcagcaatagcagaagacgtagatgcagtgactcattttgcatgatgtctgcaagagagccgggcctcccgtgtgctgtggctctcgttcaggcattgcttcagaaacttgattcttctggaattgtgcataagagggccttttagaaaaaaaaaaaaaaaagcacgtccaaggatcctgccatttcattacctctttctccgcacccgacat

SEQ ID NO: 51 - DNA, 879 bp, Homo sapiens
aaagatgctgcagctggtggggcttacaatctctgagttctggatgctggtgactgcgaagacagacagtggggatcagaagaggcctccccattctccctgggagcccaggaagagtgttgctggattaagcaggagcagcaacatttcaggacttcttgggtggaagaaagttggcagagagaacgtccacaatagagctgctcgagtcagagtcaaacctttttggaggaggggaaatcttggattgagagcgtgcctctagtcaaccatgttgatgacgtcctttctagaaaacaccctgataccttgtgcctgtgcttccaaggtggctgacgctgtgcctgtgcttccgaggtggctgacttctaactgagtgcagtggagaccccgtggtgctttacagaacagatggcagtgctgcccaagctcagaacacagctggacggatccccatttttaaaatgttgcttttaaagttccttggtctattttaagtcttagacaatagagcactaatttgtgtccacagctagggaaaaagcaccccaaatactcatgttatgtcactttcagtaccacaattcaaatagtgaaagacgatgcttccatgtaccacgtataggtctctactagtgatttttctctcatttcaacctagcttcacatatttttatggtatagctaactcaacattgattctatacaaaatatattatctaagatagtctttaaaaatatttgaattccactgggatttgtttaaacccctacacaaataattacctcttactaaaaacactgagtttggcaagtgaaattttgtaataaggtgaagattcaataaaatcttgtctcaaactaaaaaaaaaaaaaaaa

SEQ ID NO: 52 - DNA, 980 bp, Homo sapiens
taaatcacttatgatacccactgatattttggctttcctaactctttatttgccatgcagaacctcgggggatttctctcctaattcaggataaagaccaatagatggcagtgaagataatctgctttaactacagacagcatggattcctggatctcaatactatgatagtcaacatgagtggattttatcttgcttggtttatatttgcttcgcgacctaatctaattttccattttttatacctagtctgtttcatcattataaactaagaataatgatagatgttggtgaagtgtatagagatctggaactgatagagagttaagaaagaactatgttgttgttatccttctaatcattcccttacatagacgcaaaatgcgtgggttactttacatggttcccatgcaactcaaagctaatgaacatgcggtaactaacatgaaagctattccctaaatctgtcaactattttaaacttcaagctctgaccaatcctgagctcagaaagctcaaaaatcaaatattgtgaacttttcaatgcaaatttgtatggtaaagaataaataacactatttcttggttaatatatgtttgtaaattctgcctttcctctttttctatttatctgattattattattattattattattttctttgagacagagtctcactctgttgcccaggcggaggttgcggtgagctaagatcgcgccactgtactccagcctggacaacagcgagactccatctcaaaaaaaaaaggaaatgtgtatcaagaacatgattatccagcggtattttctaattcagatcatcaaactgattatatagaagagttggctttaaaatgtttgcaaatgtctctttttttttaatactggaagaaaaaatattctgttgtgtctcatacagtgcttaggatgtctttcacagagcttattaaaaagatgaaacccaaaaaaaaaaaaaaaa

SEQ ID NO: 53 - DNA, 1232 bp, Homo sapiens
aaagggctgggattacaggcatgagccaccccacctggccagcttgggcaatttactttatgtctttaagcctggtgtgtaatgttgatggtagagtggggaacaattatacctatccaatgagttgtgagtactatatgagttaaatacatgaagcatttagaatagtgccttgcgcataaatatagtaaatacattaagtactatttaagtgctaactgttgttattgttattaatattagatcactatgagtttgcacttgtagcctgtaattcaaagcaatttgaacggcggaagcataaatagataaatgatcatctaaatatggcatctcttcaacccttgttgttgttgttgtttttgccagataagagttttcaaaggttgtgccagagtacttgctttagggatattagcccaattgaaaccatttttttttttttttcattgagacagagtctctctctctgttacccaggcttagagtgcagtgatatgatcatagctcactgcagcctcaatctcccagggtcaagcagtcctcccacctccgcctcccgagtagctgggattagcccagctaattttgtagttttagtagagatggggtttctccatgttggtcagtctggtcttgaactcccgacctcaggtgatctgcccacttcggcctcccaaagtgctgggattacaggcatgagctgccacacccggccggccttagtgtacttctgtgaagtgctgctgttctgtgaaatacccaccttttccatattattttcctgttaaacagattgctctatacacattgcaacttaacggatttaaagatcccacctgagactgacaaaattttgggcttgcatcaggttccccgataatcctctttcttgtgtggtagtggaagttgaaaagtttttgagcctattcagagttgtctggattaaattaggagaagttgaagctagtttaagggaggcgtatgacagtttttctggtttggggcctgtagggtgttgaaagactagaaaggatgacatgactcataggaaatctccccgccgcctccgcttccctcactgggccatgtctccacgagctttctagattagtcacagactcctgttttcttcaggtgcctgattggtgtgtgacgagtgtagtaggtcagttgttagaggtactggtggtgtccagctcaccactggcataggaaaaaaaaaaaaaaaa

SEQ ID NO: 54 - DNA, 716 bp, Homo sapiens
  modified_base (216): a, c, g, t, unknown, or other
gcgcgcaattaaccctcactaaagggaacaaagatgtgtaactataacggtcctaaggtagcgagtcgaggtcgagctctatttaggtgacactatagaaccagaaggagacataagggtggcctttgataagaggtcacttcaaactttcagaactgacaagacgtatagcctccccccaaaaaataatgctatgggtagatggatgcttttttntgaaggaatttttagcatttcatttggaaaagttctgtgatcaaataatgctaaatgttatgacagctttcttggcgtttaaagggattctctgggtgaggggaaggggtgataaaaaaaaaaaaaaaaagtctgcttttggaacanaaatgggggacaataaccaaggntcanaaacccngagtcaaaaaattaaaagaacatcttattttaaaaaaaaagtcaacaacctgcaatgaagtcaccgtacccccataaaatcccaactgtgcatttaaatctttctaccaaaattcacttttggaccatcttatgaagttgtcaaaatttcagaggcaaacgcttaaatcaagatcaaaagccaggaggaaaaaagagctaacagtttccaacccaaactctctccgagccccctaaaaactgatttataaccctgtcatcggtaattttagaagaagcgaacactgacggacgggggcttgggaaaaccaggactccac

SEQ ID NO: 55 - protein, 72 aa, Homo sapiens
MIRPAVPPSLLRLAPTPFCPLTIFCFSPLFIKLLKIMGGHSFGLSSCTSPQQIRPNQNGVTYAKCCVIKLKL

SEQ ID NO: 56 - protein, 54 aa, Homo sapiens [sequence omitted]

SEQ ID NO: 57 - protein, 85 aa, Homo sapiens
MFVKSGWGRSGNVYLLSVLNLLTHFLNLYITLSHKLSLYHQLLPPQATGLFENIPQVFMRACLSPKIRDSYSTKAVFSDSFNSIS

SEQ ID NO: 58 - protein, 43 aa, Homo sapiens
MGISHIGQADLPALASQSAGITSMSHRAKIWFCFVVLFCFVFN

SEQ ID NO: 59 - protein, 53 aa, Homo sapiens [sequence omitted]

SEQ ID NO: 60 - protein, 97 aa, Homo sapiens
MMVRAAETMTTGSEPAFILLLLPPSALRCLQARQSSSSQPTGLPRAGPELRTGIPRARISSASPARGGSQHSSDGSFCSRRLREVLCVSPGASHSLA

SEQ ID NO: 61 - protein, 88 aa, Homo sapiens
MSIYPMLGPILVTQSCMVYAITSDLCVICDLVCISQAQFTGEHLNLKRAVWVRWFMPVIPAPWEAKAGGSRGQEIETILANTVKPRLY

SEQ ID NO: 62 - protein, 83 aa, Homo sapiens
MWYFMSLISMVLLLSPSCSDLLVISVLNLEQRRQSKVGFEPFTSPLCGDGTICHLTGYHKTEHFKNYCCAPKIIFSKCHFTPS

SEQ ID NO: 63 - protein, 48 aa, Homo sapiens
MEEKGTCIQIRKDPEERAPLGGILSLVLLQSTCCFLVLPPPPSFFLVD

SEQ ID NO: 64 - protein, 60 aa, Homo sapiens
MLHIAGLLMCILPLSSFVICVFAFLKVQSLLYPPPACSKLECLAFMFIHYCICHVKFLLP

SEQ ID NO: 65 - protein, 68 aa, Homo sapiens
MKSIFPYMQLYLLPTLFILFRSMTDIILVPVLCGHLTCLLFNSHNFQGTYYFLHIKDDETEARKKKIL

SEQ ID NO: 66 - protein, 49 aa, Homo sapiens [sequence omitted]

SEQ ID NO: 67 - protein, 61 aa, Homo sapiens
MKWLSFTPLNTQLLSVAGLGSPRPSWSRPVASIFGGSNPGRRVTGATVGECGTSWKTPEYS

SEQ ID NO: 68 - protein, 70 aa, Homo sapiens [sequence omitted]

SEQ ID NO: 69 - protein, 80 aa, Homo sapiens
MARTLEPLAKKIFKGVLVAELVGVFGAYFLFSKMHTSQDFRQTMSKKYPFILEVYYKSTEKSGMYGIRELDQKTWLNSKN

SEQ ID NO: 70 - protein, 117 aa, Homo sapiens [sequence omitted]

SEQ ID NO: 71 - protein, 85 aa, Homo sapiens [sequence omitted]

SEQ ID NO: 72 - protein, 42 aa, Homo sapiens
MRMSSFPAAVPGLSPSFMTFSQACSSIICNLLKSEKETAAPW

SEQ ID NO: 73 - protein, 58 aa, Homo sapiens
MCSSPALCLPPCKMCLCASFAFHHDCAASPAMWNDTTLHQCTKPDEKDTDWILVQLAA

SEQ ID NO: 74 - protein, 65 aa, Homo sapiens
MSEFVHFFICLRVIHTGFSMSGLYPLPASFSKFLVFLSILGSSFILGNPSFVLNITEHIFPWCLY

SEQ ID NO: 75 - protein, 68 aa, Homo sapiens
MCCIDSRFKGGLCRMCFVKNVFAGSILVKVIAILHSLLTRDTMHCGSLQGPLPKKAWVLSRFPPTETA

SEQ ID NO: 76 - protein, 213 aa, Homo sapiens
MLWLLFLTLPCLGGLHVQDPRKDTDPSIYRIHAGDVYLYGGRGLLNVSRIIVHPNYVTAGLGADVALLQLSRCRRPTACSRRVCRCWRTPSVSSPTATPQGTLATGSSSWMTCCVPAARAETPATVTPAALWSAGCGGPGAWWGWSAGATAVPCGTFPASTPTSRSTCSGSCSKSGSCPEQAGLGSHLGRLRRDQDLPPPSDLRFGLRCRPPS

SEQ ID NO: 77 - protein, 41 aa, Homo sapiens
MVVPVIPWHGQRWILSVFSVGHLMGVVGTSLQFDLHLSGDQ

SEQ ID NO: 78 - protein, 87 aa, Homo sapiens
MLVPESSSLLQASKAVHSLICGLLTCLSTISQRGRGTRLCGGAESSQQRSAKGSAATGGRQRTALPTDPDVGPGCCLESRQGQAGLP

SEQ ID NO: 79 - protein, 90 aa, Homo sapiens [sequence omitted]

SEQ ID NO: 80 - protein, 52 aa, Homo sapiens
MRSGCLKVCSLSSLFSLFCSAMERCACFRSASHHDCKFLEVSQPCFLYSLWI

SEQ ID NO: 81 - protein, 51 aa, Homo sapiens [sequence omitted]

SEQ ID NO: 82 - protein, 85 aa, Homo sapiens [sequence omitted]

SEQ ID NO: 83 - protein, 83 aa, Homo sapiens [sequence omitted]

SEQ ID NO: 84 - protein, 76 aa, Homo sapiens
MTPGKHPNCLQTLAGLIYNRPYQHLSLSLFFMRIRFSLLHSIALLCHFLQLLSFPWNMALRQLIRVLHCVISSNFY

SEQ ID NO: 85 - protein, 42 aa, Homo sapiens
MFKLFMMYASCFCFFVCFFFSFSATSVPPFRMIFTLCIFQYF

SEQ ID NO: 86 - protein, 55 aa, Homo sapiens [sequence omitted]

SEQ ID NO: 87 - protein, 76 aa, Homo sapiens
MWSFCLALGGAGILFSTAALPSNGTFLVTPPVSLPINVSLPINIATPGSPSFSCNLSLWNSQVCLSRLVAAQEGQQ

SEQ ID NO: 88 - protein, 43 aa, Homo sapiens
MEERVAIIIMRIDFYEYESMSILYLIMQILLLYIWSQSKFALN

SEQ ID NO: 89 - protein, 41 aa, Homo sapiens
MSCKIVLVDIITVFVSRNLLVISVHLHFTKGVNLSKNFTKK

SEQ ID NO: 90 - protein, 42 aa, Homo sapiens
MSHLLVVLLFIALRGSPPRGDTVQQKFTFSFCSHIKQTHPCP

SEQ ID NO: 91 - protein, 255 aa, Homo sapiens
  MOD_RES (224): Variable amino acid
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALXLSVGLVXIVVGTVLIIRGLRSVGASRHQGPL

SEQ ID NO: 92 - protein, 53 aa, Homo sapiens
MSQCSLFYLGLVVVQLLIIEVEGEEKEKRKEKKGKEKKRKEKKRKEKKRKKKK

SEQ ID NO: 93 - protein, 40 aa, Homo sapiens
  MOD_RES (21): Variable amino acid
MILHSPFFVWNIFLIKSVSFXYLFIFFTGEWREPRRRSLQ

SEQ ID NO: 94 - protein, 52 aa, Homo sapiens [sequence omitted]

SEQ ID NO: 95 - protein, 93 aa, Homo sapiens [sequence omitted]

SEQ ID NO: 96 - protein, 59 aa, Homo sapiens
MLDRGRRMSCHCCAVLFVAQALGLIMVSPACHCQSCCFIVMRHKAQTTNSTLAQALLMV

SEQ ID NO: 97 - protein, 114 aa, Homo sapiens
MYCGIQILALWERNIWERNPLGNQSPAIGDFKIFQAFLIIFLTFVSLNTFKSTFEKLLISSYQTRVRVTNPPTSLCWLVASKATVNVYQCEPDPETARKGAKFSLVCEETGGCW

SEQ ID NO: 98 - protein, 57 aa, Homo sapiens
  MOD_RES (18): Variable amino acid
MFITLISCTSDLSLSPLXQLYSFLSWTFFFLFFFFFFCFKTSVMPYQVHVIXSQCTL

SEQ ID NO: 99 - protein, 78 aa, Homo sapiens
MQGPYVGFFKKQTGVYSHVVCTAQPHSTIVNPALSVSLDRFFLFVFFFLVVFCCCCCCFTAETNQQALDDQEAFLSKL

SEQ ID NO: 100 - protein, 71 aa, Homo sapiens
  MOD_RES (14): Variable amino acid
MPAGVPMSTYLKMXAASLLAMCAGAEVVHRYYRPDLTIPEIPPKRGELKTELLGLKERKHKPQVSQQEELK

SEQ ID NO: 101 - protein, 75 aa, Homo sapiens
MVPWRWSPALSSMLEYSGVISARCGLRLLGSSDSPASASRVAGIASVHHHTLLIFVFLVETGFHHVGQAVLELLA

SEQ ID NO: 102 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 103 - protein, 80 aa, Homo sapiens [sequence omitted]

SEQ ID NO: 104 - protein, 66 aa, Homo sapiens
MRGRSPCPLENYCKDLVELRICFPHQYCFWCFFVLFYLFCFVLTSLFPSPFCCCSFLLPLQLPHKS

SEQ ID NO: 105 - protein, 86 aa, Homo sapiens
MLQLVGLTISEFWMLVTAKTDSGDQKRPPHSPWEPRKSVAGLSRSSNISGLLGWKKVGRENVHNRAARVRVKPFWRRGNLGLRACL

SEQ ID NO: 106 - protein, 55 aa, Homo sapiens
MAVKIICFNYRQHGFLDLNTMIVNMSGFYLAWFIFASRPNLIFHFLYLVCFIIIN

SEQ ID NO: 107 - protein, 154 aa, Homo sapiens
MASLQPLLLLLFLPDKSFQRLCQSTCFRDISPIETIFFFFSLRQSLSLCYPGLECSDMIIAHCSLNLPGSSSPPTSASRVAGISPANFVVLVEMGFLHVGQSGLELPTSGDLPTSASQSAGITGMSCHTRPALVYFCEVLLFCEIPTFSILFSC

SEQ ID NO: 108 - protein, 88 aa, Homo sapiens
  MOD_RES (10): Variable amino acid
MLWVDGCFFXKEFLAFHLEKFCDQIMLNVMTAFLAFKGILWVRGRGDKKKKKKSAFGTXMGDNNQGSXTXSQKIKRTSYFKKKVNNLQ

(19) United States
(12) Patent Application Publication
(10) Pub. No.: US 2009/0286954 A1
(43) Pub. Date: Nov. 19, 2009

(54) Human cDNA Clones Comprising Polynucleotides Encoding Polypeptides and Methods of Their Use

(75) Inventors: Lewis T. Williams, Mill Valley, CA (US); Keting Chu, Woodside, CA (US); Ernestine Lee, Kensington, CA (US); Kevin Hestir, Kensington, CA (US); Justin Wong, Oakland, CA (US); Stephen K. Doberstein, San Francisco, CA (US)

Correspondence Address: FivePrime/Finnegan, 901 New York Avenue NW, Washington, DC, 20001-4413, US

(73) Assignee: FIVE PRIME THERAPEUTICS, INC.

(21) Appl. No.: 12/424,373

(22) Filed: Apr. 15, 2009

Related U.S. Application Data
(63) Continuation of application No. 11/983,397, filed on Nov. 7, 2007, now abandoned.
Application No. 11/983,397 is a continuation of application No. 10/948,571, filed on Sep. 24, 2004, now abandoned.
(60) Provisional application No. 60/505,144, filed on Sep. 24, 2003.
(60) Provisional application No. 60/548,191, filed on Mar. 1, 2004.
(60) Provisional application No. 60/589,826, filed on Jul. 22, 2004.
(60) Provisional application No. 60/589,806, filed on Jul. 22, 2004.
(60) Provisional application No. 60/589,788, filed on Jul. 22, 2004.

(51) Int. Cl. C07K 14/00; C07K 14/765; C07K 16/00

(52) U.S. Cl. 530/324; 530/363; 530/387.3; 530/402

(57) ABSTRACT

The invention provides novel human full-length cDNA clones, novel polynucleotides, related polypeptides, related nucleic acid and polypeptide compositions, and related modulators, such as antibodies and small molecule modulators. The invention also provides methods to make and use these cDNA clones, polynucleotides, polypeptides, related compositions, and modulators. These methods include diagnostic, prophylactic and therapeutic applications. The compositions and methods of the invention are useful in treating proliferative disorders, e.g., cancers, and inflammatory, immune, bacterial, and viral disorders.

### CROSS REFERENCE TO RELATED APPLICATIONS

This application claims the benefit of U.S. Provisional Application 60/505,144, filed Sep. 24, 2003, and U.S. Provisional Application 60/548,191, filed Mar. 1, 2004, the disclosures of which are incorporated in their entireties. This application also incorporates U.S. Provisional 60/589,826, filed Apr. 28, 2004; U.S. Provisional (application number pending) “Inhibitory RNA Library,” filed Jul. 22, 2004; and U.S. Provisional 60/589,788, filed Jul. 22, 2004; in their entireties.

### SEQUENCE LISTING

This application contains a Sequence Listing which has been submitted via a printed paper copy, and is hereby incorporated by reference in its entirety. A computer readable version with content identical to the printed paper copy is also submitted herein.

### BACKGROUND OF THE INVENTION

1. Field of the Invention

The invention relates to cDNA clones which encode one or more polypeptide gene products. These cDNA clones encode secreted and/or transmembrane proteins. The invention provides the nucleotide and amino acid sequences of these cDNA clones as well as their tissue sources, expression patterns, an annotative description, and their predicted function. The cDNA clones of the invention are useful for investigative, diagnostic, and therapeutic purposes, as described in detail herein.

2. Background Information

Secreted proteins, also referred to as secreted factors or secreted polypeptides, include polypeptides and active fragments of polypeptides that are produced by cells and exported extracellularly. Secreted proteins also include extracellular fragments of transmembrane proteins that are proteolytically cleaved, and extracellular fragments of cell surface receptors; these fragments may be soluble. Many and widely variant biological functions are mediated by a wide variety of different types of secreted proteins. Yet, despite the sequencing of the human genome, relatively few pharmaceutically useful secreted proteins have been identified and brought to the clinic or to the market. It would be advantageous to discover novel secreted proteins or polypeptides, and their corresponding polynucleotides, which have medical utility.

Pharmaceutically useful secreted proteins of the present invention will have in common the ability to act as ligands for binding to receptors on cell surfaces in ligand/receptor interactions; to bind to ligands, soluble or otherwise; to inhibit ligand/receptor interactions; to trigger certain intracellular responses, such as inducing signal transduction to activate cells or inhibit cellular activity; to induce cellular growth, proliferation, or differentiation; to induce the production of other factors that, in turn, mediate such activities; and/or to inhibit cell activation or other cell signaling activities. The cell types having cell surface receptors responsive to secreted proteins are many and various, including, any cell type of any tissue origin or developmental state, and including normal cells and cells implicated in pathological conditions or other disorders.

Transmembrane proteins extend into or through the cell membrane's lipid bilayer; they can span the membrane once, or more than once. Transmembrane proteins that span the membrane once are designated “single transmembrane proteins” (STM), and transmembrane proteins that span the membrane more than once are designated “multiple transmembrane proteins” (MTM). A single transmembrane protein typically has one transmembrane (TM) domain, spanning a series of consecutive amino acid residues, numbered on the basis of distance from the N-terminus, with the first amino acid residue at the N-terminus as number 1. A multi-transmembrane protein typically has more than one TM domain, each spanning a series of consecutive amino acid residues, numbered in the same way as the STM protein.

Transmembrane proteins, having part of their molecules on either side of the bilayers, also have many and widely variant biological functions. They transport molecules, e.g., ions or proteins, across membranes, transduce signals across membranes, act as receptors, and function as antigens. Transmembrane proteins are often involved in cell signaling events; they can comprise signaling molecules, and/or can interact with signaling molecules.

Transmembrane proteins with extracellular fragments that can be cleaved may act as secreted proteins and bind to receptors as ligands. Transmembrane proteins embedded in the membrane may act as receptors, and may possess both a ligand-binding extracellular portion exposed on a cell surface and an intracellular portion that interacts with other cellular components upon activation. Both secreted and embedded transmembrane proteins can mediate intracellular responses and extracellular responses.

### SUMMARY OF THE INVENTION

The present invention relates generally to novel nucleic acids embodied in cDNA clones and the polypeptides they encode. Sequences encompassed by the invention include, but are not limited to, the polypeptide and polynucleotide sequences of the molecules shown in the Sequence Listing and corresponding molecular sequences found at all developmental stages of an organism, genes or gene segments designated by the Sequence Listing, and their corresponding gene products, i.e., RNA and polypeptides. Sequences encompassed by the invention also include variants of those presented in the Sequence Listing which are present in the normal physiological state, e.g., variant alleles such as SNPs, splice variants, as well as variants that are present in pathological states, such as disease-related mutations or sequences with alterations that lead to pathology. Variants of the invention include polypeptides with conservative amino acid changes; as well as complements and fragments, for example, signal peptides, mature polypeptides, biologically active fragments, Pfam domains, and structural motifs. The invention also includes vectors and host cells that can be used to produce the polypeptides of the invention and gene products of the polynucleotides of the invention, as well as methods of using these vectors and host cells to produce gene products. The invention includes antibodies that specifically bind to the molecules of the invention.

The novel amino acid molecules of the invention are secreted and/or transmembrane proteins. They can function as agonists, antagonists, ligands, and/or receptors, and they can have diagnostic, prophylactic, and therapeutic effects. The invention provides methods of making the polynucleotides and polypeptides of the invention, as well as methods of determining their presence. The invention provides diagnostic kits and methods of using the novel nucleic acids and amino acids to diagnose disease. It also provides methods of using the polynucleotides and polypeptides of the invention to modulate biological activity; this modulation finds uses in disease prophylaxis and therapy, as well as in identification of agents useful in disease prophylaxis and therapy.

### DETAILED DESCRIPTION OF THE INVENTION

### Definitions

The terms “nucleic acid molecule,” “polynucleotide,” and “nucleic acid” are used interchangeably herein to refer to polymeric forms of nucleotides of any length. The nucleic acid molecules can contain deoxyribonucleotides, ribonucleotides, and/or their analogs. Nucleotides can have any three-dimensional structure, and can perform any function, known or unknown. The terms include single-stranded, double-stranded, and triple helical molecules. “Oligonucleotide” may generally refer to polynucleotides of between about 5 and about 100 nucleotides of single- or double-stranded DNA or RNA. For the purposes of this disclosure, the lower limit of the size of an oligonucleotide is two, and there is no upper limit to the length of an oligonucleotide. Oligonucleotides are also known as oligomers or oligos and can be isolated from genes, or chemically synthesized by methods known in the art.

A “complement” of a nucleic acid molecule is a one that is comprised of its complementary base pairs. Deoxyribonucleotides with the base adenine are complementary to those with the base thymidine, and deoxyribonucleotides with the base thymidine are complementary to those with the base adenine. Deoxyribonucleotides with the base cytosine are complementary to those with the base guanine, and deoxyribonucleotides with the base guanine are complementary to those with the base cytosine. Ribonucleotides with the base adenine are complementary to those with the base uracil, and deoxyribonucleotides with the base uracil are complementary to those with the base adenine. Ribonucleotides with the base cytosine are complementary to those with the base guanine, and deoxyribonucleotides with the base guanine are complementary to those with the base cytosine.

A “nucleic acid hybridization reaction” is one in which single strands of DNA or RNA randomly collide with one another, and bind to each other only when their nucleotide sequences have some degree of complementarity. The solvent and temperature conditions can be varied in the reactions to modulate the extent to which the molecules can bind to one another. Hybridization reactions can be performed under different conditions of “stringency.” The “stringency” of a hybridization reaction as used herein refers to the conditions (e.g., solvent and temperature conditions) under which two nucleic acid strands will either pair or fail to pair to form a “hybrid” helix.

A “polymerase chain reaction” is a chemical reaction capable of amplifying DNA in vitro. It is performed using two oligonucleotide primers, which are complementary to two regions of the target DNA to be amplified, one for each strand. The primers are added to the target DNA in the presence of excess deoxynucleotides and a heat stable DNA polymerase. The target DNA can be provided to the reaction mixture in pure or relatively pure form, or it may be present as a minor component, as is typically the case when it is provided as a component of a biological sample. In a series of temperature cycles, the target DNA is repeatedly denatured at high temperature, annealed to the primer at a lower temperature, and a daughter strand extended from the primer at an intermediate temperature. As the daughter strands act as templates in subsequent temperature cycles, DNA fragments matching both primers are amplified exponentially.

A “primer” is a polynucleotide chain to which deoxyribonucleotides can be added by DNA polymerase.

A “promoter” is a nucleotide sequence present in DNA, to which RNA polymerase binds to begin transcription. The term includes a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

Heterologous promoters are derived from different genetic sources. They encompass promoters of different species, e.g., a rat promoter is heterologous to a human promoter of the corresponding gene. The term also includes promoters found in different cell or tissue types of a specimen of the same species, e.g., a promoter active in the transcription of a protein in human brain may be heterologous to a promoter active in the transcription of the same protein in human muscle. Heterologous promoters can be natural or artificial, and comprised of different elements. A promoter that “naturally regulates” is one that regulates in nature and without artificial aid. The term can include heterologous and homologous promoters. A “tissue specific promoter” is one that initiates transcription exclusively or selectively in one or a few tissue types.

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain proteins as well as multimers.

Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

A “signal peptide,” “leader sequence,” or a “signal sequence” comprises a sequence of amino acid residues, typically, at the amino terminus of a polypeptide, which directs the intracellular trafficking of polypeptides that are destined to be either secreted or membrane components. Signal peptides are generally hydrophobic and have some positively charged residues. Polypeptides that contain a signal peptides typically also contain a signal peptide cleavage site, which can be acted upon by a signal peptidase. Signal peptides can be natural or synthetic, heterologous, or homologous with the protein to which they are attached.

A “mature polypeptide” is a polypeptide that has been acted upon by a signal peptidase, for example, after secretion from the cell, or after being directed to an appropriate intracellular compartment.

An “isolated,” “purified,” or “substantially isolated” polynucleotide or polypeptide, or a polynucleotide or polypeptide in “substantially pure form,” in “substantially purified form,” in “substantial purity,” or as an “isolate,” is one that is substantially free of the sequences with which it is associated in nature, or other nucleic acid sequences that do not include a sequence or fragment of the subject polynucleotides.

By substantially free is meant that less than about 90%, less than about 80%, less than about 70%, less than about 60%, or less than about 50% of the composition is made up of materials other than the isolated polynucleotide or polypeptide. For example, the isolated polynucleotide is at least about 50%, at least about 60%, at least about 70%, at least about 80%, at least about 90%, at least about 95%, at least about 97%, or at least about 99% free of the materials with which it is associated in nature. For example, an isolated polynucleotide may be present in a composition wherein at least about 50%, at least about 60%, at least about 70%, at least about 80%, at least about 90%, at least about 95%, at least about 97%, at least about 99% of the total macromolecules (for example, polypeptides, fragments thereof, polynucleotides, fragments thereof, lipids, polysaccharides, and oligosaccharides) in the composition is the isolated polynucleotide. Where at least about 99% of the total macromolecules is the isolated polynucleotide, the polynucleotide is at least about 99% pure, and the composition comprises less than about 1% contaminant.

As used herein, an “isolated,” “purified,” or “substantially isolated” polynucleotide or polypeptide, or a polynucleotide or polypeptide in “substantially pure form,” in “substantially purified form,” in “substantial purity,” or as an “isolate,” also refers to recombinant polynucleotides and polypeptides, modified, degenerate and homologous polynucleotides and polypeptides, and chemically synthesized polynucleotides and polypeptides, which, by virtue of origin or manipulation, are not associated with all or a portion of a polynucleotide or polypeptide with which it is associated in nature, are linked to a polynucleotide or polypeptide other than that to which it is linked in nature, or do not occur in nature. For example, the subject polynucleotides are generally provided as other than on an intact chromosome, and recombinant embodiments are typically flanked by one or more nucleotides not normally associated with the subject polynucleotide on a naturally-occurring chromosome.

A “biologically active” entity, or an entity having “biological activity,” is one having structural, regulatory, or biochemical functions of a naturally occurring molecule or any function related to or associated with a metabolic or physiological process. For example, an entity demonstrates biological activity when it participates in a molecular interaction with another molecule, when it has therapeutic value in alleviating a disease condition, or when it has prophylactic value in inducing an immune response to the molecule. Biologically active polynucleotide fragments are those exhibiting activity similar, but not necessarily identical, to an activity of a polynucleotide of the present invention. The biological activity can include an improved desired activity, or a decreased undesirable activity. Biologically active polypeptide fragments are those exhibiting activity similar, but not necessarily identical, to an activity of a polypeptide of the present invention.

A “vector” is a plasmid that can be used to transfer DNA sequences from one organism to another. An “expression vector” is a cloning vector that contains regulatory sequences that allow transcription and translation of a cloned gene or genes and thus transcribe and clone DNA. Expression vectors can be used to express the polypeptides of the invention and typically include restriction sites to provide for the insertion of nucleic acid sequences encoding heterologous protein or RNA molecules. Artificially constructed plasmids, i.e., small, independently replicating pieces of extrachromosomal cytoplasmic DNA that can be transferred from one organism to another, are commonly used as cloning vectors.

The term “host cell” includes an individual cell, cell line, cell culture, or in vivo cell, which can be or has been a recipient of any polynucleotides or polypeptides of the invention, for example, a recombinant vector, an isolated polynucleotide, antibody, or fusion protein. Host cells include progeny of a single host cell, and the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, insect, amphibian, reptile, crustacean, avian, fish, plant and fungal cells. A host cell includes cells transformed, transfected, transduced, or infected in vivo or in vitro with a polynucleotide of the invention, for example, a recombinant vector. A host cell which comprises a recombinant vector of the invention may be called a “recombinant host cell.”

A “bacteriophage” is a virus with a specific affinity for one or more type of bacteria, and which infect these bacteria. Bacteriophages generally comprise a capsid or protein coat which encloses the genetic material, i.e., the DNA or RNA that enters the bacterium when a bacteriophage infects a bacterium.

“Transformation” herein is used to refer to a process by which the genetic material carried by an individual cell is altered by incorporation of exogenous DNA into its genome. “Transfection” herein means the introduction of a nucleic acid into a recipient cell and the subsequent integration into the chromosomal DNA of the recipient cells. “Transduction” is the transfer of genetic information from one cell to another via a vector.

The term “antibody” refers to protein generated by the immune system that is capable of recognizing and binding to a specific antigen; antibodies are commonly known in the art. An “epitope” is the site of an antigenic molecule to which an antibody binds.

To “proliferate” herein means to increase in number via the growth and reproduction of similar cells.

The term “responder cell” refers to any cell that exhibits a change in any biological activity, including a genetic or phenotypic event, such as a physiological, morphological, or immunogenic change, or a change in the expression of a reporter gene, where the change can be assayed, measured, monitored, tested, observed, or otherwise detected.

“Expression” of a nucleic acid molecule refers to the conversion of the information into a gene product. A gene product can be the direct transcriptional product of a gene (e.g., mRNA, tRNA, rRNA, antisense RNA, ribozyme, structural RNA, or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “modulate” encompasses an increase or a decrease, a stimulation, inhibition, or blockage in the measured activity when compared to a suitable control. “Modulation” of expression levels includes increasing the level and decreasing the level of a mRNA or polypeptide of interest encoded by a polynucleotide of the invention when compared to a control lacking the agent being tested. In some embodiments, agents of particular interest are those which inhibit a biological activity of a subject polypeptide, and/or which reduce a level of a subject polypeptide in a cell, and/or which reduce a level of a subject mRNA in a cell and/or which reduce the release of a subject polypeptide from a eukaryotic cell. In other embodiments, agents of interest are those that increase polypeptide activity.

Modulation can be effected by a modulator, i.e., a substance that binds to and/or modulates a level or activity of a polypeptide or a level of mRNA encoding a polypeptide or nucleic acid, or that modulates the activity of a cell containing a polypeptide or nucleic acid. Where the agent modulates a level of mRNA encoding a polypeptide, agents include ribozymes, antisense, and RNAi molecules. Where the agent is a substance that modulates a level of activity of a polypeptide, agents include antibodies specific for the polypeptide, peptide aptamers, small molecule drugs, agents that bind a ligand-binding site in a subject polypeptide, natural ligands, soluble receptors, agonists, antagonists, and the like. Antibody agents include antibodies that specifically bind a subject polypeptide and activate the polypeptide, such as receptor-ligand binding that initiates signal transduction; antibodies that specifically bind a subject polypeptide and inhibit binding of another molecule to the polypeptide, thus preventing activation of a signal transduction pathway; antibodies that bind a subject polypeptide to modulate transcription; antibodies that bind a subject polypeptide to modulate translation; as well as antibodies that bind a subject polypeptide on the surface of a cell to initiate antibody-dependent cytotoxicity (ADCC) or to initiate cell killing or cell growth. Small molecule drug modulators include those that bind the polypeptide to modulate activity of the polypeptide or cell containing the polypeptide in a similar fashion. Small molecule drug modulators also include those that bind the polypeptide to modulate activity of the polypeptide or a cell containing the polypeptide.

The term “agonist” refers to a substance that mimics the function of an active molecule. Agonists include, but are not limited to, small molecules, drugs such as small molecule compounds, hormones, antibodies, and neurotransmitters, as well as analogues and fragments thereof.

The term “antagonist” refers to a molecule that competes for the binding sites on a molecule with an agonist, but does not induce an active response. Antagonists include, but are not limited to, small molecules, drugs such as small molecule compounds, hormones, antibodies, and neurotransmitters, antisense molecules, RNAi, soluble receptors, as well as analogues and fragments thereof.

A “ligand” is any molecule that binds to a specific site on another molecule.

A “receptor” is a polypeptide that binds to a specific extracellular molecule and initiates a cellular response. A receptor can be part of a cell membrane, or it can be soluble; it can be on the cell surface or inside the cell. Soluble receptors include extracellular fragments of transmembrane cell surface receptors that have been proteolytically cleaved, as well as luminal fragments of receptors that have been proteolytically cleaved.

“Overexpressed” refers to a state wherein there exists any measurable increase over normal or baseline levels. For example, a molecule that is over-expressed in a disorder is one that is manifest in a measurably higher level compared to levels in the absence of the disorder.

“Diagnosis” is the identification of a disease by the detection of a property of a biological sample. Detection methods of the invention can be qualitative or quantitative. Thus, as used herein, the terms “detection,” “determination,” and the like, refer to both qualitative and quantitative determinations, and include measuring.

The terms “patient,” “subject,” and “individual,” used interchangeably herein, refer to a mammal, including, but not limited to, humans, murines, simians, felines, canines, equines, bovines, porcines, ovines, caprines, avians, mammalian farm animals, mammalian sport animals, and mammalian pets.

A “disease” is a pathological, abnormal, and/or harmful condition of an organism. The term includes conditions, syndromes, and disorders.

“Treatment,” as used herein, covers any administration or application of remedies for disease in an animal, including a human, and includes inhibiting the disease, i.e., arresting its development, or relieving the disease, i.e., causing its regression; or restoring or repairing a lost, missing, or defective function; or stimulating an inefficient process.

“Prophylaxis,” as used herein includes preventing a disease from occurring or recurring in a subject that may be predisposed to the disease but has not yet been diagnosed as having it. Treatment and prophylaxis can be administered to an organism, or to a cell in vivo, in vitro, or ex vivo, and the cell subsequently administered to the subject.

A “pharmaceutically acceptable carrier” refers to a non-toxic solid, semisolid or liquid filler, diluent, encapsulating material, or formulation auxiliary of any conventional type. A pharmaceutically acceptable carrier is non-toxic to recipients at the dosages and concentrations employed and is compatible with other ingredients of the formulation. For example, the carrier for a formulation containing polypeptides does not include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers, such as Azone, can also be included.

A “buffer” is a system that tends to resist change in pH when a given increment of hydrogen ion or hydroxide ion is added. At pH values outside the buffer zone there is less capacity to resist changes in pH. The buffering power is maximal at the pH where the concentration of the proton donor (acid) equals that of the proton acceptor (base). Buffered solutions contain conjugate acid-base pairs. A buffered solution will demonstrate a lesser change in pH than an unbuffered solution in response to addition of an acid or base. Any conventional buffer can be used with the compositions herein including but not limited to, for example, Tris, phosphate, imidazole, and bicarbonate.

A “vaccine” is a preparation of killed microorganisms, living attenuated organisms, or living virulent organisms that is administered to produce or artificially increase immunity to a particular disease. It includes a preparation containing weakened or dead microbes of the kind that cause a particular disease, administered to stimulate the immune system to produce antibodies against that disease.

### BRIEF DESCRIPTION OF THE TABLES

Table 1 provides identification of the novel human cDNA clones of the invention. Each of the sequences of the Sequence Listing is identified by an internal reference number (FP ID). Table 1 correlates this reference number with each of the sequences of the invention. Each sequence is identified by its FP ID number, a SEQ ID NO. corresponding to the nucleotide coding sequence (SEQ ID NO. (N1)), a SEQ ID NO. corresponding to the encoded polypeptide sequence (SEQ ID NO. (P1)), and a Source ID designation for the source of each novel human cDNA clone.

Table 2 lists the FP ID and the Source ID of each clone of the invention and specifies the predicted length of each protein (Predicted Protein Length), expressed as the predicted number of amino acid residues. Table 2 also specifies the result of an algorithm that predicts whether the claimed sequence is secreted (Tree Vote). This algorithm is constructed on the basis of a number of attributes including hydrophobicity, two-dimensional structure, prediction of signal sequence cleavage site, and other parameters. Based on such an algorithm, a sequence that has a secreted tree vote of approximately 0.5 is believed to be a secreted protein. Table 2 sets forth the coordinate positions of the amino acid residues comprising the signal peptide sequences (Signal Peptide Coords.) of proteins that include signal peptide sequences. Table 2 also sets forth the coordinate positions of the amino acid residues comprising the mature protein sequences (Mature Protein Coords.) of the cDNA clones of the invention following cleavage of the signal peptide. Table 2 lists alternative coordinates of the amino acid residues of the signal peptide and the mature polypeptide (Altern. Signal Peptide Coords.) (Altern. Mature Protein Coords.). In instances where the mature protein start residue overlaps the signal peptide end residue, some of the amino acid residues may be cleaved off, such that the mature protein does not start at the next amino acid residue from the signal peptides, resulting in the alternative mature protein coordinates. Table 2 also specifies the number, if any, of the transmembrane domains of each claimed sequence (TM), and the position(s), if any, of the amino acid residues comprising the transmembrane domains of each claimed sequence (TM Coords.). Finally, Table 2 shows the coordinate positions of the amino acid residues that do not comprise transmembrane regions. The coordinates shown in the Tables 2 are listed in terms of the amino acid residues beginning with “1” at the N-terminus of the polypeptide.

Table 3 designates the sequences in the public domain with the greatest similarity to the novel cDNA clones of the invention. The nucleotide sequences of the invention shown in Table 3 are identified by the FP ID and Source ID that relate to the corresponding cDNA clone. Table 3 specifies the predicted length (Predicted Protein Length) of the corresponding cDNA clone, expressed as the predicted number of amino acid residues. Table 3 also describes the characteristics of the sequence in the public National Center for Information Biotechnology (NCBI) database that displays the greatest degree of similarity to each claimed sequence. This sequence is described by its NCBI accession number (Top Hit Accession ID), the NCBI's annotation of that sequence (Top Hit Annotation), and the length of the polypeptide predicted to be encoded by the top hit (Top Hit Length). The predicted identity between the polypeptide sequence of the designated Source ID and the NCBI protein with the greatest similarity is indicated with respect to the entire length of the query (% ID Over Query Length) and with respect to the length of the hit (% ID Over Hit Length).

Table 4 is similar to Table 3, and designates the human sequences in the public domain with the greatest similarity to the sequences of the invention. The nucleotide sequences of the invention shown in Table 4 are identified by the FP ID and Source ID that relate to the corresponding cDNA clone. Table 4 specifies the predicted length (Predicted Protein Length) of the corresponding cDNA clone, expressed as the predicted number of amino acid residues. Table 4 also describes the characteristics of the human sequence in the public NCBI database that displays the greatest degree of similarity to each claimed sequence. This sequence is described by its NCBI accession number (Top Human Hit Accession ID), the NCBI's annotation of that sequence (Top Human Hit Annotation), and the length of the polypeptide predicted to be encoded by the top human hit (Top Human Hit Length). The predicted identity between the polypeptide sequence of the designated Source ID and the NCBI human protein with the greatest similarity is indicated with respect to the entire length of the query (% ID Over Query Length) and with respect to the length of the hit (% ID Over Hit Length).

Table 5 lists the Pfam domains, with their coordinate positions, present in the two clones with FP ID numbers HG1012993P1 and HG1013025P1. These two clones both comprise an MHC_II_alpha domain at position 29-110 and an ig domain at position 126-191.

Table 6 describes the three dimensional structural motifs of the three clones with FP ID numbers HG1012887P1, HG1012993P1, and HG1013025P1. Table 6 specifies the predicted length of each protein (Predicted Protein Length), expressed as the predicted number of amino acid residues. Table 6 also specifies the Tree Vote, which indicates that HG102887P1 is secreted, and HG1012993P1 are not secreted. These three clones possess signal peptides; Table 6 specifies the coordinates of the signal peptides (Signal Peptide Coords.) and the mature protein coordinates (Mature Protein Coords.). Table 6 also specifies that HG1012993P1 and HG10103025P1 are single transmembrane proteins (TM) and specifies the coordinates of their respective transmembrane regions (TM Coords.).

Table 7 identifies the tissue sources of the novel human cDNA clones. Their nucleotide sequences are identified by the FP ID and Source ID that relate to the corresponding cDNA clone. Table 7 also specifies the library, the library ID, and the tissue source (Tissue) of some of the novel cDNA clones of the invention. Some of these polypeptides are differentially expressed among different cell and tissue types, and are more highly expressed in the tissues designated in Table 7 as the source of the clone.

Table 8 predicts the function and tissue localization of selected novel cDNA clones of the invention. The FP ID and the Source ID of these clones are listed, along with their classification as secreted (SEC) or single transmembrane (STM proteins).

Table 9 predicts the tissue localization of selected novel cDNA clones of the invention. The FP ID and the Source ID of these clones are listed, along with their classification as secreted (SEC), single transmembrane (STM), or multiple transmembrane (MTM proteins).

Nucleic Acid and Polypeptide Compositions

Nucleic Acids

The present invention provides novel cDNA molecules, novel genes encoding proteins, the encoded proteins, and fragments, complements, and homologs thereof. Specifically, it provides a first nucleic acid molecule comprising a polynucleotide sequence chosen from at least one polynucleotide sequence according to SEQ ID NOS.:1-54, a complement thereof, and at least one polynucleotide sequence that encodes SEQ ID NOS.:55-108. This nucleic acid molecule can be either a DNA or an RNA molecule.

Non-limiting embodiments of nucleic acid molecules include genes or gene fragments, exons, introns, mRNA, tRNA, rRNA, siRNA, ribozymes, antisense cDNA, recombinant polynucleotides, branched polynucleotides, plasmids, vectors, isolated DNA of any sequence, isolated RNA of any sequence, nucleic acid probes, and primers. Nucleic acid molecules include splice variants of an mRNA. Nucleic acids can be naturally occurring, e.g. DNA or RNA, or can be synthetic analogs, as known in the art. Such analogs are suitable as probes because they demonstrate stability under assay conditions. A nucleic acid molecule can also comprise modified nucleic acid molecules, such as methylated nucleic acid molecules and nucleic acid molecule analogs. Analogs of purines and pyrimidines are known in the art.

Nucleic acid compositions can comprise a sequence of DNA or RNA, including one having an open reading frame that encodes a polypeptide and is capable, under appropriate conditions, of being expressed as a polypeptide. The nucleic acid compositions also can comprise fragments of DNA or RNA. The term encompasses genomic DNA, cDNA, mRNA, splice variants, antisense RNA, RNAi, siRNA, DNA comprising one or more single-nucleotide polymorphisms (SNP), and vectors comprising nucleic acid sequences of interest.

The invention also provides an isolated double-stranded nucleic acid molecule comprising a first nucleic acid molecule with one or more of the polynucleotide sequences SEQ ID NOS.:1-54, its complement, and/or a polynucleotide sequence that encodes SEQ ID NOS.:55-108; or a complement of the first nucleic acid molecule. The first polynucleotide sequence of this double stranded nucleic acid molecule may encode a biologically active fragment of a polypeptide, a signal peptide, a mature polypeptide that lacks a signal peptide, a polypeptide that lacks a signal peptide cleavage site, a polypeptide consisting essentially of a Pfam domain, and/or a polypeptide consisting essentially of a structural motif.

The invention also provides a second nucleic acid molecule comprising a second polynucleotide sequence that is at least about 70%, or about 80%, or about 90%, or about 93%, or about 95% homologous to a first nucleic acid molecule, which comprises one or more of the polynucleotide sequences SEQ ID NOS.:1-54, its complement, and/or a polynucleotide sequence that encodes SEQ ID NOS.:55-108. This second isolated nucleic acid molecule can also comprise a second polynucleotide sequence that hybridizes under high stringency conditions to a first nucleic acid molecule with one or more of the polynucleotide sequences SEQ ID NOS.:1-54, its complement, and/or a polynucleotide sequence that encodes SEQ ID NOS.:55-108. In an embodiment, the sequence of this second isolated nucleic acid is complementary to the first polynucleotide sequence. In an embodiment, a polynucleotide of the invention hybridizes under stringent hybridization conditions to a polynucleotide having the coding region of one or more of the sequences SEQ ID NOS.:1-54, or complement thereof.

The novel cDNA clones of the invention were derived from total RNA isolated from normal or diseased tissues and from normal or treated cells, e.g., stimulated peripheral blood mononuclear cells (PBMC), as shown in Table 7. These RNA samples were transcribed into cDNA using technology described by RIKEN and others, including methods of capturing the 5′ ends of DNA (“CAP trapping”) and methods to eliminate secondary structure in the mRNA using trehalose so that the entire molecule can be reverse transcribed (WO 02/28876; WO 02/070720; U.S. Pat. No. 6,627,399; U.S. Pat. No. 6,458,756; U.S. Pat. No. 6,372,437; U.S. Pat. No. 6,365,350; U.S. Pat. No. 3,344,345; U.S. Pat. No. 6,342,387, U.S. Pat. No. 6,333,156; U.S. Pat. No. 6,294,337; U.S. Pat. No. 6,265,569; U.S. Pat. No. 6,221,599; U.S. Pat. No. 6,174,669; U.S. Pat. No. 6,143,528; U.S. Pat. No. 6,074,824; and U.S. Pat. No. 6,013,488).

Libraries of the transcribed cDNA were compiled, and samples of approximately three 384-well plates from each library were sequenced at their 5′ end. Using the diversity of the library as represented by the sample as the criteria, the 5′ ends of as many as 10,000 clones from each library were sequenced. This 5′ end sequence information was the basis of an analysis that provided a clustered organization of the clones. The clusters were based on a map of the human genome including all known human genes and all known human expressed sequence tags. Multiple sequences mapping to the same locus were identified as belonging to one cluster. A cluster may include splice variants. Clones mapping to a locus comprising no previously identified genes are identified herein. These cDNA clones represent novel genes belonging to novel gene clusters. Further, samples of some of the members of the transcribed cDNA libraries were compiled, and sequenced at their 3′ end, as well as their 5′ end. A subset of these possessed contiguous 5′ end sequence and 3′ end sequence. These were assembled into full length sequences, and are identified herein as the novel cDNA clones of the Sequence Listing, and described herein.

In some embodiments, a polynucleotide of the invention comprises a nucleotide sequence of at least about 5, at least about 8, at least about 10, at least about 15, at least about 18, at least about 20, at least about 25, at least about 30, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1100, at least about 1200, at least about 1300, at least about 1400, at least about 1500, at least about 1600, or at least about 1700 contiguous nucleotides of any one of the sequences shown in SEQ ID NOS.:1-54, or the coding region thereof, or a complement thereof.

In some embodiments, a polynucleotide of the invention comprises a nucleotide sequence that encodes a polypeptide comprising an amino acid sequence of at least about 5, at least about 8, at least about 10, at least about 15, at least about 18, at least about 20, at least about 25, at least about 30, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, or at least about 800 contiguous amino acids of at least one of the sequences shown in SEQ ID NOS.:1-54 (e.g., a polypeptide encoded by at least one of the nucleotide sequences shown in SEQ ID NOS.:1-54), up to and including an entire amino acid sequence as shown in SEQ ID NOS.:55-108 (or as encoded by at least one of the nucleotide sequences shown in SEQ ID NOS.:1-54).

In an embodiment, the present invention includes a polynucleotide selected from SEQ ID NOS.:1-54, which contains approximately 300 bp of the region of the 5′ terminus of a polynucleotide sequence encoding a protein. Such a polynucleotide is useful for the purposes of clustering gene sequences to determine a gene family.

The nucleic acids of the subject invention can encode all or a part of the subject proteins. Double or single stranded fragments can be obtained from the DNA sequence by chemically synthesizing oligonucleotides in accordance with conventional methods, for example by restriction enzyme digestion or polymerase chain reaction (PCR) amplification. The use of the polymerase chain reaction has been described (Saiki et al., 1985) and current techniques have been reviewed (Sambrook et al., 1989, McPherson et al. 2000; Dieffenbach and Dveksler, 1995). For the most part, DNA fragments will be of at least about 5 nucleotides, at least about 8 nucleotides, at least about 10 nucleotides, at least about 15 nucleotides, at least about 18 nucleotides, at least about 20 nucleotides, at least about 25 nucleotides, at least about 30 nucleotides, or at least about 50 nucleotides, at least about 75 nucleotides, or at least about 100 nucleotides. Nucleic acid compositions that encode at least six contiguous amino acids (i.e., fragments of 18 nucleotides or more), for example, nucleic acid compositions encoding at least 8 contiguous amino acids (i.e., fragments of 24 nucleotides or more), are useful in directing the expression or the synthesis of peptides that can be used as immunogens (Lerner, 1982; Shinnick et al., 1983; Sutcliffe et al., 1983).

The nucleic acids of the invention include degenerate variants that can be translated, according to the standard genetic code, to provide an amino acid sequence identical to that translated from the nucleic acid sequences herein. For example, synonymous codons include GGG, GGA, GGC, and GGU, each encoding glycine. The nucleic acids of the invention also include those that encode variants of the polypeptide sequences encoded by the polynucleotides of the Sequence Listing. In some embodiments, these polynucleotides encode variant polypeptides that include insertions, additions, deletions, or substitutions, e.g., conservative amino acid substitutions, compared with the polypeptides encoded by the nucleotide sequences shown in SEQ ID NOS.:1-54, or in the Tables. Conservative amino acid substitutions include serine/threonine, valine/leucine/isoleucine, asparagine/histidine/glutamine, glutamic acid/aspartic acid, etc. (Gonnet et al., 1992).

The nucleic acids of the invention further include allelic variants. They include single nucleotide polymorphisms (SNPs), which occur frequently in eukaryotic genomes (Lander, et al. 2001). The nucleotide sequence determined from one individual of a species can differ from other allelic forms present within the population. Nucleic acids of the invention include those found in disease and/or pathological variants, as described in greater detail herein.

The nucleic acids of the invention include homologs of the polynucleotides. The source of homologous genes can be any species, e.g., primate species, particularly human; rodents, such as rats, hamsters, guinea pigs, and mice; lapines; canines; felines; cattles, such as bovines, goats, pigs, sheep, and equines; crustaceans; avians, such as chickens; reptiles; amphibians; fish; insects; plants; fungi; yeast; nematodes, etc. Among mammalian species, e.g., human and mouse, homologs can have substantial sequence similarity, e.g., at least about 60% sequence identity, at least about 75% sequence identity, or at least about 80% sequence identity among nucleotide sequences. In many embodiments of interest, homology will be at least about 75%, at least about 80%, at least about 85%, at least about 90%, at least about 93%, at least about 95%, at least about 97%, or at least about 98%; in certain embodiments of interest the homology will be as high as about 99%.

Nucleic acid molecules of the invention can comprise heterologous nucleic acid sequences, i.e., nucleic acid sequences of any length other than those specified in the Sequence Listing. For example, the subject nucleic acid molecules can be flanked on the 5′ and/or 3′ ends by heterologous nucleic acid molecules of from about 1 nucleotide to about 10 nucleotides, from about 10 nucleotides to about 20 nucleotides, from about 20 nucleotides to about 50 nucleotides, from about 50 nucleotides to about 100 nucleotides, from about 100 nucleotides to about 250 nucleotides, from about 250 nucleotides to about 500 nucleotides, or from about 500 nucleotides to about 1000 nucleotides, or more in length.

Heterologous sequences of the invention can comprise nucleotides present between the initiation codon and the stop codon, including some or all of the introns that are normally present in a native chromosome. They can further include the 3′ and 5′ untranslated regions found in the mature mRNA. They can further include specific transcriptional and translational regulatory sequences, such as promoters, enhancers, etc., including about 1 kb, about 2 kb, and possibly more, of flanking genomic DNA at either the 5′ or 3′ end of the transcribed region. Genomic DNA can be isolated as a fragment of 100 kbp or smaller; and substantially free of flanking chromosomal sequence. This genomic DNA flanking the coding region, either 3′ or 5′, or internal regulatory sequences as sometimes found in introns, may contain sequences required for proper tissue and stage-specific expression.

The sequence of the 5′ flanking region can be utilized as promoter elements, including enhancer binding sites that provide for tissue-specific expression and developmental regulation in tissues where the subject genes are expressed, providing promoters that mimic the native pattern of expression. Naturally occurring polymorphisms in the promoter region are useful for determining natural variations in expression, particularly those that may be associated with disease. Promoters or enhancers that regulate the transcription of the polynucleotides of the present invention are obtainable by use of PCR techniques using human tissues, and one or more of the present primers.

Regulatory sequences can be used to identify cis acting sequences required for transcriptional or translational regulation of expression, especially in different tissues or stages of development, and to identify cis acting sequences and trans-acting factors that regulate or mediate expression. Such transcription or translational control regions can be operably linked to a gene in order to promote expression of wild type genes or of proteins of interest in cultured cells, embryonic, fetal or adult tissues, and for gene therapy (Hooper, 1993).

The invention provides variants resulting from random or site-directed mutagenesis. Techniques for in vitro mutagenesis of cloned genes are known. Examples of protocols for site specific mutagenesis may be found in Gustin et al., 1993; Barany 1985; Colicelli et al., 1985; Prentki et al., 1984. Methods for site specific mutagenesis can be found in Sambrook et al., 1989 (pp. 15.3-15.108); Weiner et al., 1993; Sayers et al. 1992; Jones and Winistorfer; Barton et al., 1990; Marotti and Tomich 1989; and Zhu, 1989. Such mutated genes can be used to study structure-function relationships of the subject proteins, or to alter properties of the protein that affect its function or regulation. Other modifications of interest include epitope tagging, e.g., with hemagglutinin (HA), FLAG, or c-myc. For studies of subcellular localization, fluorescent fusion proteins can be used.

The invention also provides variants resulting from chemical or other modifications. Modifications in the native structure of nucleic acids, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters, and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids have modifications that replace the entire ribose phosphodiester backbone with a peptide linkage.

Sugar modifications are also used to enhance stability and affinity. The α-anomer of deoxyribose can be used, where the base is inverted with respect to the natural β-anomer. The 2′-OH of the ribose sugar can be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine, and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Mutations can be introduced into the promoter region to determine the effect of altering expression in experimentally defined systems. Methods for the identification of specific DNA motifs involved in the binding of transcriptional factors are known in the art, for example sequence similarity to known binding motifs, and gel retardation studies (Blackwell et al., 1995; Mortlock et al., 1996; Joulin and Richard-Foy, 1995).

In some embodiments, the invention provides isolated nucleic acids that, when used as primers in a polymerase chain reaction, amplify a subject polynucleotide, or a polynucleotide containing a subject polynucleotide. The amplified polynucleotide is from about 20 to about 50, from about 50 to about 75, from about 75 to about 100, from about 100 to about 125, from about 125 to about 150, from about 150 to about 175, from about 175 to about 200, from about 200 to about 250, from about 250 to about 300, from about 300 to about 350, from about 350 to about 400, from about 400 to about 500, from about 500 to about 600, from about 600 to about 700, from about 700 to about 800, from about 800 to about 900, from about 900 to about 1000, from about 1000 to about 2000, from about 2000 to about 3000, from about 3000 to about 4000, from about 4000 to about 5000, or from about 5000 to about 6000 nucleotides or more in length.

The isolated nucleic acids themselves are from about 10 to about 20, from about 20 to about 30, from about 30 to about 40, from about 40 to about 50, from about 50 to about 100, or from about 100 to about 200 nucleotides in length. Generally, the nucleic acids are used in pairs in a polymerase chain reaction, where they are referred to as “forward” and “reverse” primers.

Thus, in some embodiments, the invention provides a pair of isolated nucleic acid molecules, each from about 10 to about 200 nucleotides in length, the first nucleic acid molecule of the pair comprising a sequence of at least 10 contiguous nucleotides having 100% sequence identity to a nucleic acid sequence as shown in SEQ ID NOS.:1-54 and the second nucleic acid molecule of the pair comprising a sequence of at least 10 contiguous nucleotides having 100% sequence identity to the reverse complement of the nucleic acid sequence shown in SEQ ID NOS.:1-54, wherein the sequence of the second nucleic acid molecule is located 3′ of the nucleic acid sequence of the first nucleic acid molecule shown in SEQ ID NOS.:1-54. The primer nucleic acids are prepared using any known method, e.g., automated synthesis, and can be chosen to specifically amplify a cDNA copy of an mRNA encoding a subject polypeptide.

The subject nucleic acid compositions find use in a variety of different investigative applications. Applications of interest include identifying genomic DNA sequence using molecules of the invention, identifying homologs of molecules of the invention, creating a source of novel promoter elements, identifying expression regulatory factors, creating a source of probes and primers for hybridization applications, identifying expression patterns in biological specimens; preparing cell or animal models to investigate the function of the molecules of the invention, and preparing in vitro models to investigate the function of the molecules of the invention.

The isolated nucleic acids of the invention can be used as probes to detect and characterize gross alteration in a genomic locus, such as deletions, insertions, translocations, and duplications, e.g., by applying fluorescence in situ hybridization (FISH) techniques to examine chromosome spreads (Andreeff et al., 1999). These nucleic acids are also useful for detecting smaller genomic alterations, such as deletions, insertions, additions, translocations, and substitutions (e.g., SNPs).

When used as probes to detect nucleic acid molecules capable of hybridizing with nucleic acids described in the Sequence Listing, the nucleic acid molecules can be flanked by heterologous sequences of any length. When used as probes, a subject nucleic acid can include nucleotide analogs that incorporate labels that are directly detectable, such as radiolabels or fluorescent labels, or nucleotide analogs that incorporate labels that can be visualized in a subsequent reaction.

Fluorescent labels also include a green fluorescent protein (GFP), e.g., a humanized version of a GFP, e.g., wherein codons of the naturally-occurring nucleotide sequence are changed to more closely match the human codon bias; a GFP derived from Aequoria victoria or a derivative thereof, e.g., a humanized derivative such as Enhanced GFP, available commercially, e.g., from Clontech, Inc.; other fluorescent mutants of a GFP from Aequoria victoria, e.g., as described in U.S. Pat. Nos. 6,066,476; 6,020,192; 5,985,577; 5,976,796; 5,968,750; 5,968,738; 5,958,713; 5,919,445; 5,874,304; a GFP from another species such as Renilla reniformis, Renilla mulleri, or Ptilosarcus guernyi, as previously described (WO 99/49019; Peelle et al., 2001), humanized recombinant GFP (hrGFP) (Stratagene®); and any of a variety of fluorescent and colored proteins from Anthozoan species, (e.g., Matz et al., 1999).

Probes can also contain fluorescent analogs, including commercially available fluorescent nucleotide analogs that can readily be incorporated into a subject nucleic acid. These include deoxyribonucleotides and/or ribonucleotide analogs labeled with Cy3, Cy5, Texas Red, Alexa Fluor dyes, rhodamine, cascade blue, or BODIPY, and the like.

Suitable radioactive labels include, e.g., ³²P, ³⁵S, or ³H. For example, probes can contain radiolabeled analogs, including those commonly labeled with ³²P or ³⁵S, such as α-³²P-dATP, dTTP, dCTP, and dGTP; γ-³⁵S-GTP and α-³⁵S-dATP, and the like.

In some embodiments, the first and/or the second nucleic acid molecules comprise a detectable label. The label can be a radioactive molecule, fluorescent molecule or another molecule, e.g., hapten, as described in detail above. Further, the label can be a two stage system, where the amplified DNA is conjugated to another molecule, i.e., biotin, digoxin, or a hapten, that has a high affinity binding partner, i.e., avidin, antidigoxin, or a specific antibody, respectively, and the binding partner conjugated to a detectable label. The label can be conjugated to one or both of the primers. Alternatively, the pool of nucleotides used in the amplification is labeled, so as to incorporate the label into the amplification product.

Conditions that increase stringency of both DNA/DNA and DNA/RNA hybridization reactions are widely known and published in the art. See, for example, Sambrook, 2001, and examples provided above. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where 1×SSC is 0.15 M NaCl and 15 mM citrate buffer); and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

For example, high stringency conditions include hybridization in 50% formamide, 5×SSC, 0.2 μg/μl poly(dA), 0.2 μg/μl human cot 1 DNA, and 0.5% SDS, in a humid oven at 42° C. overnight, followed by successive washes in 1×SSC, 0.2% SDS at 55° C. for 5 minutes, followed by washing at 0.1×SSC, 0.2% SDS at 55° C. for 20 minutes. Further examples of high stringency conditions include hybridization at 50° C. and 0.1×SSC; overnight incubation at 42° C. in a solution containing 50% formamide, 1×SSC, 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. High stringency conditions can also include aqueous hybridization (e.g., free of formamide) in 6×SSC, 1% (SDS) at 65° C. for about 8 hours (or more), followed by one or more washes in 0.2×SSC, 0.1% SDS at 65° C. Highly stringent hybridization conditions are hybridization conditions that are at least as stringent as any one of the above representative conditions. Other stringent hybridization conditions are known in the art and can also be employed to identify nucleic acids of this particular embodiment of the invention.

Conditions of reduced stringency, suitable for hybridization to molecules encoding structurally and functionally related proteins, or otherwise serving related or associated functions, are the same as those for high stringency conditions but with a reduction in temperature for hybridization and washing to lower temperatures (e.g., room temperature or from about 22° C. to 25° C.). For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature. Low stringency conditions include, for example, aqueous hybridization at 50° C. and 6×SSC and washing at 25° C. in 1×SSC.

The specificity of a hybridization reaction allows any single-stranded sequence of nucleotides to be labeled with a radioisotope or chemical and used as a probe to find a complementary strand, even in a cell or cell extract that contains millions of different DNA and RNA sequences. Probes of this type are widely used to detect the nucleic acids corresponding to specific genes, both to facilitate the purification and characterization of the genes after cell lysis and to localize them in cells, tissues, and organisms.

Moreover, by carrying out hybridization reactions under conditions of reduced stringency, a probe prepared from one gene can be used to find homologous evolutionary relatives—both in the same organism, where the relatives form part of a gene family, and in other organisms, where the evolutionary history of the nucleotide sequence can be traced. A person skilled in the art would recognize how to modify the conditions to achieve the requisite degree of stringency for a particular hybridization.

Polypeptides

The invention provides novel polypeptides and related polypeptide compositions. Generally, a polypeptide of the invention refers to a polypeptide which has the amino acid sequence set forth in one or more of SEQ ID NOS.:55-108, as well as polypeptides comprising the amino acid sequences of SEQ ID NOS.:55-108 and polypeptides comprising an amino acid sequences which have at least 70%, at least 80%, at least 85%, at least 90%, at least 93%, at least 95%, at least 98%, or at least 99% identity to that of SEQ ID NOS.:55-108, over their entire length. Specifically, the invention provides one or more amino acid molecule comprising an amino acid sequence according to SEQ ID NOS.:55-108. In particular embodiments, a polypeptide of the invention has an amino acid sequence substantially identical to the sequence of any polypeptide encoded by a polynucleotide sequence shown in SEQ ID NOS.:1-54. The novel polypeptides of the invention also include fragments thereof, and variants, as discussed in more detail below.

In an embodiment, the invention provides an amino acid molecule comprising an amino acid sequence with a sequence of SEQ ID NO.:1-54, or a fragment thereof, comprising a signal peptide, a mature polypeptide that lacks a signal peptide, a polypeptide lacking a signal peptide cleavage site, a biologically active fragment of a polypeptide, a biologically active fragment consisting essentially of a Pfam domain, and a biologically active fragment consisting essentially of a structural motif. Also provided are polypeptides that are substantially identical to at least one amino acid sequence shown in the Sequence Listing, or a fragment thereof, whereby substantially identical is meant that the protein has an amino acid sequence identity to the reference sequence of at least about 75%, at least about 80%, at least about 85%, at least about 90%, at least about 93%, at least about 95%, at least about 97%, at least about 98%, or at least about 99%.

In some embodiments, a polypeptide of the invention comprises at least about 5, at least about 8, at least about 10, at least about 15, at least about 18, at least about 20, at least about 25, at least about 30, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800 contiguous amino acid residues of one or more of the sequences according to SEQ ID NOS.:55-108, up to and including the entire amino acid sequence.

Fragments of the subject polypeptides, as well as polypeptides comprising such fragments, are also provided. Fragments of polypeptides of interest will typically be at least about 5, at least about 8, at least about 10, at least about 15, at least about 18, at least about 20, at least about 25, at least about 30, at least about 50, at least about 75, at least about 100, at least about 150, at least about 200, at least about 250, or at least 300 amino acids in length or longer, where the fragment will have a stretch of amino acids that is identical to the subject protein of at least about 5, at least about 8, at least about 10, at least about 15, at least about 18, at least about 20, at least about 25, at least about 30, or at least about 50 amino acids in length.

In an embodiments, fragments exhibit one or more activities associated with a corresponding naturally occurring polypeptide. Fragments find utility in, for example, generating antibodies to the full-length polypeptide, in methods of screening for candidate agents that bind to and/or modulate polypeptide activity; and in diagnostic, therapeutic, and/or prophylactic methods. Specific fragments of interest include those with enzymatic activity, those with biological activity, including the ability to serve as an epitope or immunogen, and fragments that bind to other proteins or to nucleic acids.

The proteins of the subject invention (e.g., polypeptides encoded by the nucleotide sequences shown in SEQ ID NOS.:1-54, and polypeptide sequences shown in SEQ ID NOS.:55-108) have been separated from their naturally occurring environment and are present in a non-naturally occurring environment. In certain embodiments, the proteins are present in a composition where they are more concentrated than in their naturally occurring environment. For example, isolated polypeptides are provided.

Variants and derivatives of native proteins that retain a desired biological activity are also within the scope of the present invention. These variants and derivatives include polypeptides substantially homologous to native proteins, but with an amino acid sequence different from that of the native protein because of one or a plurality of deletions, insertions, or substitutions. In an embodiment, the biological activity of a variant is essentially equivalent to the biological activity of the native protein. Variants may be obtained by mutations of native nucleotide sequences. Polypeptide-encoding DNA sequences of the present invention encompass sequences that comprise one or more additions, deletions, or substitutions of nucleotides when compared to a native DNA sequence, but that encode a protein essentially biologically equivalent to a native protein. The variant amino acid or DNA sequence preferably is at least about 70%, at least about 75%, at least about 80%, at least about 85%, at least about 90%, at least about 93%, at least about 95%, at least about 97%, at least about 98%, or at least about 99% identical to a native sequence. The degree of homology (percent identity) between a native and a mutant sequence may be determined, for example, by comparing the two sequences using computer programs commonly employed for this purpose. Homologues can comprise polypeptides of other species, including mammals, such as: primates, rodents, e.g., mice, rats, hamsters, guinea pigs; domestic animals, e.g., sheep, pig, horse, cow, goat, rabbit, dog, cat; and humans, as well as non-mammalian species, e.g., avian, reptile and amphibian, insect, crustacean, fish, plant, fungus, and protozoa. Homology can be measured, e.g., with the “GAP” program (part of the Wisconsin Sequence Analysis Package available through the Genetics Computer Group, Inc. (Madison Wis.)), where the parameters are: Gap weight: 12; length weight: 4.

Homologs are identified by any of a number of methods. By using probes, particularly labeled probes of DNA sequences, one can isolate homologous or related genes, as described in detail above. Briefly, a fragment of the provided cDNA can be used as a hybridization probe against a cDNA library from the target organism of interest, under various stringency conditions, e.g., low stringency conditions. The probe can be a large fragment, or one or more short degenerate primers, and is typically labeled. Sequence identity can be determined by hybridization under stringent conditions, as described in detail above. Nucleic acids having a region of substantial identity or sequence similarity to the provided nucleic acid sequences, for example allelic variants, related genes, or genetically altered versions of the gene, bind to the provided sequences under less stringent hybridization conditions.

Alterations of the native amino acid sequence may be accomplished by any of a number of known techniques. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene having particular codons altered according to the substitution, deletion, or insertion required (Walder and Walder, 1986; Bauer et al., 1985; Craik, 1985; and U.S. Pat. Nos. 4,518,584 and 4,737,462)

Variants may comprise conservatively substituted sequences, meaning that one or more amino acid residues of a native polypeptide are replaced by different residues, but that the conservatively substituted polypeptide retains a desired biological activity that is essentially equivalent to that of a native polypeptide. Examples of conservative substitutions include substitution of amino acids that do not alter secondary and/or tertiary structure. Other examples involve substitution of amino acids outside the receptor-binding domain, when the desired biological activity is the ability to bind to a receptor on target cells. A given amino acid may be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Advantageously, the conserved amino acids are not altered when generating conservatively substituted sequences. If altered, amino acids found at equivalent positions in other members of the protein family, when known, are substituted.

In some embodiments, a subject polypeptide is present as an oligomer, including homodimers, homotrimers, homotetramers, and multimers that include more than four monomeric units. Oligomers also include heteromultimers, e.g., heterodimers, heterotrimers, heterotetramers, etc. where the subject polypeptide is present in a complex with proteins other than the subject polypeptide. Where the multimer is a heteromultimer, the subject polypeptide can be present in a 1:1 ratio, a 1:2 ratio, a 2:1 ratio, or other ratio, with the other protein(s).

Oligomers may be formed by disulfide bonds between cysteine residues on different polypeptides, or by non-covalent interactions between polypeptide chains, for example. In other embodiments, oligomers comprise from two to four polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of polypeptides attached thereto, as described in more detail below.

Polypeptides of the invention can be obtained from naturally-occurring sources or produced synthetically. The sources of naturally occurring polypeptides will generally depend on the species from which the protein is to be derived, i.e., the proteins will be derived from biological sources that express the proteins. The subject proteins can also be derived from synthetic means, e.g., by expressing a recombinant gene encoding a protein of interest in a suitable system or host or enhancing endogenous expression, as described in more detail below. Further, small peptides can be synthesized in the laboratory by techniques well known in the art.

Specifically, the invention provides one or more amino acid molecule comprising at least one amino acid sequence of SEQ ID NOS.:55-108 or a fragment thereof, wherein the polypeptide functions as an agonist, an antagonist, a ligand, and/or a receptor.

The sequences of the invention encompass a variety of different types of secreted and transmembrane nucleic acids and polypeptides with different structures and functions. These polypeptides may reside within the cell, or extracellularly. They may be secreted from the cell, or reside in the plasma membrane or the membrane of any of the intracellular organelles. Many and widely variant biological functions are mediated by a wide variety of different types of secreted and transmembrane proteins. Yet, despite the sequencing of the human genome, relatively few pharmaceutically useful secreted and transmembrane proteins have been identified. It would be advantageous to discover novel secreted and transmembrane proteins or polypeptides, and their corresponding polynucleotides, which have medical utility. Pharmaceutically useful secreted proteins and transmembrane of the present invention will have in common the ability to act as ligands for binding to receptors on cell surfaces in ligand/receptor interactions, to trigger certain intracellular responses, such as inducing signal transduction to activate cells or inhibit cellular activity, to induce cellular growth, proliferation, or differentiation, or to induce the production of other factors that, in turn, mediate such activities.

The cell types having cell surface receptors responsive to secreted proteins are various, including, for example, stem cells; progenitor cells; and precursor cells and mature cells of the hematopoietic, hepatic, neural, lung, heart, thymic, splenic, epithelial, pancreatic, adipose, gastrointestinal, colonic, optic, olfactory, bone and musculoskeletal lineages. Further, the hematopoietic cells can be red blood cells or white blood cells, including cells of the B lymphocytic (B cell), T lymphocytic (T cell), dendritic, megakaryocytic, natural killer (NK), macrophagic, eosinophilic, and basophilic lineages. The cell types responsive to secreted proteins also include normal cells or cells implicated in disorders or other pathological conditions.

As an example, certain of the secreted and/or transmembrane proteins of the present invention regulate cell division and/or differentiation, regulate the immune response, and/or are involved in the pathogenesis of a variety of diseases and disorders. Certain of the secreted proteins of the invention can function as cytochrome oxidases, permeases, and proteases. Certain of the transmembrane proteins of the invention can function as histocompatibility antigens, mucins, and dehydrogenases. The predicted functions of the secreted and/or transmembrane proteins of the invention are provided in greater detail in Tables 3, 4, 8, and 9.

Certain of the secreted and/or transmembrane proteins of the present invention are useful for diagnosis, prophylax is, or treatment of disorders in subjects that are deficient in such secreted proteins or require regeneration of certain tissues, the proliferation of which is dependent on such secreted or transmembrane proteins, or requires an inhibition or activation of growth that is dependent on such secreted or transmembrane proteins. Examples of such disorders include cancer, such as breast cancer, colon cancer, lung adenocarcinoma, lung squamous cell carcinoma, and prostate cancer; immune diseases, such as autoimmunity; inflammatory diseases, such as inflammatory bowel disease; lung diseases, such as asthma, and others, as shown in greater detail in Table 8.

The secreted proteins of the invention are present in the cell culture medium of cells from which they are synthesized and secreted. The invention provides a cell culture medium comprising one or more polypeptide molecule comprising a polypeptide sequence according to SEQ ID NO.:55-108. This cell culture medium can comprise responder cells chosen from one or more of T cells, B cells, NK cells, dendritic cells, macrophages, muscle cells, stem cells, epithelial skin cells, fat cells, blood cells, brain cells, bone marrow cells, endothelial cells, retinal cells, bone cells, kidney cells, pancreatic cells, liver cells, spleen cells, prostate cells, cervical cells, ovarian cells, breast cells, lung cells, liver cells, soft tissue cells, colorectal cells, cells of the gastrointestinal tract, and cancer cells.

The invention also provides cell culture medium in which the responder cells proliferate in the medium. In an embodiment at least one activity of the responder cells is inhibited in the medium. The invention provides a cell culture comprising cells transfected with a first nucleic acid molecule comprising a polynucleotide sequence chosen from a polynucleotide sequence according to SEQ ID NOS.:1-54, a complement thereof, and/or at least one polynucleotide sequence that encodes SEQ ID NOS.:55-108. This cell culture may further comprise responder cells chosen from one or more of T cells, B cells, NK cells, dendritic cells, macrophages, muscle cells, stem cells, epithelial skin cells, fat cells, blood cells, brain cells, bone marrow cells, endothelial cells, retinal cells, bone cells, kidney cells, pancreatic cells, liver cells, spleen cells, prostate cells, cervical cells, ovarian cells, breast cells, lung cells, liver cells, soft tissue cells, colorectal cells, cells of the gastrointestinal tract, and cancer cells. In an embodiment, the responder cells proliferate in this cell culture. The invention also provides such a cell culture, wherein at least one activity of the responder cells is inhibited in the cell culture.

The secreted and/or transmembrane proteins of the invention can encode or comprise polypeptides belonging to different protein families (Pfam). The Pfam system is an organization of protein sequence classification and analysis, based on conserved protein domains; it can be publicly accessed in a number of ways, for example, at http://Pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman et al., 2002). Sequences of the invention can encode or be comprised of more than one Pfam.

HG1012993P1 and HG1013025 possess Pfam domains comprising immunoglobulin (ig) domains (Table 5), which are characteristically found in the immunoglobulin superfamily, a large superfamily comprised of hundreds of proteins with various functions (http://Pfam.wustl.edu/cgi-bin/getdesc?name=ig) (Williams and Barclay, 1988). Ig domains are involved in protein-protein and protein-ligand interactions; their presence is predictive that HG1012993P1 and HG1013025 are involved in protein-protein and protein-ligand interactions.

HG1012993P1 and HG1013025 also possess Pfam domains and three dimensional structural motifs comprising class II histocompatibility antigen alpha domains. This domain is located on the A chain of the MHC class II glycoprotein, beginning at approximately residue 4 and ending at approximately residue 84. Their presence is predictive that HG1012993P1 and HG1013025 may function in a manner similar to that of the major histocompatibility antigen alpha domain (http://pfam.wustl.edu/cgi-binlgetdesc?name=MHC_II_alpha) (Janeway et al., 2001).

A structural analysis of the polypeptides of the invention has identified several three-dimensional motifs in HG1012887P1, HG1012993P1, and HG1013025P1 in addition to the above-described Pfam domains. As shown in Table 6, HG1012887P1 has a trypsin-like serine protease motif. Trypsin-like serine proteases are multifunctional peptidases that cleave peptides at serine residues. They are known to function as epithelial tumor antigens (http://pfam.wustl.edu/cgi-bin/getdesc?name=Trypsin) (Rawlings and Barrett, 1994). Its presence is predictive that HG1012887P1 has one or more functions of a trypsin-like serine protease.

Also as shown in Table 6, HG1012993P1 and HG1013025P1 possess a MHC antigen-recognition domain structural motif. The MHC antigen recognition domain can distinguish peptides bound by particular allelic variants of an MHC molecule. MHC antigen recognition domains are polymorphic regions of the molecule, located at a site on the molecule distant from the membrane. Their presence is predictive that HG1012993P1 and HG1013025P1 have one or more functions of a MHC antigen recognition domain.

As further shown in Table 6, HG1012993P1 and HG1013025P1 possess a WW domain, a short, conserved region characterized by two conserved tryptophan residues and a conserved proline residue. This domain has approximately 35-40 residues and may be repeated several times. It binds to proteins that possess characteristic proline motifs, and is often associated with other domains that mediate signal transduction (http://pfam.wustl.edu/cgi-bin/getdesc?name=WW) (Pirozi et al., 1997). Their presence is predictive that HG1012993P1 and HG1013025P1 have one or more functions of a WW domain.

HG1012887, herein referred to as SEQ ID NO.:22 and SEQ ID NO.:77, has a predicted length of 213 amino acids. It's Tree Vote of 0.96 identifies it as a secreted protein. HG1012887 has multiple signal peptide and mature protein coordinates, as shown in Table 2. The protein in the NCBI database with which it displays the greatest similarity is a murine serine protease type 2, which is involved in uterine implantation. It was identified from a placenta library.

HG1012993, herein referred to as SEQ ID NO.:37 and SEQ ID NO.:91, has a predicted length of 255 amino acids. It is a single transmembrane protein; amino acids 219-241 span the membrane. HG1012993 has multiple signal peptide and mature protein coordinates, as shown in Table 2. The protein in the NCBI database with which it displays the greatest similarity is a human MHC class II histocompatibility antigen HLA-DQ alpha chain precursor, with which is shares 99% identity, as shown in Tables 3 and 4. HG1012993 was identified from a breast library.

HG1013025, herein referred to as SEQ ID NO.:48 and SEQ ID NO.:102, also has a predicted length of 255 amino acids. It is a single transmembrane protein; amino acids 218-240 span the membrane. HG1013025 has multiple signal peptide and mature protein coordinates, as shown in Table 2. The protein in the NCBI database with which it displays the greatest similarity is, like HG1012993, a human MHC class II histocompatibility antigen HLA-DQ alpha chain precursor, with which it shares 100% identity, as shown in Tables 3 and 4. HG1013025 was identified from a tonsil library.

The secreted and/or transmembrane proteins of the invention can be screened for functional activities in appropriate functional assays, as is conventional in the art. Such assays include, for example, in vitro and in vivo assays for factors that stimulate the proliferation or differentiation of stem cells, progenitor cells, or precursor cells into T cells, B cells, pancreatic islet cells, bone cells, neuronal cells, etc.

The protein expression systems described below can produce fusion proteins that incorporate the polypeptides of the invention. The invention provides an isolated amino acid molecule with a first polypeptide comprising SEQ ID NO:55-108 or one or more of its biologically active fragments or variants, and a second molecule. This second molecule can facilitate production, secretion, and/or purification. It can confer a longer half-life to the first polypeptide when administered to an animal. Second molecules suitable for use in the invention include, e.g., polyethylene glycol (PEG), human serum albumin, fetuin, and/or one or more of their fragments as discussed below. The invention can also provide a nucleic acid molecule with a second nucleotide sequence that encodes a fusion partner. This second nucleotide sequence can be operably linked to the first nucleotide sequence.

Thus, the invention provides polypeptide fusion partners. They may be part of a fusion molecule, e.g., a polynucleotide or polypeptide, which represents the joining of all of or portions of more than one gene. For example, a fusion protein can be the product obtained by splicing strands of recombinant DNA and expressing the hybrid gene. A fusion molecule can be made by genetic engineering, e.g., by removing the stop codon from the DNA sequence of a first protein, then appending the DNA sequence of a second protein in frame. The DNA sequence will then be expressed by a cell as a single protein. Typically this is accomplished by cloning a cDNA into an expression vector in frame with an existing gene. The invention provides fusion proteins with heterologous and homologous leader sequences, fusion proteins with a heterologous amino acid sequence, and fusion proteins with or without N-terminal methionine residues. The fusion partners of the invention can be either N-terminal fusion partners or C-terminal fusion partners.

As noted above, suitable fusion partners include, but are not limited to, albumin and fetuin (Yao et al., 2004; Chu, pending U.S. provisional application filed Jul. 22, 2004, entitled Fusion Polypeptides of Human Fetuin and Therapeutically Active Polypeptides). These fusion partners can include any variant of albumin, fetuin, or any fragment thereof. The natural fetuin polypeptides of the invention encompass all known isoforms and splice variants of fetuin A and B. The fetuin variants of the invention encompass any fetuin polypeptide with a high plasma half-life which is obtained by modification, such as by mutation, deletion, or addition. The invention encompasses all fetuin variants with a high plasma half-life obtained by in vitro modification of a polypeptide encoded by a fetuin polynucleotide. It includes non-natural sequences isolated from random peptide libraries. It also includes natural or artificial post-translational modifications, such as prenylation, glycosylation, e.g., with sialic acid, and the like. Modifications can be performed by any technique known in the art, such as commonly employed genetic engineering techniques. Such modified polypeptides can show, e.g., enhanced activity or increased stability. In addition, they may be purified in higher yields and show better solubility than the corresponding natural polypeptide, at least under certain purification and storage conditions.

Fusion polypeptides can be secreted from the cell by the incorporation of leader sequences that direct the protein to the membrane for secretion. These leader sequences can be specific to the host cell, and are known to skilled artisans; they are also cited in the references. The invention includes appropriate restriction enzyme sites for vector cloning. In addition to facilitating the secretion of these fusion proteins, the invention provides for facilitating their production. This can be accomplished in a number of ways, including producing multiple copies, employing strong promoters, and increasing their intracellular stability, e.g., by fusion with beta-galactosidase.

The invention also provides for facilitating the purification of these fusion proteins. Fusion with a selectable marker can facilitate purification by affinity chromatography. For example, fusion with the selectable marker glutathione S-transferase (GST) produces polypeptides that can be detected with antibodies directed against GST, and isolated by affinity chromatography on glutathione-sepharose; the GST marker can then be removed by thrombin cleavage. Polypeptides that provide for binding to metal ions are also suitable for affinity purification. For example, a fusion protein that incorporates Hiss, where n is between three and ten, inclusive, e.g., a 6×His-tag can be used to isolate a protein by affinity chromatography using a nickel ligand.

Suitable fusion partners that can be used to detect the fusion protein include all polypeptides that can bind to an antibody specific to the fusion partner (e.g., epitope tags, such as c-myc, hemagglutinin, and the FLAG® peptide, which is highly antigenic and provides an epitope reversibly bound by a specific monoclonal antibody, thus providing the fusion protein with a rapid assay and easy purification method); polypeptides that provide a detectable signal (e.g., a fluorescent protein, e.g., a green fluorescent protein, a fluorescent protein from an Anthozoan species; β-galactosidase; and luciferase). Also by way of example, where the fusion partner provides an immunologically recognizable epitope, an epitope-specific antibody can be used to quantitatively detect the level of polypeptide. In some embodiments, the fusion partner provides a detectable signal, and in these embodiments, the detection method is chosen based on the type of signal generated by the fusion partner. For example, where the fusion partner is a fluorescent protein, fluorescence is measured.

Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al., 2001; “humanized” recombinant GFP (hrGFP) (Stratagene); any of a variety of fluorescent and colored proteins from Anthozoan species, as described in, e.g., Matz et al., 1999.

Where the fusion partner is an enzyme that yields optically detectable product, the product can be detected using an appropriate means. For example, β-galactosidase can, depending on the substrate, yield a colored product that can detected with a spectrophotometer, and the protein luciferase can yield a luminescent product detectable with a luminometer.

The fusion partners of the invention can also include linkers, i.e., fragments of synthetic DNA containing a restriction endonuclease recognition site that can be used for splicing genes. These can include polylinkers, which contain several restriction enzyme recognition sites. A linker may be part of a cloning vector. It may be located either upstream or downstream of the therapeutic protein, and it may be located either upstream or downstream of the fusion partner.

Gene manipulation techniques have enabled the development and use of recombinant therapeutic proteins with fusion partners that impart desirable pharmacokinetic properties. Recombinant human serum albumin fused with synthetic heme protein has been reported to reversibly carry oxygen (Chuang et al., 2002). The long half-life and stability of human serum albumin (HSA) make it an attractive candidate for fusion to short-lived therapeutic proteins (U.S. Pat. No. 6,686,179).

For example, the short plasma half-life of unmodified interferon alpha makes frequent dosing necessary over an extended period of time, in order to treat viral and proliferative disorders. Interferon alpha fused with HSA has a longer half life and requires less frequent dosing than unmodified interferon alpha; the half-life was 18-fold longer and the clearance rate was approximately 140 times slower (Osborn et al., 2002). Interferon beta fused with HSA also has favorable pharmacokinetic properties, its half life was reported to be 36-40 hours, compared to 8 hours for unmodified interferon beta (Sung et al., 2003). A HSA-interleukin-2 fusion protein has been reported to have both a longer half-life and favorable biodistribution compared to unmodified interleukin-2. This fusion protein was observed to target tissues where lymphocytes reside to a greater extent than unmodified interleukin 2, suggesting that it exerts greater efficacy (Yao et al., 2004).

The Fc receptor of human immunoglobulin G subclass 1 has also been used as a fusion partner for a therapeutic molecule. It has been recombinantly linked to two soluble p75 tumor necrosis factor (TNF) receptor molecules. This fusion protein has been reported to have a longer circulating half-life than monomeric soluble receptors, and to inhibit TNFα-induced proinflammatory activity in the joints of patients with rheumatoid arthritis (Goldenberg, 1999). This fusion protein has been used clinically to treat rheumatoid arthritis, juvenile rheumatoid arthritis, psoriatic arthritis, and ankylosing spondylitis (Nanda and Bathon, 2004).

The peptides of the invention, including the fusion proteins, can be modified with or covalently coupled to one or more of a variety of hydrophilic polymers to increase their solubility and circulation half-life. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky 1995; Monfardini et al., 1995; U.S. Pat. Nos. 4,791,192; 4,670,417; 4,640,835; 4,496,689; 4,301,144; 4,179,337 and WO 95/34326.

An embodiment of the invention encompasses polypeptides of the invention in the form of oligomers, such as dimers, trimers, or higher oligomers. Oligomers may be formed by disulfide bonds between cysteine residues on different polypeptides, or by non-covalent interactions between polypeptide chains. Oligomers may also comprise from two to four polypeptides joined via covalent or non-covalent interactions between peptide moieties fused to the polypeptides. These moieties may be peptide linkers (spacers) or peptides that can promote oligomerization; accordingly, the invention provides oligomers comprising two or more polypeptides joined through peptide linkers. Fusion proteins comprising multiple polypeptides separated by peptide linkers can be produced using conventional recombinant DNA technology. Oligomeric polypeptides can also be prepared with a leucine zipper domain, which promotes oligomerization. Among the known leucine zippers are naturally occurring peptides and derivatives thereof that form dimers or trimers. Examples of leucine zipper domains suitable for producing soluble oligomeric proteins are those described in application WO 94/10308.

Conjugating biomolecules with polyethylene glycol (PEG), a process known as pegylation, increases the circulating half-life of therapeutic proteins (Molineux, 2002). Polyethylene glycols are nontoxic water-soluble polymers that, owing to their large hydrodynamic volume, create a shield around the pegylated drug, thus protecting it from renal clearance, enzymatic degradation, and recognition by cells of the immune system.

Pegylated agents have improved pharmacokinetics that permit dosing schedules that are more convenient and more acceptable to patients. This improved pharmacokinetic profile may decrease adverse effects caused by the large variations in peak-to-trough plasma drug concentrations associated with frequent administration and by the immunogenicity of unmodified proteins (Harris et al., 2001). In addition, pegylated proteins may have reduced immunogenicity because PEG-induced steric hindrance can prevent immune recognition (Harris et al., 2001).

Polypeptides of the invention can be isolated by any appropriate means known in the art. For example, convenient protein purification procedures can be employed (e.g., Deuthscher et al., 1990). In general, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography, and the like.

The invention also provides a method of making a polypeptide of the invention by providing a nucleic acid molecule that comprises a polynucleotide sequence encoding a polypeptide of the invention, introducing the nucleic acid molecule into an expression system, and allowing the polypeptide to be produced. Briefly, the methods generally involve introducing a nucleic acid construct into a host cell in vitro and culturing the host cell under conditions suitable for expression, then harvesting the polypeptide, either from the culture medium or from the host cell, (e.g., by disrupting the host cell), or both, as described in detail above. The invention also provides methods of producing a polypeptide using cell-free in vitro transcription/translation methods, which are well known in the art, also as provided above.

Specifically, the invention provides a method of making a polypeptide by providing a nucleic acid molecule that comprises a polynucleotide sequence encoding one or more polypeptide comprising the polypeptide sequence chosen from at least one amino acid sequence according to SEQ ID NOS.:55-108; introducing the nucleic acid molecule into an expression system; and allowing the polypeptide to be produced. It also provides a method of making a polypeptide by providing a composition comprising a host cell transformed, transduced, transfected, or infected with a nucleic acid molecule comprising at least one polynucleotide sequence of SEQ ID NO.:1-54, or at least one polynucleotide sequence that encodes SEQ ID NO.:55-108; culturing the host cell to produce the polypeptide; and allowing the polypeptide to be produced.

The present invention also provides methods of producing a subject polypeptide and provides antibodies that specifically bind to a subject polypeptide. The present invention further provides screening methods for identifying agents that modulate a level or an activity of a subject polypeptide or polynucleotide. The present invention thus also provides agents that modulate a level or an activity of a subject polypeptide or polynucleotide, as well as compositions, including pharmaceutical compositions, comprising a subject agent.

Libraries and Arrays

The present invention further features a library of polynucleotides, wherein at least one of the polynucleotides comprises the sequence information of a polynucleotide of the invention. In specific embodiments, the library is provided on a nucleic acid array. In some embodiments, the library is provided in computer-readable format.

The sequence information contained in either a biochemical or an electronic library of polynucleotides can be used in a variety of ways, e.g., as a resource for gene discovery, as a representation of sequences expressed in a selected cell type (e.g., cell type markers), or as markers of a given disorder or disease state. In general, a disease marker is a representation of a gene product that is present in all cells affected by disease either at an increased or decreased level relative to a normal cell (e.g., a cell of the same or similar type that is not substantially affected by disease). For example, a polynucleotide sequence in a library can be a polynucleotide that represents an mRNA, polypeptide, or other gene product encoded by the polynucleotide, that is either over-expressed or under-expressed in one cell compared to another (e.g., a first cell type compared to a second cell type; a normal cell compared to a diseased cell; a cell not exposed to a signal or stimulus compared to a cell exposed to that signal or stimulus; and the like).

The polynucleotide libraries of the invention generally comprise a collection of sequence information of a plurality of polynucleotide sequences, where at least one of the polynucleotides has a sequence shown in SEQ ID NOS.:1-54. By plurality is meant at least two, at least three, or at least any integer up to and including all of the sequences in the Sequence Listing. The information may be provided in either biochemical form (e.g., as a collection of polynucleotide molecules), or in electronic form (e.g., as a collection of polynucleotide sequences stored in a computer-readable form, as in a computer-based system, a computer data file, and/or as a part of a computer program). The length and number of polynucleotides in the library will vary with the nature of the library, e.g., depending upon whether the library is, e.g., an oligonucleotide array, a cDNA array, or a computer database of the sequence information.

For example, a library of sequence information embodied in electronic form comprises an accessible computer data file that may contain the representative nucleotide sequences of genes that are differentially expressed (e.g., over-expressed or under-expressed) as between, e.g., a first cell type compared to a second cell type (e.g., expression in a brain cell compared to expression in a kidney cell); a normal cell compared to a diseased cell (e.g., a non-cancerous cell compared to a cancerous cell); a cell not exposed to an internal or external signal or stimulus compared to a cell exposed to that signal or stimulus (e.g., a cell contacted with a ligand compared to a control cell not contacted with the ligand); and the like. Other combinations and comparisons of cells will be readily apparent to the ordinarily skilled artisan. Biochemical embodiments of the library include a collection of nucleic acid molecules that have the sequences of the genes in the library, where the nucleic acids can correspond to the entire gene in the library or to a fragment thereof, as described in greater detail below.

Where the library is an electronic library, the nucleic acid sequence information can be present in a variety of media. For example, the nucleic acid sequences of any of the polynucleotides shown in SEQ ID NOS.:1-54 can be recorded on computer readable media of a computer-based system, e.g., any medium that can be read and accessed directly by a computer. One of skill in the art can readily appreciate how any of the presently known computer readable mediums can be used to create a manufacture comprising a recording of the present sequence information. Any convenient data storage structure can be chosen, based on the means used to access the stored information. A variety of data processor programs and formats can be used for storage, e.g., word processing text file, database format, etc. In addition to the sequence information, electronic versions of the libraries of the invention can be provided in conjunction or connection with other computer-readable information and/or other types of computer-based files (e.g., searchable files, executable files, etc, including, but not limited to, for example, search program software, etc.).

By providing the nucleotide sequence in computer readable form in a computer-based system, the information can be accessed for a variety of purposes. Computer software to access sequence information is publicly available. Conventional bioinformatics tools can be utilized to analyze sequences to determine sequence identity, sequence similarity, and gap information. For example, the gapped BLAST (Altschul et al., 1990, Altschul et al., 1997), and BLAZE (Brutlag et al., 1993) search algorithms on a Sybase system, or the TeraBLAST (TimeLogic, Crystal Bay, Nev.) program optionally running on a specialized computer platform available from TimeLogic, can be used to identify open reading frames (ORFs) within the genome that contain homology to ORFs from other organisms. Homology between sequences of interest can be determined using the local homology algorithm of Smith and Waterman, 1981, as well as the BestFit program (Rechid et al., 1989), and the FastDB algorithm (FastDB, 1988; described in Current Methods in Sequence Comparison and Analysis, Macromolecule Sequencing and Synthesis, Selected Methods and Applications, pp. 127-149, 1988, Alan R. Liss, Inc).

Alignment programs that permit gaps in the sequence include Clustalw (Thompson et al., 1994), FASTA3 (Pearson, 2000) Align0 (Myers and Miller, 1988), and TCoffee (Notredame et al., 2000). Other methods for comparing and aligning nucleotide and protein sequences include, for example, BLASTX (NCBI), the Wise package (Birney and Durbin, 2000), and FASTX (Pearson, 2000). These algorithms determine sequence homology between nucleotide and protein sequences without translating the nucleotide sequences into protein sequences. Other techniques for alignment are also known in the art (Doolittle, et al., 1996; BLAST, available from the National Center for Biotechnology Information; FASTA, available in the Genetics Computing Group (GCG) package, from Madison, Wis., USA, a wholly owned subsidiary of Oxford Molecular Group, Inc.; Schlessinger, 1988a; Schlessinger, 1988b; and Needleman and Wunch, 1970).

Sequence similarity is calculated based on a reference sequence, which may be a subset of a larger sequence, such as a conserved motif, coding region, flanking region, etc. The reference sequence is usually at least about 18 nucleotides long, at least about 30 nucleotides long, or may extend to the complete sequence that is being compared.

One parameter for determining percent sequence identity is the percentage of the alignment in the region of strongest alignment between a target and a query sequence. Methods for determining this percentage involve, for example, counting the number of aligned bases of a query sequence in the region of strongest alignment and dividing this number by the total number of bases in the region. For example, 10 matches divided by 11 total residues gives a percent sequence identity of approximately 90.9%.

A variety of structural formats for the input and output means can be used to input and output the information in the computer-based systems of the present invention. One format for an output means ranks the relative expression levels of different polynucleotides. Such presentation provides a skilled artisan with a ranking of relative expression levels to determine a gene expression profile.

As discussed above, the library of the invention also encompasses biochemical libraries of the polynucleotides shown in SEQ ID NOS.:1-54 or one of its complements, fragments, or variants, e.g., collections of nucleic acids representing the provided polynucleotides. The biochemical libraries can take a variety of forms, e.g., a solution of cDNAs, a pattern of probe nucleic acids stably associated with a surface of a solid support (i.e., an array) and the like. Of particular interest are nucleic acid arrays in which one or more of the polynucleotide sequences shown in SEQ ID NOS.:1-54 is represented on the array. A variety of different array formats, as described in more detail below, have been developed and are known to those of skill in the art. The arrays of the subject invention find use in a variety of applications, including gene expression analysis, drug screening, mutation analysis, and the like, as disclosed in the herein-listed exemplary patent documents.

In addition to the above nucleic acid libraries, analogous libraries of polypeptides are also provided, where the polypeptides of the library will represent at least a portion of the polypeptides encoded by a gene corresponding to one or more of the sequences shown in SEQ ID NOS.:1-54.

Further, analogous libraries of antibodies are also provided, where the libraries comprise antibodies or fragments thereof, both of which are described in more detail below, that specifically bind to at least a portion of at least one of the subject polypeptides. Further, antibody libraries may comprise antibodies or fragments thereof that specifically inhibit binding of a subject polypeptide to its ligand or substrate, or that specifically inhibit binding of a subject polypeptide as a substrate to another molecule. Moreover, corresponding nucleic acid libraries are also provided, comprising polynucleotide sequences that encode the antibodies or antibody fragments described above.

The nucleic acid molecules and the amino acid molecules of the invention can be bound to a substrate. They can be attached covalently, attached to a surface of the support, or applied to a derivatized surface in a chaotropic agent that facilitates denaturation and adherence, e.g., by noncovalent interactions, or some combination thereof. The nucleic acids can be bound to a substrate to which a plurality of other nucleic acids are concurrently bound, such that hybridization to each of the plurality of the bound nucleic acids is separately detectable. The substrate can be porous or solid, planar or non-planar, unitary or distributed, and the bond between the nucleic acid and the substrate can be covalent or non-covalent. The substrate can be in the form of microbeads or nanobeads. Substrates include, but are not limited to, a membrane, such as nitrocellulose, nylon, positively-charged derivatized nylon; a solid substrate such as glass, amorphous silicon, crystalline silicon, plastics (including e.g., polymethylacrylic, polyethylene, polypropylene, polyacrylate, polymethyl methacrylate, polyvinyl chloride, polytetrafluoroethylene, polystyrene, polycarbonate, polyacetal, polysulfone, cellulose acetate, or mixtures thereof).

Arrays of the invention can include all of the devices referred to as microarrays in Schena, 1999; Bassett et al., 1999; Bowtell, 1999; Brown and Botstein, 1999; Chakravarti, 1999; Cheung et al., 1999; Cole et al., 1999; Collins, 1999; Debouck and Goodfellow, 1999; Duggan et al., 1999; Hacia, 1999; Lander, 1999; Lipshutz et al., 1999; Southern, et al., 1999; Schena, 2000; Brenner et al, 2000; Lander, 2001; Steinhaur et al., 2002; and Espejo et al, 2002. Protein and antibody microarrays include arrays of polypeptides or proteins, including but not limited to, polypeptides or proteins obtained by purification, fusion proteins, and antibodies, and can be used for specific binding studies. Nucleic acid microarrays include both oligonucleotide arrays (DNA chips) containing expressed sequence tags (“ESTs”) and arrays of larger DNA sequences representing a plurality of genes bound to the substrate, either one of which can be used for hybridization studies.

The invention provides an array comprising one or more nucleic acids comprising the product of a polymerase chain reaction which uses two of the 3′ untranslated gene regions of a gene that comprises one or more polynucleotide sequence according to SEQ ID NOS.:1-54 as primers. Specifically, the invention provides the 3′ untranslated region of a gene that comprises one or more polynucleotide sequences according to SEQ ID NOS.:1-54.

In an embodiment, a microarray chip of the invention detects a polynucleotide, such as an mRNA encoding a polypeptide, with a pair of nucleic acids that function as “forward” and “reverse” primers that specifically amplify a cDNA copy of the mRNA. The “forward” and “reverse” primers are provided as a pair of isolated nucleic acid molecules, each from about 20 to about 30 contiguous nucleotides in length, from about 20 to about 25 contiguous nucleotides in length, from about 20 to 23 contiguous nucleotides in length, and from about 20 to 22 contiguous nucleotides in length. The first nucleic acid molecule of the pair comprises a sequence having either 100% sequence identity or sequence homology to at least one nucleic acid sequence corresponding to the 3′ untranslated region of SEQ ID NOS.:1-54. The second nucleic acid molecule of the pair comprises a sequence having either 100% sequence identity or sequence homology to at least one nucleic acid sequence corresponding to the reverse complement of the 3′ untranslated region of SEQ ID NOS.:1-54. The sequence of said second nucleic acid molecule is located 3′ of the nucleic acid sequence of the first nucleic acid molecule shown in SEQ ID NOS.:1-54. The pair of isolated nucleic acid molecules are useful in a polymerase chain reaction or in any other method known in the art to amplify a nucleic acid that has sequence identity to the sequences shown in SEQ ID NOS.:1-54, particularly when cDNA is used as a template. These primer nucleic acids can be prepared using any known method, e.g., automated synthesis, and can be chosen to specifically amplify a cDNA copy of an mRNA encoding a polypeptide of the Sequence Listing. In an embodiment, one or both members of the pair of nucleic acid molecules comprise a detectable label.

Expression of the Human cDNA Clones

The invention provides, as expression systems, any composition that permits protein synthesis when an expression vector is provided to the system. Expression systems are well-known by those skilled in the art. They include cell-free expression systems, e.g., wheat germ extract systems, rabbit reticulocyte lysate systems, and frog oocyte systems. They also include systems that utilize host cells, such as E. coli expression systems, yeast expression systems, insect expression systems, and mammalian expression systems, such as in CHO cells or 293 cells. The expression systems of the invention may also comprise translation systems, which support the processes by which the sequence of nucleotides in a messenger RNA molecule directs the incorporation of amino acids into a protein or polypeptide. Expression and translation systems of the invention may allow polypeptide synthesis, i.e., permit the incorporation of amino acids into a protein or polypeptide.

The invention provides vectors, i.e., plasmids that can be used to transfer DNA sequences from one organism to another or to express a gene of interest. It provides both recombinant plasmid vectors and recombinant expression vectors. These recombinant vectors, or constructs, which can include nucleic acids of the invention, are useful for propagating a nucleic acid in a cell free expression system or host cell. Plasmid vectors can transfer nucleic acid between host cells derived from disparate organisms; these are known in the art as shuttle vectors. Plasmid vectors can also insert a subject nucleic acid into a host cell's chromosome; these are known in the art as insertion vectors.

Expression vectors of the invention are cloning vectors that contain regulatory sequences that allow transcription and translation of a cloned gene or genes and thus transcribe and clone DNA. They can be used to express the polypeptides of the invention and typically include restriction sites to provide for the insertion of nucleic acid sequences encoding heterologous protein or RNA molecules. Artificially constructed plasmids, i.e., small, independently replicating pieces of extrachromosomal cytoplasmic DNA that can be transferred from one organism to another, are commonly used as cloning vectors.

Vectors can express either sense or antisense RNA transcripts of the invention in vitro (e.g., in a cell-free system or within an in vitro cultured host cell); these are known in the art as expression vectors. Expression vectors can also produce a subject polypeptide encoded by a subject nucleic acid. The expression vectors of the invention include both prokaryotic and eukaryotic expression vectors. The expression vectors of the invention provide a transcriptional and translational initiation region, which may be inducible or constitutive, where the coding region is under the transcriptional control of the transcriptional initiation region, and a transcriptional and translational termination region. These control regions can be native to a gene encoding the subject peptides, or can be derived from exogenous sources. Prior to vector insertion, a DNA of interest is obtained in a form substantially free of other nucleic acid sequences. The DNA can be recombinant, and flanked by one or more nucleotides with which it is not normally associated on a naturally occurring chromosome.

The expression vectors of the invention will generally have convenient restriction sites located near the promoter sequence to provide for the insertion of nucleic acid sequences encoding heterologous proteins. A selectable marker operative in the expression host can be present. Expression cassettes can be prepared comprising a transcription initiation region, the gene or fragment thereof, and a transcriptional termination region.

Expressed proteins and polypeptides can be obtained from naturally occurring sources or produced synthetically. For example, the proteins can be derived from biological sources that express the proteins. The proteins can also be derived synthetically, e.g., by expressing a recombinant gene encoding a protein of interest in a suitable host. Convenient protein purification procedures can be employed (Deutscher, 1990). For example, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography.

Specifically, the invention provides a vector comprising the nucleic acid molecule comprising one or more polynucleotide sequence of SEQ ID NOS.:1-54, a complement thereof, a fragment thereof, a variant thereof, or at least one polynucleotide sequence that encodes SEQ ID NOS.:55-108, a fragment thereof, or a variant thereof; and a promoter that drives the expression of the nucleic acid molecule. The invention also provides that the promoter of such a vector can be naturally contiguous to the nucleic acid molecule; not naturally contiguous to the nucleic acid molecule; inducible; conditionally active, such as the cre-lox promoter, constitutive; and/or tissue specific.

Promoters of the invention provide DNA regulatory regions capable of binding RNA polymerase and initiating transcription of an operably linked downstream (5′ to 3′ direction) coding sequence. Promoters of the invention include those comprising the minimum number of bases or elements necessary to initiate transcription of a gene of interest at levels detectable above background. Within the promoter region may exist a transcription initiation site, as well as protein binding domains (consensus sequences) responsible for the binding of RNA polymerase. Eucaryotic promoters will often, but not always, contain “TATA” boxes and “CAT” boxes.

The invention includes heterologous and homologous promoters. Heterologous promoters are derived from a different gene, cell, tissue, or genetic sources different from those to which they are operably linked. These encompass promoters of different species, e.g., a rat promoter is heterologous to a human gene when the rat promoter is operatively linked to the human gene. Heterologous promoters can be natural, i.e., they regulate in nature and without artificial aid, or they can be artificial. The invention also includes tissue specific promoters, which initiate transcription exclusively or selectively in one or a few tissue types.

In some embodiments, the promoter is a heterologous promoter, for example one that naturally encodes the polypeptide of SEQ ID NO:55-108. In some embodiments, the promoter is tissue specific, i.e., it only permits transcription from selected tissues. For example, the α-1 antitrypsin promoter is selective for lung tissue, albumin promoters are selective for hepatocytes, tyrosine hydrolase promoters are selective for melanocytes, villin promoters are selective for intestinal epithelium, glial fibrillary acidic protein promoters are selective for astrocytes, myelin basic protein promoters are selective for glial cells, and the immunoglobulin gene enhancer promoter is selective for B lymphocytes.

Promoters of the invention vary in strength; promoter sequences at which RNA polymerase initiates transcription at a high frequency are classified as “strong,” and those with a low frequency of initiation as “weak.” Promoters of the invention can be naturally occurring or engineered sequences. They include constitutive promoters, which are active unless repressed. They also include inducible promoters, which function as promoters upon receiving a predetermined stimulus. They further include conditionally active promoters, which are active only under defined circumstances, e.g., the cre-lox promoter.

Some promoters are “constitutive,” and direct transcription in the absence of regulatory influences. Some promoters are “tissue specific,” and initiate transcription exclusively or selectively in one or a few tissue types; these are described in further detail below. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline for example, rtTA a reverse tetracycline controlled transactivator.

The invention includes DNA sequences that allow for the expression of biologically active fragments of the polypeptides of the invention. These include functional epitopes or domains, at least about 8 amino acids in length, at least about 15 amino acids in length, or at least about 25 amino acids in length, or any of the above-described fragments, up to and including the complete open reading frame of the gene. After introduction of these DNA sequences, the cells containing the construct can be selected by means of a selectable marker, and the selected cells expanded and used as expression-competent host cells.

Cell-Free Expression Systems

Cell-free translation systems can be employed to produce proteins of the invention using RNAs derived from the DNA constructs of the present invention. Appropriate cloning and expression vectors, e.g., those containing SP6 or T7 promoters for use with prokaryotic and eukaryotic hosts, are known (Sambrook et al., 2001). These DNA constructs can be used to produce proteins in a rabbit reticulocyte lysate system, with wheat germ extracts, or with a frog oocyte system.

Expression in Host Cells

The invention provides a host cell comprising the nucleic acid sequence of SEQ ID NOS.:1-54. It provides a recombinant host cell comprising one or more vector with one or more nucleic acid molecules comprising one or more polynucleotide sequence of SEQ ID NOS.:1-54, a complement thereof, a fragment thereof, a variant thereof, or at least one polynucleotide sequence that encodes SEQ ID NOS.:55-108, a fragment thereof, or a variant thereof. It also provides a recombinant host cell comprising one or more isolated polynucleic acid molecule comprising one or more nucleotide sequence encoding a sense or antisense sequence of an amino acid molecule with a first polypeptide comprising the amino acid sequence of SEQ. ID. NOS.:55-108 or one or more biologically active fragments thereof. Host cells of the invention can be prokaryotic cell, a eucaryotic cell, a human cell, a mammalian cell, an insect cell, a fish cell, a plant cell, and a fungal cell.

Host cells of the invention include an individual cell, cell line, cell culture, or in vivo cell, which can be or has been a recipient of any polynucleotides or polypeptides of the invention, for example, a recombinant vector, an isolated polynucleotide, antibody, or fusion protein. Host cells include progeny of a single host cell; the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, such as human, non-human primate, and rodent; insect; amphibian; reptile; crustacean; avian; fish; plant; and fungal cells. A host cell includes cells transformed, transfected, transduced, or infected in vivo or in vitro with a polynucleotide of the invention, for example, a recombinant vector. The invention provides recombinant host cells, which comprise a recombinant vector of the invention.

Host cells of the invention can express proteins and polypeptides in accordance with conventional methods, the method depending on the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g., COS 7 cells, can be used as the expression host cells. In some situations, it is desirable to express eukaryotic genes in eukaryotic cells, where the encoded protein will benefit from native folding and post-translational modifications.

When any of the above-referenced host cells, or other appropriate host cells or organisms, are used to duplicate and/or express the polynucleotides of the invention, the resulting duplicated nucleic acid, RNA, expressed protein, or polypeptide, is within the scope of the invention as a product of the host cell or organism. The product can be recovered by any appropriate means known in the art.

The sequence of a gene, including promoter regions and coding regions, can be mutated in various ways known in the art to generate targeted changes in promoter strength or in the sequence of the encoded protein. The DNA sequence or protein product of such a mutation will usually be substantially similar to the sequences provided herein, for example, will differ by at least one nucleotide or amino acid, respectively, and may differ by at least two nucleotides or amino acids. The sequence changes may be substitutions, insertions, deletions, or a combination thereof. Deletions may further include larger changes, such as deletions of a domain or exon. Other modifications of interest include epitope tagging, e.g., with the FLAG system or hemagglutinin.

Techniques for in vitro mutagenesis of cloned genes are known. Examples of protocols for site specific mutagenesis may be found in Gustin and Burk, 1993; Barany, 1985; Colicelli et al., 1985; and Prentki and Krisch, 1984. Methods for site specific mutagenesis can be found in Sambrook et al., 2001; Weiner et al., 1993; Sayers et al., 1992; Jones and Winistorfer, 1992; Barton et al., 1990; Marotti and Tomich, 1989; and Zhu, 1989. Such mutated genes may be used to study structure-function relationships of the subject proteins, or to alter properties of the protein that affect its function or regulation.

One may also provide for gene expression, e.g., a subject gene or variants thereof, in cells or tissues where it is not normally expressed, at levels not normally present in such cells or tissues, or at abnormal times of development. One may also generate host cells (including host cells in transgenic animals, Pinkert, 1994) that comprise a heterologous nucleic acid molecule which encodes a polypeptide which functions to modulate expression of an endogenous promoter or other transcriptional regulatory region.

DNA constructs for homologous recombination will comprise at least a portion of the human gene or of a gene native to the species of the host animal, wherein the gene has the desired genetic modification(s), and includes regions of homology to the target locus. DNA constructs for random integration need not include regions of homology to mediate recombination. Conveniently, markers for positive and negative selection are included. Methods for generating cells having targeted gene modifications through homologous recombination are known in the art. For various techniques for transfecting mammalian cells, see Keown et al., 1990.

Specific cellular expression systems of interest include plants, bacteria, yeast, insect cells and mammalian cell-derived expression systems. Representative systems from each of these categories are provided below.

Plants

Expression systems in plants include those described in U.S. Pat. No. 6,096,546 and U.S. Pat. No. 6,127,145.

Bacteria

Expression systems in bacteria include those described by Chang et al., 1978; Goeddel et al., 1979; Goeddel et al., 1980; EP 0 036,776; U.S. Pat. No. 4,551,433; DeBoer et al., 1983; and Siebenlist et al., 1980.

Yeast

Expression systems in yeast include those described by Hinnen et al., 1978; Ito et al., 1983; Kurtz et al., 1986; Kunze et al., 1985; Gleeson et al., 1986; Roggenkamp et al., 1984; Das et al., 1984; De Louvencourt et al., 1983; Van den Berg et al., 1990; Kunze et al., 1985; Cregg et al., 1985; U.S. Pat. Nos. 4,837,148 and 4,929,555; Beach and Nurse, 1981; Davidow et al., 1987; Gaillardin et al., 1987; Ballance et al., 1983; Tilburn et al., 1983; Yelton et al., 1984; Kelly and Hynes, 1985; EP 0 244,234; WO 91/00357; and U.S. Pat. No. 6,080,559.

Insects

Expression systems for heterologous genes in insects includes those described in U.S. Pat. No. 4,745,051; Friesen et al., 1986; EP 0 127,839; EP 0 155,476; Vlak et al., 1988; Miller et al., 1988; Carbonell et al., 1988; Maeda et al., 1985; Lebacq-Verbeyden et al., 1988; Smith et al., 1985); Miyajima et al., 1987; and Martin et al., 1988. Numerous baculoviral strains and variants and corresponding permissive insect host cells are described in Luckow et al., 1988, Miller et al., 1988, and Maeda et al., 1985.

Mammals

Mammalian expression systems include those described in Dijkema et al., 1985; Gorman et al., 1982; Boshart et al., 1985; and U.S. Pat. No. 4,399,216. Additional features of mammalian expression are facilitated as described in Ham and McKeehan, 1979; Barnes and Sato, 1980 U.S. Pat. Nos. 4,767,704, 4,657,866, 4,927,762, 4,560,655, WO 90/103430, WO 87/00195, and U.S. RE 30,985.

Accordingly, the invention provides an isolated amino acid molecule comprising a polypeptide sequence with the amino acid sequence of SEQ ID NOS.: 55-108, a complement thereof, a fragment thereof, or a variant thereof. This polypeptide can be encoded by SEQ ID NOS.:1-54, or one or more of its biologically active fragments, and/or variants thereof.

The polypeptides of the invention can be optimized for expression in each of the expression systems described above. The invention provides an isolated amino acid molecule comprising a polypeptide with the amino acid sequence or one or more of its biologically active fragments, and/or a variant thereof, wherein the polypeptide is encoded by SEQ ID NO.:1-54 or one or more of its biologically active fragments, and wherein the polypeptide sequence is optimized for expression in a cell-free expression system, an E. coli expression system, a yeast expression system, an insect expression system, and/or a mammalian cell expression system. For example, particular sequences can be introduced into the expression vector which optimize the expression of the protein in a yeast vector; other sequences can optimize the expression of the protein in a plant vector, and so forth. These sequences are known to skilled artisans and are described in the cited references.

The invention provides a host cell transformed, transfected, transduced, or infected with one or more of the nucleic acid sequences of SEQ ID NOS.:1-54, one or more complements and/or biologically active fragments thereof, and/or one or more polynucleotide sequence that encodes SEQ ID NOS.:55-108. It also provides a recombinant host cell comprising one or more isolated polynucleic acid molecules comprising one or more nucleotide sequences encoding a sense or antisense sequence of an amino acid molecule with a first polypeptide comprising the amino acid sequence of SEQ. ID. NOS.:55-108 or one or more biologically active fragments thereof. It further provides a recombinant host cell comprising an amino acid molecule comprising a first polypeptide with an amino acid sequence of one or more of SEQ. ID. NOS.:55-108 or a biologically active fragment thereof.

Transgenic Animals

The polypeptides of the invention can also be expressed in animals, for example, transgenic animals. Animals of any species, including, but not limited to, mice, rats, rabbits, hamsters, guinea pigs, pigs, micro-pigs, goats, sheep, cows, and non-human primates, e.g., baboons, monkeys, and chimpanzees, may be used to generate transgenic animals. In a specific embodiment, techniques described herein or otherwise known in the art, are used to express polypeptides of the invention in humans, as part of a gene therapy protocol, as discussed in greater detail below.

Any technique known in the art may be used to introduce the transgene (i.e., polynucleotides of the invention) into animals to produce founder lines of transgenic animals. Such techniques include, but are not limited to, pronuclear microinjection (Paterson et al., 1994; Carver et al., 1993; Wright et al., 1991; and Hoppe et al., U.S. Pat. No. 4,873,191, 1989); retrovirus mediated gene transfer into germ lines (Van der Putten et al., 1985); blastocysts or embryos; gene targeting in embryonic stem cells (Thompson et al., 1989); electroporation of cells or embryos (Lo, 1983); introduction of the polynucleotides of the invention using a gene gun (see, e.g., Ulmer et al., 1993); introducing nucleic acid constructs into embryonic pluripotent stem cells and transferring the stem cells back into the blastocyst; and sperm-mediated gene transfer (Lavitrano et al., 1989). For a review of such techniques, see Gordon, 1989. See also, U.S. Pat. No. 5,464,764; U.S. Pat. No. 5,631,153; U.S. Pat. No. 4,736,866; and U.S. Pat. No. 4,873,191. Any technique known in the art may be used to produce transgenic clones containing polynucleotides of the invention, for example, nuclear transfer into enucleated oocytes of nuclei from cultured embryonic, fetal, or adult cells induced to quiescence (Campell et al., 1996; Wilmut et al., 1997).

The present invention provides for transgenic animals that carry the transgene in all their cells, as well as animals which carry the transgene in some, but not all their cells, i.e., mosaic animals or chimeras. The transgene may be integrated as a single transgene or as multiple copies, such as in concatamers, e.g., head-to-head tandem or head-to-tail tandem genes. The transgene may also be selectively introduced into and activated in a particular cell type by following, for example, the teaching of Lakso et al. (Lakso et al., 1992). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al., 1994. The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polynucleotides and polypeptides of the invention, studying conditions and/or disorders associated with aberrant expression, and in screening for compounds effective in ameliorating such conditions and/or disorders.

Accordingly, the invention provides an animal comprising a nucleic acid molecule with at least one polynucleotide sequence of SEQ ID NO.:1-54, a complement thereof. a fragment thereof, a variant thereof, or a polynucleotide sequence that encodes SEQ ID NO.:55-108 or one of its fragments or variants. The invention also provides an animal comprising at least one amino acid molecule comprising an amino acid sequence chosen from SEQ ID NO.:55-108 or one of its fragments or variants. The invention further provides a genetically modified mouse with a deletion, substitution, or modification of one or more polynucleotide sequence of SEQ ID NOS.:1-54 or one or more of the amino acids of SEQ ID NOS.:55-108 that prevents or reduces expression of the sequence, and results in a mouse deficient in or completely lacking one or more gene products of that sequence.

The animals may comprise a nucleic acid or amino acid molecule of the invention for research and/or treatment purposes. These may comprise a nucleic acid or amino acid molecule of the invention as a result of their introduction into a blastocyst. They may comprise a nucleic acid or amino acid molecule of the invention after treatment with a therapeutic composition, as described in more detail below. Embodiments of the animals of the invention include the animals comprising a the reporter system, as described in greater detail below.

Reporter Systems

The invention provides reporter systems for cellular functions activated by gene expression; these systems include activity-specific promoters linked to “readouts” which can be produced efficiently by introducing the reporter systems into non-human animals. The reporter systems can be introduced into embryonic stem (ES) cells, which can then be incorporated into one or more blastocysts, which can in turn be implanted into pseudo-pregnant non-human animals to produce chimeric animals expressing the reporter in a broad range of tissues.

Through this approach, transfecting a single ES cell can produce multiple transfected cell types, some of which may be otherwise difficult to transfect in their differentiated state. Substantially all the tissues of the resulting chimera have the potential to activate the reporter system upon responding to specific exogenous signals. The reporter systems can be specific for a single cell activity or can be expressed upon activation of any of the multiple activities. The reporter systems can also be specific for multiple integrated activities, for example, signal transduction pathways by including the relevant combination of pathway components, e.g., transcription factor binding sites. The different cell types of the chimeric animals can be used to detect activation, for example, by growth or differentiation factors that bind to cell surface receptors and activate an activity detected by the reporter. The cells can also be used in vivo and in vitro to measure the effect of signal transduction modulators, such as small molecules, or antibody agonists or antagonists of the pathway detected by the reporter system.

The invention provides an embryonic stem cell comprising one or more of SEQ ID NOS.:1-54 or a complement or fragment thereof, introduced at a gene locus such that the polynucleotide is expressed in more than one cell type upon differentiation of the embryonic stem cell. Transfected ES cells can be used to make chimeric animals that express the reporter in various specified tissues, such as by use of tissue-specific promoters. These chimeric animals can be used to test or determine which tissues respond to protein factors or small molecules administered to the animals. This in vivo reporter system can be used to test drug efficacy, toxicity, pharmacokinetics, and metabolism.

Examples of suitable tissue-specific promoters include the astrocyte-specific (CNS) promoter for glial fibrillary acidic protein (GFAP), a brain-specific promoter; kidney androgen regulated protein (KAP), the kidney-specific promoter for kidney androgen regulated protein (KAP); the adipocyte-specific promoter for adipocyte specific protein (ap2), the blood vessel endothelium-specific promoter for vascular endothelial growth factor receptor 2 (VEGFR2), the liver-specific promoter for albumin, the pancreas-specific promoter for pancreatic duodenal homeobox 1 (PDX1), the muscle-specific promoter for muscle creatine kinase (MCK), the bone-specific promoter for osteocalcin, the cartilage-specific promoter for type II collagen, the lung-specific promoter for surfactant protein C(SP-C), the cardiac-specific promoter alpha-myosin heavy chain (α-MHC), and the intestinal epithelial-specific promoter fatty acid binding protein (FABP).

The astrocyte-specific (CNS) promoter for glial fibrillary acidic protein (GFAP) has been described by Miura et al., 1990. The promoter sequence and transcriptional startpoint of the GFAP gene have been characterized; the cis elements for astrocyte specific expression are located within 256 base pairs from the transcription startpoint. DNase I footprinting has shown three trans-acting factor binding sites, GFI, GFII, and GFIII, which have AP-2, NFI, and cyclic AMP-responsive element motifs, respectively (Miura et al., 1990).

The kidney-specific promoter for kidney androgen regulated protein (KAP) has been described by Ding et al., 1997. Transgenic mice with an exogenous 1542-base pair fragment of the kidney androgen-regulated protein (KAP) promoter specifically targeted inducible expression to the kidney. In situ hybridization demonstrated that expression of KAP mRNA was restricted to proximal tubule epithelial cells in the renal cortex (Ding et al., 1997).

The adipocyte-specific promoter for adipocyte specific protein (ap2), which is dysregulated in various forms of obesity, has structural similarity to tumor necrosis factor (TNF) alpha, and is involved in whole body energy homeostasis. It has been described by Hunt et al. to contain sequence information necessary for differentiation-dependent expression in adipocytes (Hunt et al., 1986).

The blood vessel endothelium-specific promoter for vascular endothelial growth factor receptor 2 (VEGFR2) was described by Ronicke et al., 1996. Using RNase protection and primer extension analyses, they revealed a single transcriptional start site located 299 base pairs upstream from the translational start site in an initiator-like pyrimidine-rich sequence. The 5′-flanking region was found to be rich in GC residues and lacking a typical TATA or CAAT box. A luciferase reporter construct containing a fragment from nucleotides −1900 to +299 showed strong endothelium-specific activity in transfected bovine aortic endothelial cells. Deletion analyses revealed that endothelium-specific VEGFR expression was stimulated by the 5′-untranslated region of the first exon, which contains an activating element between nucleotides +137 and +299. In addition, two endothelium-specific negative regulatory elements were identified between nucleotides −4100 and −623. Two strong general activating elements were observed to be present in the region between nucleotides −96 and −37, which contains one potential NFκB and three potential transcription factor binding sites. This study showed that VEGFR expression in endothelial cells is regulated by an endothelium-specific activating element in the long 5′-untranslated region of the first exon and by negative regulatory elements located further upstream (Ronicke et al., 1996).

The liver-specific promoter for albumin was described by Power et al., 1994, who cloned the bovine serum albumin (bSA) promoter. It functions efficiently in the differentiated, but not dedifferentiated, liver cells. Footprint analysis of the promoter revealed seven sites of DNA protein interaction extending from −31 to −213. The deletion of one of these sites, extending from −170 to −236, results in a four fold increase in promoter activity (Power et al., 1994).

The pancreas-specific promoter for pancreatic duodenal homeobox 1 (PDX1) was described by Melloul et al., 2002. Upstream sequences of the gene up to about −6 kb were demonstrated to show islet-specific activity in transgenic mice, and several distinct sequences that conferred beta-cell-specific expression were identified. A conserved region localized to the proximal promoter around an E-box motif was found to bind members of the upstream stimulatory factor family of transcription factors (Melloul et al., 2002).

The muscle-specific promoter for muscle creatine kinase (MCK) was described by Larochelle et al., 1997 as having relatively small size, good efficiency, and muscle specificity. They generated replication-defective adenovirus recombinants with luciferase or beta-galactosidase reporter genes driven by a truncated (1.35 kb) MCK promoter/enhancer region that demonstrated efficient and muscle-specific transgene expression after local injection into muscle (Larochelle et al., 1997).

The bone-specific promoter for osteocalcin was described by Bortell, et al., who found protein-DNA interactions at the vitamin D responsive element of the rat osteocalcin gene at nucleotides −466 to −437. They also found a vitamin D-responsive increase in osteocalcin gene transcription accompanied by enhanced non-vitamin D receptor-mediated protein-DNA interactions in the “TATA” box region (nucleotides −44 to +23), which contains a potential glucocorticoid responsive element. An osteocalcin CCAAT box was described at nucleotides −99 to −76 (Bortell et al., 1992).

The cartilage-specific promoter for type II collagen was described by Osaki et al., 2003. Luciferase reporter constructs containing sequences of the type II collagen promoter spanning −6368 to +125 base pairs were reported to be inhibited by the type II collagen inhibitor interferon-gamma. The interferon-gamma response was retained in the type II collagen core promoter region spanning −45 to +11 base pairs, containing the TATA-box and GC-rich sequences.

The intestinal epithelial-specific fatty acid binding protein promoter (FABP) was described by Sweetser et al. as both cell-specific and exhibiting regional differences in its expression within continuously regenerating small intestinal epithelium. Sequences located within 277 nucleotides of the start site of intestinal FABP transcription were reported to be sufficient to limit reporter gene (human growth hormone) expression to the intestine. Nucleotides −278 to −1178 of the intestinal FABP gene mediated its expression in the distal jejunum and ileum (Sweetser et al., 1988).

The lung-specific promoter for surfactant protein C (SP-C) was described by Glasser et al. This group identified the transcriptional start site and a TATAA consensus element located 29 base pairs five prime to exon 1 (Glasser et al., 1990).

The cardiac-specific promoter alpha-myosin heavy chain (α-MHC) was described by Molkentin et al. They reported that sequences from −344 to −156 directed cardiac-muscle specific expression from a heterologous promoter, and this region included a CARG box. They also reported that α-MHC sequences from −86 to +16 promoted activity from two heterologous enhancers in a muscle-specific fashion, and that mutational analysis of an E-box and a CARG box within the promoter revealed that they act as negative and positive regulatory elements, respectively (Molkentin et al., 1996).

The invention also provides a system for conducting in vivo and in vitro testing of the cellular function of a gene product. The system provides targeting a gene to a locus, e.g., the ROSA 26 locus in mouse ES cells and allowing the transfected DNA to proliferate and differentiate in vitro. The ROSA 26 locus directs the ubiquitous expression of the heterologous gene (U.S. Pat. No. 6,461,864). For example, the effect of the transfected DNA on healthy or diseased cells can be monitored in vitro. Differentiation of cells, e.g., cardiomyocytes, hepatocytes, skeletal myocytes, etc. can be monitored by morphologic, histologic, and/or physiologic criteria.

The tissues of the chimeric mice or their progeny can be isolated and studied, or cells and/or cell lines can be isolated from the tissues and studied. Tissues and cells from any organ in the body, including heart, liver, lung, kidney, spleen, thymus, muscle, skin, blood, bone marrow, prostate, breast, stomach, brain, spinal cord, pancreas, ovary, testis, eye, and lymph node are suitable for use.

This in vivo reporter system can be used to test drug efficacy, toxicity, pharmacokinetics, and metabolism. Examining reporter gene expression in cells, tissues, and animals that have been treated with a candidate therapeutic agent provides information about the effect of the candidate agent on the signal transduction system or systems.

Diagnostic Kits and Methods

The invention provides a kit comprising one or more of a polynucleotide, polypeptide, or modulator composition, such as an antibody composition, which may include instructions for its use. Such kits are useful in diagnostic applications, for example, to detect the presence and/or level of a polypeptide in a biological sample by specific antibody interaction. Specifically, the invention provides a diagnostic kit comprising a nucleic acid molecule that comprises a sequence of at least 6, at least 7, at least 8, or at least 9 contiguous nucleotides chosen from a nucleic acid molecule comprising a polynucleotide sequence according to SEQ ID NOS.:1-54, or their complements, fragments, or variants, or a polynucleotide sequence that encodes a polypeptide sequence according to SEQ ID NOS.:55-108, or their fragments or variants.

A kit, or pharmaceutical pack, of the invention can comprise one or more containers filled with one or more of the ingredients of the pharmaceutical compositions of the invention, as described in more detail below. Associated with such container(s) can be a notice in the form prescribed by a governmental agency regulating the manufacture, use, or sale of pharmaceuticals or biological products, which notice reflects approval by the agency of manufacture, use, or sale for human administration.

Kits that detect a polynucleotide can comprise a moiety that specifically hybridizes to a polynucleotide of the invention. The primer nucleic acids can be prepared using any known method, e.g., automated synthesis. In some embodiments, one or both members of the pair of nucleic acid molecules comprise a detectable label. Kits of the invention for detecting a subject polypeptide will comprise a moiety that specifically binds to a polypeptide of the invention; the moiety includes, but is not limited to, a polypeptide-specific antibody.

Kits for detecting polynucleotides can also comprise a pair of nucleic acids in a suitable storage medium, e.g., a buffered solution, in a suitable container. The pair of isolated nucleic acid molecules serve as primers in an amplification reaction, e.g., a polymerase chain reaction. The kit can further include additional buffers, reagents for polymerase chain reaction, e.g., deoxynucleotide triphosphates (dNTP), a thermostable DNA polymerase, a solution containing Mg²⁺ ions, e.g., MgCl₂, and other components well known to those skilled in the art for carrying out a polymerase chain reaction. The kit can further include instructions for use, which may be provided in a variety of forms, e.g., printed information, or compact disc. The kit may further include reagents necessary to extract DNA from a biological sample and reagents for generating a cDNA copy of an mRNA. The kit may optionally provide additional useful components, including, but not limited to, buffers, developing reagents, labels, reacting surfaces, means for detections, control samples, standards, and interpretive information.

The kits of the invention can detect one or more molecules of the invention present in biological samples, including biological fluids such as blood, serum, plasma, urine, cerebrospinal fluid, tears, saliva, lymph, dialysis fluid, lavage fluid, semen, and other liquid samples of biological origin. A biological sample can include cells and their progeny, including cells in situ, cells ex vivo, cells in culture, cell supernatants, and cell lysates. It can include organ or tissue culture derived fluids, tissue biopsy samples, tumor biopsy samples, stool samples, and fluids extracted from cells and tissues. Cells dissociated from solid tissues, tissue sections, and cell lysates are also included. A biological sample can comprise a sample that has been manipulated after its procurement, such as by treatment with reagents, solubilization, or enrichment for certain components, such as polynucleotides or polypeptides. Biological samples suitable for use in the kit also include derivatives and fractions of biological samples.

The kits are useful in diagnostic applications. For example, the kit is useful to determine whether a given DNA sample isolated from an individual comprises an expressed nucleic acid, a polymorphism, or other variant. The kit can be used to detect a specific disorder or disease, i.e., a pathological, abnormal, and/or harmful condition which can be identified by symptoms or other identifying factors as diverging from a healthy or a normal state, including syndromes, conditions, and injuries and their resulting damage, e.g., trauma, skin ulcers, surgical wounds, and burns.

The invention provides a method of diagnosing a disease, disorder, syndrome, or condition chosen from cancer, proliferative, inflammatory, immune, metabolic, genetic, bacterial, and viral diseases, disorders, syndromes, or conditions in a patient by providing an antibody that specifically recognizes, binds to, and/or modulates the biological activity of at least one polypeptide encoded by a nucleic acid molecule comprising a polynucleotide sequence according to SEQ ID NOS.:1-54, a complement or variant thereof, or at least one polynucleotide sequence that encodes SEQ ID NOS.:55-108, or a biologically active fragment or variant thereof, allowing the antibody to contact a patient sample; and detecting specific binding between the antibody and an antigen in the sample to determine whether the subject has such a disease.

The invention also provides a method of diagnosing a disease, disorder, syndrome, or condition chosen from cancer, proliferative, inflammatory, immune, bacterial, and viral diseases, disorders, syndromes, or conditions in a patient by providing a polypeptide that specifically binds to an antibody, or biologically active fragment of an antibody, which specifically recognizes, binds to, and/or modulates the biological activity of at least one polypeptide encoded by a molecule of the invention; allowing the polypeptide to contact a patient sample; and detecting specific binding between the polypeptide and any interacting molecule in the sample to determine whether the subject has cancer, a proliferative, inflammatory, immune, bacterial, or viral disease, disorder, syndrome, or condition.

The invention also provides a method for determining the presence or measuring the level of a polypeptide that specifically binds to an antibody of the invention. This method involves allowing the antibody to interact with a sample, and determining whether interaction between the antibody and any polypeptide in the sample has occurred. Antibodies that specifically bind to at least one subject polypeptide are useful in diagnostic assays, e.g., to detect the presence of a subject polypeptide. Similarly, the invention features a method of determining the presence of an antibody to a polypeptide of the invention, by providing the polypeptide, allowing the antibody and the polypeptide to interact, and determining whether interaction has occurred.

Specifically, the invention provides a method of determining the presence of a nucleic acid molecule comprising a polynucleotide sequence chosen from at least one polynucleotide sequence according to SEQ ID NOS.:1-54, a complement thereof, a fragment thereof, a variant thereof, a polynucleotide sequence that encodes SEQ ID NOS.:55-108, a fragment thereof, and a variant thereof, or a complement of such a nucleic acid molecule by providing a complement to the nucleic acid molecule or providing a complement to the complement of the nucleic acid molecule; allowing the molecules to interact; and determining whether interaction has occurred.

The invention further provides a method of determining the presence of an antibody to an amino acid molecule comprising a polypeptide sequence chosen from amino acid sequence according to SEQ ID NOS.:55-108, a complement thereof, a fragment thereof, and a variant thereof in a sample, by providing the amino acid molecule; allowing the amino acid molecule to interact with any specific antibody in the sample; and determining whether interaction has occurred.

The invention also provides a method of diagnosing cancer, proliferative, inflammatory, immune, viral, bacterial, or metabolic disorder in a patient, by allowing an antibody specific for a polypeptide of the invention to contact a patient sample, and detecting specific binding between the antibody and any antigen in the sample to determine whether the subject has cancer, proliferative, inflammatory, immune, viral, bacterial, or metabolic disorder.

The invention further provides a method of diagnosing cancer, proliferative, inflammatory, immune, viral, bacterial, or metabolic disorder in a patient, by allowing a polypeptide of the invention to contact a patient sample, and detecting specific binding between the polypeptide and any interacting molecule in the sample to determine whether the subject has cancer, proliferative, inflammatory, immune, viral, bacterial, or metabolic disorder.

The invention provides diagnostic kits and methods for diagnosing disease states based on the detected presence, amount, and/or biological activity of polynucleotides or polypeptides in a biological sample. These detection methods can be provided as part of a kit which detects the presence amount, and/or biological activity of a polynucleotide or polypeptide in a biological sample. Procedures using these kits can be performed by clinical laboratories, experimental laboratories, medical practitioners, or private individuals.

Diagnostic methods in which the level of expression is of interest will typically involve determining whether a specific nucleic acid or amino acid molecule is present, and/or comparing its abundance in a sample of interest with that of a control value to determine any relative differences. These differences can then be measured qualitatively and/or quantitatively, and differences related to the presence or absence of an abnormal expression pattern. A variety of different methods for determining the presence or absence of a nucleic acid or polypeptide in a biological sample are known to those of skill in the art; particular methods of interest include those described by Soares, 1997; Pietu et al., 1996; Stolz and Tuan, 1996; Zhao et al., 1995; Chalifour et al., 1994; Raval, 1994; McGraw, 1984; and Hong, 1982. Also of interest are the methods disclosed in WO 97/27317.

Where the kit provides for mRNA detection, detection of hybridization, when compared to a suitable control, is an indication of the presence in the sample of a subject polynucleotide. Appropriate controls include, for example, a sample which is known not to contain subject polynucleotide mRNA, and use of a labeled polynucleotide of the same “sense” as a subject polynucleotide mRNA. Conditions which allow hybridization are known in the art and described in greater detail above. Detection can be accomplished by any known method, including, but not limited to, in situ hybridization, PCR, RT-PCR, and “Northern” or RNA blotting, or combinations of such techniques, using a suitably labeled subject polynucleotide. Specific hybridization can be determined by comparison to appropriate controls.

Where the kit provides for polypeptide detection, it can include one or more specific antibodies. In some embodiments, the antibody specific to the polypeptide is detectably labeled. In other embodiments, the antibody specific to the polypeptide is not labeled; instead, a second, detectably-labeled antibody is provided that binds to the specific antibody. The kit may further include blocking reagents, buffers, and reagents for developing and/or detecting the detectable marker. The kit may further include instructions for use, controls, and interpretive information.

Detection of specific binding of an antibody, when compared to a suitable control, is an indication that a subject polypeptide is present in the sample. Suitable controls include a sample known not to contain a subject polypeptide, and a sample contacted with an antibody not specific for the subject polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. These methods are known to those skilled in the art (Harlow et al., 1998; Harlow and Lane, 1988).

Where the kit provides for specific antibody detection, it can include one or more polypeptides. In some embodiments, the polypeptide is detectably labeled. In other embodiments, the polypeptide is not labeled; instead, a detectably-labeled ligand or second antibody is provided that specifically binds to the polypeptide. The kit may further include blocking reagents, buffers, and reagents for developing and/or detecting the detectable marker. The kit may further include instructions for use, controls, and interpretive information.

The invention further provides for kits with unit doses of an active agent. These agents are described in more detail below. In some embodiments, the agent is provided in oral or injectable doses. Such kits can comprise a receptacle containing the unit doses and an informational package insert describing the use and attendant benefits of the drugs in treating a condition of interest.

The present invention provides methods for diagnosing disease states based on the detected presence and/or level of polynucleotide or polypeptide in a biological sample, and/or the detected presence and/or level of biological activity of the polynucleotide or polypeptide. These detection methods can be provided as part of a kit. Thus, the invention further provides kits for detecting the presence and/or a level of a polynucleotide or polypeptide in a biological sample and/or or the detected presence and/or level of biological activity of the polynucleotide or polypeptide. Procedures using these kits can be performed by clinical laboratories, experimental laboratories, medical practitioners, or private individuals.

Therapeutic Compositions and Methods

Therapeutic Compositions

Use of SEQ ID NOS.:1-108 has therapeutic applications for the diseases and disorders discussed above. Compositions based on these sequences, biologically active fragments, and variants thereof, can be formulated using well-known reagents and methods, and can be provided in formulation with pharmaceutically acceptable excipients, a wide variety of which are known in the art (Gennaro, 2003). Therapeutic compounds comprising these sequences can be formulated into preparations in solid, semi-solid, liquid, or gaseous forms, such as tablets, capsules, powders, granules, ointments, solutions, suppositories, injections, inhalants, and aerosols.

Typically, such a composition will contain from less than 1% to about 95% of the active ingredient, preferably about 10% to about 50%. Generally, between about 100 mg and 500 mg will be administered to a child and between about 500 mg and 5 grams will be administered to an adult. Administration is generally by injection and often by injection to a localized area. Administration may be performed by stereotactic injection. The frequency of administration will be determined by the care giver based on patient responsiveness. Other effective dosages can be readily determined by one of ordinary skill in the art through routine trials establishing dose response curves.

In order to calculate the effective amount of subject polynucleotide or polypeptide agent, those skilled in the art could use readily available information with respect to the amount of agent necessary to have a the desired effect. The amount of an agent necessary to increase a level of active subject polynucleotide or polypeptide can be calculated from in vitro experimentation. The amount of agent will, of course, vary depending upon the particular agent used.

Other effective dosages can be readily determined by one of ordinary skill in the art through routine trials establishing dose response curves, for example, the amount of agent necessary to increase a level of active subject polypeptide can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the dose, those skilled in the art can use readily available information with respect to the amount necessary to have the desired effect, depending upon the particular agent used.

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e., in food or by immersion in buffer containing the RNA (Gaudilliere et al., 2002; O'Neil et al., 2001; WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

Therapeutic and Related Methods

Identifying Interactive Biological Molecules

The present polynucleotides, polypeptides, and modulators find use in therapeutic agent screening/discovery applications, such as screening for receptors or competitive ligands, for use, for example, as small molecule therapeutic drugs. Also provided are methods of modulating a biological activity of a polypeptide and methods of treating associated disease conditions, particularly by administering modulators of the present polypeptides, such as small molecule modulators, antisense molecules, and specific antibodies.

Formation of a binding complex between a subject polypeptide and an interacting polypeptide or other macromolecule (e.g., DNA, RNA, lipids, polysaccharides, and the like) can be detected using any known method. Suitable methods include: a yeast two-hybrid system (Zhu et al., 1997; Fields and Song, 1989; U.S. Pat. No. 5,283,173; Chien et al. 1991); a mammalian cell two-hybrid method; a fluorescence resonance energy transfer (FRET) assay; a bioluminescence resonance energy transfer (BRET) assay; a fluorescence quenching assay; a fluorescence anisotropy assay (Jameson and Sawyer, 1995); an immunological assay; and an assay involving binding of a detectably labeled protein to an immobilized protein.

Immunological assays, and assays involving binding of a detectably labeled protein to an immobilized protein can be performed in a variety of ways. For example, immunoprecipitation assays can be designed such that the complex of protein and an interacting polypeptide is detected by precipitation with an antibody specific for either the protein or the interacting polypeptide.

FRET detects formation of a binding complex between a subject polypeptide and an interacting polypeptide. It involves the transfer of energy from a donor fluorophore in an excited state to a nearby acceptor fluorophore. For this transfer to take place, the donor and acceptor molecules must be in close proximity (e.g., less than 10 nanometers apart, usually between 10 and 100 Å apart), and the emission spectra of the donor fluorophore must overlap the excitation spectra of the acceptor fluorophore. In these embodiments, a fluorescently labeled subject protein serves as a donor and/or acceptor in combination with a second fluorescent protein or dye.

Fluorescent proteins can be produced by generating a construct comprising a protein and a fluorescent fusion partner. These are well-known in the art, as described above, including green fluorescent protein (GFP), i.e., a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; other fluorescent mutants of a GFP from Aequoria victoria, e.g., as described in U.S. Pat. Nos. 6,066,476; 6,020,192; 5,985,577; 5,976,796; 5,968,750; 5,968,738; 5,958,713; 5,919,445; 5,874,304; a GFP from another species such as Renilla reniformis, Renilla mulleri, or Ptilosarcus guernyi, as previously described (WO 99/49019; Peelle et al., 2001), “humanized” recombinant GFP (hrGFP) (Stratagene®); any of a variety of fluorescent and colored proteins from Anthozoan species, (e.g., Matz et al., 1999); as well as proteins labeled with other fluorescent dyes, fluorescein and it derivatives, e.g., fluorescein isothiocyanate (FITC), 6-carboxyfluorescein (6-FAM), 6-carboxy-2′,4′,7′,4,7-hexachlorofluorescein (HEX), 5-carboxyfluorescein (5-FAM), 2′,7′-dimethoxy-4′,5′-dichloro-6-carboxyfluorescein (JOE); rhodamine dyes, e.g., Texas red, phycoerythrin, tetramethylrhodamine, rhodamine, 6-carboxy-X-rhodamine (ROX); coumarin and its derivatives, e.g., 7-amino-4-methylcoumarin, aminocoumarin; bodipy dyes, such as Bodipy FL; cascade blue; Oregon green; eosins and erythrosins; cyanine dyes, e.g., allophycocyanin, Cy3, Cy5, and N,N,N′,N′-tetramethyl-6-carboxyrhodamine (TAMRA); macrocyclic chelates of lanthanide ions, e.g., quantum dye, etc; and chemiluminescent molecules, e.g., luciferases.

Fluorescent subject proteins can also be generated by producing the subject protein in an auxotrophic strain of bacteria which requires addition of one or more amino acids in the medium for growth. A subject protein-encoding construct that provides for expression in bacterial cells is introduced into the auxotrophic strain, and the bacteria are cultured in the presence of a fluorescent amino acid, which is incorporated into the subject protein produced by the bacterium. The subject protein is then purified from the bacterial culture using standard methods for protein purification.

BRET is a protein-protein interaction assay based on energy transfer from a bioluminescent donor to a fluorescent acceptor protein. The BRET signal is measured by the ratio of the amount of light emitted by the acceptor to the amount of light emitted by the donor. The ratio of these two values increases as the two proteins are brought into proximity. The BRET assay has been described in the literature (U.S. Pat. Nos. 6,020,192; 5,968,750; 5,874,304; Xu, et al. 1999). BRET assays can be performed by analyzing transfer between a bioluminescent donor protein and a fluorescent acceptor protein. Interaction between the donor and acceptor proteins can be monitored by a change in the ratio of light emitted by the bioluminescent and fluorescent proteins. In this application, the subject protein serves as donor and/or acceptor protein.

Fluorescence anisotropy is a measurement of the rotational mobility of a multi-molecular complex. It can be used to generate information about the binding of one molecule to another, including the affinity and specificity of binding sites. It can be applied to polypeptides or nucleic acids of the present invention.

Fluorescence quenching measurements are useful in detecting protein multimerization, such as where the subject protein interacts with at least a second protein and, for example, where multimerization interaction is affected by a test agent. As used herein, the term “multimerization” refers to formation of dimers, trimers, tetramers, and higher multimers of the subject protein. Whether a subject protein forms a complex with one or more additional protein molecules can be determined using any known assay, including assays as described above for interacting proteins. Formation of multimers can also be detected using non-denaturing gel electrophoresis, where multimerized subject protein migrates more slowly than monomeric subject protein. Formation of multimers can also be detected using fluorescence quenching techniques.

Formation of multimers can also be detected by analytical ultracentrifugation, for example through glycerol or sucrose gradients, and subsequent visualization of a subject protein in gradient fractions by Western blotting or staining of SDS-polyacrylamide gels. Multimers are expected to sediment at defined positions in such gradients. Formation of multimers can also be detected using analytical gel filtration, e.g., in HPLC or FPLC systems, e.g., on columns such as Superdex 200 (Pharmacia Amersham Inc.). Multimers run at defined positions on these columns, and fractions can be analyzed as above. The columns are highly reproducible, allowing one to relate the number and position of peaks directly to the multimerization status of the protein.

Detecting mRNA Levels and Monitoring Gene Expression

The present invention provides methods for detecting the presence of mRNA in a biological sample. The methods can be used, for example, to assess whether a test compound affects gene expression, either directly or indirectly. The present invention provides diagnostic methods to compare the abundance of a nucleic acid with that of a control value, either qualitatively or quantitatively, and to relate the value to a normal or abnormal expression pattern.

Methods of measuring mRNA levels are known in the art (Pietu, 1996; Zhao, 1995; Soares, 1997; Raval, 1994; Chalifour, 1994; Stolz, 1996; Hong, 1982; McGraw, 1984; WO 97/27317). These methods generally comprise contacting a sample with a polynucleotide of the invention under conditions that allow hybridization and detecting hybridization, if any, as an indication of the presence of the polynucleotide of interest. Appropriate controls include the use of a sample lacking the polynucleotide mRNA of interest, or the use of a labeled polynucleotide of the same “sense” as a polynucleotide mRNA of interest. Detection can be accomplished by any known method, including, but not limited to, in situ hybridization, PCR, RT-PCR, and “Northern” or RNA blotting, or combinations of such techniques, using a suitably labeled subject polynucleotide. A variety of labels and labeling methods for polynucleotides are known in the art and can be used in the assay methods of the invention. A common method employed is use of microarrays which can be purchased or customized, for example, through conventional vendors such as Affymetrix.

In some embodiments, the methods involve generating a cDNA copy of an mRNA molecule in a biological sample, and amplifying the cDNA using an isolated primer pairs as described above, i.e., a set of two nucleic acid molecules that serve as forward and reverse primers in an amplification reaction (e.g., a polymerase chain reaction). The primer pairs are chosen to specifically amplify a cDNA copy of an mRNA encoding a polypeptide. A detectable label can be included in the amplification reaction, as provided above. Methods using PCR amplification can be performed on the DNA from a single cell, although it is convenient to use at least about 10⁵ cells.

The present invention provides methods for monitoring gene expression. Changes in a promoter or enhancer sequence that can affect gene expression can be examined in light of expression levels of the normal allele by various methods known in the art. Methods for determining promoter or enhancer strength include quantifying the expressed natural protein, and inserting the variant control element into a vector with a quantitative reporter gene such as β-galactosidase, luciferase, or chloramphenicol acetyltransferase (CAT).

Detecting Polymorphisms and Mutations

Biochemical studies can determine whether a sequence polymorphism in a coding region or control region is associated with disease. Disease-associated polymorphisms can include deletion or truncation of the gene, mutations that alter expression level, or mutations that affect protein function, etc. A number of methods are available to analyze nucleic acids for the presence of a specific sequence, e.g., a disease associated polymorphism. Genomic DNA can be used when large amounts of DNA are available. Alternatively, the region of interest is cloned into a suitable vector and grown in sufficient quantity for analysis. Cells that express the gene provide a source of mRNA, which can be assayed directly or reverse transcribed into cDNA for analysis. The nucleic acid can be amplified by conventional techniques, i.e., PCR, to provide sufficient amounts for analysis. (Saiki et al., 1988; Sambrook et al., 1989, pp. 14.2-14.33). Alternatively, various methods are known in the art that utilize oligonucleotide ligation as a means of detecting polymorphisms (Riley et al., 1990; Delahunty et al., 1996).

The sample nucleic acid, e.g., an amplified or cloned fragment, is analyzed by one of a number of methods known in the art. The nucleic acid can be sequenced by dideoxy nucleotide sequencing, or other methods, and the sequence of bases compared to a wild-type sequence. Hybridization with the variant sequence can also be used to determine its presence, e.g., by Southern blots, dot blots, etc. The hybridization pattern of a control and variant sequence to an array of oligonucleotide probes immobilized on a solid support, as described in U.S. Pat. No. 5,445,934, or WO 95/35505, can also be used as a means of detecting the presence of variant sequences. Single strand conformational polymorphism (SSCP) analysis, denaturing gradient gel electrophoresis (DGGE), and heteroduplex analysis in gel matrices can detect variation as alterations in electrophoretic mobility resulting from conformational changes created by DNA sequence alterations. Alternatively, where a polymorphism creates or destroys a recognition site for a restriction endonuclease, the sample can be digested with that endonuclease, and the products fractionated according to their size to determine whether the fragment was digested. Fractionation can be performed by gel or capillary electrophoresis, for example with acrylamide or agarose gels.

Screening for mutations in a gene can be based on the functional or antigenic characteristics of the protein. Protein truncation assays are useful in detecting deletions that might affect the biological activity of the protein. Various immunoassays designed to detect polymorphisms in proteins can be used in screening. Where many diverse genetic mutations lead to a particular disease phenotype, functional protein assays have proven to be effective screening tools. The activity of the encoded protein can be determined by comparison with the wild-type protein.

Detecting and Monitoring Polypeptide Presence and Biological Activity

The present invention provides methods for detecting the presence and/or biological activity of a subject polypeptide in a biological sample. The assay used will be appropriate to the biological activity of the particular polypeptide. Thus, e.g., where the biological activity is an enzymatic activity, the method will involve contacting the sample with an appropriate substrate, and detecting the product of the enzymatic reaction on the substrate. Where the biological activity is binding to a second macromolecule, the assay detects protein-protein binding, protein-DNA binding, protein-carbohydrate binding, or protein-lipid binding, as appropriate, using well known assays. Where the biological activity is signal transduction (e.g., transmission of a signal from outside the cell to inside the cell) or transport, an appropriate assay is used, such as measurement of intracellular calcium ion concentration, measurement of membrane conductance changes, or measurement of intracellular potassium ion concentration.

The present invention also provides methods for detecting the presence or measuring the level of a normal or abnormal polypeptide in a biological sample using a specific antibody. The methods generally comprise contacting the sample with a specific antibody and detecting binding between the antibody and molecules of the sample. Specific antibody binding, when compared to a suitable control, is an indication that a polypeptide of interest is present in the sample. Suitable controls include a sample known not to contain the polypeptide, and a sample contacted with a non-specific antibody, e.g., an anti-idiotype antibody.

A variety of methods to detect specific antibody-antigen interactions are known in the art, e.g., standard immunohistological methods, immunoprecipitation, enzyme immunoassay, and radioimmunoassay. The specific antibody can be detectably labeled, either directly or indirectly, as described at length herein, and cells are permeabilized to stain cytoplasmic molecules. Briefly, antibodies are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, specific-binding pairs may be used, involving, e.g., a second stage antibody or reagent that is detectably-labeled, as described above. Such reagents and their methods of use are well known in the art.

Alternatively, a biological sample can be brought into contact with an immobilized antibody on a solid support or carrier, such as nitrocellulose, that is capable of immobilizing cells, cell particles, or soluble proteins. The antibody can be attached (coupled) to an insoluble support, such as a polystyrene plate or a bead. After contacting the sample, the support can then be washed with suitable buffers, followed by contacting with a detectably-labeled specific antibody. Detection methods are known in the art and will be chosen as appropriate to the signal emitted by the detectable label. Detection is generally accomplished in comparison to suitable controls, and to appropriate standards.

The present invention further provides methods for detecting the presence and/or levels of enzymatic activity of a subject polypeptide in a biological sample. The methods generally involve contacting the sample with a substrate that yields a detectable product upon being acted upon by a subject polypeptide, and detecting a product of the enzymatic reaction. Further, polypeptides that are subsets of the complete sequences of the subject proteins may be used to identify and investigate parts of the protein important for function.

The present invention further includes methods for monitoring activity of a polypeptide through observation of phenotypic changes in a cell containing such polypeptide, such as growth or differentiation, or the ability of such a cell to secrete a molecule that can be detected, such as through chemical methods or through its effect on another cell, such as cell activation.

Modulating in RNA and Peptides in Biological Samples

The present invention provides screening methods for identifying agents that modulate the level of a mRNA molecule of the invention, agents that modulate the level of a polypeptide of the invention, and agents that modulate the biological activity of a polypeptide of the invention. In some embodiments, the assay is cell-free; in others, it is cell-based. Where the screening assay is a binding assay, one or more of the molecules can be joined to a label, where the label can directly or indirectly provide a detectable signal.

The invention provides a method of identifying an agent that modulates the biological activity of a polypeptide by providing a polypeptide or one or more of it biologically active fragments or variants, wherein the polypeptide comprises at least one amino acid sequence according to SEQ ID NOS.:55-108, allowing at least one agent to contact the polypeptide; and selecting an agent that binds the polypeptide or affects the biological activity of the polypeptide. This method can be practiced with a polypeptide expressed on a cell surface.

The invention provides a modulator composition comprising a modulator and a pharmaceutically acceptable carrier, wherein the modulator is obtainable by a method of identifying an agent that modulates the biological activity of a polypeptide by providing a polypeptide or one or more of it biologically active fragments or variants, wherein the polypeptide comprises at least one amino acid sequence according to SEQ ID NOS.:55-108, allowing at least one agent to contact the polypeptide; and selecting an agent that binds the polypeptide or affects the biological activity of the polypeptide. This modulator can be an antibody.

As discussed above, the invention encompasses endogenous polynucleotides of the invention that encode mRNA and/or polypeptides of interest. Again as discussed previously, the invention also encompasses exogenous polynucleotides that encode mRNA or polypeptides of the invention. For example, the polynucleotide can reside within a recombinant vector which is introduced into the cell. For example, a recombinant vector can comprise an isolated transcriptional regulatory sequence which is associated in nature with a nucleic acid, such as a promoter sequence operably linked to sequences coding for a polypeptide of the invention; or the transcriptional control sequences can be operably linked to coding sequences for a polypeptide fusion protein comprising a polypeptide of the invention fused to a polypeptide that facilitates detection.

In these embodiments, the candidate agent is combined with a cell possessing a polynucleotide transcriptional regulatory element operably linked to a polypeptide-coding sequence of interest, e.g., a subject cDNA or its genomic component; and determining the agent's effect on polynucleotide expression, as measured, for example by the level of mRNA, polypeptide, or fusion polypeptide.

In other embodiments, for example, a recombinant vector can comprise an isolated polynucleotide transcriptional regulatory sequence, such as a promoter sequence, operably linked to a reporter gene (e.g., β-galactosidase, CAT, luciferase, or other gene that can be easily assayed for expression). In these embodiments, the method for identifying an agent that modulates a level of expression of a polynucleotide in a cell comprises combining a candidate agent with a cell comprising a transcriptional regulatory element operably linked to a reporter gene; and determining the effect of said agent on reporter gene expression.

Known methods of measuring mRNA levels can be used to identify agents that modulate mRNA levels, including, but not limited to, PCR with detectably-labeled primers. Similarly, agents that modulate polypeptide levels can be identified using standard methods for determining polypeptide levels, including, but not limited to an immunoassay such as ELISA with detectably-labeled antibodies.

A wide variety of cell-based assays can also be used to identify agents that modulate eukaryotic or prokaryotic mRNA and/or polypeptide levels. Examples include transformed cells that over-express a cDNA construct and cells transformed with a polynucleotide of interest associated with an endogenously-associated promoter operably linked to a reporter gene. A control sample would comprise, for example, the same cell lacking the candidate agent. Expression levels are measured and compared in the test and control samples.

The cells used in the assay are usually mammalian cells, including, but not limited to, rodent cells and human cells. The cells can be primary cell cultures or can be immortalized cell lines. Cell-based assays generally comprise the steps of contacting the cell with a test agent, forming a test sample, and, after a suitable time, assessing the agent's effect on macromolecule expression. That is, the mammalian cell line is transformed or transfected with a construct that results in expression of the polynucleotide, the cell is contacted with a test agent, and then mRNA or polypeptide levels are detected and measured using conventional assays.

A suitable period of time for contacting the agent with the cell can be determined empirically, and is generally a time sufficient to allow entry of the agent into the cell and to allow the agent to have a measurable effect on subject mRNA and/or polypeptide levels. Generally, a suitable time is between about 10 minutes and about 24 hours, including about 1 to about 8 hours. Alternatively, incubation periods may be between about 0.1 and about 1 hour, selected for example for optimum activity or to facilitate rapid high-throughput screening. Where the polypeptide is expressed on the cell surface, however, a shorter length of time may be sufficient. Incubations are performed at any suitable temperature, i.e., between about 4° C. and about 40° C. The contact and incubation steps can be followed by a washing step to remove unbound components, i.e., a label that would give rise to a background signal during subsequent detection of specifically-bound complexes.

A variety of assay configurations and protocols are known in the art. For example, one of the components can be bound to a solid support, and the remaining components contacted with the support bound component. Remaining components may be added at different times or at substantially the same time. Further, where the interacting protein is a second subject protein, the effect of the test agent on binding can be determined by determining the effect on multimization of the subject protein.

The present invention further provides methods of identifying agents that modulate a biological activity of a polypeptide of the invention. The method generally comprises contacting a test agent with a sample containing a subject polypeptide and assaying a biological activity of the subject polypeptide in the presence of the test agent. An increase or a decrease in the assayed biological activity in comparison to the activity in a suitable control (e.g., a sample comprising a subject polypeptide in the absence of the test agent) is an indication that the substance modulates a biological activity of the subject polypeptide. The mixture of components is added in any order that provides for the requisite interaction.

External and internal processes that can affect modulation of a macromolecule of the invention include, but are not limited to, infection of a cell by a microorganism, including, but not limited to, a bacterium (e.g., Mycobacterium spp., Shigella, or Chlamydia), a protozoan (e.g., Trypanosoma spp., Plasmodium spp., or Toxoplasma spp.), a fungus, a yeast (e.g., Candida spp.), or a virus (including viruses that infect mammalian cells, such as human immunodeficiency virus, foot and mouth disease virus, Epstein-Barr virus, and viruses that infect plant cells); change in pH of the medium in which a cell is maintained or a change in internal pH; excessive heat relative to the normal range for the cell or the multicellular organism; excessive cold relative to the normal range for the cell or the multicellular organism; an effector molecule such as a hormone, a cytokine, a chemokine, a neurotransmitter; an ingested or applied drug; a ligand for a cell-surface receptor; a ligand for a receptor that exists internally in a cell, e.g., a nuclear receptor; hypoxia; light; dark; sleep patterns; electrical charge; ion concentration of the medium in which a cell is maintained or an internal ion concentration, exemplary ions including sodium ions, potassium ions, chloride ions, calcium ions, and the like; presence or absence of a nutrient; metal ions; a transcription factor; mitogens, including, but not limited to, lipopolysaccharide (LPS), pokeweed mitogen; antigens; a tumor suppressor; and cell-cell contact and must be taken into consideration in the screening assay.

A variety of other reagents can be included in the screening assay. These include salts, neutral proteins, e.g. albumin, detergents, and other compounds that facilitate optimal binding and/or reduce non-specific or background interactions. Reagents that improve the efficiency of the assay, such as protease inhibitors, nuclease inhibitors, or anti-microbial agents, etc., can be used.

Accordingly, the present invention provides a method for identifying an agent, particularly a biologically active agent that modulates the level of expression of a nucleic acid in a cell, the method comprising: combining a candidate agent to be tested with a cell comprising a nucleic acid that encodes a polypeptide, and determining the agent's effect on polypeptide expression.

Some embodiments will detect agents that decrease the biological activity of a molecule of the invention. Maximal inhibition of the activity is not always necessary, or even desired, in every instance to achieve a therapeutic effect. Agents that decrease a biological activity can find use in treating disorders associated with the biological activity of the molecule. Alternatively, some embodiments will detect agents that increase a biological activity. Agents that increase a biological activity of a molecule of the invention can find use in treating disorders associated with a deficiency in the biological activity. Agents that increase or decrease a biological activity of a molecule of the invention can be selected for further study, and assessed for physiological attributes, i.e., cellular availability, cytotoxicity, or biocompatibility, and optimized as required. For example, a candidate agent is assessed for any cytotoxic activity it may exhibit toward the cell used in the assay using well-known assays, such as trypan blue dye exclusion, an MTT ([3-(4,5-dimethylthiazol-2-yl)-2,5-diphenyl-2H-tetrazolium bromide]) assay, and the like.

Candidate agents are obtained from a wide variety of sources including libraries of synthetic or natural compounds. Numerous means are available for random and directed synthesis of a wide variety of organic compounds and biomolecules, including expression of randomized oligonucleotides and oligopeptides. For example, random peptide libraries obtained by yeast two-hybrid screens (Xu et al., 1997), phage libraries (Hoogenboom et al., 1998), or chemically generated libraries. Alternatively, libraries of natural compounds in the form of bacterial, fungal, plant and animal extracts are available or readily produced, including antibodies produced upon immunization of an animal with subject polypeptides, or fragments thereof, or with the encoding polynucleotides. Additionally, natural or synthetically produced libraries and compounds are readily modified through conventional chemical, physical and biochemical means, and can be used to produce combinatorial libraries. Further, known pharmacological agents can be subjected to directed or random chemical modifications, such as acylation, alkylation, esterification, and amidification, etc, to produce structural analogs.

Modulating the Expression of cDNA Clones

The present invention further features a method of identifying an agent that modulates the level of a subject polypeptide (or an mRNA encoding a subject polypeptide) in a cell. The method generally involves contacting a cell (e.g., a eukaryotic cell) that produces the subject polypeptide with a test agent; and determining the effect, if any, of the test agent on the level of the polypeptide in the cell.

The present invention further features a method of identifying an agent that modulates biological activity of a subject polypeptide. The methods generally involve contacting a subject polypeptide with a test agent; and determining the effect, if any, of the test agent on the activity of the polypeptide. In certain embodiments, a polypeptide is expressed on a cell surface. In certain embodiments, the agent or modulator is an antibody, for example, where an antibody binds to the polypeptide or affects its biological activity. In other embodiments, the agent or modulator is an inhibitory RNA molecule. The present invention further features biologically active agents (or modulators) identified using a method of the invention.

The present invention also features a method of modulating biological activity using an agent selectable by the above methods. Generally, methods of the invention can encompass modulating biological activity by contacting an agent with a first human or a non-human host cell, thereby modulating the activity of the first host cell or a second host cell. In one example, contacting the agent with the first human or non-human host cell results in the recruitment of a second host cell. The agent may, as described in more detail below, be an antibody or antibody fragment of the invention.

The modulation can comprise directly enhancing cell activity, indirectly enhancing cell activity, directly inhibiting cell activity, or indirectly inhibiting cell activity. The cell activity that is modulated can include transcription, translation, cell cycle control, signal transduction, intracellular trafficking, cell adhesion, cell mobility, proteolysis, cell growth, differentiation, and/or activities corresponding to the predicted function of the cDNA clone of the invention, as described in the Tables and throughout the specification. The modulation can result in cell death or apoptosis, or inhibition of cell death or apoptosis, as well as cell growth, cell proliferation, or cell survival, or inhibition of cell growth, cell proliferation, or cell survival.

Either the first or the second host cell can be a human or a non-human host cell. Either the first or the second host cell can be an immune cell, e.g., a T cell, B cell, NK cell, dendritic cell, macrophage, muscle cell, stem cell, skin cell, fat cell, blood cell, brain cell, bone marrow cell, endothelial cell, retinal cell, bone cell, kidney cell, pancreatic cell, liver cell, spleen cell, prostate cell, cervical cell, ovarian cell, breast cell, lung cell, liver cell, soft tissue cell, colorectal cell, other cell of the gastrointestinal tract, or a cancer cell.

The invention provides a method of modulating the expression of a cellular component by introducing a nucleic acid molecule that encodes an isolated amino acid molecule comprising a first polypeptide with the amino acid sequence of SEQ. ID. NOS.:55-108 or one or more of its biologically active fragments or variants into the cell; introducing an inhibitory modulator of transcription of the nucleic acid molecule into the cell, introducing an inhibitory modulator of translation of the polypeptide with the amino acid sequence of SEQ ID NOS.:55-108 or one or more of its biologically active fragments into the cell, or introducing an inhibitory modulator of the activity of this polypeptide into the cell; introducing the polypeptide with the amino acid sequence of SEQ ID NOS.:55-108 or one or more of its biologically active fragments or variants into the cell; and incubating the cell in the presence of this polypeptide. Inhibitors effective in practicing this method include RNAi molecules, antisense molecules, natural inhibitors of polypeptides with the amino acid sequence SEQ ID NOS.:55-108 or biologically active fragments or variants thereof, antibodies directed specifically against the polypeptides with the amino acid sequence SEQ ID NOS.:55-108 or biologically active fragments, and nucleic acid molecules encoding polypeptides with the amino acid sequence SEQ ID NOS.:55-108 or biologically active fragments or variants thereof. The invention also includes an inhibitor of the activity of a polypeptide with the amino acid sequence SEQ ID NOS.:55-108 or biologically active fragments or variants thereof.

The invention also provides a method of modulating cell growth, differentiation, function, or other activity in an animal in need of such modulation by administering a composition with a therapeutically effective amount of a modulator, e.g., a polypeptide with the amino acid sequence of SEQ. ID. NOS.:55-108 or one or more active fragment or variant thereof, a polypeptide encoded by SEQ. ID. NOS.:1-54 or one or more active fragment or variant thereof, or an agonist or antagonist thereof. The cell growth, differentiation, function, or activity can be associated with cancer, other proliferative disorders, such as psoriasis, developmental disorders, including disorders of B-cell development; disorders of cellular differentiation, including lymphoid and monocyte differentiation; disorders of stem cell renewal; disorders of cell survival; immune disorders including disorders of B-cell function, B-cell activation, B-cell homing, B-cell maturation, and autoimmunity, both T-cell and B-cell mediated; hematopoeisis, including lymphopoeisis and monopoeisis; inflammatory disorders, such as inflammatory bowel disease and ulcerative colitis; gastrointestinal disorders, including celiac disease; obesity; thyroid disorders such as Grave's disease and Hashimoto's disease, infectious diseases, including disorders caused by viruses and bacteria, fertility, type II diabetes, lung diseases such as asthma and chronic obstructive pulmonary disease; and endocrine disorders such as Addison's disease and disorders of peptide modulation. In an embodiment of this method, the antagonist is an antibody.

Specifically, the present invention provides a method of treating a disease, disorder, syndrome, or condition in a subject by administering a nucleic acid composition comprising a pharmaceutically acceptable carrier or a buffer and one or more nucleic acid molecule comprising a polynucleotide sequence chosen from at least one polynucleotide sequence according to SEQ ID NOS.:1-54, a complement thereof, a fragment thereof, or a variant thereof. The invention also provides a method of treating a disease, disorder, syndrome, or condition in a subject by administering a double-stranded isolated nucleic acid molecule comprising a nucleic acid molecule such as described above, and its complement. The invention further provides a method of treating a disease, disorder, syndrome, or condition in a subject by administering a nucleic acid composition comprising a polynucleotide sequence that encodes SEQ ID NOS.:55-108, a fragment thereof, and a variant thereof or the nucleic acid molecule of a vector comprising a nucleic acid molecule comprising a polynucleotide sequence chosen from at least one polynucleotide sequence according to SEQ ID NOS.:1-54, a complement thereof, a fragment thereof, a variant thereof, a polynucleotide sequence that encodes SEQ ID NOS.:55-108, a fragment thereof, and a variant thereof; and a promoter that drives the expression of the nucleic acid molecule. The invention a method of treating a disease, disorder, syndrome, or condition in a subject by administering a nucleic acid composition comprising a host cell transformed, transfected, transduced, or infected with a nucleic acid molecule comprising a polynucleotide sequence chosen from at least one polynucleotide sequence according to SEQ ID NOS.:1-54, a complement thereof, a fragment thereof, a variant thereof, a polynucleotide sequence that encodes SEQ ID NOS.:55-108, a fragment thereof, or a variant thereof.

The invention provides a polypeptide composition comprising the amino acid molecule of comprising a polypeptide sequence chosen from amino acid sequence according to SEQ ID NOS.:55-108, a complement thereof, a fragment thereof, and a variant thereof, and a pharmaceutically acceptable carrier or a buffer. The invention also provides an antibody composition comprising an antibody or a biologically active fragment of an antibody that specifically recognizes, binds to, and/or modulates the biological activity of at least one polypeptide encoded by a nucleic acid molecule comprising a polynucleotide sequence chosen from at least one polynucleotide sequence according to SEQ ID NOS.:1-54, a complement thereof, a fragment thereof, a variant thereof, a polynucleotide sequence that encodes SEQ ID NOS.:55-108, a fragment thereof, or a variant thereof; and a pharmaceutically acceptable carrier.

The therapeutic compositions can be administered in a variety of ways. These include oral, buccal, rectal, parenteral, including intranasal, intramuscular, intravenous, intra-arterial, intraperitoneal, intradermal, transdermal, subcutaneous, intratracheal, intracardiac, intraventricular, intracranial, intrathecal, etc., and administration by implantation. The agents may be administered daily, weekly, or monthly, as appropriate as conventionally determined.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules, or capsules, for example, with conventional additives, such as lactose, mannitol, corn starch or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art (Gennaro, 2003). The composition or formulation to be administered will, in any event, contain a quantity of the polypeptide adequate to achieve the desired state in the subject being treated.

A variety of patients are treatable according to the subject methods. The host, or patient, may be from any animal species, and will generally be mammalian, e.g., a primate such as a monkey, chimpanzee, and, particularly, a human; rodent, including mice, rats, hamsters, and guinea pigs; rabbits; cattle, including equines, bovines, pigs, sheep, and goats; canines; and felines; etc. Animal models are of interest for experimental investigations; they provide a model for treating human disease.

Antisense RNA, siRNA, and Peptide Aptamers

In an embodiment of the invention, antisense reagents can be used to down-regulate gene expression. The antisense reagent can be one or more antisense oligonucleotide, particularly synthetic antisense oligonucleotides with chemical modifications of native nucleic acids, or nucleic acid constructs that express antisense molecules, e.g., RNA based on one or more of SEQ ID NOS.:1-54. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNAse H, or by steric hindrance. One or a combination of antisense molecules can be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, usually not more than about 35 nucleotides in length, and usually not more than about 50, and not more than about 500, where the length is governed by efficiency of inhibition, specificity, including absence of cross-reactivity, and the like. Short oligonucleotides, of from 7 to 8 bases in length, can be strong and selective inhibitors of gene expression (Wagner et al., 1996).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides can be chemically synthesized by methods known in the art (Wagner et al., 1993; Milligan et al., 1993). Preferred oligonucleotides are chemically modified from the native phosphodiester stricture, in order to increase their intracellular stability and binding affinity. A number of such modifications have been described in the literature, which modifications alter the chemistry of the backbone, sugars or heterocyclic bases.

As an alternative to antisense inhibitors, catalytic nucleic acid compounds, e.g., ribozymes, antisense conjugates, interfering RNA, etc. can be used to inhibit gene expression. Ribozymes can be synthesized in vitro and administered to the patient, or encoded in an expression vector, from which the ribozyme is synthesized in the targeted cell (WO 95/23225; Beigelman et al., 1995). Examples of oligonucleotides with catalytic activity are described in WO 95/06764. Conjugates of anti-sense ODN with a metal complex, e.g., terpyridylCu(II), capable of mediating mRNA hydrolysis are described in Bashkin et al., 1995.

Small interfering RNA (siRNA) can also be used as an inhibitor. Small interfering RNA can be used to screen for biologically active agents by administering siRNA compositions to cells, monitoring for a change in a readable biological activity, and repeating the administration and monitoring with a subset of the plurality of siRNA compositions to determine which silenced gene is responsible for the change, then identifying the transcriptional or translational gene product of the silenced gene. The transcriptional or translational product so identified may represent a biologically active agent, responsible for the change which is determined by the readable biological activity.

The invention provides methods of producing libraries of siRNA molecules by enzymatically engineering DNA, including generating siRNAs by intra-molecular sense- and antisense single-stranded DNA ligation. Libraries of siRNA molecules can also be produced by two converging, opposing RNA polymerase III promoters (Kaykas and Moon, 2004, Zhang and Williams, U.S. patent application for Small Interfering RNA Libraries, 2004). The resulting siRNA can selectively inhibit gene expression relevant to a specific cell, tissue, protein family, or disease (Zhang and Williams, U.S. patent application for Small Interfering RNA Libraries, 2004).

Small interfering RNA compositions, including the libraries of the invention, can be used to screen populations of transfected cells for phenotypic changes. Cells with the desired phenotype can be recovered, and the siRNA construct can be characterized. The screening can be performed using oligonucleotides specific to any open reading frame, including enzymatically fragmented, open reading frames, e.g., with restriction endonucleases. The screening can also be performed using random siRNA libraries, including enzymatically fragmented libraries, e.g., with restriction endonucleases.

The invention provides a method of using siRNA to identify one or more specific siRNA molecules effective against one or more polypeptides of the invention or fragments thereof. This method can be performed by administering the composition to cells expressing the mRNA, monitoring for a change in a readable biological activity, e.g., activity relevant to a disease condition, and repeating the administration and monitoring with a subset of a plurality of siRNA molecules, thereby identifying one or more specific siRNA molecules effective against one or more genes relevant to a disease condition. This method includes using one or more siRNA molecules for treating or preventing a disease, by administering the identified siRNA to patient in an amount effective to inhibit one or more genes relevant to the disease. This method can be performed, e.g., by gene therapy, described in more detail below, by administering an effective amount of the identified specific siRNA to a patient. This method can also be performed by administering an effective amount of the identified specific siRNA to a patient by administering a nucleic acid vaccine, either with or without an adjuvant, also described in more detail below. The siRNA molecules and compositions of the invention can be also used in diagnosing a given disease or abnormal condition by administering any of the siRNA molecules or compositions of the invention to a biological sample and monitoring for a change in a readable biological activity to identify the disease or abnormal condition.

Another suitable agent for reducing an activity of a subject polypeptide is a peptide aptamer. Peptide aptamers are peptides or small polypeptides that act as dominant inhibitors of protein function; they specifically bind to target proteins, blocking their function (Kolonin and Finley, 1998). Due to the highly selective nature of peptide aptamers, they may be used not only to target a specific protein, but also to target specific functions of a given protein (e.g., a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Antibodies

In some embodiments of the invention, polypeptide expression is modulated by an antibody. The invention provides an antibody that specifically recognizes, binds to and/or modulates the biological activity of at least one polypeptide encoded by a nucleic acid molecule with the sequence of SEQ ID NOS.:1-54, a fragment or variant thereof, a polynucleotide sequence that encodes SEQ ID NOS.:55-108, or a fragment or variant thereof. In an embodiment, this antibody is provided as an antibody composition comprising a pharmaceutically acceptable carrier. Antibodies of the invention are provided as components of host cells, and in kits, as discussed above.

The invention provides a method of modulating biological activity by providing an antibody that specifically recognizes, binds to and/or modulates the biological activity of at least one polypeptide encoded by a nucleic acid molecule with the sequence of SEQ ID NOS.:1-54, a polypeptide with the sequence of SEQ ID NO.:55-108, or a biologically active fragment thereof; and contacting the antibody with a first human or a non-human host cell thereby modulating the activity of the first human or non-human animal host cell, or a second host cell. This modulation of biological activity can includes enhancing cell activity directly, enhancing cell activity indirectly, inhibiting cell activity directly, and inhibiting cell activity indirectly. The present invention further features an antibody that specifically inhibits binding of a polypeptide to its ligand or substrate. It also features an antibody that specifically inhibits binding of a polypeptide as a substrate to another molecule.

The invention provides antibodies that can distinguish the variant sequences of the invention from currently known sequences. These antibodies can distinguish polypeptides that differ by no more than one amino acid (U.S. Pat. No. 6,656,467). They have high affinity constants, i.e., in the range of approximately 10⁻¹⁰M, and are produced, for example, by genetically engineering appropriate antibody gene sequences, according to the method described by Young et al., in U.S. Pat. No. 6,656,467.

The invention further provides a host cell that can produce an antibody of the invention or a fragment thereof. The antibody may also be secreted by the cell. The host cell can be a prokaryotic or eukaryotic cell, e.g., a hybridoma. The invention also provides a bacteriophage or other virus particle comprising an antibody of the invention, or a fragment thereof. The bacteriophage or other virus particle may display the antibody or fragment thereof on its surface, and the bacteriophage itself may exist within a bacterial cell. The antibody may also comprise a fusion protein with a viral or bacteriophage protein.

The invention further provides transgenic multicellular organisms, e.g., plants or non-human animals, as well as tissues or organs, comprising a polynucleotide sequence encoding a subject antibody or fragment thereof. The organism, tissues, or organs will generally comprise cells producing an antibody of the invention, or a fragment thereof.

Another aspect of the present invention features a library of antibodies or fragments thereof, wherein at least one antibody or fragment thereof specifically binds to at least a portion of a polypeptide comprising an amino acid sequence according to SEQ ID NOS.:55-108, and/or wherein at least one antibody or fragment thereof interferes with at least one activity of such polypeptide or fragment thereof. In certain embodiments, the antibody library comprises at least one antibody or fragment thereof that specifically inhibits binding of a subject polypeptide to its ligand or substrate, or that specifically inhibits binding of a subject polypeptide as a substrate to another molecule. The present invention also features corresponding polynucleotide libraries comprising at least one polynucleotide sequence that encodes an antibody or antibody fragment of the invention. In specific embodiments, the library is provided on a nucleic acid array or in computer-readable format.

In another aspect, the present invention features a method of making an antibody by immunizing a host animal (Coligan, 2002). In this method, a polypeptide or a fragment thereof, a polynucleotide encoding a polypeptide, or a polynucleotide encoding a fragment thereof, is introduced into an animal in a sufficient amount to elicit the generation of antibodies specific to the polypeptide or fragment thereof, and the resulting antibodies are recovered from the animal. Initial immunizations can be performed using either polynucleotides or polypeptides. Subsequent booster immunizations can also be performed with either polynucleotides or polypeptides. Initial immunization with a polynucleotide can be followed with either polynucleotide or polypeptide immunizations, and an initial immunization with a polypeptide can be followed with either polynucleotide or polypeptide immunizations.

The host animal will generally be a different species than the immunogen, e.g., a human protein used to immunize mice. Methods of antibody production are well known in the art (Coligan, 2002; Howard and Bethell, 2000; Harlow et al., 1998; Harlow and Lane, 1988). The invention thus also provides a non-human animal comprising an antibody of the invention. The animal can be a non-human primate, (e.g., a monkey) a rodent (e.g., a rat, a mouse, a hamster, a guinea pig), a chicken, cattle (e.g., a sheep, a goat, a horse, a pig, a cow), a rabbit, a cat, or a dog.

The present invention also features a method of making an antibody by isolating a spleen from an animal injected with a polypeptide or a fragment thereof, a polynucleotide encoding a polypeptide, or a polynucleotide encoding a fragment thereof, and recovering antibodies from the spleen cells. Hybridomas can be made from the spleen cells, and hybridomas secreting specific antibodies can be selected.

The present invention further features a method of making a polynucleotide library from spleen cells, and selecting a cDNA clone that produces specific antibodies, or fragments thereof. The cDNA clone or a fragment thereof can be expressed in an expression system that allows production of the antibody or a fragment thereof, as provided herein.

The immunogen can comprise a nucleic acid, a complete protein, or fragments and derivatives thereof, or proteins expressed on cell surfaces. Pfam domains and structural motifs can be used as immunogens. Proteins domains, e.g., extracellular, cytoplasmic, or luminal domains can be used as immunogens. Immunogens comprise all or a part of one of the subject proteins, where these amino acids contain post-translational modifications, such as glycosylation, found on the native target protein. Immunogens comprising protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies are prepared by conventional techniques. These include immunizing the host animal in vivo with the target protein (or immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil & water emulsions, e.g., Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host is collected, followed by separation of the serum from blood cells. The immunoglobulin present in the resultant antiserum can be further fractionated using known methods, such as ammonium salt fractionation, or DEAE chromatography and the like.

Cytokines can also be used to help stimulate immune response. Cytokines act as chemical messengers, recruiting immune cells that help the killer T-cells to the site of attack. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

The method of producing polyclonal antibodies can be varied in some embodiments of the present invention. For example, instead of using a single substantially isolated polypeptide as an immunogen, one may inject a number of different immunogens into one animal for simultaneous production of a variety of antibodies. In addition to protein immunogens, the immunogens can be nucleic acids (e.g., in the form of plasmids or vectors) that encode the proteins, with facilitating agents, such as liposomes, microspheres, etc, or without such agents, such as “naked” DNA.

Antibodies can also be prepared using a library approach. Briefly, mRNA is extracted from the spleens of immunized animals to isolate antibody-encoding sequences. The extracted mRNA may be used to make cDNA libraries. Such a cDNA library may be normalized and subtracted in a manner conventional in the art, for example, to subtract out cDNA hybridizing to mRNA of non-immunized animals. The remaining cDNA may be used to create proteins and for selection of antibody molecules or fragments that specifically bind to the immunogen. The cDNA clones of interest, or fragments thereof, can be introduced into an in vitro expression system to produce the desired antibodies, as described herein.

In a further embodiment, polyclonal antibodies can be prepared using phage display libraries, which are conventional in the art. Specifically, the invention provides a bacteriophage that displays an antibody or a fragment of an antibody that can specifically recognize, bind to and/or modulate the biological activity of at least one polypeptide encoded by a polynucleotide with the sequence of SEQ ID NOS.:1-54 or a biological fragment thereof. The invention also provides a bacterial cell comprising such a bacteriophage. In this method, a collection of bacteriophages displaying antibody properties on their surfaces are made to contact subject polypeptides, or fragments thereof. Bacteriophages displaying antibody properties that specifically recognize the subject polypeptides are selected, amplified, for example, in E. coli, and harvested. Such a method typically produces single chain antibodies, which are further described below.

Phage display technology can be used to produce Fab antibody fragments, which can be then screened to select those with strong and/or specific binding to the protein targets. The screening can be performed using methods that are known to those of skill in the art, for example, ELISA, immunoblotting, immunohistochemistry, or immunoprecipitation. Fab fragments identified in this manner can be assembled with an Fc portion of an antibody molecule to form a complete immunoglobulin molecule.

Monoclonal antibodies are also produced by conventional techniques, such as fusing an antibody-producing plasma cell with an immortal cell to produce hybridomas. Suitable animals will be used, e.g., to raise antibodies against a mouse polypeptide of the invention, the host animal will generally be a hamster, guinea pig, goat, chicken, or rabbit, and the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, e.g., affinity chromatography using antigen, e.g., the subject protein, bound to an insoluble support, i.e., protein A sepharose, etc.

The antibody can be produced as a single chain, instead of the normal multimeric structure of the immunoglobulin molecule. Single chain antibodies have been previously described (i.e., Jost et al., 1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., 1994; Hassanzadeh et al., 1998). Inducible expression vectors can be constructed with intrabodies that react specifically with a protein of the invention. These vectors can be introduced into host cells and model organisms.

The invention also provides “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected, for example, on the basis of phage binding to selected antigens, can be fused to a Fc fragment of an immunoglobulin for use as a therapeutic, as described, for example, in U.S. Pat. No. 5,116,964 or WO 99/61630. Antibodies of the invention can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies of the invention can be administered to mammals, and the present invention includes such administration, particularly for therapeutic and/or diagnostic purposes in humans.

Antibodies may be administered by injection systemically, such as by intravenous injection; or by injection or application to the relevant site, such as by direct injection into a tumor, or direct application to the site when the site is exposed in surgery; or by topical application, such as if the disorder is on the skin, for example.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, e.g., constant region genes (e.g., Grosveld and Kolias, 1992; Murphy and Carter, 1993; Pinkert, 1994; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, e.g., WO 92/02190). Humanized antibodies can also be produced by immunizing mice that make human antibodies, such as Abgenix xenomice, Medarex's mice, or Kirin's mice, and can be made using the technology of Protein Design Labs, Inc. (Fremont, Calif.) (Coligan, 2002). Both polyclonal and monoclonal antibodies made in non-human animals may be humanized before administration to human subjects.

The antibodies can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (e.g., WO 98/50433; WO 98/24893 and WO 99/53049).

Chimeric immunoglobulin genes constructed with immunoglobulin cDNA are known in the art (Liu et al. 1987a; Liu et al. 1987b). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant (c) regions genes are known in the art (Kabat et al., 1991). Human C region genes are readily available from known clones. The choice of isotype will be guided by the desired effector functions, such as complement fixation, or antibody-dependent cellular cytotoxicity. IgG1, IgG3 and IgG4 isotypes, and either of the kappa or lambda human light chain constant regions can be used. The chimeric, humanized antibody is then expressed by conventional methods.

Consensus sequences of heavy (H) and light (L) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama, et al. 1983), Rous sarcoma virus LTR (Gorman et al. 1982), and Moloney murine leukemia virus LTR (Grosschedl et al. 1985), or native immunoglobulin promoters.

Antibody fragments, such as Fv, F(ab′)2, and Fab can be prepared by cleavage of the intact protein, e.g., by protease or chemical cleavage. These fragments can include heavy and light chain variable regions. Alternatively, a truncated gene can be designed, e.g., a chimeric gene encoding a portion of the F(ab′)₂ fragment that includes DNA sequences encoding the CH1 domain and hinge region of the H chain, followed by a translational stop codon.

The antibodies of the present invention may be administered alone or in combination with other molecules for use as a therapeutic, for example, by linking the antibody to cytotoxic agent or radioactive molecule. Radioactive antibodies that are specific to a cancer cell, disease cell, or virus-infected cell may be able to deliver a sufficient dose of radioactivity to kill such cancer cell, disease cell, or virus-infected cell. The antibodies of the present invention can also be used in assays for detection of the subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably-labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷Cu, ⁹⁰Y, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, ad ²⁴⁴Cm; enzymes having detectable products (e.g., luciferase, β-galactosidase, and the like); fluorescers and fluorescent labels, e.g., as provided herein, fluorescence emitting metals, e.g., ¹⁵²Eu, or others of the lanthanide series, attached to the antibody through metal chelating groups such as EDTA; chemiluminescent compounds, e.g., luminol, isoluminol, or acridinium salts; and bioluminescent compounds, e.g., luciferin, or aequorin (green fluorescent protein), specific binding molecules, e.g., magnetic particles, microspheres, nanospheres, and the like.

Alternatively, specific-binding pairs may be used, involving, e.g., a second stage antibody or reagent that is detectably-labeled and that can amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

Gene Therapy

Gene therapy of the invention can be performed in vitro or in vivo. In vivo gene therapy can be accomplished by directly transfecting or transducing a nucleic acid of the invention, i.e., SEQ ID NOS.:1-54 and/or one or more of its complements, variants, or biologically active fragments into the patient's target cells. In vitro gene therapy can be accomplished by transfecting or transducing a nucleic acid of the invention into cells in vitro and then administering them to the patient. Transfection of a nucleic acid of the invention involves its direct introduction into the cell. Transduction of a nucleic acid of the invention involves its introduction into the cell via a vector.

For example, an siRNA of SEQ ID NO.:1-54 can be used in gene therapy to transiently or permanently alter the cellular phenotype of patients in need of such treatment (Bast et al., 2000). Gene therapy with siRNA can suppress the disease phenotype, e.g., by down-regulating genes that contribute to disease progression, by reversing the transformed phenotype, and/or by inducing cell death. In vivo gene therapy can be accomplished by directly transfecting or transducing siRNA into the patient's target cells. In vitro gene therapy can be accomplished by transfecting or transducing siRNA into cells in vitro and then administering them to the patient. Transfection of siRNA involves its direct introduction into the cell. Transduction of siRNA involves its introduction into the cell via a vector.

Both viral and non-viral vectors are suitable for therapeutic use in the invention. Suitable viral vectors include retroviruses, adenoviruses, herpes viruses, and adeno-associated viruses. Viral vectors can enter cells by receptor-mediated processes and deliver nucleic acids to the cell interior. Non-viral delivery systems suitable for therapeutic use include transfecting plasmids into cells, e.g., by calcium phosphate precipitation and electroporation. The siRNA compositions of the invention may also be introduced into the target cell in vitro by microinjection. They may be introduced into target cells by vesicle fusion e.g., with cationic liposomes with the plasma membrane. They may be directly injected into a target tissue. Direct injection techniques include particle-mediated nucleic acid transfer by physical force, i.e., by a particle bombardment device, or “gene gun” (Tang et al., 1992) as described above.

The invention also provides a method for administering a nucleic acid vaccine by administering an effective amount of the siRNA molecules or compositions of the invention to a patient. Administration of a vaccine of the invention can lead to the persistent expression and release of the therapeutic immunogen over a period of time. The siRNA vaccines may induce humoral responses. They may also induce cellular responses, for example, by stimulating T-cells that recognize and kill cells, e.g., tumor cells, directly. (Heiser et al., 2002; Mitchell and Nair, 2000). Nucleic acid sequences of the invention can be introduced into tissues or host cells by any number of routes, including viral infection, microinjection, or fusion of vesicles. Both viral and non-viral vectors are suitable for use in the invention. Suitable viral vectors include retroviruses, adenoviruses, herpes viruses, and adeno-associated viruses. Viral vectors can enter cells by receptor-mediated processes and deliver nucleic acids to the cell interior. Non-viral delivery systems suitable for the invention include transfecting plasmids into cells, e.g., by calcium phosphate precipitation and electroporation.

The invention provides a method of gene therapy comprising providing a polynucleotide comprising a nucleic acid molecule encoding the antibody of the invention as described above; and administering the polynucleotide to a subject.

The nucleic acid and amino acid molecules of the invention can be used to develop treatments for any disorder mediated either directly or indirectly by physiologically defective or insufficient amounts of these nucleic acid and amino acid molecules. Specifically, the invention provides methods of prophylaxis or therapeutic treatment of an animal in need of such treatment by providing compositions comprising one or more polynucleotides or polypeptides with the sequence SEQ ID NO.:1-54 or SEQ ID NO.:55-108, or biologically active fragments or variants of either, and administering a therapeutically effective amount to the animal. The method can be applied to a human or non-human animal, for example, a human patient. These prophylactic and treatment methods can be used, for example, after the animal, e.g., the human patient, has undergone chemotherapy and/or radiotherapy. These methods can employ a polypeptide that has been mutated to optimize its activity, as described in more detail above.

In some embodiments the molecules of the invention are altered such that the peptide antigens encoded by the RNA are more highly antigenic than in their native state. (Yu and Restifo, 2002). Some embodiments of the present invention use viral vectors from non-mammalian natural hosts, i.e., avian pox viruses. Alternative embodiments include genetically engineered influenza viruses, and the use of “naked” plasmid nucleic acid vaccines that contain no associated protein. (Yu and Restifo, 2002).

All of the methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines. In some embodiments, nucleic acid vaccines encode an alphaviral replicase enzyme. This recently discovered approach to vaccine therapy successfully combines therapeutic antigen production with the induction of the apoptotic death of the tumor cell (Yu and Restifo, 2002).

Furthermore, adjuvants may be used in conjunction with the vaccines disclosed herein. Adjuvants help boost the general immune response, for example, concentrating immune cells to the specific area where they are needed. They can be added to a cancer vaccine or administered separately, and in some embodiments, a viral vector can be engineered to display adjuvant proteins on its surface.

Cytokines can also be used to help stimulate the immune response, as noted above. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

Stem cells provide attractive targets for gene therapy because of their capacity for self renewal and their wide systemic distribution. Correcting a defective gene in a stem cell corrects the defect in the undifferentiated progeny and the differentiated progeny. Because stem cells disseminate throughout the organism, stem cells can be treated in situ or ex vivo, and, post-treatment, travel to their functional site. Sustained expression of transgenes at clinically relevant levels in the progeny of stem cells may provide novel and potentially curative treatments for a wide range of inherited and acquired diseases (Hawley, 2001).

Treating Disorders of Cell Development

Where a sequence of the invention is involved in modulating cell death, e.g., during development, an agent of the invention is useful for treating conditions or disorders relating to cell death (e.g., DNA damage, cell death, and apoptosis). Cell death-related indications that can be treated using the methods of the invention to reduce cell death in a eukaryotic cell, include, but are not limited to, cell death associated with Alzheimer's disease, Parkinson's disease, rheumatoid arthritis, autoimmune thyroiditis, septic shock, sepsis, stroke, central nervous system inflammation, intestinal inflammation, osteoporosis, ischemia, reperfusion injury, cardiac muscle cell death associated with cardiovascular disease, polycystic kidney disease, cell death of endothelial cells in cardiovascular disease, degenerative liver disease, multiple sclerosis, amyotropic lateral sclerosis, cerebellar degeneration, ischemic injury, cerebral infarction, myocardial infarction, acquired immunodeficiency syndrome (AIDS), myelodysplastic syndromes, aplastic anemia, male pattern baldness, and head injury damage. Also included are conditions in which DNA damage to a cell is induced by external conditions, including but not limited to irradiation, radiomimetic drugs, hypoxic injury, chemical injury, and damage by free radicals. Also included are any hypoxic or anoxic conditions, e.g., conditions relating to or resulting from ischemia, myocardial infarction, cerebral infarction, stroke, bypass heart surgery, organ transplantation, and neuronal damage.

DNA damage can be detected using any known method, including, but not limited to, a Comet assay (commercially available from Trevigen, Inc.), which is based on alkaline lysis of labile DNA at sites of damage, and immunological assays using antibodies specific for aberrant DNA structures, e.g., 8-OHdG.

Cell death can be measured using any known method, and is generally measured using any of a variety of known methods for measuring cell viability. Such assays are generally based on entry into the cell of a detectable compound (or a compound that becomes detectable upon interacting with, or being acted on by, an intracellular component) that would normally be excluded from a normal, living cell by its structurally and functionally intact cell membrane. Such compounds include substrates for intracellular enzymes, including, but not limited to, a fluorescent substrate for esterase; dyes that are excluded from living cells, including, but not limited to, trypan blue; and DNA-binding compounds, including, but not limited to, an ethidium compound such as ethidium bromide and ethidium homodimer, and propidium iodide.

Apoptosis, or programmed cell death, is a regulated process leading to cell death via a series of well-defined morphological changes. Programmed cell death provides a balance for cell growth and multiplication, eliminating unnecessary cells. The default state of the cell is to remain alive. A cell enters the apoptotic pathway when an essential factor is removed from the extracellular environment or when an internal signal is activated. Genes and proteins of the invention that suppress the growth of tumors by activating cell death provide the basis for treatment strategies for hyperproliferative disorders and conditions.

Apoptosis can be assayed using any known method. Assays can be conducted on cell populations or an individual cell, and include morphological assays and biochemical assays. A non-limiting example of a method of determining the level of apoptosis in a cell population is TUNEL (TdT-mediated dUTP nick-end labeling) labeling of the 3′-OH free end of DNA fragments produced during apoptosis (Gavrieli et al., 1992). The TUNEL method consists of catalytically adding a nucleotide, which has been conjugated to a chromogen system, a fluorescent tag, or the 3′-OH end of the 180-bp (base pair) oligomer DNA fragments, in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus).

Another marker that is currently available is annexin, sold under the trademark APOPTEST™. This marker is used in the “Apoptosis Detection Kit,” which is also commercially available, e.g., from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. Other methods of testing for apoptosis are known in the art and can be used, including, e.g., the method disclosed in U.S. Pat. No. 6,048,703.

Treating Cancer and Proliferative Conditions

The therapeutic compositions and methods of the invention can be used in the treatment of cancer, i.e., an abnormal malignant cell or tissue growth, e.g., a tumor. In an embodiment, the compositions and methods of the invention kill tumor cells. In an embodiment, they inhibit tumor development. Cancer is characterized by the proliferation of abnormal cells that tend to invade the surrounding tissue and metastasize to new body sites. The growth of cancer cells exceeds that of and is uncoordinated with the normal cells and tissues. In an embodiment, the compositions and methods of the invention inhibit the progression of premalignant lesions to malignant tumors.

Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can have one or more than one neoplastic cell type. Some characteristics that can, in some instances, apply to cancer cells are that they are morphologically different from normal cells, and may appear anaplastic; they have a decreased sensitivity to contact inhibition, and may be less likely than normal cells to stop moving when surrounded by other cells; and they have lost their dependence on anchorage for cell growth, and may continue to divide in liquid or semisolid surroundings, whereas normal cells must be attached to a solid surface to grow.

The fusion proteins and conjugates described above can be used to treat cancer. In an embodiment, a fusion protein or conjugate can additionally comprise a tumor-targeting moiety. Suitable moieties include those that enhance delivery of an therapeutic molecule to a tumor. For example, compounds that selectively bind to cancer cells compared to normal cells, selectively bind to tumor vasculature, selectively bind to the tumor type undergoing treatment, or enhance penetration into a solid tumor are included in the invention. Tumor targeting moieties of the invention can be peptides. Nucleic acid and amino acid molecules of the invention can be used alone or as an adjunct to cancer treatment. For example, a nucleic acid or amino acid molecules of the invention may be added to a standard chemotherapy regimen. It may be combined with one or more of the wide variety of drugs that have been employed in cancer treatment, including, but are not limited to, cisplatin, taxol, etoposide, Novantrone (mitoxantrone), actinomycin D, camptohecin (or water soluble derivatives thereof), methotrexate, mitomycins (e.g., mitomycin C), dacarbazine (DTIC), and anti-neoplastic antibiotics such as doxorubicin and daunomycin. Drugs employed in cancer therapy may have a cytotoxic or cytostatic effect on cancer cells, or may reduce proliferation of the malignant cells. Drugs employed in cancer treatment can also be peptides. A nucleic acid or amino acid molecules of the invention can be combined with radiation therapy. A nucleic acid or amino acid molecules of the invention may be used adjunctively with therapeutic approaches described in De Vita et al., 2001. For those combinations in which a nucleic acid or amino acid molecule of the invention and a second anti-cancer agent exert a synergistic effect against cancer cells, the dosage of the second agent may be reduced, compared to the standard dosage of the second agent when administered alone. A method for increasing the sensitivity of cancer cells comprises co-administering a nucleic acid or amino acid molecule of the invention with an amount of a chemotherapeutic anti-cancer drug that is effective in enhancing sensitivity of cancer cells. Co-administration may be simultaneous or non-simultaneous administration. A nucleic acid or amino acid molecule of the invention may be administered along with other therapeutic agents, during the course of a treatment regimen. In one embodiment, administration of a nucleic acid or amino acid molecule of the invention and other therapeutic agents is sequential. An appropriate time course may be chosen by the physician, according to such factors as the nature of a patient's illness, and the patient's condition.

The invention also provides a method for prophylactic or therapeutic treatment of a subject needing or desiring such treatment by providing a vaccine, that can be administered to the subject. The vaccine may comprise one or more of a polynucleotide, polypeptide, or modulator of the invention, for example an antibody vaccine composition, a polypeptide vaccine composition, or a polynucleotide vaccine composition, useful for treating cancer, proliferative, inflammatory, immune, metabolic, bacterial, or viral disorders.

For example, the vaccine can be a cancer vaccine, and the polypeptide can concomitantly be a cancer antigen. The vaccine may be an anti-inflammatory vaccine, and the polypeptide can concomitantly be an inflammation-related antigen. The vaccine may be a viral vaccine, and the polypeptide can concomitantly be a viral antigen. In some embodiments, the vaccine comprises a polypeptide fragment, comprising at least one extracellular fragment of a polypeptide of the invention, and/or at least one extracellular fragment of a polypeptide of the invention minus the signal peptide, for the treatment, for example, of proliferative disorders, such as cancer. In certain embodiments, the vaccine comprises a polynucleotide encoding one or more such fragments, administered for the treatment, for example, of proliferative disorders, such as cancer. Further, the vaccine can be administered with or without an adjuvant.

Tumors that can be treated using the methods of the instant invention include carcinomas, e.g., colorectal, prostate, breast, bone, kidney, skin, melanoma, ductal, endometrial, stomach or other organ of the gastrointestinal tract, pancreatic, mesothelioma, dysplastic oral mucosa, invasive oral cancer, non-small cell lung carcinoma (“NSCL”), transitional and squamous cell urinary carcinoma; brain cancer and neurological malignancies, e.g., neuroblastoma, glioblastoma, astrocytoma, and gliomas; lymphomas and leukemias such as myeloid leukemia, myelogenous leukemia, hematological malignancies, such as childhood acute leukemia, non-Hodgkin's lymphomas, chronic lymphocytic leukemia, malignant cutaneous T-cell lymphoma, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, and human follicular lymphoma; cancers of the reproductive system, e.g., cervical and ovarian cancers and testicular cancers; liver cancers including hepatocellular carcinoma (“HCC”) and tumors of the biliary duct; multiple myelomas; tumors of the esophageal tract; other lung cancers and tumors including small cell and clear cell; Hodgkin's lymphomas; adenocarcinoma; and sarcomas, including soft tissue sarcomas.

In some embodiments, a protein of the present invention is involved in the control of cell proliferation, and an agent of the invention inhibits undesirable cell proliferation. Such agents are useful for treating disorders that involve abnormal cell proliferation, including, but not limited to, cancer, psoriasis, and scleroderma. Whether a particular agent and/or therapeutic regimen of the invention is effective in reducing unwanted cellular proliferation, e.g., in the context of treating cancer, can be determined using standard methods. For example, the number of cancer cells in a biological sample (e.g., blood, a biopsy sample, and the like), can be determined. The tumor mass can be determined using standard radiological or biochemical methods.

Immunotherapeutic Approaches to Proliferative Conditions

The polynucleotides, polypeptides, and modulators of the present invention find use in immunotherapy of hyperproliferative disorders, including cancer, neoplastic, and paraneoplastic disorders. That is, the subject molecules can correspond to tumor antigens, of which 1770 have been identified to date (Yu and Restifo, 2002). Immunotherapeutic approaches include passive immunotherapy and vaccine therapy and can accomplish both generic and antigen-specific cancer immunotherapy.

Passive immunity approaches involve antibodies of the invention that are directed toward specific tumor-associated antigens. Such antibodies can eradicate systemic tumors at multiple sites, without eradicating normal cells. In some embodiments, the antibodies are combined with radioactive components, as provided above, for example, combining the antibody's ability to specifically target tumors with the added lethality of the radioisotope to the tumor DNA.

Useful antibodies comprise a discrete epitope or a combination of nested epitopes, i.e., a 10-mer epitope and associated peptide multimers incorporating all potential 8-mers and 9-mers, or overlapping epitopes (Dutoit et al., 2002). Thus a single antibody can interact with one or more epitopes. Further, the antibody can be used alone or in combination with different antibodies, that all recognize either a single or multiple epitopes.

Neutralizing antibodies can provide therapy for cancer and proliferative disorders. Neutralizing antibodies that specifically recognize a secreted protein or peptide of the invention can bind to the secreted protein or peptide, e.g., in a bodily fluid or the extracellular space, thereby modulating the biological activity of the secreted protein or peptide. For example, neutralizing antibodies specific for secreted proteins or peptides that play a role in stimulating the growth of cancer cells can be useful in modulating the growth of cancer cells. Similarly, neutralizing antibodies specific for secreted proteins or peptides that play a role in the differentiation of cancer cells can be useful in modulating the differentiation of cancer cells.

Vaccine therapy involves the use of polynucleotides, polypeptides, or agents of the invention as immunogens for tumor antigens (Machiels et al., 2002). For example, peptide-based vaccines of the invention include unmodified subject polypeptides, fragments thereof, and MHC class I and class II-restricted peptide (Knutson et al., 2001), comprising, for example, the disclosed sequences with universal, nonspecific MHC class II-restricted epitopes. Peptide-based vaccines comprising a tumor antigen can be given directly, either alone or in conjunction with other molecules. The vaccines can also be delivered orally by producing the antigens in transgenic plants that can be subsequently ingested (U.S. Pat. No. 6,395,964).

In some embodiments, antibodies themselves can be used as antigens in anti-idiotype vaccines. That is, administering an antibody to a tumor antigen stimulates B cells to make antibodies to that antibody, which in turn recognize the tumor cells

Nucleic acid-based vaccines can deliver tumor antigens as polynucleotide constructs encoding the antigen. Vaccines comprising genetic material, such as DNA or RNA, can be given directly, either alone or in conjunction with other molecules. Administration of a vaccine expressing a molecule of the invention, e.g., as plasmid DNA, leads to persistent expression and release of the therapeutic immunogen over a period of time, helping to control unwanted tumor growth.

In some embodiments, nucleic acid-based vaccines encode subject antibodies. In such embodiments, the vaccines (e.g., DNA vaccines) can include post-transcriptional regulatory elements, such as the post-transcriptional regulatory acting RNA element (WPRE) derived from Woodchuck Hepatitis Virus. These post-transcriptional regulatory elements can be used to target the antibody, or a fusion protein comprising the antibody and a co-stimulatory molecule, to the tumor microenvironment (Pertl et al., 2003).

Besides stimulating anti-tumor immune responses by inducing humoral responses, vaccines of the invention can also induce cellular responses, including stimulating T-cells that recognize and kill tumor cells directly. For example, nucleotide-based vaccines of the invention encoding tumor antigens can be used to activate the CD8⁺ cytotoxic T lymphocyte arm of the immune system.

In some embodiments, the vaccines activate T-cells directly, and in others they enlist antigen-presenting cells to activate T-cells. Killer T-cells are primed, in part, by interacting with antigen-presenting cells, i.e., dendritic cells. In some embodiments, plasmids comprising the nucleic acid molecules of the invention enter antigen-presenting cells, which in turn display the encoded tumor-antigens that contribute to killer T-cell activation. Again, the tumor antigens can be delivered as plasmid DNA constructs, either alone or with other molecules.

In further embodiments, RNA can be used. For example, dendritic cells can be transfected with RNA encoding tumor antigens (Heiser et al., 2002; Mitchell and Nair, 2000). This approach overcomes the limitations of obtaining sufficient quantities of tumor material, extending therapy to patients otherwise excluded from clinical trials. For example, a subject RNA molecule isolated from tumors can be amplified using RT-PCR. In some embodiments, the RNA molecule of the invention is directly isolated from tumors and transfected into dendritic cells with no intervening cloning steps.

In some embodiments the molecules of the invention are altered such that the peptide antigens are more highly antigenic than in their native state. These embodiments address the need in the art to overcome the poor in vivo immunogenicity of most tumor antigens by enhancing tumor antigen immunogenicity via modification of epitope sequences (Yu and Restifo, 2002).

Another recognized problem of cancer vaccines is the presence of preexisting neutralizing antibodies. Some embodiments of the present invention overcome this problem by using viral vectors from non-mammalian natural hosts, i.e., avian pox viruses. Alternative embodiments that also circumvent preexisting neutralizing antibodies include genetically engineered influenza viruses, and the use of “naked” plasmid DNA vaccines that contain DNA with no associated protein. (Yu and Restifo, 2002).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

For example, in some embodiments, nucleic acid vaccines encode an alphaviral replicase enzyme, in addition to tumor antigens. This recently discovered approach to vaccine therapy successfully combines therapeutic antigen production with the induction of the apoptotic death of the tumor cell (Yu and Restifo, 2002).

In certain other embodiments, a DNA or RNA vaccine of the present invention can also be directed against the production of blood vessels in the vicinity of the tumor, a process called antiangiogenesis, thereby depriving the cancer cells of nutrients. For example, the antiangiogenic molecules angiostatin (a fragment of plasminogen), endostatin (a fragment of collagen XVIII), interferon-γ, interferon-γ inducible protein 10, interleukin 12, thrombospondin, platelet factor-4, calreticulin, or its protein fragment vasostatin can be used to treat tumors by suppressing neovascularization and thereby inhibiting growth (Cheng et al., 2001). The antiangiogenesis approach can be used alone, or in conjunction with molecules directed to tumor antigens.

Inflammation and Immunity

In other embodiments, e.g., where the subject polypeptide is involved in modulating inflammation or immune function, the invention provides agents for treating such inflammation or immune disorders. Disease states that are treatable using formulations of the invention include various types of arthritis such as rheumatoid arthritis and osteoarthritis, autoimmune thyroiditis, various chronic inflammatory conditions of the skin, such as psoriasis, the intestine, such as inflammatory bowel disease, insulin-dependent diabetes, autoimmune diseases such as multiple sclerosis (MS), intestinal immune disorders and systemic lupus erythematosis (SLE), allergic diseases, transplant rejections, adult respiratory distress syndrome, atherosclerosis, ischemic diseases due to closure of the peripheral vasculature, cardiac vasculature, and vasculature in the central nervous system (CNS). After reading the present disclosure, those skilled in the art will recognize other disease states and/or symptoms which might be treated and/or mitigated by the administration of formulations of the present invention.

Neutralizing antibodies can provide immunosuppressive therapy for inflammatory and autoimmune disorders. Neutralizing antibodies can be used to treat disorders such as, for example, multiple sclerosis, rheumatoid arthritis, inflammatory bowel disease, transplant rejection, and psoriasis. Neutralizing antibodies that specifically recognize a secreted protein or peptide of the invention can bind to the secreted protein or peptide, e.g., in a bodily fluid or the extracellular space, thereby modulating the biological activity of the secreted protein or peptide. For example, neutralizing antibodies specific for secreted proteins or peptides that play a role in activating immune cells are useful as immunosuppressants.

Apoptosis, or programmed cell death, is a regulated process leading to cell death via a series of well-defined morphological changes. Programmed cell death provides a balance for cell growth and multiplication, eliminating unnecessary cells. The default state of the cell is to remain alive. A cell enters the apoptotic pathway when an essential factor is removed from the extracellular environment or when an internal signal is activated. Genes and proteins of the invention that suppress the growth of tumors by activating cell death provide the basis for treatment strategies for hyperproliferative disorders and conditions.

Other Pathological Conditions

Other pathological conditions that can be treated using the methods of the instant invention include infectious diseases, e.g., by using polypeptides of the invention to enhance immune function or act as adjuvants in vaccines, including cancer vaccines; disorders of hematopoeisis and/or cell differentiation; disorders of growth and differentiation that are affected by one or more growth factors; disorders of ion channels, e.g., cystic fibrosis; tissue or organ hypertrophy; viral disorders, including acquired immunodeficiency syndrome (AIDS); angiogenesis; metastasis; metabolic disorders such as diabetes and obesity; osteoporosis; neurodegenerative diseases; cardiovascular disorders such as congestive heart failure and stroke; male erectile dysfunction, disorders that can be treated by enhancing regeneration of neural cells, bone cells, skin cells, pancreatic islet cells, or lymphocytes, etc.; and other disorders described throughout the specification.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1 Expression in E. coli

Sequences can be expressed in E. coli. Any one or more of the sequences according to SEQ ID NOS.:1-54 can be expressed in E. coli by subcloning the entire coding region, or a selected portion thereof, into a prokaryotic expression vector. For example, the expression vector pQE16 from the QIA expression prokaryotic protein expression system (Qiagen, Valencia, Calif.) can be used. The features of this vector that make it useful for protein expression include an efficient promoter (phage T5) to drive transcription, expression control provided by the lac operator system, which can be induced by addition of IPTG (isopropyl-beta-D-thiogalactopyranoside), and an encoded 6×His tag coding sequence. The latter is a stretch of six histidine amino acid residues which can bind very tightly to a nickel atom. This vector can be used to express a recombinant protein with a 6×His. tag fused to its carboxyl terminus, allowing rapid and efficient purification using Ni-coupled affinity columns.

The entire or the selected partial coding region can be amplified by PCR, then ligated into digested pQE16 vector. The ligation product can be transformed by electroporation into electrocompetent E. coli cells (for example, strain M15-[pREP4] from Qiagen), and the transformed cells may be plated on ampicillin-containing plates. Colonies may then be screened for the correct insert in the proper orientation using a PCR reaction employing a gene-specific primer and a vector-specific primer. Also, positive clones can be sequenced to ensure correct orientation and sequence. To express the proteins, a colony containing a correct recombinant clone can be inoculated into L-Broth containing 100 μg/ml of ampicillin, and 25 μg/ml of kanamycin, and the culture allowed to grow overnight at 37 degrees C. The saturated culture may then be diluted 20-fold in the same medium and allowed to grow to an optical density of 0.5 at 600 nm. At this point, IPTG can be added to a final concentration of 1 mM to induce protein expression. After growing the culture for an additional 5 hours, the cells may be harvested by centrifugation at 3000 times g for 15 minutes.

The resultant pellet can be lysed with a mild, nonionic detergent in 20 mM Tris HCl (pH 7.5) (B PER™ Reagent from Pierce, Rockford, Ill.), or by sonication until the turbid cell suspension turns translucent. The resulting lysate can be further purified using a nickel-containing column (Ni-NTA spin column from Qiagen) under non-denaturing conditions. Briefly, the lysate will be adjusted to 300 mM NaCl and 10 mM imidazole, then centrifuged at 700 times g through the nickel spin column to allow the His-tagged recombinant protein to bind to the column. The column will be washed twice with wash buffer (for example, 50 mM NaH₂ PO₄, pH 8.0; 300 mM NaCl; 20 mM imidazole) and eluted with elution buffer (for example, 50 mM NaH2 PO4, pH 8.0; 300 mM NaCl; 250 mM imidazole). All the above procedures will be performed at 4 degrees C. The presence of a purified protein of the predicted size can be confirmed with SDS-PAGE.

### Example 2

### Expression in Mammalian Cells

The sequences encoding the polypeptides of Example 1 can be cloned into the pENTR vector (Invitrogen) by PCR and transferred to the mammalian expression vector pDEST12.2 per manufacturer's instructions (Invitrogen). Introduction of the recombinant construct into the host cell can be effected by transfection with Fugene 6 (Roche) per manufacturer's instructions. The host cells containing one of polynucleotides of the invention can be used in conventional manners to produce the gene product encoded by the isolated fragment (in the case of an ORF). A number of types of cells can act as suitable host cells for expression of the proteins. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

### Example 3

### Expression in Cell-Free Translation Systems

Cell-free translation systems can also be employed to produce proteins using RNAs derived from the DNA constructs of the present invention. Appropriate cloning and expression vectors containing SP6 or T7 promoters for use with prokaryotic and eukaryotic hosts have been described (Sambrook et al., 1989). These DNA constructs can be used to produce proteins in a rabbit reticulocyte lysate system or in a wheat germ extract system.

Specific expression systems of interest include plant, bacterial, yeast, insect cell and mammalian cell derived expression systems. Expression systems in plants include those described in U.S. Pat. No. 6,096,546 and U.S. Pat. No. 6,127,145. Expression systems in bacteria include those described by Chang et al., 1978, Goeddel et al., 1979, Goeddel et al., 1980, EP 0 036,776, U.S. Pat. No. 4,551,433; DeBoer et al., 1983, and Siebenlist et al., 1980.

Mammalian expression is further accomplished as described in Dijkema et al., 1985, Gorman et al., 1982, Boshart et al., 1985, and U.S. Pat. No. 4,399,216. Other features of mammalian expression are facilitated as described in Ham and Wallace, Meth. Enz., 1979, Barnes and Sato, 1980, U.S. Pat. Nos. 4,767,704, 4,657,866, 4,927,762, 4,560,655, WO 90/103430, WO 87/00195, and U.S. RE 30,985.

### Example 4

### Expression of the Secreted Factors in Yeast

Primers can be designed to amplify the secreted factors using PCR and cloned into pENTR/D-TOPO vectors (Invitrogen, Carlsbad. CA). The secreted factors in pENTR/D-TOPO can be cloned into the yeast expression vector pYES-DEST52 by Gateway LR reaction (Invitrogen, Carlsbad, Calif.). The resulting yeast expression vectors can be transformed into INVSc1 strain from Invitrogen to express the secreted factors according to the manufacturer's protocol (Invitrogen, Carlsbad Calif.). The expressed secreted factors will have a 6×His tag at the C-terminal. Expressed protein can be purified with ProBond™ resin (Invitrogen, Carlsbad, Calif.).

Expression systems in yeast include those described in Hinnen et al., 1978, Ito et al., 1983, Kurtz et al., 1986, Kunze et al., 1985, Gleeson et al., 1986, Roggenkamp et al., 1986, Das et al., 1984, De Louvencourt et al., 1983, Van den Berg et al., 1990, Kunze et al., 1985, Cregg et al. 1985, U.S. Pat. No. 4,837,148, U.S. Pat. No. 4,929,555, Beach and Nurse, 1981, Davidow et al., 1985, Gaillardin et al., 1985, Ballance et al., 1983, Tilburn et al., 1983, Yelton et al., 1984, Kelly and Hynes, 1985, EP 0 244,234, and WO 91/00357.

### Example 5

### Expression of Secreted Factors in Baculovirus

The secreted factors in pENTR/D-TOPO can be cloned into Baculovirus expression vector pDEST10 by Gateway LR reaction (Invitrogen, Carlsbad, Calif.). The secreted factors can be expressed by the Bac-to-Bac expression system from Invitrogen (Carlsbad Calif.), briefly described as follows. The expression vectors containing the secreted factors are transformed into competent DH10Bac™ E. coli strain and selected for transposition. The resulting E. coli contain recombinant bacmid that contains the secreted factor. High molecular weight DNA can be isolated from the E. coli containing the recombinant bacmid and then transfected into insect cells with Cellfectin reagent. The expressed secreted factors will have a 6×His tag at N-terminal. Expressed protein will be purified by ProBond™ resin (Invitrogen, Carlsbad, Calif.).

Expression of heterologous genes in insects can be accomplished as described in U.S. Pat. No. 4,745,051; Doerfler et al., 1087; Friesen et al., 1986; EP 0 127,839, EP 0 155,476, Vlak et al., 1988, Miller et al., 1988, Carbonell et al. 1988, Maeda et al., 1985, Lebacq-Verheyden et al., 1988, Smith et al., 1985, Miyajima et al.; and Martin et al., 1988. Numerous baculoviral strains and variants and corresponding permissive insect host cells from hosts have been previously described (Setlow et al., 1986, Luckow et al., 1988; Miller et al., 1986; Maeda et al., 1985).

### Example 6

### Primer Design

To design the forward primer for PCR amplification, the melting point of the first 20 to 24 bases of the primer can be calculated by counting total A and T residues, then multiplying by 2. To design the reverse primer for PCR amplification, the melting point of the first 20 to 24 bases of the reverse complement, with the sequences written from 5-prime to 3-prime can be calculated by counting the total G and C residues, then multiplying by 4. Both start and stop codons can be present in the final amplified clone. The length of the primers is such to obtain melting temperatures within 63 degrees C. to 68 degrees C. Adding the bases “CACC” to the forward primer renders it compatible for cloning the PCR product with the TOPO pENTR/D (Invitrogen, CA).

### Example 7

### Reverse Transcriptase Reaction

cDNA can be prepared by the following method. Between 200 ng and 1.0 μg mRNA is added to 2 μl DMSO and the volume adjusted to 11 μl with DEPC-treated water. One μl Oligo dT is added to the tube, and the mixture is heated at 70° C. for 5 min., quickly chilled on ice for 2 min., and the mixture is collected at the bottom of the tube by brief centrifugation. The following 1^{st} strand components are then added to the mRNA mixture: 2 μl 10× Stratascript (Stratagene, CA) 1^{st} strand buffer, 1 μl 0.1 M DTT, 1 μl 10 mM dNTP mix (10 mM each of dG, dA, dT and dCTP), 1 μl RNAse inhibitor, 3 μl Stratascript RT (50 U/μl). The contents are gently mixed and the mixture collected by brief centrifugation. The mixture is incubated in a 42° C. water bath for 1 hour, placed in a 70° C. water bath for 15 min. to stop the reaction, transferred to ice for 2 min., and centrifuged briefly in a microfuge to collect the reaction product at the bottom of the reaction vessel. Two μl RNAse H is then added to the tube, the contents are mixed well, incubated at 37° C. in a water bath for 20 min., and centrifuged briefly in a microfuge to collect the reaction product at the bottom of the reaction vessel. The reaction mixture can proceed directly to PCR or be stored at −20° C.

### Example 8

### Full Length PCR

Full length PCR can be achieved by placing the products of the reaction described in Example 7, with primers diluted to 5 μM in water, into a reaction vessel and adding a reaction mixture composed of 1×Taq buffer, 25 mM dNTP, 10 ng cDNA pool, TaqPlus (Stratagene, CA) (5 u/ul), PfuTurbo (Stratagene, CA) (2.5 u/ul), water. The contents of the reaction vessel are then mixed gently by inversion 5-6 times, placed into a reservoir where 2 μl F₁/R₁ primers are added, the plate sealed and placed in the thermocycler. The PCR reaction is comprised of the following eight steps. Step 1: 95° C. for 3 min. Step 2: 94° C. for 45 sec. Step 3: 0.5° C./sec to 56-60° C. Step 4: 56-60° C. for 50 sec. Step 5: 72° C. for 5 min. Step 6: Go to step 2, perform 35-40 cycles. Step 7: 72° C. for 20 min. Step 8: 4° C.

The products can then be separated on a standard 0.8 to 1.0% agarose gel at 40 to 80 V, the bands of interest excised by cutting from the gel, and stored at −20° C. until extraction. The material in the bands of interest can be purified with QIAquick 96 PCR Purification Kit (Qiagen, CA) according to the manufacturer instructions. Cloning can be performed with the Topo Vector pENTR/D-TOPO vector (Invitrogen, CA) according to the manufacturer's instructions.

### REFERENCES

The specification is most thoroughly understood in light of the following references, all of which are hereby incorporated by reference in their entireties. The disclosures of the patents and other references cited above are also hereby incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.
Agou, F., Quevillon, S., Kerjan, P., Latreille, M. T., Mirande, M. (1996) Functional replacement of hamster lysyl-tRNA synthetase by the yeast enzyme requires cognate amino acid sequences for proper tRNA recognition. Biochemistry 35:15322-15331.
Agrawal, S., Crooke, S. T. eds. (1998) Antisense Research and Application (Handbook of Experimental Pharmacology, Vol 131). Springer-Verlag New York, Inc.
Alberts, B., Bray, D., Lewis, J., Raff, M., Roberts, K., Watson, J. D. (1994) Molecular Biology of the Cell. 3^{rd} ed. Garland Publishing, Inc.
Alexander, D. R. (2000) The CD45 tyrosine phosphatase: a positive and negative regulator of immune cell function. Semin. Immunol 12:349-359.
Allison, A. C. (2000) Immunosuppressive drugs: the first 50 years and a glance forward. Immunopharmacology 47:63-83.
Altschul, S. F., Gish, W., Miller, W., Myers, E. W., Lipman, D. J. (1990) Basic alignment search tool. J. Mol. Biol. 215:403-410.
Altschul, S. F., Madden, T. L., Schäffer, A. A., Zhang, J., Zheng, Z., Miller, W., Lipman, D. J. (1997) Gapped BLAST and PSI-BLAST: a new generation of protein database search programs. Nucleic Acids Res. 25:3389-3402.
Amor, J. C., Harrison, D. H., Kahn, R. A., Ringe, D. (1994) Structure of the human ADP-ribosylation factor 1 complexed with GDP. Nature 372:704-708.
Andreeff, M., Pinkel, D. eds. (1999) Introduction to Fluorescence In Situ Hybridization: Principles and Clinical Applications. John Wiley & Sons.
Andres, D. A., Shao, H., Crick, D. C., Finlin, B. S. (1997) Expression cloning of a novel farnesylated protein, RDJ2, encoding a DnaJ protein homologue. Arch. Biochem. Biophys. 346:113-124.
Ansel, H. C., Allen, L., Popovich, N. G. eds. (1999) Pharmaceutical Dosage Forms and Drug Delivery Systems. 7^{th} ed. Lippencott Williams and Wilkins Publishers.
Aubry, M., Marineau, C., Zhang, F. R., Zahed, L., Figlewicz, D., Delattre, O., Thomas, G., de Jong, P. J., Julien, J. P., Rouleau, G. A. (1992) Cloning of six new genes with zinc finger motifs mapping to short and long arms of human acrocentric chromosome 22 (p and q11.2). Genomics 13:641-648.
Ausubel, F., Brent. R., Kingston, R. E., Moore, D. D., Seidman, I. G., Smith, J. A., eds. (1999) Short Protocols in Molecular Biology. 4^{th} ed. Wiley & Sons.
Baksh, S., Burakoff, S. J. (2000) The role of calcineurin in lymphocyte activation. Semin. Immunol. 12:405-415.
Ballance, D. J., Buxton, F. P., Turner, G. (1983) Transformation of Aspergillus nidulans by the orotidine-5′-phosphate decarboxylase gene of Neurospora crassa. Biochem. Biophys. Res. Commun. 112:284-289.
Barany, F. (1985) Single-stranded hexameric linkers: a system for in-phase insertion mutagenesis and protein engineering. Gene 37:111-123.
Barnes, D., Sato, G. (1980) Methods for growth of cultured cells in serum-free medium. Anal. Biochem. 102:255-270.
Barton, M. C., Hoekstra, M. F., Emerson, B. M. (1990) Site-directed, recombination-mediated mutagenesis of a complex gene locus. Nucleic Acids Res. 18:7349-7355.
Bashkin, J. K., Sampath, U., Frolova, E. (1995) Ribozyme mimics as catalytic antisense reagents. Appl. Biochem. Biotechnol. 54:43-56.
Bassett, D. E., Eisen, M. B., Boguski, M. S. (1999) Gene expression informatics—it's all in your mine. Nature Genetics 21:51-55.
Bast, R. C., Kufe, D. W., Pollock, R. E., Weichselbaum, R. R., Holland, J. F., Frei, E., eds. (2000) Cancer Medicine. 5th ed. B. C. Decker, Inc.
Bateman, A., Birney, E., Cerruti, L., Durbin, R., Etwiller, L., Eddy, S. R., Griffiths-Jones, S., Howe, K. L., Marshall, M., Sonnhammer, E. L. L. (2000) Nucleic Acids Research 30:276-280.
Battini, R., Ferrari, S., Kaczmarek, L., Calabretta, B., Chen, S. T., Baserga, R. (1987) Molecular cloning of a cDNA for a human ADP/ATP carrier which is growth-regulated. J. Biol. Chem. 262:4355-4359.
Bauer, C. E., Hesse, S. D., Waechter-Brulla, D. A., Lynn, S. P., Gumport, R. I., Gardner, J. F. (1985) A genetic enrichment for mutations constructed by oligodeoxynucleotide-directed mutagenesis. Gene 37:73-81.
Beach, D., Durkacz, B., Nurse, P. (1982) Functionally homologous cell cycle control genes in budding and fission yeast. Nature 300:706-709.
Beigelman, L., Karpeisky, A., Matulic-Adamic, J., Haeberli, P., Sweedler, D., Usman, N. (1995) Synthesis of 2′-modified nucleotides and their incorporation into hammerhead ribozymes. Nucleic Acids Res. 23:4434-4442.
Bennett, J. (2000) Gene therapy for retinitis pigmentosa. Curr. Opin. Mol. Ther. 2:420-425.
Berinstein, N. L. (2002) Carcinoembryonic antigen as a target for therapeutic anticancer vaccines: a review. J. Clin. Oncol. 20:2197-2207.
Bibikova, M., Beumer, K., Trautman, J. K., Carroll, D. (2003) Enhancing gene targeting with designed zinc finger nucleases. Science 300:764.
Birney, E., Durbin, R. (2000) Using GeneWise in the Drosophila annotation experiment. Genome Res. 10:547-548.
Blackwell, J. M., Barton, C. H., White, J. K., Searle, S., Baker, A. M., Williams, H., Shaw, M. A. (1995) Genomic organization and sequence of the human NRAMP gene: identification and mapping of a promoter region polymorphism. Mol. Med. 1: 194-205.
Bodzioch, M., Orso, E., Klucken, J., Langmann, T., Bottcher, A., Diederich, W., Drobnik, W., Barlage, S., Buchler, C., Porsch-Ozcurumez, M., Kaminski, W. E., Hahmann, H. W., Oette, K., Rothe, G., Aslanidis, C., Lackner, K. J., Schmitz, G. (1999) The gene encoding ATP-binding cassette transporter 1 is mutated in Tangier disease. Nat. Genet. 1999 22:347-351.
Bonifaci, N., Moroianu, J., Radu, A., Blobel, G. (1997) Karyopherin beta2 mediates nuclear import of a mRNA binding protein. Proc. Natl. Acad. Sci. 94:5055-5060.
Bortell, R. Owen, T. A., Bidwell, J. P., Gavazzo, P., Breen, E., van Wijnen, A. J., DeLuca, H. F., Stein, J. L., Lian, J. B., Stein, G. S. (1992) Vitamin D-responsive protein-DNA interactions at multiple promoter regulatory elements that contribute to the level of rat osteocalcin gene expression. Proc. Nail. Acad. Sci. 89:6119-6123.
Boshart, M., Weber, F., Jahn, G., Dorsch-Hasler, K., Fleckenstein, B., Schaffner, W. (1985) A very strong enhancer is located upstream of an immediate early gene of human cytomegalovirus. Cell 41:521-530.
Bowtell, D. D. L. (1999) Options available—from start to finish—for obtaining expression data by microarray. Nature Genetics 21:25-32.
Brenner, S., Williams, S. R., Vermass, E. H., Storck, T., Moon, K., McCollum, C., Mao, J. I., Luo, S., Kirchner, J. J., Eletr, S., DuBridge, R. B., Burcham, T., Albrecht, G. (2000) In vitro cloning of complex mixtures of DNA on microbeads: physical separation of differentially expressed cDNAs. Proc. Natl. Acad. Sci. USA 97:1665-1670.
Brock, G. (2000) Sildenafil citrate (Viagra®). Drugs Today 36:125-134.
Brown, J. R., Daar, I. O., Krug, J. R., Maquat, L. E. (1985) Characterization of the functional gene and several processed pseudogenes in the human triosephosphate isomerase gene family. Mol. Cell. Biol. 5:1694-1706.
Brown, P. O, Botstein, D. (1999) Exploring the new world of the genome with DNA microarrays. Nature Genetics 21:33-37.
Brunelleschi, S., Penengo, L., Santoro, M. M., Gaudino, G. (2002) Receptor tyrosine kinases as target for anti-cancer therapy. Curr. Pharm. Des. 8:1959-1972.
Brutlag, D. L., Dautricourt, J. P., Diaz, R., Fier, J., Moxon, B., Stamm, R. (1993). BLAZE: An implementation of the Smith-Waterman comparison algorithm on a massively parallel computer. Computers and Chemistry 17:203-207.
Campbell, K. H., McWhir, J., Ritchie, W. A., Wilmut, I. (1996) Sheep cloned by nuclear transfer from a cultured cell line. Nature 380:64-66.
Carbonell, L. F., Hodge, M. R., Tomalski, M. D., Miller, L. K. (1988) Synthesis of a gene coding for an insect-specific scorpion neurotoxin and attempts to express it using baculovirus vectors. Gene 73:409-418.
Carver, A. S., Dalrymple, M. A., Wright, G., Cottom, D. S., Reeves, D. B., Gibson, Y. H., Keenan, J. L., Barrass, J. D., Scott, A. R., Colman, A., et al. (1993) Transgenic livestock as bioreactors: stable expression of human alpha-1-antitrypsin by a flock of sheep. Biotechnology (N.Y.) 11:1263-1270.
Chakravarty, A. (1999) Population genetics—making sense out of sequence. Nature Genetics 21:56-60.
Chalifour, L. E., Fahmy, R., Holder, E. L., Hutchinson, E. W., Osterland, C. K., Schipper, H. M., Wang, E. (1994) A method for analysis of gene expression patterns. Anal. Biochem. 216: 299-304.
Chalut, C., Gallois, Y., Poterszman, A., Moncollin, V., Egly, J. M. (1995) Genomic structure of the human TATA-box-binding protein (TBP). Gene 161:277-282.
Chang, A. C., Nunberg, J. H., Kaufman, R T, Erlich, H. A., Schimke, R. T., Cohen, S. N. (1978) Phenotypic expression in E. coli of a DNA sequence coding for mouse dihydrofolate reductase. Nature 275:617-624.
Chang, M. S., Chang, C. L., Huang, C. J., Yang, Y. C. (2000) p 29, a novel GCIP-interacting protein, localizes in the nucleus. Biochem. Biophys. Res. Commun. 279:732-737.
Chen, F. W., Ioannou, Y. A. (1998) Ribosomal proteins in cell proliferation and apoptosis. Int. Rev. Immunol. 18:429-448.
Chen, S. Y., Bagley, J., Marasco, W. A. (1994) Intracellular antibodies as a new class of therapeutic molecule for gene therapy. Hum. Gene Ther. 5:595-601.
Cheng, W. F., Hung, C. F., Chai, C. Y., Hsu, K. F., He, L., Ling, M., Wu, T. C. (2001) Tumor-specific immunity and angiogenesis generated by a DNA vaccine encoding calreticulin linked to a tumor antigen. J. Clin. Invest. 108:669-678.
Cheung, V. G., Morley, M., Aquilar, F., Massimi, A., Kucherlapati, R., Childs, G. (1999) Making and reading microarrays. Nature Genetics 21:15-19.
Chien, C., Bartel, P. L., Sternglanz, R., Fields S. (1991) The two-hybrid system: A method to identify and clone genes for proteins that interact with a protein of interest. Proc. Natl. Acad. Sci. 88:9578-9581.
Christa, L., Simon, M. T., Flinois, J. P., Gebhardt, R., Brechot, C., Lasserre, C. (1994) Overexpression of glutamine synthetase in human primary liver cancer. Gastroenterology 106:1312-1320.
Chuang, V. T., Kragh-Hansen, U., Otagiri, M. (2002) Pharmaceutical strategies utilizing recombinant human serum albumin. Pharm. Res. 19:569-577.
Clark, C. M., Karlawish, J. H. (2003) Alzheimer disease: current concepts and emerging diagnostic and therapeutic strategies. Ann. Intern. Med. 138:400-410.
Coffin, J. M., Hughes, S. H., Varmus, H. E. (1997) Retroviruses. Cold Spring Harbor Laboratory Press.
Cole, K. A., Krizman, D. B., Emmert-Buck, M. R. (1999) The genetics of cancer—a 3D model. Nature Genetics 21:38-41.
Colicelli, J., Lobel, L. I., Goff, S. P. (1985) A temperature-sensitive mutation constructed by “linker insertion” mutagenesis. Mol. Gen. Genet. 199:537-539.
Coligan, J. E., Kruisbeek, A. M., Margulies, D. H., Shevach, E. M., Strober, W. (eds.) (2002) Current Protocols in Immunology, John Wiley and Sons, Inc.
Collins, F. S. (1999) Microarrays and macroconsequences. Nature Genetics 21:2.
Comuzzie, A. G., Allison, D. B. (1998) The search for human obesity genes. Science 280:1374-1377.
Cormand, B., Montfort, M., Chabas, A., Vilageliu, L., Grinberg, D. (1997) Genetic fine localization of the beta-glucocerebrosidase (GBA) and prosaposin (PSAP) genes: implications for Gaucher disease. Hum. Genet. 100:75-79.
Craik, C. S. (1985) Use of oligonucleotides for site-specific mutagenesis. Biotechniques 3:12-19.
Cregg, J. M., Barringer, K. J., Hessler, A. Y., Madden, K. R. (1985) Pichia pastoris as a host system for transformations. Mol. Cell. Biol. 5:3376-3385.
Crooke, S. T. (1996) Progress in antisense therapeutics. Med. Res. Rev. 16:319-344.
Crouch, R. J. (1990) Ribonuclease H: from discovery to 3D structure. New Biol. 2:771-777.
Curcio, L. D., Bouffard, D. Y., Scanlon, K. J. (1997) Oligonucleotides as modulators of cancer gene expression. Pharmacol. Ther. 74:317-332.
Das, S., Kellermann, E., Hollenberg, C. P. (1.984) Transformation of Kluyveromyces fragilis. J. Bacteriol. 158:1165-1167.
Davidow, L. S., Kaczmarek, F. S., DeZeeuw, J. R., Conlon, S. W., Lauth, M. R., Pereira, D. A., Franke, A. E. (1987) The Yarrowia lipolytica LEU2 gene. Curr. Genet. 11:377-383.
de Boer, H. A., Comstock, L. J., Vasser, M. (1993) The tac promoter: a functional hybrid derived from the trp and lac promoters. Proc. Nail. Acad. Sci. 80:21-25.
De Louvencourt, L., Fukuhara, H., Heslot, H., Wesolowski, M. (1983) Transformation of Kluyveromyces lactis by killer plasmid DNA. J. Bacteriol. 154:737-742.
De Vita, V. T., Jr., Hellman, S., Rosenberg, S. A., (2001) Cancer: Principles & Practice of Oncology. Lippincott Williams & Wilkins.
Deasy, B. M., Huard, J. (2002) Gene therapy and tissue engineering based on muscle-derived stem cells. Curr. Opin. Mol. Ther. 4:382-389.
Delahunty, C., Ankener, W., Deng, Q., Eng, J., Nickerson, D. A. (1996) Testing the feasibility of DNA typing for human identification by PCR and an oligonucleotide ligation assay. Am. J. Human Genetics 58: 1239-1246.
Deutscher, M. P., Simon, M. I., Abelson, J. N., eds. (1990) Guide to Protein Purification: Methods in Enzymology. (Methods in Enzymology Series, Vol 182). Academic Press.
Dieffenbach, C. W., Dveksler, G. S., eds. (1995) PCR Primer: A Laboratory Manual. Cold Spring Harbor Laboratory Press.
Dijkema, R., van der Meide, P. H., Pouwels, P. H., Caspers, M., Dubbeld, M., Schellekens, H. (1985) Cloning and expression of the chromosomal immune interferon gene of the rat. EMBO J. 4:761-767.
Ding, Y., Davisson, R. L., Hardy, D. O., Zhu, L. J., Merrill, D. C., Catterall, J. F., Sigmund, C. D. (1997) The kidney androgen-regulated protein promoter confers renal proximal tubule cell-specific and highly androgen-responsive expression on the human angiotensinogen gene in transgenic mice. J. Biol. Chem. 272:28,142-28,148.
Doerfler, W., Bohm, P., eds. (1987) The Molecular Biology Of Baculoviruses. Springer-Verlag, Inc.
Doll, A., Grzeschik, K. H. (2001) Characterization of two novel genes, WBSCR20 and WBSCR22, deleted in Williams-Beuren syndrome. Cytogenet. Cell Genet. 95:20-27.
Doolittle, R. F., Abelson, J. N., Simon, M. I., eds. (1996) Computer Methods for Macromolecular Sequence Analysis. 1st ed. Academic Press.
Ducrest, A. L., Suzutorisz, H., Lingner, J., Nabholz, M. (2002) Regulation of the human telomerase reverse transcriptase gene. Oncogene 21:541-52.
Dutoit, V., Taub, R. N., Papadopoulos, K. P., Talbot, S., Keohan, M. L., Brehm, M., Gnjatic, S., Harris, P. E., Bisikirska, B., Guillaume, P., Cerottini, J. C., Hesdorffer, C. S., Old, L. J., Valmori, D. (2002) Multiepitope CD8⁺ T cell response to an NY-ESO-1 peptide vaccine results in imprecise tumor targeting. J. Clin. Invest. 108:1813-1822.
Eglisson, V., Gudnason, V., Jonasdottir, A., Ingvarsson, S., Andresdottir, V. (1986) Catabolite repressive effects of 5-thio-D-glucose on Saccharomyces cerevisiae. J. Gen. Microbiol. 132:3309-3313.
Ehrhardt, G. R., Korherr, C., Wieler, J. S., Knaus, M., Schrader, J. W. (2001) A novel potential effector of M-Ras and p21 Ras negatively regulates p21 Ras-mediated gene induction and cell growth. Oncogene 20:188-197.
Espejo, A., Cote, J., Bednarek, A., Richard, S., Bedford, M. T. (2002) A protein-domain microarray identifies novel protein-protein interactions. Biochem. J. 367:697-702.
Everett, R. D., Meredith, M., Orr, A., Cross, A., Kathoria, M., Parkinson, J. (1997) A novel ubiquitin-specific protease is dynamically associated with the PML nuclear domain and binds to a herpesvirus regulatory protein. EMBO J. 16:1519-1530.
Fanning, A. S., Anderson, J. M. (1999) Protein modules as organizers of membrane structure. Curr. Opin. Cell Biol. 11:432-439.
Fields, S., Song, O. (1989) A novel genetic system to detect protein-protein interactions. Nature 340:245-246.
Filali, M., Liu, X. Cheng, N., Abbott, D., Leontiev, V., Engelhardt J. F. (2002) Mechanisms of submucosal gland morphogenesis in the airway. Novartis Found. Symp. 248:38-45; discussion 45-50, 277-282.
Fisch, P., Forster, A., Sherrington, P. D., Dyer, M. J., Rabbitts, T. H. (1993) The chromosomal translocation t(X;14)(q28;q11) in T-cell pro-lymphocytic leukaemia breaks within one gene and activates another. Oncogene 8:3271-3276.
Fishman, P. S., Oyler, G. A. (2002) Significance of the parkin gene and protein in understanding Parkinson's disease. Curr. Neurol. Neurosci. Rep. 2:296-302.
Forgac, M. (1999) Structure and properties of the vacuolar (H+)-ATPases. J. Biol. Chem. 274:12,951-12,954.
Frank, I. (2002) Antivirals against HIV-1. Clin. Lab. Med. 22:741-757.
Friesen, P. D., Miller, L. K. (1986) The regulation of baculovirus gene expression. Curr. Top. Microbiol. Immunol. 131:31-49.
Frithz, G., Ericsson, P., Ronquist, G. (1976) Serum adenylate kinase activity in the early phase of acute myocardial infarction. Ups J Med Sci. 81:155-158.
Funakoshi, I., Kato, H., Horie, K., Yano, T., Hori, Y., Kobayashi, H., Inoue, T., Suzuki, H., Fukui, S., Tsukahara, M., et al. (1992) Molecular cloning of cDNAs for human fibroblast nucleotide pyrophosphatase. Arch. Biochem. Biophys. 295:180-187.
Furth, P. A., Shamay, A., Wall, R. J., Hennighausen, L. (1992) Gene transfer into somatic tissues by jet injection. Anal. Biochem. 205:365-368.
Gaillardin, C., Ribet, A. M. (1987) LEU2 directed expression of beta-galactosidase activity and phleomycin resistance in Yarrowia lipolytica. Curr. Genet. 11:369-375.
Gao, X., Nawaz, Z. (2002) Progesterone receptors—animal models and cell signaling in breast cancer: Role of steroid receptor coactivators and corepressors of progesterone receptors in breast cancer. Breast Cancer Res. 4:182-186.
Gao, Y., Melki, R., Walden, P. D., Lewis, S. A., Ampe, C., Rommelaere, H., Vandekerckhove, J., Cowan, N. J. (1994) A novel cochaperonin that modulates the ATPase activity of cytoplasmic chaperonin. J. Cell Biol. 125:989-996.
Gaudilliere, B., Shi, Y., Bormi, A. (2002) RNA interference reveals a requirement for MEF2A in activity-dependent neuronal survival. J. Biol. Chem. 277:46,442-46,446 [epub Sep. 13, 2002, ahead of print].
Gavrieli, Y., Sherman, Y., Ben-Sasson, S. A. (1992) Identification of programmed cell death in situ via specific labeling of nuclear DNA fragmentation. J. Cell Biol. 119:493-501.
Geffen D. B., Man S. (2002) New drugs for the treatment of cancer, 1990-2001. Isr. Med. Assoc. J. 4:1124-31.
Gennaro, A. R. (2003) Remington: The Science and Practice of Pharmacy with Facts and Comparisons: Drugfacts Plus. 20th ed., Lippincott Williams & Williams.
Ghofrani, H. A., Rose, F., Schermuly, R. T., Olschewski, H., Wiedemann, R., Kreckel, A., Weissmann, N., Ghofrani, S., Enke, B., Seeger, W., Grimminger, F. (2003) Oral sildenafil as long-term adjunct therapy to inhaled iloprost in severe pulmonary arterial hypertension. J. Am. Coll. Cardiol. 42:158-164.
Gillingham, A. K., Pfeifer, A. C., Munro, S. (2002) CASP, the alternatively spliced product of the gene encoding the CCAAT-displacement protein transcription factor, is a Golgi membrane protein related to giantin. Mol. Biol. Cell 13:3761-3774.
Gingras, M. C., Lapillonne, H., Margolin, J. F. (2002) TREM-1, MDL-1, and DAP12 expression is associated with a mature stage of myeloid development. Mol. Immunol. 38:817-824.
Girschick, H. J., Grammer, A. C., Nanki, T., Vazquez, E., Lipsky, P. E. (2002) Expression of recombination activating genes 1 and 2 in peripheral B cells of patients with systemic lupus erythematosus. Arthritis. Rheum. 46:1255-1263.
Glasser, S. W., Korfhagen, T. R., Bruno, M. D., Dey, C., Whitsett, J. A. (1990) Structure and expression of the pulmonary surfactant protein SP-C gene in the mouse. J. Biol. Chem. 265:21,986-21,991.
Gmeiner, W. H., Horita, D. A. (2001) Implications of SH3 domain structure and dynamics for protein regulation and drug design. Cell Biochem. Biophys. 35:127-140.
Goeddel, D. V., Heyneker, H. L., Hozumi, T., Arentzen, R., Itakura, K., Yansura, D. G., Ross, M. J., Mizzari, G., Crea, R., Seeburg, P. H. (1979) Direct expression in E. coli of a DNA sequence coding for human growth hormone. Nature 281:544-548.
Goeddel, D. V., Shephard, H. M., Yelverton, E., Leung, D., Crea, R., Sloma, A., Pestka, S. (1980) Synthesis of human fibroblast interferon by E. coli. Nucleic Acids Res. 8:4057-4074.
Goldenberg, M. M. (1999) Etanercept, a novel drug for the treatment of patients with severe, active rheumatoid arthritis. Clin. Ther. 21:75-87.
Goldstein, L. S. B., Yang, Z. (2000) Microtubule-based transport systems in neurons: the roles of kinesins and dyneins. Annu. Rev. Neurosci. 23:39-71.
Golovkina, T. V., Chervonsky, A., Dudley, J. P., Ross, S. R. (1992) Transgenic moue mammary tumor virus superantigen expression prevents viral infection. Cell 69:637-645.
Gonnet, G. H., Cohen, M. A., Benner, S. A. (1992) Exhaustive matching of the entire protein sequence database. Science 256:1443-1445.
Gordan, J. D., Vonderheide, R. H. (2002) Universal tumor antigens as targets for immunotherapy. Cytotherapy 4:317-327.
Gordon, J. W. (1989) Transgenic animals. Int. Rev. Cytol. 115:171-229.
Gorman, C. M., Merlino, G. T., Willingham, M. C., Pastan, I., Howard, B. H. (1982) The Rous sarcoma virus long terminal repeat is a strong promoter when introduced into a variety of eucaryotic cells by DNA-mediated transfection. Proc. Natl. Acad. Sci. 79:6777-6781.
Gorman, C. M., Merlino, G. T., Willingham, M. C., Pastan, I., Howard, B. H. (1982) The Rous sarcoma virus long terminal repeat is a strong promoter when introduced into a variety of eucaryotic cells by DNA-mediated transfection. Proc. Natl. Acad. Sci. 79:6777-6781.
Gray, T. A., Hernandez, L., Carey, A. H., Schaldach, M. A., Smithwick, M. J., Rus, K. M., Graves, J. A., Stewart, C. L., Nicholls, R. D. (2002) The ancient source of a distinct gene family encoding proteins featuring RING and C(3)H zinc-finger motifs with abundant expression in developing brain and nervous system. Genomics. 66:76-86.
Griffiths, A. J. F., Miller, J. H., Suzuki, D. T., Lewontin, R. C., Gelbart, W. M. (1999) Introduction to Genetic Analysis. 7^{th} ed. W.H. Freeman.
Griffiths, M., Beaumont, N., Yao, S. Y., Sundaram, M., Boumah, C. E., Davies, A., Kwong, F. Y., Coe, I., Cass, C. E., Young, J. D., Baldwin, S. A. (1997) Cloning of a human nucleoside transporter implicated in the cellular uptake of adenosine and chemotherapeutic drugs. Nat. Med. 3:89-93.
Grosschedl, R., Baltimore, D. (1985) Cell-type specificity of immunoglobulin gene expression is regulated by at least three DNA sequence elements. Cell 41:885-897.
Grosveld, F., Kollias, G., eds. (1992) Transgenic Animals. 1^{st} ed. Academic Press.
Gu, H., Marth, J. D., Orban, P. C., Mossmann, H., Rajewsky, K. (1994) Deletion of a DNA polymerase beta gene segment in T cells using cell type-specific gene targeting. Science. 265: 103-106.
Gustin, K., Burk, R. D. (1993) A rapid method for generating linker scanning mutants utilizing PCR. Biotechniques 14:22-24.
Hacia, J. G. (1999) Resequencing and mutational analysis using oligonucleotide microarrays. Nature Genetics 21:42-47.
Hadano, S., Yanagisawa, Y., Skaug, J., Fichter, K., Nasir, J., Martindale, D., Koop, B. F., Scherer, S. W., Nicholson, D. W., Rouleau, G. A., Ikeda, J., Hayden, M. R. (2001) Cloning and characterization of three novel genes, ALS2CR1, ALS2CR2, and ALS2CR3, in the juvenile amyotrophic lateral sclerosis (ALS2) critical region at chromosome 2q33-q34: candidate genes for ALS2. Genomics 71:200-213.
Hall, M., Mickey, D. D., Wenger, A. S., Silverman, L. M. (1985) Adenylate kinase: an oncodevelopmental marker in an animal model for human prostatic cancer. Clin. Chem. 31:1689-1691.
Ham, R. G., McKeehan, W. L. (1979) Media and growth requirements. Methods Enzymol. 58:44-93.
Hanada, T., Lin, L., Tibaldi, E. V., Reinherz, E. L., Chishti, A. H. (2000) GAKIN, a novel kinesin-like protein associates with the human homologue of the Drosophila discs large tumor suppressor in T lymphocytes. J. Biol. Chem. 275:28,774-28,784.
Harlow, E., Lane, D., eds. (1988) Antibodies: A Laboratory Manual. Cold Spring Harbor Laboratory.
Harlow, E., Lane, D., Harlow, E., eds. (1998) Using Antibodies: A Laboratory Manual: Portable Protocol NO. I. Cold Spring Harbor Laboratory.
Harris, J. M., Martin, N. E., Modi, M. (2001) Pegylation: a novel process for modifying pharmacokinetics. Clin. Pharmacokinet. 40:539-551.
Hartmann, G., Endres, S., eds. (1999) Manual of Antisense Methodology (Perspectives in Antisense Science). 1^{st} ed. Kluwer Law International.
Hassanzadeh, G. H. G., De Silva, K. S., Dambly-Chudiere, C., Brys, L., Ghysen, A., Hamers, R., Muyldermans, S., De Baetselier, P. (1998) Isolation and characterization of single-chain Fv genes encoding antibodies specific for Drosophila Poxn protein. FEBS Lett. 437:75-80.
Hawes, J. W., Jaskiewicz, J., Shimomura, Y., Huang, B., Bunting, J., Harper, E. T., Harris, R. A. (1996) Primary structure and tissue-specific expression of human beta-hydroxyisobutyryl-coenzyme A hydrolase. J. Biol. Chem. 271:26,430-26,434.
Hawley, R. G. (2001) Progress toward vector design for hematopoeitic stem cell gene therapy. Curr. Gene Ther. 1:1-17.
Heath, J. K., White, S. J., Johnstone, C. N., Catimel, B., Simpson, R. J., Moritz, R. L., Tu, G. F., Ji, H., Whitehead, R. H., Groenen, L. C., Scott, A. M., Ritter, G., Cohen, L., Welt, S., Old, L. J., Nice, E. C., Burgess, A. W. (1997) The human A33 antigen is a transmembrane glycoprotein and a novel member of the immunoglobulin superfamily. Proc. Natl. Acad. Sci. 94:469-474.
Heiser, A., Coleman, D., Dannull, J., Yancey, D., Maurice, M. A., Lallas, C. D., Dahm, P., Niedzwiecki, D., Gilboa, E., Vieweg, J. (2002) Autologous dendritic cells transfected with prostate-specific antigen RNA stimulate CTL responses against metastatic prostate tumors. J. Clin. Invest. 109:409-417.
Henningson, C. T. Jr., Stanislaus, M. A., Gewirtz, A. M. (2003) Embryonic and adult stem cell therapy. J. Allergy Clin. Immunol. 111:S745-S753.
Hinnen, A., Hicks, J. B., Fink, G. R. (1978) Transformation of yeast. Proc. Natl. Acad. Sci. 75:1929-1933.
Hirsch, D. S., Pirone, D. M., Burbelo, P. D. (2001) A new family of Cdc42 effector proteins, CEPs, function in fibroblast and epithelial cell shape changes. J. Biol. Chem. 276:875-883.
Ho, L. W., Carmichael, J., Swartz, J., Wyttenbach, A., Rankin, J., Rubinsztein, D. C. (2001) The molecular biology of Huntington's disease. Psychol. Med. 31:3-14.
Hollis, G. F., Evans, R. J., Stafford-Hollis, J. M., Korsmeyer, S. J., McKearn, J. P. (1989) Immunoglobulin lambda light-chain-related genes 14.1 and 16.1 are expressed in pre-B cells and may encode the human immunoglobulin omega light-chain protein. Proc. Natl. Acad. Sci. 86:5552-5556.
Hong, G. F. (1982) Sequencing of large double-stranded DNA using the dideoxy sequencing technique. Biosci. Rep. 2:907-912.
Hoogenboom, H. R., de Bruin, A. P., Hufton, S. E., Hoet, R. M., Arends, J. W., Roovers, R. C. (1998) Antibody phage display technology and its applications. Immunotechnology 4:1-20.
Hooper, M. L. (1993) Embryonal Stem Cells: Introducing Planned Changes into the Animal Germline. Gordon & Breach Science Pub.
Hoozemans, J. J., Veerhuis, R., Rozemuller, A. J., Eikelenboom, P. (2002) The pathological cascade of Alzheimer's disease: the role of inflammation and its therapeutic implications. Drugs Today (Barc) 38:429-443.
Houseman, B. T., Huh, J. H., Kron, S. J., Mrksich, M. (2002) Peptide chips for the quantitative evaluation of protein kinase activity. Nature Biotechnol. 20:270-274.
Howard, G. C., Bethell, D. R. (2000) Basic Methods in Antibody Production and Characterization. CRC Press.
Hunt, C. R., Ro, J. H., Dobson, D. E., Min, H. Y., Spiegelman, B. M. (1986) Adipocyte P2 gene: developmental expression and homology of 5′-flanking sequences among fat cell-specific genes. Adipocyte P2 gene: developmental expression and homology of 5′-flanking sequences among fat cell-specific genes. Proc. Natl. Acad. Sci. 83:3786-3790.
Huynh, D. P., Yang, H. T., Vakharia, H., Nguyen, D., Pulst, S. M. (2003) Expansion of the polyQ repeat in ataxin-2 alters its Golgi localization, disrupts the Golgi complex and causes cell death. Hum. Mol. Genet. 12:1485-1496.
Ikeda, A., Nishina, P. M., Naggert, J. K. (2002) The tubby-like proteins, a family with roles in neuronal development and function. J. Cell Sci. 115(Pt 1):9-14.
Ito, H., Fukuda, Y., Murata, K., Kimura, A. (1978) Transformation of intact yeast cells treated with alkali cations. J. Bacteriol. 153:163-168.
Jameson, D. M., Sawyer, W. H. (1995) Fluorescence anisotropy applied to biomolecular interactions. Methods Enzymol. 246:283-300.
Janeway, C. A., Travers, P. Walport, M. Shlomchik, M. (2001) Immunobiology. 5^{th} ed. Garland Publishing.
Jeffery, P., Zhu, J. (2002) Mucin-producing elements and inflammatory cells. Novartis Found. Symp. 248:51-75, 277-82.
Jimbo, T., Kawasaki, Y., Koyama, R., Sato, R., Takada, S., Haraguchi, K., Akiyama, T. (2002) Identification of a link between the tumour suppressor APC and the kinesin superfamily. Nat. Cell Biol. 4:323-327.
Joberty, G., Perlungher, R. R., Macara, I. G. (1999) The Borgs, a new family of Cdc42 and TC10 GTPase-interacting proteins. Mol. Cell. Biol. 19:6585-6597.
Johns, T. G., Bernard, C. C. (1997) Binding of complement component Clq to myelin oligodendrocyte glycoprotein: a novel mechanism for regulating CNS inflammation. Mol. Immunol. 34:33-38.
Jolliffe, C. N., Harvey, K. F., Haines, B. P., Parasivam, G., Kumar, S. (2000) Identification of multiple proteins expressed in murine embryos as binding partners for the WW domains of the ubiquitin-protein ligase Nedd4. Biochem. J. 351:557-565.
Jones, D. H., Winistorfer, S. C. (1992) Recombinant circle PCR and recombination PCR for site-specific mutagenesis without PCR product purification. Biotechniques 12:528-530.
Jones, P., ed. (1998a) Vectors: Cloning Applications: Essential Techniques, John Wiley & Son, Ltd.
Jones, P., ed. (1998b) Vectors: Expression Systems: Essential Techniques, John Wiley & Son, Ltd.
Jost, C. R., Kurucz, I., Jacobus, C. M., Titus, J. A., George, A. J., Segal, D. M. (1994) Mammalian expression and secretion of functional single-chain Fv molecules. J. Biol. Chem. 269:26,267-26,273.
Joulin, V., Richard-Foy, H. (1995) A new approach to isolate genomic control regions. Application to the GATA transcription factor family. Eur. J. Biochem. 232:620-626.
Jurcic, J. G., Cathcart, K., Pinilla-Ibarz, J., Scheinberg, D. A. (2000) Advances in immunotherapy of hematlogic malignancies: cellular and humoral approaches. Curr. Opin. Hematol. 7:247-254.
Jury, J. A., Perry, A. C., Hall, L. (1999) Identification, sequence analysis and expression of transcripts encoding a putative metalloproteinase, eMDC II, in human and macaque epididymis. Mol. Hum. Reprod. 5:1127-1134.
Kabat, E. A., Wu, T. T. (1991) Identical V region amino acid sequences and segments of sequences in antibodies of different specificities. Relative contributions of VH and VL genes, minigenes, and complementarity-determining regions to binding of antibody-combining sites. J. Immunol. 147:1709-1719.
Kamitani, T., Nguyen, H. P., Yeh, E. T. (1997) Preferential modification of nuclear proteins by a novel ubiquitin-like molecule. J. Biol. Chem. 272:14,001-14,004.
Kantoff, P. W., Halabi, S., Farmer, D. A., Hayes, D. F., Vogelzang, N. A., Small, E. J. (2001) Prognostic significance of reverse transcriptase polymerase chain reaction for prostate-specific antigen in men with hormone-refractory prostate cancer. J. Clin. Oncol. 9:3025-3028.
Kao, P. N., Chen, L., Brock, G., Ng, J., Kenny, J., Smith, A. J., Corthesy, B. (1994) Cloning and expression of cyclosporin A- and FK506-sensitive nuclear factor of activated T-cells: NF45 and NF90. J. Biol. Chem. 269:20,691-20,699.
Karanazanashvili, G., Abrahamsson, P. (2003) Prostate specific antigen and human glandular kallikrein 2 in early detection of prostate cancer. J. Urol. 169:445-457.
Kari, C., Chan, T. O., Rocha de Quadros, M., Rodeck, U. (2003) Targeting the epidermal growth factor receptor in cancer: apoptosis takes center stage. Cancer Res. 63:1-5.
Kaykas, A., Moon, R. T. (2004) A plasmid-based system for expressing small interfering RNA libraries in mammalian cells. BMC Cell Biol. 5:16-26.
Kelly, J. M., Hynes, M. J. (1985) Transformation of Aspergillus niger by the mdS gene of Aspergillus nidulans. EMBO J. 4:475-479.
Kenmochi, N., Kawaguchi, T., Rozen, S., Davis, E., Goodman, N., Hudson, T. J., Tanaka, T., Page, D. C. (1998) A map of 75 human ribosomal protein genes. Genome Res. 8:509-523.
Keown, W. A., Campbell, C. R., Kucherlapati, R. S. (1990) Methods for introducing DNA into mammalian cells. Methods Enzymol. 185:527-537.
Kibbe, A. H., ed. (2000) Handbook of Pharmaceutical Excipients. 3^{rd} ed. Pharmaceutical Press.
Kirkpatrick, K. L., Mokbel, K. (2001) The significance of human telomerase reverse transcriptase (hTERT) in cancer. Eur. J. Surg. Oncol. 27:754-760.
Kirsch, K. H., Georgescu, M. M., Ishimaru, S., Hanafusa, H. (1999) CMS: an adapter molecule involved in cytoskeletal rearrangements. Proc. Natl. Acad. Sci. 96:6211-6216.
Kiryu-Seo, S., Sasaki, M., Yokohama, H., Nakagomi, S., Hirayama, T., Aoki, S., Wada, K., Kiyama, H. (2000) Damage-induced neuronal endopeptidase (DINE) is a unique metallopeptidase expressed in response to neuronal damage and activates superoxide scavengers. Proc. Natl. Acad. Sci. 97:4345-4350.
Klarman, G. J., Hawkins, M. E., Le Grice, S. F. (2002) Uncovering the complexities of retroviral ribonuclease H reveals its potential as a therapeutic target. AIDS Rev. 4: 183-194.
Knutson, K. L., Schiffman, K., Disis, M. L. (2001) Immunization with a HER-2/neu helper peptide vaccine generates HER-2/neu CD8 T-cell immunity in cancer patients. J. Clin. Invest. 107:477-484.
Kobayashi, M., Takezawa, S., Hara, K., Yu, R. T., Umesono, Y., Agata, K., Taniwaki, M., Yasuda. K., Umesono, K. (1999) Identification of a photoreceptor cell-specific nuclear receptor. Proc. Natl. Acad. Sci. 96:4814-4819.
Kolonin, M. G., Finley, R. L. Jr. (1998) Targeting cyclin-dependent kinases in Drosophila with peptide aptamers. Proc. Natl. Acad. Sci. 95:14,266-14,271.
Korner, C., Knauer, R., Stephani, U., Marquardt, T., Lehle, L., von Figura, K. (1999) Carbohydrate deficient glycoprotein syndrome type IV: deficiency of dolichyl-P-Man: Man(5)GlcNAc(2)-PP-dolichyl mannosyltransferase. EMBO J. 18:6816-6822.
Kothapalli, R., Buyuksal, I., Wu, S. Q., Chegini, N., Tabibzadeh, S. (1997) Detection of ebaf, a novel human gene of the transforming growth factor beta superfamily association of gene expression with endometrial bleeding. J. Clin. Invest. 99:2342-2350.
Kovalenko, O. V., Golub, E. I, Bray-Ward, P., Ward, D. C., Radding, C. M. (1997) A novel nucleic acid-binding protein that interacts with human rad51 recombinase. Nucleic Acids Res. 25:4946-4953.
Kratzschmar, J., Lum, L., Blobel, C. P. (1996) Metargidin, a membrane-anchored metalloprotease-disintegrin protein with an RGD integrin binding sequence. J. Biol. Chem. 271:4593-4596.
Ku, D. H., Kagan, J., Chen, S. T., Chang, C. D., Baserga, R., Wurzel, J. (1990) The human fibroblast adenine nucleotide translocator gene. Molecular cloning and sequence. J. Biol. Chem. 265:16,060-16,063.
Kuisle, O., Quiñoá, E., Rigura, R. (1999) Solid phase synthesis of depsides and depsipeptides. Tetrahedron Lett. 40:1203-1206.
Kunze, G. et al., (1985) Transformation of the industrially important yeasts Candida maltosa and Pichia guilliermondii. J. Basic Microbiol. 25:141-144.
Kurtz, M. B., Cortelyou, M. W., Kirsch, D. R. (1986) Integrative transformation of Candida albicans, using a cloned Candida ADE2 gene. Mol. Cell. Biol. 6:142-149.
Kyo, S., Takakura, M., Inoue, M. (2000) Telomerase activity in cancer as a diagnostic and therapeutic target. Histol. Histopathol. 15:813-824.
Lakso, M., Sauer, B., Mosinger, B. Jr., Lee, E. J., Manning, R. W., Yu, S. H., Mulder, K. L., Westphal, H. (1992) Targeted oncogene activation by site-specific recombination in transgenic mice. Proc. Natl. Acad. Sci. 89:6232-6236.
Lander, E. S. (1999) Array of hope. Nature Genetics 21:3-4.
Lander, E. S., Linton, L. M., Birren, B., Nusbaum, C., Zody, M. C., Baldwin, J., Devon, K., Dewar, K., Doyle, M., FitzHugh, W., Funke, R., Gage, D., Harris, K., Heaford, A., Howland, J., Kann, L., Lehoczky, J., LeVine, R., McEwan, P., McKeman, K., Meldrim, J., Mesirov, J. P., Miranda, C., Morris, W., Naylor, J., Raymond, C., Rosetti, M., Santos, R., Sheridan, A., Sougnez, C., Stange-Thomann, N., Stojanovic, N., Subramanian, A., Wyman, D., Rogers, J., Sulston, J., Ainscough, R., Beck, S., Bentley, D., Burton, J., Clee, C., Carter, N., Coulson, A., Deadman, R., Deloukas, P., Dunham, A., Dunham, I., Durbin, R., French, L., Grafham, D., Gregory, S., Hubbard, T., Humphray, S., Hunt, A., Jones, M., Lloyd, C., McMurray, A., Matthews, L., Mercer, S., Milne, S., Mullikin, J. C., Mungall, A., Plumb, R., Ross, M., Showukeen, R., Sims, S., Waterston, R. H., Wilson, R. K., Hillier, L. W., McPherson, J. D., Marra, M. A., Mardis, E. R., Fulton, L. A., Chinwalla, A. T., Pepin, K. H., Gish, W. R., Chissoe, S. L., Wendl, M. C., Delehaunty, K. D., Miner, T. L., Delehaunty, A., Kramer, J. B., Cook, L. L., Fulton, R. S., Johnson, D. L., Minx, P. J., Clifton, S. W., Hawkins, T., Branscomb, E., Predki, P., Richardson, P., Wenning, S., Slezak, T., Doggett, N., Cheng, J. F., Olsen, A., Lucas, S., Elkin, C., Uberbacher, E., Frazier, M., Gibbs, R. A., Muzny, D. M., Scherer, S. E., Bouck, J. B., Sodergren, E. J., Worley, K. C., Rives, C. M., Gorrell, J. H., Metzker, M. L., Naylor, S. L., Kucherlapati, R. S., Nelson, D. L., Weinstock, G. M., Sakaki, Y., Fujiyama, A., Hattori, M., Yada, T., Toyoda, A., Itoh, T., Kawagoe, C., Watanabe, H., Totoki, Y., Taylor, T., Weissenbach, J., Heilig, R., Saurin, W., Artiguenave, F., Brottier, P., Bruls, T., Pelletier, E., Robert, C., Wincker, P., Smith, D. R., Doucette-Stamm, L., Rubenfield, M., Weinstock, K., Lee, H. M., Dubois, J., Rosenthal, A., Platzer, M., Nyakatura, G., Taudien, S., Rump, A., Yang, H., Yu, J., Wang, J., Huang, G., Gu, J., Hood, L., Rowen, L., Madan, A., Qin, S., Davis, R. W., Federspiel, N. A., Abola, A. P., Proctor, M. J., Myers, R. M., Schmutz, J., Dickson, M., Grimwood, J., Cox, D. R., Olson, M. V., Kaul, R., Raymond, C., Shimizu, N., Kawasaki, K., Minoshima, S., Evans, G. A., Athanasiou, M., Schultz, R., Roe, B. A., Chen, F., Pan, H., Ramser, J., Lehrach, H., Reinhardt, R., McCombie, W. R., de la Bastide, M., Dedhia, N., Blocker, H., Hornischer, K., Nordsiek, G., Agarwala, R., Aravind, L., Bailey, J. A., Bateman, A., Batzoglou, S., Birney, E., Bork, P., Brown, D. G., Burge, C. B., Cerutti, L., Chen, H. C., Church, D., Clamp, M., Copley, R. R., Doerks, T., Eddy, S. R., Eichler, E. E., Furey, T. S., Galagan, J., Gilbert, J. G., Hannon, C., Hayashizaki, Y., Haussler, D., Hermjakob, H., Hokamp, K., Jang, W., Johnson, L. S., Jones, T. A., Kasif, S., Kaspryzk, A., Kennedy, S., Kent, W. J., Kitts, P., Koonin, E. V., Korf, I., Kulp, D., Lancet, D., Lowe, T. M., McLysaght, A., Mikkelsen, T., Moran, J. V., Mulder, N., Pollara, V. J., Ponting, C. P., Schuler, G., Schultz, J., Slater, G., Smit, A. F., Stupka, E., Szustakowski, J., Thierry-Mieg, D., Thierry-Mieg, J., Wagner, L., Wallis, J., Wheeler, R., Williams, A., Wolf, Y. I., Wolfe, K. H., Yang, S. P., Yeh, R. F., Collins, F., Guyer, M. S., Peterson, J., Felsenfeld, A., Wetterstrand, K. A., Patrinos, A., Morgan, M. J., Szustakowki, J., de Jong, P., Catanese, J. J., Osoegawa, K., Shizuya, H., Choi, S., Chen, Y. J.; International Human Genome Sequencing Consortium. (2001) Initial sequencing and analysis of the human genome Nature 409:860-921.
Larochelle, N., Lochmuller, H., Zhao, J., Jani, A., Hallauer, P., Hastings, K. E., Massie, B., Prescott, S., Petrof, B. J., Karpati, G., Nalbantoglu, J. (1997) Gene Ther. 4:465-472.
Lasham, A., Moloney, S., Hale, T., Horner, C., Zhang, Y. F., Murison, J. G., Braithwaite, A. W., Watson, J. (2003) The Y-box binding protein YB1: A potential negative regulator of the p53 tumor suppressor. J. Biol. Chem. Epub ahead of print, Jun. 30, 2003.
Lashkari, A., Smith, A. K., Graham, J. M. Jr. (1999) Williams-Beuren syndrome: an update and review for the primary physician. Clin. Pediatr. 38:189-208.
Lavedan, C. (1998) The synuclein family. Genome Res. 8:871-880.
Lavitrano, M., Camaioni, A., Fazio, V. M., Dolci, S., Farace, M. G., Spadafora, C. (1989) Sperm cells as vectors for introducing foreign DNA into eggs: genetic transformation of mice. Cell 57:717-723.
Lebacq-Verheyden, A. M., Kasprzyk, P. G., Raum, M. G., Van Wyke Coelingh, K., Lebacq, J. A., Battey, J. F. (1988) Posttranslational processing of endogenous and of baculovirus-expressed human gastrin-releasing peptide precursor. Mol. Cell. Biol. 8:3129-3135.
Lees-Miller, S. P., Anderson, C. W. (1989) Two human 90-kDa heat-shock proteins are phosphorylated in vivo at conserved serines that are phosphorylated in vitro by casein kinase II. J. Biol. Chem. 264:2431-2437.
Lerch, M. M., Gorelick, F. S. (2000) Early trypsinogen activation in acute pancreatitis. Med. Clin. North Amer. 84:549-563.
Lerner, R. A. (1982) Tapping the immunological repertoire to produce antibodies of predetermined specificity. Nature 299:592-596.
Li, E., Bestagno, M., Burrone, O. (1996) Molecular cloning and characterization of a transmembrane surface antigen in human cells. Eur. J. Biochem. 238:631-638.
Li, H., Pamukcu, R., Thompson, W. J. (2002) beta-Catenin signaling: therapeutic strategies in oncology. Cancer Biol. Ther. 1:621-625.
Lim, D., Orlova, M., Goff, S. P. (August 2002) Mutations of the RNase H C helix of the Moloney murine leukemia virus reverse transcriptase reveal defects in polypurine tract recognition. J. Virol. 76:8360-8373.
Lin, B., Rommens, J. M., Graham, R. K., Kalchman, M., MacDonald, H., Nasir, J., Delaney, A., Goldberg, Y. P., Hayden, M. R. (1993) Differential 3′ polyadenylation of the Huntington disease gene results in two mRNA species with variable tissue expression. Hum. Mol. Genet. 2:1541-1545.
Lin, W. J., Gary, J. D., Yang, M. C., Clarke, S., Herschman, H. R. (1996) The mammalian immediate-early TIS21 protein and the leukemia-associated BTG1 protein interact with a protein-arginine N-methyltransferase. J. Biol. Chem. 271:15,034-15,044.
Lin, X., SikkiNK cells, R. A., Rusnak, F., Barber, D. L. (1999) Inhibition of calcineurin phosphatase activity by a calcineurin B homologous protein. J. Biol. Chem. 274:36,125-36,131.
Linnenbach, A. J., Seng, B. A., Wu, S., Robbins, S., Scollon, M., Pyrc, J. J., Druck, T., Huebner, K. (1993) Retroposition in a family of carcinoma-associated antigen genes. Mol. Cell Biol. 13:1507-1515.
Linstedt, A. D., Hauri, H. P. (1993) Giantin, a novel conserved Golgi membrane protein-containing a cytoplasmic domain of at least 350 kDa. Mol. Biol. Cell 4:679-693.
Lipshutz, R. J., Fodor, S. P. A., Gingeras, T. R., Lockhart, D. J. (1999) High density synthetic oligonucleotide arrays. Nature Genetics 21:20-24.
Liu A. Y., Robinson R. R., Hellstrom K. E., Murray E. D. Jr., Chang C. P., Hellstrom I. (1987a) Chimeric mouse-human IgG1 antibody that can mediate lysis of cancer cells. Proc. Natl. Acad. Sci. 84:3439-3443.
Liu, A. Y., Robinson, R. R., Murray, E. D. Jr., Ledbetter, J. A., Hellstrom, I., Hellstrom, K. E. (1987b) Production of a mouse-human chimeric monoclonal antibody to CD20 with potent Fc-dependent biologic activity. J. Immunol. 139:3521-3526.
Lodish, H., Berk, A., Zipursky, S. L., Matsudaira, P., Baltimore, D., Darness, J. (1999) Molecular Cell Biology. 4th ed. W H Freeman & Co.
Loeffen, J. L., Triepels, R. H., van den Heuvel, L. P., Schuelke, M., Buskens, C. A., Smeets, R. J., Trijbels, J. M., SmeitiNK cells, J. A. (1998) cDNA of eight nuclear encoded subunits of NADH:ubiquinone oxidoreductase: human complex 1 cDNA characterization completed. Biochem. Biophys. Res. Commun. 253:415-422.
Los, M., Burek, C. J., Stroh, C., Benedyk, K., Hug, H., Mackiewicz. (2003) Anticancer drugs of tomorrow: apoptotic pathways as targets for drug design. Drug Discov. Today 15:67-77.
Lovering R, Trowsdale J. (1991) A gene encoding 22 highly related zinc fingers is expressed in lymphoid cell lines. Nucleic Acids Res. 19:2921-2928.
Luckow, V., Summers, M. (1988) Trends in the development of baculovirus expression vectors. Bio/Technology 6:47-55.
MacBeath, G., Schreiber. S. L. (2000) Printing proteins as microarrays for high-throughput function determination. Science 289:1760-1763.
Machesky, L. M., Reeves, E., Wientjes, F., Mattheyse, F. J., Grogan, A., Totty, N. F., Burlingame, A. L., Hsuan, J. J., Segal, A. W. (1999) Mammalian actin-related protein 2/3 complex localizes to regions of lamellipodial protrusion and is composed of evolutionarily conserved proteins. Biochem. J. 328:105-112.
Machiels, J. P., van Baren, N., Marchand, M. (2002) Peptide-based cancer vaccines. Semin. Oncol. 29:494-502.
Mackay, A., Jones, C., Dexter, T., Silva, R. L., Bulmer, K., Jones, A., Simpson, P., Harris, R. A., Jat, P. S., Neville, A. M., Reis, L. F., Lakhani, S. R., O'Hare, M. J. (2003) cDNA microarray analysis of genes associated with ERBB2 (HER2/neu) overexpression in human mammary luminal epithelial cells. Oncogene 22:2680-2688.
Maeda, S., Kawai, T., Obinata, M., Fujiwara, H., Horiuchi, T., Saeki, Y., Sato, Y., Furusawa, M. (1985) Production of human alpha-interferon in silkworm using a baculovirus vector. Nature 315:592-594.
Mahajan, M. A., Murray, A., Samuels, H. H. (2002) NRC-interacting factor 1 is a novel cotransducer that interacts with and regulates the activity of the nuclear hormone receptor coactivator NRC. Mol. Cell Biol. 22:6883-6894.
Mahimkar, R. M., Baricos, W. H., Visaya, O., Pollock, A. S., Lovett, D. H. (2000) Identification, cellular distribution and potential function of the metalloprotease-disintegrin MDC9 in the kidney. J. Am. Soc. Nephrol., 11:595-603.
Mahnensmith, R. L., Aronson, P. S. (1985) Interrelationships among quinidine, amiloride, and lithium as inhibitors of the renal Na+-H+ exchanger. J. Biol. Chem. 260:12,586-12,592.
Manning, G., Whyte, D. B., Martinez, R., Hunter, T., Sudarsanam, S. (2002) The protein kinase complement of the human genome. Science 298:1912-1934.
Marotti, K. R., Tomich, C. S. (1989) Simple and efficient oligonucleotide-directed mutagenesis using one primer and circular plasmid DNA template. (1989) Gene Anal. Tech. 6:67-70.
Martel-Pelletier, J., Welsch, D. J., and Pelleteir, J. P. (2001) Metalloproteases and inhibitors in arthritic diseases. Best Pract. Res. Clin. Rheumatol. 15:805-829.
Martin, B. M., Tsuji, S., LaMarca, M. E., Maysak, K., Eliason, W., Ginns, E. I. (1988) Glycosylation and processing of high levels of active human glucocerebrosidase in invertebrate cells using a baculovirus expression vector. DNA 7:99-106.
Massari, M. E., Rivera, R. R., Voland, J. R., Quong, M. W., Breit, T. M., van Dongen, J. J, de Smit, O., Murre, C. (1998) Characterization of ABF-1, a novel basic helix-loop-helix transcription factor expressed in activated B lymphocytes. Mol. Cell Biol. 18:3130-3139.
Matz, M. V., Fradkov, A. F., Labas, Y. A., Savitsky, A. P., Zaraisky, A. G., Markelov, M. L., Lukyanov, S. A. (1999) Fluorescent proteins from nonbioluminescent Anthozoa species. Nat. Biotechnol. 17:969-973.
Mayer, B. J. (2001) SH3 domains: complexity in moderation. J. Cell Sci. 114:1253-1263.
Mayer, T. U., Kapoor, T. M., Haggarty, S. J., King, R. W., Schreiber, S. L., Mitchison, T. J. (1999) Small molecule inhibitor of mitotic spindle bipolarity identified in a phenotype-based screen. Science 286:971-974.
McGraw, R. A. III (1984) Dideoxy DNA sequencing with end-labeled oligonucleotide primers. Anal. Biochem. 143:298-303.
McKusick, V. A. (2003) OMIM: Online Mendelian Inheritance in Man http:www.ncbi.nlm.nih.gov, #104300.
McPherson, M. J., Møller, S. G., Benyon, R., Howe, C. (2000) PCR Basics: From Background to Bench. Springer Verlag.
Melloul, D., Marshak, S., Cerasi, E. (2002) Regulation of pdx-1 gene expression. Diabetes 51 Suppl 3:S320-325.
Merla, G., Ucla, C., Guipponi, M., Reymond, A. (2002) Identification of additional transcripts in the Williams-Beuren syndrome critical region. Hum. Genet. 110:429-438.
Miki, H., Setou, M., Kaneshiro, K., Hirokawa, N. (2001) All kinesin superfamily protein, KIF, genes in mouse and human. Proc. Natl. Acad. Sci. 98:7004-7011.
Milam, A. H., Rose, L., Cideciyan, A. V., Barakat, M. R., Tang, W. X., Gupta, N., Aleman, T. S., Wright, A. F., Stone, E. M., Sheffield, V. C., Jacobson, S. G. (2002) The nuclear receptor NR2E3 plays a role in human retinal photoreceptor differentiation and degeneration. Proc. Natl. Acad. Sci. 99:473-478.
Miller, L. K. (1988) Baculoviruses as gene expression vectors. Ann. Rev. Microbiol. 42:177-199.
Milligan, J. F., Matteucci, M. D., Martin, J. C. (1993) Current concepts in antisense drug design. J. Med. Chem. 36:1923-1937.
Mitch, W. E., Goldberg, A. L. (1996) Mechanisms of muscle wasting. The role of the ubiquitin-proteasome pathway. N. Engl. J. Med. 335:1897-1905.
Mitchell, D. A., Nair, S. K. (2000) RNA-transfected dendritic cells in cancer immunotherapy. J. Clin. Invest. 106:1065-1069.
Miura, M., Tamura, T., Mikoshiba, K. (1990) Cell-specific expression of the mouse glial fibrillary acidic protein gene: identification of the cis- and trans-acting promoter elements for astrocyte-specific expression. J. Neurochem. 5:1180-1188.
Miyajima A. (2002) Functional analysis of yeast homologue gene associated with human DNA helicase causative syndromes. Kokuritsu Iyakuhin Shokuhin Eisei Kenkyusho Hokoku 120:53-74.
Miyajima, A., Schreurs, J., Otsu, K., Kondo, A., Arai, K., Maeda, S. (1987) Use of the silkworm, Bombyx mori, and an insect baculovirus vector for high-level expression and secretion of biologically active mouse interleukin-3. Gene 58:273-281.
Molineux G. (2002) Pegylation: engineering improved pharmaceuticals for enhanced therapy. Cancer Treat. Rev. 28 Suppl A:13-16.
Molkentin, J. D., Jobe, S. M., Markham, B. E. (1996) Alpha-myosin heavy chain gene regulation: delineation and characterization of the cardiac muscle-specific enhancer and muscle-specific promoter. J. Mol. Cell Cardiol. 28:1211-1225.
Monfardini, C., Schiavon, O., Caliceti, P., Morpurgo, M., Harris, J. M., Veronese, F. M. (1995) A branched monomethoxypoly(ethylene glycol) for protein modification. Bioconjugate Chem. 6:62-69.
Mori, N. (1997) Neuronal growth-associated proteins in neural plasticity and brain aging. Nihon Shinkei Seishin Yakurigaku Zasshi 17:159-167.
Mortlock, D. P., Nelson, M. R., Innis, J. W. (1996) An efficient method for isolating putative promoters and 5′ transcribed sequences from large genomic clones. Genome Res. 6:327-335.
Murphy, D., Carter, D. A., eds. (1993) Transgenesis Techniques: Principles and Protocols. Humana Press.
Myers, E. W., Miller, W. (1988) Optimal alignments in linear space. Comput. Appl. Biosci. 4:11-7.
Nagata, K., Kawase, H., Handa, H., Yano, K., Yamasaki, M., Ishimi, Y., Okuda, A., Kikuchi, A., Matsumoto, K. (1995) Replication factor encoded by a putative oncogene, set, associated with myeloid leukemogenesis. Proc. Natl. Acad. Sci. 92:4279-4283.
Nanda, S., Bathon, J. M. (2004) Etanercept: a clinical review of current and emerging indications. Expert Opin. Pharmacother. 5:1175-1186.
Naora, H. (1999) Involvement of ribosomal proteins in regulating cell growth and apoptosis: translational modulation or recruitment for extraribosomal activity? Immunol. Cell Biol. 77:197-205.
Needleman, S. B., Wunch, C. D. (1970) A general method applicable to the search for similarities in the amino acid sequence of two proteins. J. Mol. Biol. 48:443-453.
Nelson, N., Harvey, W. R. (1999) Vacuolar and plasma membrane proton-adenosine triphosphatases. Physiol. Rev. 79:361-385.
Nishiyama, H., Higashitsuji, H., Yokoi, H., Itoh, K., Danno, S., Matsuda, T., Fujita, J. (1997) Cloning and characterization of human CIRP (cold-inducible RNA-binding protein) cDNA and chromosomal assignment of the gene. Gene 204:115-120.
Noma, T., Fujisawa, K., Yamashiro, Y., Shinohara, M., Nakazawa, A., Gondo, T., Ishihara, T., Yoshinobu, K. (2001) Structure and expression of human mitochondrial adenylate kinase targeted to the mitochondrial matrix. Biochem. J. 358:225-232.
Notredame, C., Higgins, D., Hering a, J. (2000) T-Coffee: A novel method for multiple sequence alignments. J. Molec. Biol. 302:205-217.
Okayama, H., Berg, P. (1983) A cDNA cloning vector that permits expression of cDNA inserts in mammalian cells. Mol. Cell. Biol. 3:280-289.
Oksenberg, J. R., Barcellos, L. F., Hauser, S. L. (1999) Genetic aspects of multiple sclerosis. Semin. Neurol. 19:281-288.
Oliver, C. J., Shenolikar, S. (1998) Physiologic importance of protein phosphatase inhibitors. Frontiers in Bioscience 3:961-972.
O'Neil, N. J., Martin, R. L., Tomlinson, M. L., Jones, M. R., Coulson, A., Kuwabara, P. E. (2001) RNA-mediated interference as a tool for identifying drug targets. Am. J. Pharmacogenomics 1:45-53.
O'Neill, L. A. (2002) Signal transduction pathways activated by the IL-1 receptor/toll-like receptor superfamily. Curr. Top. Microbiol. Immunol. 270:47-61.
Osaki, M., Tan, L., Choy, B. K., Yoshida, Y., Cheah, K. S., Auron, P. E., Goldring, M. B. (2003) The TATA-containing core promoter of the type II collagen gene (COL2A1) is the target of interferon-gamma-mediated inhibition in human chondrocytes: requirement for Stat1 alpha, Jak1 and Jak2. Biochem. J. 369:103-315.
Osborn, B. L., Olsen, H. S., Nardelli, B., Murray, J. H., Zhou, J. X., Garcia, A., Moody, G., Zaritskaya, L. S., Sung, C. (2002) Pharmacokinetic and pharmacodynamic studies of a human serum albumin-interferon-alpha fusion protein in cynomolgus monkeys. J. Pharmacol. Exp. Ther. 303:540-548.
Page, D. C., Silber, S. Brown, L. G. (1999) Men with infertility caused by AZFc deletion can produce sons by intracytoplasmic sperm injection, but are likely to transmit the deletion and infertility. Hum. Reprod. 14:1722-1726.
Pan, C. X., Koeneman, K. S. (1999) A novel tumor-specific gene therapy for bladder cancer. Med. Hypothesis 53:130-135.
Pang, T., Wakabayashi, S., Shigekawa, M. (2001) Calcineurin homologous protein as an essential cofactor for Na+/H+ exchangers. J. Biol. Chem. 276:17,367-17,372.
Pang, T., Wakabayashi, S., Shigekawa, M. (2002) Expression of calcineurin B homologous protein 2 protects serum deprivation-induced cell death by serum-independent activation of Na+/H+ exchanger. J. Biol. Chem. 277:43,771-43,777.
Papagerakis, S., Shabana, A. H., Depondt, J., Gehanno, P., Forest, N. (2003) Immunohistochemical localization of plakophilins (PKP1, PKP2, PKP3, and p0071) in primary oropharyngeal tumors: correlation with clinical parameters. Hum. Pathol. 34:565-572.
Paterson, T., Innes, J., Moore, S. (1994) Approaches to maximizing stable expression of alpha 1-antitrypsin in transformed CHO cells. Appl. Microbiol. Biotechnol. 40:691-698.
Pearson, W. R. (2000) Flexible sequence similarity searching with the FASTA3 program package. Methods Mol. Biol. 132:185-219.
Peattie, D. A., Harding, M. W., Fleming, M. A., DeCenzo, M. T., Lippke, J. A., Livingston, D. J., Benasutti, M. (1992) Expression and characterization of human FKBP52, an immunophilin that associates with the 90-kDa heat-shock protein and is a component of steroid receptor complexes. Proc. Natl. Acad. Sci. 89:10,974-10,978.
Peelle, B., Gururaja, T. L., Payan, D. G., Anderson, D. C. (2001) Characterization and use of green fluorescent proteins from Renilla mulleri and Ptilosarcus guernyi for the human cell display of functional peptides. J. Protein Chem. 20:507-519.
Pepin, K., Momose, F., Ishida, N., Nagata, K. (2001) Molecular cloning of horse Hsp90 cDNA and its comparative analysis with other vertebrate Hsp90 sequences. J. Vet. Med. Sci. 63:115-124.
Perez Calvo, J. I., Inigo Gil, P., Giraldo Castellano, P., Torralba Cabeza, M. A., Civeira, F., Lario Garcia, S., Pocovi, M., Lara Garcia, S. (2000) Transforming growth factor beta (TGF-beta) in Gaucher's disease. Preliminary results in a group of patients and their carrier and non-carrier relatives Med. Clin. (Barc) 115:601-604.
Perron, H., Garson, J. A., Bedin, F., Beseme, F., Paranhos-Baccala, G., Komurian-Pradel, F., Mallet, F., Tuke, P. W., Voisset, C., Blond, J. L., Lalande, B., Seigneurin, J. M., Mandrand, B., The Collaborative Research Group on Multiple Sclerosis (1997) Molecular identification of a novel retrovirus repeatedly isolated from patients with multiple sclerosis. Proc. Natl. Acad. Sci. 94:7583-7588.
Perry, A. C., Jones, R., Hall, L. (1995) Analysis of transcripts encoding novel members of the mammalian metalloprotease-like, disintegrin-like, cysteine-rich (MDC) protein family and their expression in reproductive and non-reproductive monkey tissues. Biochem. J. 312(Pt 1):239-244.
Pertl, U., Wodrich, H., Ruelmann, J. M., Gillies, S. D., Lode, H. N., Reisfeld, R. A. (2003) Immunotherapy with a posttranscriptionally modified DNA vaccine induces complete protection against metastatic neuroblastoma. Blood 101:649-654.
Pfutzer, R. H., Whitcomb, D. C. (2001) SPINK1 mutations are associated with multiple phenotypes. Pancreatology 1:457-460.
Phillips, M. I., ed. (1999a) Antisense Technology, Part A. Methods in Enzymology Vol. 313. Academic Press, Inc.
Phillips, M. I., ed. (1999b) Antisense Technology, Part B. Methods in Enzymology Vol. 314. Academic Press, Inc.
Pietu, G., Alibert, O., Guichard, V., Lamy, B., Bois, F., Mariage-Sampson, R., Hougatte, R., Soularue, P., Auffray, C. (1996) Novel gene transcripts preferentially expressed in human muscles revealed by quantitative hybridization of a high density cDNA array. Genome Res. 6:492-503.
Pinkert, C. A., ed. (1994) Transgenic Animal Technology: A Laboratory Handbook. Academic Press.
Pirozzi, G., McConnell, S. J., Uveges, A. J., Carter, J. M., Sparks, A. B., Kay, B. K., Fowlkes, D. M. (1997) Identification of novel human WW domain-containing proteins by cloning of ligand targets. J. Biol. Chem. 272:14,611-14,616.
Pisegna, J. R., WaNK cells, S. A. (1996) Cloning and characterization of the signal transduction of four splice variants of the human pituitary adenylate cyclase activating polypeptide receptor. Evidence for dual coupling to adenylate cyclase and phospholipase C. J. Biol. Chem. 271:17,267-17,274.
Pourquie, O. (2003) The segmentation clock: converting embryonic time into spatial pattern. Science 301:328-330.
Power, S. C., Cereghini, S., Rollier, A., Gannon, F. (1994) Isolation and functional analysis of the promoter of the bovine serum albumin gene. Biochem. Biophys. Res. Commun. 203:1447-1456.
Prentki, P., Krisch, H. M. (1984) In vitro insertional mutagenesis with a selectable DNA fragment. Gene 29:303-313.
Price, N. T., Hall, L., Proud, C. G. (1993) Cloning of cDNA for the beta-subunit of rabbit translation initiation factor-2 using PCR. Biochim. Biophys. Acta 1216:170-172.
Qin, J., Li., L. (2003) Molecular anatomy of the DNA damage and replication checkpoints. Radiat. Res. 159:139-148.
Racevskis, J., Dill, A., Stockert, R., Fineberg, S. A. (1996) Cloning of a novel nucleolar guanosine 5′-triphosphate binding protein autoantigen from a breast tumor. Cell. Growth Differ. 7:271-280.
Ramalho-Santos, M. (2002) “Stemness” Science 298:597-600.
Raval, P. (1994) Qualitative and quantitative determination of mRNA. J. Pharmacol. Toxicol. Methods 32:125-127.
Rawlings, N. D., Barrett, A. J. (1994) Families of serine peptidases. Methods Enzymol. 244:19-61.
Rebbe, N. F., Ware, J., Bertina, R. M., Modrich, P., Stafford, D. W. (1987) Nucleotide sequence of a cDNA for a member of the human 90-kDa heat-shock protein family. Gene 53:235-245.
Rebhan, M., Chalifa-Caspi, V., Prilusky, J., Lancet, D. (1997) GeneCards: encyclopedia for genes, proteins and diseases. Weizmann Institute of Science, Bioinformatics Unit and Genome Center (Rehovot, Israel) GeneCard for [gene] [Last Update] World Wide Web URL: http://bioinformatics.weizmann.ac.il/cards-bin/carddisp?[gene].
Rechid, R., Vingron, M., Argos, P. (1989) A new interactive protein sequence alignment program and comparison of its results with widely used algorithms. Comput. Appl. Biosci. 5:107-113.
Rehli, M., Krause, S. W., Kreutz, M., Andreesen, R. (1995) Carboxypeptidase M is identical to the MAX. 1 antigen and its expression is associated with monocyte to macrophage differentiation. J. Biol. Chem. 270:15644-15649.
Remington, J. P. (1985) Remington's Pharmaceutical Sciences. 17th ed. Mack Publishing Co.
Reya, T. (2003a) Regulation of hematopoeitic stem cell self-renewal. Recent Prog. Horm. Res. 58:283-295.
Ribardo, D. A., Peterson, J. W., Chopra, A. K. (2002) Phospholipase A2-activating protein—an important regulatory molecule in modulating cyclooxygenase-2 and tumor necrosis factor production during inflammation. Indian J. Exp. Biol. 40:129-138.
Riley, J., Butler, R., Ogilvie, D., Finniear, R., Jenner, D., Powell, S., Anand, R., Smith, J. C., Markham, A. F. (1990) A novel, rapid method for the isolation of terminal sequences from yeast artificial chromosome (YAC) clones. Nuc. Acids Res. 18:2887-2890.
Ritter, R. C., Brenner, L. A., Tamura, C. S. (1994) Endogenous CCK and the peripheral neural substrates of intestinal satiety. Ann. N.Y. Acad. Sci. 713:255-267.
Robertson, H. M. (1996) Members of the pogo superfamily of DNA-mediated transposons in the human genome. Mol. Gen. Genet. 252:761-766.
Robertson, H. M., Zumpano, K. L. (1997) Molecular evolution of an ancient mariner transposon, Hsmar1, in the human genome. Gene 205:203-217.
Roepman, R., Bernoud-Hubac, N., Schick, D. E., Maugeri, A., Berger, W., Ropers, H. H., Cremers, F. P., Ferreira, P. A. (2000) The retinitis pigmentosa GTPase regulator (RPGR) interacts with novel transport-like proteins in the outer segments of rod photoreceptors. Hum. Mol. Genet. 9:2095-2105.
Roessler, B. J., Nosal, J. M., Smith, P. R., Heidler, S. A., Palella, T. D., Switzer, R. L., Becker, M. A. (1993) Human X-linked phosphoribosylpyrophosphate synthetase superactivity is associated with distinct point mutations in the PRPS1 gene. J. Biol. Chem. 268:26476-26481.
Roggenkamp, R., Janowicz, Z., Stanikowski, B., Hollenberg, C. P. (1984) Biosynthesis and regulation of the peroxisomal methanol oxidase from the methylotrophic yeast Hansenula polymorpha. Mol. Gen. Genet. 194:489-493.
Ronicke, V., Risau, W., Breier, G. (1996) Characterization of the endothelium-specific murine vascular endothelial growth factor receptor-2 (Flk-1) promoter. Circ. Res. 79:277-285.
Rosato, R. R., Grant, S. (2003) Histone deacetylase inhibitors in cancer therapy. Cancer Biol. Ther. 2:30-37.
Rosen, R. C., McKenna, K. E. (2002) PDE-5 inhibition and sexual response: pharmacological mechanisms and clinical outcomes. Ann. Rev. Sex Res. 13:36-88.
Rowland, J. M. (2002) Molecular genetic diagnosis of pediatric cancer: current and emerging methods. Pediatr. Clin. North Am. 49:1415-1435.
Saha, S., Bardelli, A., Buckhaults, P., Velculescu, V. E., Rago, C., St Croix, B., Romans, K. E., Choti, M. A., Lengauer, C., Kinzier, K. W., Vogelstein, B. (2001) A phosphatase associated with metastasis of colorectal cancer. Science 294:1343-1346.
Saiki, R. K, Gelfand, D. H., Stoffel, S., Scharf, S. J., Higuchi, R., Horn, G. T., Mullis, K. B., Erlich, H. A. (1988) Primer-directed enzymatic amplification of DNA with amplification of DNA with a thermostable DNA polymerase. Science 239:487-491.
Sambrook, J., Russell, D. W., Sambrook, J. (2001) Molecular Cloning, A Laboratory Manual. 3^{nd} ed., Cold Spring Harbor Laboratory Press.
Sanchez, E. R., Faber, L. E., Henzel, W. J., Pratt, W. B. (1990) The 56-59-kilodalton protein identified in untransformed steroid receptor complexes is a unique protein that exists in cytosol in a complex with both the 70- and 90-kilodalton heat-shock proteins. Biochemistry 29:5145-5152.
Sayers, J. R., Krekel, C., Eckstein, F. (1992) Rapid high-efficiency site-directed mutagenesis by the phosphothioate approach. Biotechniques 13:592-596.
Schaeferling, M., Schiller, S., Paul, H., Kruschina, M., Pavlickova, M., Meerkamp, M., Giammasi, C., Kambhampati, D. (2002) Application of self-assembly techniques in the design of biocompatible protein microarray surfaces. Electrophoresis 23:3097-3105.
Schaffer, J. E., Lodish, H. F. (1994) Expression cloning and characterization of a novel adipocyte long chain fatty acid transport protein. Cell 79:393-395.
Schena, M., ed. (1999) DNA Microarrays: A Practical Approach. Oxford Univ. Press.
Schena, M., ed. (2000) Microarray Biochip Technology. 1st ed. Eaton Publishing Co.
Schlesinger, D. H. (1988a) MacRomolecular Sequencing and Synthesis: Selected Methods and Applications. Wiley-Liss.
Schlesinger, D. H., ed. (1988b) Current Methods in Sequence Comparison and Analysis, Macromolecule Sequencing and Synthesis, Selected Methods and Applications, pp. 127-149, Alan R. Liss, Inc.
Schonthal, A. H. (2001) Role of serine/threonine protein phosphatase 2A in cancer. Cancer Lett. 170:1-13.
Seelig, H. P., Schranz, P., Schroter, H., Wiemann, C. Renz, M. (1994) Macrogolgin—a new 376 kD Golgi complex outer membrane protein as target of antibodies in patients with rheumatic diseases and HIV infections. J. Autoimmun. 7:67-91.
Selkoe, D. J. (2001) Presenilin, Notch, and the genesis and treatment of Alzheimer's disease. Proc. Natl. Acad. Sci. 98:11,039-11,041.
Setlow, J., Hollaender, A., eds. (1986) Genetic Engineering: Principles and Methods. Plenum Pub. Corp.
Shamay, M., Barak, O., Doitsh, G., Ben-Dor, I., Shaul, Y. (2002) Hepatitis B virus pX interacts with HBXAP, a PHD finger protein to coactivate transcription. J. Biol. Chem. 277:9982-9988.
Shao, H., Andres, D. A. (2000) A novel RalGEF-like protein, RGL3, as a candidate effector for rit and Ras. J. Biol. Chem. 275:26,914-26,924.
Sheppard, P., Kindsvogel, W., Xu, W., Henderson, K., Schlutsmeyer, S., Whitmore, T. E., Kuestner, R., Garrigues, U., Birks, C., Roraback, J., Ostrander, C., Dong, D., Shin, J., Presnell, S., Fox, B., Haldeman, B., Cooper, E., Taft, D., Gilbert, T., Grant, F. J., Tackett, M., Krivan, W., McKnight, G., Clegg, C., Foster, D., Klucher, K. M. (2003) IL-28, IL-29 and their class II cytokine receptor IL-28R. Nat. Immunol. 4:63-68.
Sheppard, P. O., Bishop, P. D. (2002) Nucleic Acid Molecules that Encode Human Zven1. U.S. Pat. No. 6,485,938.
Sheppard, P. O., Bishop, P. D. (2004) Human Zven1 Proteins. U.S. Pat. No. 6,756,479.
Shinnick, T. M., Sutcliffe, J. G., Green, N., Lerner, R. A. (1983) Synthetic peptide immunogens as vaccines. Ann. Rev. Microbiol. 37:425-446.
Shorter, J., Beard, M. B., Seemann, J., Dirac-Svejstrup, A. B., Warren, G. (2002) Sequential tethering of Golgins and catalysis of SNAREpin assembly by the vesicle-tethering protein p 115. J. Cell Biol. 157:45-62.
Siebenlist, U., Simpson, R. B., Gilbert, W. (1980) E. coli RNA polymerase interacts homologously with two different promoters. Cell 20:269-281.
Siegal, G. J., Agranoff, B. W., Albers, R. W., Fisher, S. K., Uhler, M. D., eds. (1999) Basic Neurochemistry, Molecular, Cellular, and Medical Aspects. 6th ed. Lippencott, Williams & Wilkins.
Sladek, R., Bader, J. A., Giguere, V. (1997) The orphan nuclear receptor estrogen-related receptor alpha is a transcriptional regulator of the human medium-chain acyl coenzyme A dehydrogenase gene. Mol. Cell Biol. 17:5400-5409.
Slavin, S., Or, R., Aker, M., Shapira, M. Y., Panigrahi, S., Symeonidis, A., Cividalli, G., Nagler, A. (2001) Nonmyeloablative stem cell transplantation for the treatment of cancer and life-threatening nonmalignant disorders: past accomplishments and future goals. Cancer Chemother. Pharmacol. 48:S79-S84.
Sinit, A. F., Riggs, A. D. (1996) Tiggers and DNA transposon fossils in the human genome. Proc. Natl. Acad. Sci. 93:1443-1448.
Smith, G. E., Ju, G., Ericson, B. L., Moschera, J., Lahm, H. W., Chizzonite, R., Summers, M. D. (1985) Modification and secretion of human interleukin 2 produced in insect cells by a baculovirus expression vector. Proc. Natl. Acad. Sci. 82:8404-8408.
Smith, T. F., Waterman, M. S. (1981) Comparison of biosequences. Adv. Appl. Math. 2:482-489.
Soares, M. B. (1997) Identification and cloning of differentially expressed genes. Curr. Opin. Biotechnol. 8:542-546.
Soejima, H., Kawamoto, S., Akai, J., Miyoshi, O., Arai, Y., Morohka, T., Matsuo, S., Niikawa, N., Kimura, A., Okubo, K., Mukai, T. (2001) Isolation of novel heart-specific genes using the BodyMap database. Genomics. 74:115-120.
Soulier, S., Vilotte, J. L., L'Huillier, P. J., Mercier, J. C. (1996) Developmental regulation of murine integrin beta 1 subunit- and Hsc73-encoding genes in mammary gland: sequence of a new mouse Hsc73 cDNA. Gene 172:285-289.
Southern, E., Mir, K., Shchepinov, M. (1999) Molecular interactions on microarrays. Nature Genetics 21:5-9.
Stein, C. A., Kreig, A. M., eds. (1998) Applied Antisense Oligonucleotide Technology. Wiley-Liss.
Steinhaur, C., Wingren, C., Hager, A. C., Borrebaeck, C. A. (2002) Single framework recombinant antibody fragments designed for protein chip applications. Biotechniques, Supp.: 38-45.
Stetler-Stevenson, W. G., Liotta, L. A., Kleiner, D. E. Jr. (1993) Extracellular matrix 6: role of matrix metalloproteinases in tumor invasion and metastasis. FASEB J. 7:1434-1441.
Stewart, Z. A., Westfall, M. D., Pietenpol, J. A. (2003) Cell-cycle dysregulation and anticancer therapy. Trends Pharmacol. Sci. 24:139-145.
Stolz, L. E., Tuan, R. S. (1996) Hybridization of biotinylated oligo(dT) for eukaryotic mRNA quantitation. Mol. Biotechnol. 6:225-230.
Sturm, A., Dignass, A. U. (2002) Modulation of gastrointestinal wound repair and inflammation by phospholipids. Biochim. Biophys. Acta 1582:282-288.
Stutz, F., Bachi, A., Doerks. T., Braun, I. C., Seraphin, B., Wilm, M., Bork, P., Izaurralde, E. (2000) REF, an evolutionary conserved family of hnRNP-like proteins, interacts with TAP/Mex67p and participates in mRNA nuclear export. RNA 6:638-650.
Suh, Y. H., Checker, F. (2002) Amyloid precursor protein, presenilins, and alpha-synuclein: molecular pathogenesis and pharmacological applications in Alzheimer's disease. Pharmacol. Rev. 54:469-525.
Sung, C., Nardelli, B., LaFleur, D. W., Blatter, E., Corcoran, M., Olsen, H. S., Birse, C. E., Pickeral, O. K., Zhang, J., Shah, D., Moody, G., Gentz, S., Beebe. L., Moore, P. A. (2003) An IFN-beta-albumin fusion protein that displays improved pharmacokinetic and pharmacodynamic properties in nonhuman primates. J. Interferon Cytokine Res. 23:25-36.
Sutcliffe, J. G., Shinnick, T. M., Green, N., Lerner, R. A. (1983) Antibodies that react with predetermined sites on proteins. Science 219:660-666.
Sweetser, D. A., Hauft, S. M., Hoppe, P. C., Birkenmeier, E. H., Gordon, J. I. (1988) Transgenic mice containing intestinal fatty acid-binding protein-human growth hormone fusion genes exhibit correct regional and cell-specific expression of the reporter gene in their small intestine Proc. Natl. Acad. Sci. 85:9611-9615.
Tan, J., Town, T., Paris, D., Mori, T., Suo, Z., Crawford, F., Mattson, M. P., Flavell, R. A., Mullan, M. (1999) Microglial activation resulting from CD40-CD40L interaction after beta-amyloid stimulation. Science 286:2352-2355.
Tang, D. C., DeVit, M., Johnston, S. A. (1992) Genetic immunization is a simple method for eliciting an immune response. Nature 356:152-154.
Tekur, S., Pawlak, A., Guellaen, G., Hecht, N. B. (1999) Contrin, the human homologue of a germ-cell Y-box-binding protein: cloning, expression, and chromosomal localization. J. Androl. 20:135-144.
Terada, R., Yamamoto, K., Hakoda, T., Shimada, N., Okano, N., Baba, N., Ninomiya, Y., Gershwin, M. E., Shiratori, Y. (2003) Stromal cell-derived factor-1 from biliary epithelial cells recruits CXCR4-positive cells: implications for inflammatory liver diseases. Lab. Invest. 83:665-672.
Thompson, J. D., Higgins, D. G., Gibbon, T. J. (1994) CLUSTAL W: improving the sensitivity of progressive multiple sequence alignment through sequence weighting, position-specific gap penalties and weight matrix choice. Nucleic Acids Res. 22:4673-80.
Thompson, S., Clarke, A. R., Pow, A. M., Hooper, M. L., Melton, D. W. (1989) Germ line transmission and expression of a corrected HPRT gene produced by gene targeting in embryonic stem cells. Cell 56:313-321.
Tilburn, J., Scazzocchio, C., Taylor, G. G., Zabicky-Zissman, J. H., Lockington, R. A., Davies, R. W. (1983) Transformation by integration in Aspergillus nidulans. Gene 26:205-221.
Trounson, A. (2002) Human embryonic stem cells: mother of all cell and tissue types. Reprod. Biomed. Online 4 Suppl. 1:58-63.
Tsuda, T., Gallup, M., Jany, B., Gum, J., Kim, Y., Basbaum, C. (1993) Characterization of a rat airway cDNA encoding a mucin-like protein. Biochem. Biophys. Res. Commun. 195:363-373.
Tukey, R. H., Pendurthi, U. R, Nguyen, N. T., Green, M. D., Tephly, T. R. (1993) Cloning and characterization of rabbit liver UDP-glucuronosyltransferase cDNAs. Developmental and inducible expression of 4-hydroxybiphenyl UGT2B13. J. Biol. Chem. 268:15,260-15,266.
Ulmer, J. B., Donnelly, J. J., Parker, S. E., Rhodes, G. H., Felgner, P. L., Dwarki, V. J., Gromkowski, S. H., Deck, R. R., DeWitt, C. M., Friedman, A., et al. (1993) Heterologous protection against influenza by injection of DNA encoding a viral protein. Science 259:1745-1749.
Vainberg, I. E., Lewis, S. A., Rommelaere, H., Ampe, C., Vandekerckhove, J., Klein, H. L., Cowan, N. J. (1998) Prefoldin, a chaperone that delivers unfolded proteins to cytosolic chaperonin. Cell 93:863-873.
Vale, R. D. (2003) The molecular motor toolbox for intracellular transport. Cell 112:467-480.
Vallejo, M., Ron, D., Miller, C. P., Habener, J. F. (1993) C/ATF, a member of the activating transcription factor family of DNA-binding proteins, dimerizes with CAAT/enhancer-binding proteins and directs their binding to cAMP response elements. Proc. Natl. Acad. Sci. 90:4679-4683.
van den Berg, J. A., van der Laken, K. J., van Ooyen, A. J., Renniers, T. C., Rietveld, K., Schaap, A., Brake, A. J., Bishop, R. J., Schultz, K., Moyer, D. (1990) Kluyveromyces as a host for heterologous gene expression: expression and secretion of prochymosin. Bio/Technology 8:135-139.
Van den Berghe, L., Laurell, H., Huez, I., Zanibellato, C., Prats, H., Bugler, B. (2000) FIF [fibroblast growth factor-2 (FGF-2)-interacting-factor], a nuclear putatively antiapoptotic factor, interacts specifically with FGF-2. Mol. Endocrinol. 14:1709-1724.
Van Den Blink, B., Ten Hove T., Van Den Brink G. R., Peppelenbosch M. P., Van Deventer S. J. (2002) From extracellular to intracellular targets, inhibiting MAP kinases in treatment of Crohn's disease. Ann. N.Y. Acad. Sci. 973:349-58.
van der Putten, H., Botteri, F. M., Miller, A. D., Rosenfeld, M. G., Fan, H., Evans, R. M., Verma, I. M. (1985) Efficient insertion of genes into the mouse germ line via retroviral vectors. Proc. Natl. Acad. Sci. 82:6148-6152.
van der Spoel, A. C., Jeyakumar, M., Butters, T. D., Charlton, H. M., Moore, H. D., Dwek, R. A., Platt, F. M. (2002) Reversible infertility in male mice after oral administration of alkylated imino sugars: a nonhormonal approach to male contraception. Proc. Natl. Acad. Sci. 99:17173-17178.
Van Eerdewegh, P., Little, R. D., Dupuis, J., Del Mastro, R. G., Falls, K., Simon, J., Torrey, D., Pandit, S., McKenny, I., Braunschweiger, K., Walsh, A., Liu, Z., Hayward, B., Folz, C., Manning, S. P., Bawa, A., Saracino, L., Thackston, M., Benchekroun, Y., Capparell, N., Wang, M., Adair, R., Feng, Y., Dubois, J., FitzGerald, M. G., Huang, H., Gibson, R., Allen, K. M., Pedan, A., Danzig, M. R., Umland, S. P., Egan, R. W., Cuss, F. M., Rorke, S., Clough, J. B. Holloway, J. W., Holgate, S. T., Keith, T. P. (2002) Association of the ADAM33 gene with asthma and bronchial hyperresponsiveness. Nature. 418:426-430.
Van Laar, J. M., Tyndall, A. (2003) Intense immunosuppression and stem-cell transplantation for patients with severe rheumatic autoimmune disease: a review. Cancer Control 10:57-65.
Verhey, K. J., Meyer, D., Deehan, R., Blenis, J., Schnapp, B. J., Rapoport, T. A., Margolis, B. (2001) Cargo of kinesin identified as JIP scaffolding proteins and associated signaling molecules. J. Cell Biol. 152:959-970.
Vlak, J. M., Klinkenberg, F. A., Zaal, K. J., Usmany, M., Klinge-Roode, E. C., Geervliet, J. B., Roosien, J., van Lent, J. W. (1988) Functional studies on the p10 gene of Autographa californica nuclear polyhedrosis virus using a recombinant expressing a p10-beta-galactosidase fusion gene. J. Gen. Virol. 69:765-776.
Voisset, C., Bouton, O., Bedin, F., Duret, L., Mandrand, B., Mallet, F., Paranhos-Baccala. G. (2000) Chromosomal distribution and coding capacity of the human endogenous retrovirus HERV-W family. AIDS Res. Hum. Retroviruses 16:731-740.
Wagner, R. W., Matteucci, M. D., Grant, D., Huang, T., Froehler, B. C. (1996) Potent and selective inhibition of gene expression by an antisense heptanucleotide. Nat. Biotechnol. 14:840-844.
Wagner, R. W., Matteucci, M. D., Lewis, J. G., Gutierrez, A. J., Moulds, C., Froehler, B. C. (1993) Antisense gene inhibition by oligonucleotides containing C-5 propyne pyrimidines. Science 260:1510-1513.
Walder, R. Y., Walder, J. A. (1986) Oligodeoxynucleotide-directed mutagenesis using the yeast transformation system. Gene 4:133-139.
Walker, J. E., Arizmendi, J. M., Dupuis, A., Fearnley, I. M., Finel, M., Medd, S. M., Pilkington, S. J., Runswick, M. J., Skehel, J. M. (1992) Sequences of 20 subunits of NADH:ubiquinone oxidoreductase from bovine heart mitochondria. Application of a novel strategy for sequencing proteins using the polymerase chain reaction. J. Mol. Biol. 226:1051-1072.
Walsh, A. C., Feulner, J. A., Reilly, A. (2001) Evidence for functionally significant polymorphism of human glutamate cysteine ligase catalytic subunit: association with glutathione levels and drug resistance in the National Cancer Institute tumor cell line panel. Toxicol. Sci. 61:218-223.
Wang, J., Kirby, C. E., Herbst, R. (2002) The tyrosine phosphatase PRL-1 localizes to the endoplasmic reticulum and the mitotic spindle and is required for normal mitosis. J. Biol. Chem. 277:46659-46668.
Wang, M. S., Schinzel, A., Kotzot, D., Balmer, D., Casey, R., Chodirker, B. N., Gyftodimou, J., Petersen, M. B., Lopez-Rangel, E., Robinson, W. P. (1999) Molecular and clinical correlation study of Williams-Beuren syndrome: No evidence of molecular factors in the deletion region or imprinting affecting clinical outcome. Am. J. Med. Genet. 86:34-43.
Wax, S. D., Rosenfield, C. L., Taubman, M. B. (1994) Identification of a novel growth factor-responsive gene in vascular smooth muscle cells. J. Biol. Chem. 269:13,041-13,047.
Wei, S., Charmley, P., Concannon, P. (1997) Organization, polymorphism, and expression of the human T-cell receptor AV1 subfamily. Immunogenetics 45:405-412.
Weiner, H. L., Selkoe, D. J. (2002) Inflammation and therapeutic vaccination in CNS diseases. Nature 420:879-884.
Weiner, M. P., Felts, K. A., Simcox, T. G., Braman, J. C. (1993) A method for the site-directed mono- and multi-mutagenesis of double-stranded DNA. Gene 126:35-41.
Weinstein, M. E., Grossman, A., Perle, M. A., Wilmot, P. L., Verma, R. S., Silver, R. T., Arlin, Z., Allen, S. L., Amorosi, E., Waintraub, S. E., et al. (1988) The karyotype of Philadelphia chromosome-negative, bcr rearrangement-positive chronic myeloid leukemia. Cancer Genet Cytogenet. 35:223-229.
Weishaar, R. E., Cain, M. H., Bristol, J. A. (1985) A new generation of phosphodiesterase inhibitors: multiple molecular forms of phosphodiesterase and the potential for drug selectivity. J. Med. Chem. 28:537-545.
Weissman, I. L. (2000) Translating stem and progenitor cell biology to the clinic: barriers and opportunities. Science 287:1442-1446.
Weng, S., Gu, K., Hammond, P. W., Lohse, P., Rise, C., Wagner, R. W., Wright, M. C., Kuimelis, R. G. (2002) Generating addressable protein microarrays with PROfusion covalent mRNA-protein fusion technology. Proteomics 2:48-57.
Wenger, R. H., Rochelle, J. M., Seldin, M. F., Kohler, G., Nielsen, P. J. (1993) The heat stable antigen (mouse CD24) gene is differentially regulated but has a housekeeping promoter. J. Biol. Chem. 268:23,345-23,352.
Werner, T., Brack-Werner, R., Leib-Mosch, C., Backhaus, H., Erfle, V., Hehlmann, R. (1990) S71 is a phylogenetically distinct endogenous retroviral element with structural and sequence homology to mimian sarcoma virus (SSV). Virology 174:225-238.
Wick, G., Kromer, G., Neu, N., Fassler, R., Ziemiecki, A., Muller, R. G., Ginzel, M., Beladi, I., Kuhr, T., Hala, K. (1987) The multi-factorial pathogenesis of autoimmune disease. Immunol. Lett. 16:249-257.
Wieczorek, H., Brown, D., Grinstein, S., Ehrenfeld, J., Harvey, W. R. (1999) Animal plasma membrane energization by proton-motive V-ATPases. Bioessays 21:637-648.
Wieser, R. (2002) Rearrangements of chromosomal band 3q21 in myeloid leukemia. Leuk. Lymphoma 43:59-65.
Williams, A. F., Barclay, A. N. (1988) The immunoglobulin superfamily—domains for cell surface recognition. Annu. Rev. Immunol. 6:381-405.
Wilmut, I., Schnieke, A. E., McWhir, J., Kind, A. J., Campbell, K. H. (1997) Viable offspring derived from fetal and adult mammalian cells. Nature 385:810-813.
Winssinger, N., Ficarro, S., Schultz, P. G., and Harris, J. L. (2002) Profiling protein function with small molecule microarrays. Proc. Natl. Acad. Sci. 99:11,139-11,144.
Wojtowicz-Praga, S. (1999) Clinical potential of matrix metalloprotease inhibitors. Drugs R. D. 1:117-129.
Wright, G., Carver, A., Cottom, D., Reeves, D., Scott, A., Simons, P., Wilmut, I., Garner, I., Colman, A. (1991) High level expression of active human alpha-1-antitrypsin in the milk of transgenic sheep. Biotechnology (N.Y.) 9:830-834.
Wu, A. M., Gallo, R. C. (1975) Reverse Transcriptase. CRC Crit. Rev. Biochem. 3:289-347.
Xu, C. W., Mendelsohn, A. R., Brent, R. (1997) Cells that register logical relationships among proteins. Proc. Natl. Acad. Sci. (USA) 94:12,473-12,478.
Xu, Y., Piston, D. W., Johnson, C. H. (1999) A bioluminescence resonance energy transfer (BRET) system: Application to interacting circadian clock proteins. Proc. Natl. Acad. Sci. 96:151-156.
Yang, N., Shigeta, H., Shi, H., Teng, C. T. (1996) Estrogen-related receptor, hERR1, modulates estrogen receptor-mediated response of human lactoferrin gene promoter. J. Biol. Chem. 271:5795-5804.
Yao, Z., Dai, W., Perry, J., Brechbiel, M. W., Sung, C. (2004) Effect of albumin fusion on the biodistribution of interleukin-2. Cancer Immunol. Immunother. 53:404-410; Epub Nov. 18, 2003.
Yelton, M. M., Hamer, J. E., Timberlake, W. E. (1984) Transformation of Aspergillus nidulans by using a trpC plasmid. Proc. Natl. Acad. Sci. 81:1470-1474.
Yoshihama, M., Uechi, T., Asakawa, S., Kawasaki, K., Kato, S., Higa, S., Maeda N., Minoshima, S., Tanaka, T., Shimizu, N., Kenmochi, N. (2002) The human ribosomal protein genes: sequencing and comparative analysis of 73 genes. Genome Res. 12:379-390.
Yu, L., Zhang, Z., Loewenstein, P. M., Desai, K., Tang, Q., Mao, D., Symington, J. S., Green, M. (1995) Molecular cloning and characterization of a cellular protein that interacts with the human immunodeficiency virus type 1 Tat transactivator and encodes a strong transcriptional activation domain. J. Virol. 69:3007-3016.
Yu, Z., Restifo, N. P. (2002) Cancer vaccines: progress reveals new complexities. J. Clin. Invest. 110:289-294.
Zallipsky, S. (1995) Functionalized poly(ethylene glycols) for preparation of biologically relevant conjugates. Bioconjugate Chem., 6:150-165.
Zhang, Q., Acland, G. M., Wu, W. X., Johnson, J. L., Pearce-Kelling, S., Tulloch, B., Vervoort, R., Wright, A. F., Aguirre, G. D. (2002) Different RPGR exon ORF15 mutations in Canids provide insights into photoreceptor cell degeneration. Hum. Mol. Genet. 11:993-1003.
Zhang, W. M., Popova, S. N., Bergman, C., Velling, T., Gullberg, M. K., Gullberg, D. (2002) Analysis of the human integrin alpha11 gene (ITGA11) and its promoter. Matrix Biol. 21:513-523.
Zhao, H., Grabowski, G. A. (2002) Gaucher disease: Perspectives on a prototype lysosomal disease. Cell Mol. Life. Sci. 59:694-707.
Zhao, N., Hashida, H., Takhshi, N., Misumi, Y., Sakaki, Y. (1995) High-density cDNA filter analysis: a novel approach for large-scale quantitative analysis of gene expression. Gene 156:207-215.
Zhao, Y., Hong, D. H., Pawlyk, B., Yue, G., Adamian, M., Grynberg, M., Godzik, A., Li, T. (2003) The retinitis pigmentosa GTPase regulator (RPGR)-interacting protein: Subserving RPGR function and participating in disk morphogenesis. Proc. Natl. Acad. Sci. 100:3965-3970
Zhu, D. L. (1989) Oligonucleotide-directed cleavage and repair of a single stranded vector: a method of site-specific mutagenesis. Anal. Biochem. 177:120-124.
Zhu, H., Bilgin, M., Bangham, R., Hall, D., Casamayor, P., Bertone, P., Lan, N., Jansen, R., Bidlingmaier, S., Houfek, T., Mitchell, T., Miller, P., Dean, R. A., Gerstein, M., Snyder, M. (2001) Global analysis of protein activities using proteome chips. Science 293:2101-2105.
Zhu, H., Klemic, J. F., Chang, S., Bertone, P., Casamayor, A., Klemic, K. G., Smith, D., Gerstien, M., Reed, M. A., Snyder, M. (2000) Analysis of yeast protein kinases using protein chips. Nat. Genetics 26:283-289.
Zhu, H., Snyder, M. (2003) Protein chip technology. Curr. Opin. Chem. Biol. 7:55-63.
Zhu, J., Kahn, C. R. (1997) Analysis of a peptide hormone-receptor interaction in the yeast two-hybrid system Proc. Natl. Acad. Sci. 94:13,063-13,068.
Zorzano, A., Kaliman, P., Guma, A., Palacin, M. (2003) Intracellular signals involved in the effects of insulin-like growth factors and neuregulins on myofibre formation. Cell Signal. 15:141-149.

TABLE 1
Identification of Novel Human cDNA Clones

| FP ID | SEQ ID NO.: (N1) | SEQ ID NO.: (P1) | Source ID |
|---|---|---|---|
| HG1012781 | SEQ ID NO.: 1 | SEQ ID NO.: 55 | CLN00016650 |
| HG1012782 | SEQ ID NO.: 2 | SEQ ID NO.: 56 | CLN00017433 |
| HG1012785 | SEQ ID NO.: 3 | SEQ ID NO.: 57 | CLN00019493 |
| HG1012793 | SEQ ID NO.: 4 | SEQ ID NO.: 58 | CLN00024961 |
| HG1012798 | SEQ ID NO.: 5 | SEQ ID NO.: 59 | CLN00039449 |
| HG1012800 | SEQ ID NO.: 6 | SEQ ID NO.: 60 | CLN00040108 |
| HG1012809 | SEQ ID NO.: 7 | SEQ ID NO.: 61 | CLN00060395 |
| HG1012814 | SEQ ID NO.: 8 | SEQ ID NO.: 62 | CLN00071567 |
| HG1012827 | SEQ ID NO.: 9 | SEQ ID NO.: 63 | CLN00087149 |
| HG1012834 | SEQ ID NO.: 10 | SEQ ID NO.: 64 | CLN00110621 |
| HG1012840 | SEQ ID NO.: 11 | SEQ ID NO.: 65 | CLN00116457 |
| HG1012842 | SEQ ID NO.: 12 | SEQ ID NO.: 66 | CLN00118287 |
| HG1012844 | SEQ ID NO.: 13 | SEQ ID NO.: 67 | CLN00120717 |
| HG1012858 | SEQ ID NO.: 14 | SEQ ID NO.: 68 | CLN00137844 |
| HG1012860 | SEQ ID NO.: 15 | SEQ ID NO.: 69 | CLN00141249 |
| HG1012861 | SEQ ID NO.: 16 | SEQ ID NO.: 70 | CLN00141940 |
| HG1012864 | SEQ ID NO.: 17 | SEQ ID NO.: 71 | CLN00144017 |
| HG1012875 | SEQ ID NO.: 18 | SEQ ID NO.: 72 | CLN00150953 |
| HG1012876 | SEQ ID NO.: 19 | SEQ ID NO.: 73 | CLN00151148 |
| HG1012882 | SEQ ID NO.: 20 | SEQ ID NO.: 74 | CLN00155728 |
| HG1012884 | SEQ ID NO.: 21 | SEQ ID NO.: 75 | CLN00155800 |
| HG1012887 | SEQ ID NO.: 22 | SEQ ID NO.: 76 | CLN00158047 |
| HG1012888 | SEQ ID NO.: 23 | SEQ ID NO.: 77 | CLN00158725 |
| HG1012894 | SEQ ID NO.: 24 | SEQ ID NO.: 78 | CLN00165897 |
| HG1012898 | SEQ ID NO.: 25 | SEQ ID NO.: 79 | CLN00167288 |
| HG1012901 | SEQ ID NO.: 26 | SEQ ID NO.: 80 | CLN00169841 |
| HG1012909 | SEQ ID NO.: 27 | SEQ ID NO.: 81 | CLN00192537 |
| HG1012913 | SEQ ID NO.: 28 | SEQ ID NO.: 82 | CLN00196720 |
| HG1012919 | SEQ ID NO.: 29 | SEQ ID NO.: 83 | CLN00204715 |
| HG1012921 | SEQ ID NO.: 30 | SEQ ID NO.: 84 | CLN00212212 |
| HG1012933 | SEQ ID NO.: 31 | SEQ ID NO.: 85 | CLN00223392 |
| HG1012935 | SEQ ID NO.: 32 | SEQ ID NO.: 86 | CLN00223851 |
| HG1012956 | SEQ ID NO.: 33 | SEQ ID NO.: 87 | CLN00270184 |
| HG1012957 | SEQ ID NO.: 34 | SEQ ID NO.: 88 | CLN00270227 |
| HG1012981 | SEQ ID NO.: 35 | SEQ ID NO.: 89 | CLN00234852 |
| HG1012982 | SEQ ID NO.: 36 | SEQ ID NO.: 90 | CLN00136882 |
| HG1012993 | SEQ ID NO.: 37 | SEQ ID NO.: 91 | CLN00188160 |
| HG1013000 | SEQ ID NO.: 38 | SEQ ID NO.: 92 | CLN00111867 |
| HG1013001 | SEQ ID NO.: 39 | SEQ ID NO.: 93 | CLN00075810 |
| HG1013003 | SEQ ID NO.: 40 | SEQ ID NO.: 94 | CLN00020198 |
| HG1013004 | SEQ ID NO.: 41 | SEQ ID NO.: 95 | CLN00018201 |
| HG1013006 | SEQ ID NO.: 42 | SEQ ID NO.: 96 | CLN00169943 |
| HG1013007 | SEQ ID NO.: 43 | SEQ ID NO.: 97 | CLN00187739 |
| HG1013011 | SEQ ID NO.: 44 | SEQ ID NO.: 98 | CLN00139890 |
| HG1013017 | SEQ ID NO.: 45 | SEQ ID NO.: 99 | CLN00088225 |
| HG1013018 | SEQ ID NO.: 46 | SEQ ID NO.: 100 | CLN00140475 |
| HG1013023 | SEQ ID NO.: 47 | SEQ ID NO.: 101 | CLN00132470 |
| HG1013025 | SEQ ID NO.: 48 | SEQ ID NO.: 102 | CLN00235393 |
| HG1013033 | SEQ ID NO.: 49 | SEQ ID NO.: 103 | CLN00141615 |
| HG1013048 | SEQ ID NO.: 50 | SEQ ID NO.: 104 | CLN00148376 |
| HG1013049 | SEQ ID NO.: 51 | SEQ ID NO.: 105 | CLN00153052 |
| HG1013052 | SEQ ID NO.: 52 | SEQ ID NO.: 106 | CLN00064053 |
| HG1013069 | SEQ ID NO.: 53 | SEQ ID NO.: 107 | CLN00022964 |
| HG1013080 | SEQ ID NO.: 54 | SEQ ID NO.: 108 | CLN00024767 |

TABLE 2
Structural Charactersitics of Novel Human cDNA Clones

| | | | | | | Altern. | Altern. | | | |
|---|---|---|---|---|---|---|---|---|---|---|
| | | Predicted | | Signal | Mature | Signal | Mature | | | |
| | | Protein | Tree | Peptide | Protein | Peptide | Protein | | TM | non-TM |
| FP ID | Source ID | Length | Vote | Coords. | Coords. | Coords. | Coords | TM | Coords. | Coords. |
| HG1012781 | CLN00016650 | 72 | 0.5 | (1-29) | (30-72) | | | 0 | | (1-72) |
| HG1012782 | CLN00017433 | 54 | 0.6 | | (1-54) | (6-18) | (19-54) | 0 | | (1-54) |
| HG1012785 | CLN00019493 | 85 | 0.32 | | (1-85) | | | 1 | (13-32) | (1-12) |
| | | | | | | | | | | (33-85) |
| HG1012793 | CLN00024961 | 43 | 0.19 | (24-41) | (42-43) | | | 1 | (20-42) | (1-19) |
| | | | | | | | | | | (43-43) |
| HG1012798 | CLN00039449 | 53 | 0.5 | | (1-53) | | | 0 | | (1-53) |
| HG1012800 | CLN00040108 | 97 | 0.58 | (9-37) | (38-97) | (15-27) | (28-97) | 0 | | (1-97) |
| | | | | | | (19-31) | (32-97) | | | |
| | | | | | | (20-32) | (33-97) | | | |
| | | | | | | (17-29) | (30-97) | | | |
| HG1012809 | CLN00060395 | 88 | 0.54 | | (1-88) | (25-37) | (38-88) | 0 | | (1-88) |
| HG1012814 | CLN00071567 | 83 | 0.95 | (1-19) | (20-83) | (9-21) | (22-83) | 0 | | (1-83) |
| HG1012827 | CLN00087149 | 48 | 0.15 | (18-35) | (36-48) | (21-33) | (34-48) | 1 | (20-39) | (1-19) |
| | | | | | | (22-34) | (35-48) | | | (40-48) |
| HG1012834 | CLN00110621 | 60 | 0.59 | (9-29) | (30-60) | (4-16) | (17-60) | 2 | (5-27) | (1-4) |
| | | | | | | (8-20) | (21-60) | | (42-59) | (28-41) |
| | | | | | | (11-23) | (24-60) | | | (60-60) |
| HG1012840 | CLN00116457 | 68 | 0.61 | (8-22) | (23-68) | | | 1 | (10-32) | (1-9) |
| | | | | | | | | | | (33-68) |
| HG1012842 | CLN00118287 | 49 | 0.04 | | (1-49) | | | 1 | (26-48) | (1-25) |
| | | | | | | | | | | (49-49) |
| HG1012844 | CLN00120717 | 61 | 0.92 | (1-17) | (18-61) | (15-27) | (28-61) | 0 | | (1-61) |
| HG1012858 | CLN00137844 | 70 | 0.04 | | (1-70) | | | 1 | (42-64) | (1-41) |
| | | | | | | | | | | (65-70) |
| HG1012860 | CLN00141249 | 80 | 0.01 | | (1-80) | | | 1 | (15-32) | (1-14) |
| | | | | | | | | | | (33-80) |
| HG1012781 | CLN00016650 | 72 | 0.5 | (1-29) | (30-72) | | | 0 | | (1-72) |
| HG1012861 | CLN00141940 | 117 | 0.03 | | (1-117) | | | 1 | (93-115) | (1-92) |
| | | | | | | | | | | (116-117) |
| HG1012864 | CLN00144017 | 85 | 0.67 | (8-20) | (21-85) | (20-32) | (33-85) | 0 | | (1-85) |
| HG1012875 | CLN00150953 | 42 | 0.6 | (1-23) | (24-42) | | | 0 | | (1-42) |
| HG1012876 | CLN00151148 | 58 | 0.65 | (1-26) | (27-58) | (16-28) | (29-58) | 0 | | (1-58) |
| HG1012882 | CLN00155728 | 65 | 0.27 | (20-43) | (44-65) | | | 2 | (4-26) | (1-3) |
| | | | | | | | | | (33-55) | (27-32) |
| | | | | | | | | | | (56-65) |
| HG1012884 | CLN00155800 | 68 | 0.09 | | (1-68) | | | 1 | (21-38) | (1-20) |
| | | | | | | | | | | (39-68) |
| HG1012887 | CLN00158047 | 213 | 0.96 | (1-17) | (18-213) | (5-15) | (16-213) | 0 | | (1-213) |
| | | | | | | (3-13) | (14-213) | | | |
| | | | | | | (4-14) | (15-213) | | | |
| | | | | | | (2-12) | (13-213) | | | |
| | | | | | | (1-11) | (12-213) | | | |
| HG1012888 | CLN00158725 | 41 | 0.55 | | (1-41) | | | 1 | (13-35) | (1-12) |
| | | | | | | | | | | (36-41) |
| HG1012894 | CLN00165897 | 87 | 0.54 | (1-32) | (33-87) | (24-36) | (37-87) | 0 | | (1-87) |
| | | | | | | (19-31) | (32-87) | | | |
| HG1012898 | CLN00167288 | 90 | 0.34 | (1-24) | (25-90) | | | 3 | (12-34) | (1-11) |
| | | | | | | | | | (38-60) | (35-37) |
| | | | | | | | | | (67-89) | (61-66) |
| | | | | | | | | | | (90-90) |
| HG1012901 | CLN00169841 | 52 | 0.89 | (1-33) | (34-52) | (9-21) | (22-52) | 0 | | (1-52) |
| HG1012909 | CLN00192537 | 51 | 1 | (1-24) | (25-51) | (14-26) | (27-51) | 1 | (7-29) | (1-6) |
| | | | | | | | | | | (30-51) |
| HG1012913 | CLN00196720 | 85 | 0.5 | (5-27) | (28-85) | (13-25) | (26-85) | 2 | (13-35) | (1-12) |
| | | | | | | | | | (50-72) | (36-49) |
| | | | | | | | | | | (73-85) |
| HG1012919 | CLN00204715 | 83 | 0.53 | (16-30) | (31-83) | (3-15) | (16-83) | 0 | | (1-83) |
| HG1012781 | CLN00016650 | 72 | 0.5 | (1-29) | (30-72) | | | 0 | | (1-72) |
| HG1012921 | CLN00212212 | 76 | 0.02 | | (1-76) | | | 1 | (34-56) | (1-33) |
| | | | | | | | | | | (57-76) |
| HG1012933 | CLN00223392 | 42 | 0.83 | (6-23) | (24-42) | (13-25) | (26-42) | 1 | (12-34) | (1-11) |
| | | | | | | (14-26) | (27-42) | | | (35-42) |
| HG1012935 | CLN00223851 | 55 | 0.82 | (4-25) | (26-55) | (11-23) | (24-55) | 0 | | (1-55) |
| | | | | | | (17-29) | (30-55) | | | |
| HG1012956 | CLN00270184 | 76 | 0.83 | (1-18) | (19-76) | (16-24) | (25-76) | 0 | | (1-76) |
| | | | | | | (2-10) | (11-76) | | | |
| | | | | | | (1-9) | (10-76) | | | |
| HG1012957 | CLN00270227 | 43 | 0.06 | (20-38) | (39-43) | | | 1 | (20-37) | (1-19) |
| | | | | | | | | | | (38-43) |
| HG1012981 | CLN00234852 | 41 | 0.29 | | (1-41) | (19-31) | (32-41) | 1 | (5-27) | (1-4) |
| | | | | | | | | | | (28-41) |
| HG1012982 | CLN00136882 | 42 | 0.96 | (1-18) | (19-42) | (3-15) | (16-42) | 0 | | (1-42) |
| HG1012993 | CLN00188160 | 255 | 0.13 | (1-24) | (25-255) | (7-19) | (20-255) | 1 | (219-241) | (1-218) |
| | | | | | | (11-23) | (24-255) | | | (242-255) |
| HG1013000 | CLN00111867 | 53 | 0.65 | (1-23) | (24-53) | | | 0 | | (1-53) |
| HG1013001 | CLN00075810 | 40 | 0.49 | | (1-40) | | | 1 | (7-26) | (1-6) |
| | | | | | | | | | | (27-40) |
| HG1013003 | CLN00020198 | 52 | 0.08 | | (1-52) | (23-35) | (36-52) | 1 | (15-34) | (1-14) |
| | | | | | | | | | | (35-52) |
| HG1013004 | CLN00018201 | 93 | 0.6 | (1-23) | (24-93) | | | 1 | (7-29) | (1-6) |
| | | | | | | | | | | (30-93) |
| HG1013006 | CLN00169943 | 59 | 0.63 | (1-27) | (28-59) | (9-21) | (22-59) | 0 | | (1-59) |
| | | | | | | (11-23) | (24-59) | | | |
| | | | | | | (21-33) | (34-59) | | | |
| HG1013007 | CLN00187739 | 114 | 0.02 | | (1-114) | | | 1 | (33-50) | (1-32) |
| | | | | | | | | | | (51-114) |
| HG1013011 | CLN00139890 | 57 | 0.39 | (24-37) | (38-57) | | | 1 | (15-37) | (1-14) |
| | | | | | | | | | | (38-57) |
| HG1012781 | CLN00016650 | 72 | 0.5 | (1-29) | (30-72) | | | 0 | | (1-72) |
| HG1013017 | CLN00088225 | 78 | 0.03 | | (1-78) | | | 1 | (41-63) | (1-40) |
| | | | | | | | | | | (64-78) |
| HG1013018 | CLN00140475 | 71 | 0.56 | (13-29) | (30-71) | (11-23) | (24-71) | 0 | | (1-71) |
| HG1013023 | CLN00132470 | 75 | 0.53 | (13-38) | (39-75) | | | 0 | | (1-75) |
| HG1013025 | CLN00235393 | 255 | 0.14 | (1-24) | (25-255) | (7-19) | (20-255) | 1 | (218-240) | (1-217) |
| | | | | | | (11-23) | (24-255) | | | (241-255) |
| HG1013033 | CLN00141615 | 80 | 0.98 | (10-32) | (33-80) | (12-24) | (25-80) | 1 | (10-29) | (1-9) |
| | | | | | | (18-30) | (31-80) | | | (30-80) |
| | | | | | | (14-26) | (27-80) | | | |
| HG1013048 | CLN00148376 | 66 | 0.03 | (22-44) | (45-66) | | | 1 | (29-51) | (1-28) |
| | | | | | | | | | | (52-66) |
| HG1013049 | CLN00153052 | 86 | 0.84 | (1-18) | (19-86) | | | 0 | | (1-86) |
| HG1013052 | CLN00064053 | 55 | 0.03 | | (1-55) | | | 1 | (13-35) | (1-12) |
| | | | | | | | | | | (36-55) |
| HG1013069 | CLN00022964 | 154 | 0.98 | (1-17) | (18-154) | (7-19) | (20-154) | 0 | | (1-154) |
| HG1013080 | CLN00024767 | 88 | 0.01 | | (1-88) | | | 1 | (20-42) | (1-19) |
| | | | | | | | | | | (43-88) |

TABLE 3
Top Hit Annotations for Novel Human cDNA Clones

| | | Pre- | | | | | % ID | % ID |
|---|---|---|---|---|---|---|---|---|
| | | dicted | | | | Number | Over | Over |
| | | Protein | | | Top Hit | of | Query | Hit |
| FP ID | Source ID | Length | Top Hit Accession ID | Top Hit Annotation | Length | Matches | Length | Length |
| HG1012781P1 | CLN00016650 | 72 | gi\|29246459\|gb\|EAA38055.1\| | GLP_327_33046_34074 | 342 | 26 | 36% | 8% |
| | | | | [Giardia lamblia ATCC 50803] | | | | |
| HG1012793P1 | CLN00024961 | 43 | gi\|34533355\|dbj\|BAC86672.1\| | unnamed protein product | 204 | 23 | 53% | 11% |
| | | | | [Homo sapiens] | | | | |
| HG1012800P1 | CLN00040108 | 97 | gi\|38102410\|gb\|EAA49251.1\| | hypothetical protein | 482 | 32 | 33% | 7% |
| | | | | MG00909.4 [Magnaporthe | | | | |
| | | | | grisea 70-15] | | | | |
| HG1012827P1 | CLN00087149 | 48 | gi\|30697506\|ref\|NP_176909.2\| | 24 kDa vacuolar protein, | 873 | 15 | 31% | 2% |
| | | | | putative [Arabidopsis thaliana] | | | | |
| HG1012840P1 | CLN00116457 | 68 | gi\|48526542\|gb\|AAT45470.1\| | cytochrome oxidase subunit 1 | 528 | 22 | 32% | 4% |
| | | | | [Cryptococcus neoformans var. | | | | |
| | | | | neoformans] | | | | |
| HG1012842P1 | CLN00118287 | 49 | gi\|34876703\|ref\|XP_347116.1\| | hypothetical protein | 188 | 20 | 41% | 11% |
| | | | | XP_347115 [Rattus norvegicus] | | | | |
| HG1012844P1 | CLN00120717 | 61 | gi\|13938494\|gb\|AAH07394.1\| | MGC16291 protein | 114 | 22 | 36% | 19% |
| | | | | [Homo sapiens] | | | | |
| HG1012860P1 | CLN00141249 | 80 | gi\|26380023\|dbj\|BAC25024.1\| | unnamed protein product | 88 | 63 | 79% | 72% |
| | | | | [Mus musculus] | | | | |
| HG1012864P1 | CLN00144017 | 85 | gi\|46106170\|ref\|ZP_00199861.1\| | COG0477: Permeases of the | 148 | 33 | 39% | 22% |
| | | | | major facilitator superfamily | | | | |
| | | | | [Rubrobacter xylanophilus DSM | | | | |
| | | | | 9941] | | | | |
| HG1012882P1 | CLN00155728 | 65 | gi\|10802913\|gb\|AAG23661.1\| | NADH dehydrogenase subunit | 500 | 20 | 31% | 4% |
| | | | | 2 [Thraustochytrium aureum] | | | | |
| HG1012884P1 | CLN00155800 | 68 | gi\|37782452\|gb\|AAP34472.1\| | LP3428 [Homo sapiens] | 80 | 28 | 41% | 35% |
| HG1012887P1 | CLN00158047 | 213 | gi\|16716593\|ref\|NP_444490.1\| | implantation serine protease 2 | 279 | 71 | 33% | 25% |
| | | | | [Mus musculus] | | | | |
| HG1012894P1 | CLN00165897 | 87 | gi\|26328355\|dbj\|BAC27918.1\| | unnamed protein product | 771 | 29 | 33% | 4% |
| | | | | [Mus musculus] | | | | |
| HG1012898P1 | CLN00167288 | 90 | gi\|34528160\|dbj\|BAC85462.1\| | unnamed protein product | 138 | 34 | 38% | 25% |
| | | | | [Homo sapiens] | | | | |
| HG1012901P1 | CLN00169841 | 52 | gi\|9858152\|gb\|AAG01019.1\| | airway mucin Muc-5ac | 178 | 19 | 37% | 11% |
| | | | | [Mesocricetus auratus] | | | | |
| HG1012909P1 | CLN00192537 | 51 | gi\|49120618\|ref\|XP_412364.1\| | predicted protein [Aspergillus | 467 | 17 | 33% | 4% |
| | | | | nidulans FGSC A4] | | | | |
| HG1012913P1 | CLN00196720 | 85 | gi\|25396150\|pir\|\|F88924 | protein R02C2.2 [imported] - | 484 | 27 | 32% | 6% |
| | | | | Caenorhabditis elegans | | | | |
| HG1012921P1 | CLN00212212 | 76 | gi\|47216147\|emb\|CAG10021.1\| | unnamed protein product | 1453 | 28 | 37% | 2% |
| | | | | [Tetraodon nigroviridis] | | | | |
| HG1012933P1 | CLN00223392 | 42 | gi\|1084987\|pir\|\|S51910 | cryptogene protein G4 - | 169 | 16 | 38% | 9% |
| | | | | Leishmania tarentolae (strain | | | | |
| | | | | LEM125) | | | | |
| HG1012956P1 | CLN00270184 | 76 | gi\|47219080\|emb\|CAG00219.1\| | unnamed protein product | 1113 | 27 | 36% | 2% |
| | | | | [Tetraodon nigroviridis] | | | | |
| HG1012993P1 | CLN00188160 | 255 | gi\|87115\|pir\|\|A29312 | MHC class II histocompatibility | 255 | 253 | 99% | 99% |
| | | | | antigen HLA-DQ alpha chain | | | | |
| | | | | precursor - human | | | | |
| HG1013000P1 | CLN00111867 | 53 | gi\|32416700\|ref\|XP_328828.1\| | predicted protein [Neurospora | 94 | 19 | 36% | 20% |
| | | | | crassa] | | | | |
| HG1013004P1 | CLN00018201 | 93 | gi\|37182988\|gb\|AAQ89294.1\| | DSLR655 [Homo sapiens] | 93 | 93 | 100% | 100% |
| HG1013018P1 | CLN00140475 | 71 | gi\|41149720\|ref\|XP_370705.1\| | hypothetical protein | 140 | 70 | 99% | 50% |
| | | | | XP_374993 [Homo sapiens] | | | | |
| HG1013025P1 | CLN00235393 | 255 | gi\|87115\|pir\|\|A29312 | MHC class II histocompatibility | 255 | 255 | 100% | 100% |
| | | | | antigen HLA-DQ alpha chain | | | | |
| | | | | precursor - human | | | | |
| HG1013033P1 | CLN00141615 | 80 | gi\|21757056\|dbj\|BAC05007.1\| | unnamed protein product | 157 | 36 | 45% | 23% |
| | | | | [Homo sapiens] | | | | |
| HG1013048P1 | CLN00148376 | 66 | gi\|26351913\|dbj\|BAC39593.1\| | unnamed protein product | 101 | 22 | 33% | 22% |
| | | | | [Mus musculus] | | | | |
| HG1013049P1 | CLN00153052 | 86 | gi\|49645061\|emb\|CAG98633.1\| | unnamed protein product | 106 | 26 | 30% | 25% |
| | | | | [Kluyveromyces lactis] | | | | |
| HG1013052P1 | CLN00064053 | 55 | gi\|42521049\|ref\|NP_966964.1\| | hypothetical protein WD1252 | 197 | 18 | 33% | 9% |
| | | | | [Wolbachia endosymbiont of | | | | |
| | | | | Drosophila melanogaster] | | | | |
| HG1013069P1 | CLN00022964 | 154 | gi\|34531284\|dbj\|BAC86100.1\| | unnamed protein product | 129 | 73 | 47% | 57% |
| | | | | [Homo sapiens] | | | | |

TABLE 4
Top Human Hit Annotations for Novel Human cDNA Clones

| | | | | | Top | | % ID | |
|---|---|---|---|---|---|---|---|---|
| | | Predicted | | | Human | Number | Over | % ID Over |
| | | Protein | Top Human Hit | Top Human Hit | Hit | of | Query | Human Hit |
| FP ID | Source ID | Length | Accession ID | Annotation | Length | Matches | Length | Length |
| HG1012793 | CLN00024961 | 43 | gi\|34533355\|dbj\|BAC86672.1\| | unnamed protein product | 204 | 23 | 53% | 11% |
| | | | | [Homo sapiens] | | | | |
| HG1012844 | CLN00120717 | 61 | gi\|13938494\|gb\|AAH07394.1\| | MGC16291 protein | 114 | 22 | 36% | 19% |
| | | | | [Homo sapiens] | | | | |
| HG1012884 | CLN00155800 | 68 | gi\|37782452\|gb\|AAP34472.1\| | LP3428 [Homo sapiens] | 80 | 28 | 41% | 35% |
| HG1012898 | CLN00167288 | 90 | gi\|34528160\|dbj\|BAC85462.1\| | unnamed protein product | 138 | 34 | 38% | 25% |
| | | | | [Homo sapiens] | | | | |
| HG1012993 | CLN00188160 | 255 | gi\|87115\|pir\|\|A29312 | MHC class II | 255 | 253 | 99% | 99% |
| | | | | histocompatibility antigen | | | | |
| | | | | HLA-DQ alpha chain | | | | |
| | | | | precursor - human | | | | |
| HG1013004 | CLN00018201 | 93 | gi\|37182988\|gb\|AAQ89294.1\| | DSLR655 | 93 | 93 | 100% | 100% |
| | | | | [Homo sapiens] | | | | |
| HG1013018 | CLN00140475 | 71 | gi\|41149720\|ref\|XP_370705.1\| | hypothetical protein | 140 | 70 | 99% | 50% |
| | | | | XP_374993 [Homo | | | | |
| | | | | sapiens] | | | | |
| HG1013025 | CLN00235393 | 255 | gi\|87115\|pir\|\|A29312 | MHC class II | 255 | 255 | 100% | 100% |
| | | | | histocompatibility antigen | | | | |
| | | | | HLA-DQ alpha chain | | | | |
| | | | | precursor - human | | | | |
| HG1013033 | CLN00141615 | 80 | gi\|21757056\|dbj\|BAC05007.1\| | unnamed protein product | 157 | 36 | 45% | 23% |
| | | | | [Homo sapiens] | | | | |
| HG1013069 | CLN00022964 | 154 | gi\|34531284\|dbj\|BAC86100.1\| | unnamed protein product | 129 | 73 | 47% | 57% |
| | | | | [Homo sapiens] | | | | |

TABLE 5
Pfam Domains of Novel Human cDNA Clones

| FP ID | Source ID | Pfam | Coordinates |
|---|---|---|---|
| HG1012993P1 | CLN00188160 | MHC_II_alpha | (29-110) |
| HG1012993P1 | CLN00188160 | ig | (126-191) |
| HG1013025P1 | CLN00235393 | MHC_II_alpha | (29-110) |
| HG1013025P1 | CLN00235393 | ig | (126-191) |

TABLE 6
Structural Motifs in Novel Human cDNA Clones

| | | | Predicted | | Signal | | | | |
|---|---|---|---|---|---|---|---|---|---|
| Patent Protein | | | Protein | Tree | Peptide | Mature Protein | | | |
| ID | Source ID | Structual Motifs | Length | Vote | Coords | Coords. | TM | TM Coords. | Pfam |
| HG1012887P1 | CLN00158047 | Trypsin-like serine | 213 | 0.96 | (1-17) | (18-213) | 0 | | none |
| | | proteases | | | | | | | |
| HG1012993P1 | CLN00188160 | Class II histocompatibility | 255 | 0.13 | (1-24) | (25-255) | 1 | (219-241) | MHC_II_alpha; |
| | | antigen, alpha domain | | | | | | | ig |
| HG1012993P1 | CLN00188160 | Immunoglobulin | | | | | | | MHC_II_alpha; |
| | | | | | | | | | ig |
| HG1012993P1 | CLN00188160 | MHC antigen-recognition | | | | | | | MHC_II_alpha; |
| | | domain | | | | | | | ig |
| HG1012993P1 | CLN00188160 | WW domain | | | | | | | MHC_II_alpha; |
| | | | | | | | | | ig |
| HG1013025P1 | CLN00235393 | Class II histocompatibility | 255 | 0.14 | (1-24) | (25-255) | 1 | (218-240) | MHC_II_alpha; |
| | | antigen, alpha domain | | | | | | | ig |
| HG1013025P1 | CLN00235393 | Immunoglobulin | | | | | | | MHC_II_alpha; |
| | | | | | | | | | ig |
| HG1013025P1 | CLN00235393 | MHC antigen-recognition | | | | | | | MHC_II_alpha; |
| | | domain | | | | | | | ig |
| HG1013025P1 | CLN00235393 | WW domain | | | | | | | MHC_II_alpha; |
| | | | | | | | | | ig |

TABLE 7
Tissue Sources of the Novel Human cDNA Clones

| FP ID | Source ID | Library | Library ID | Tissue |
|---|---|---|---|---|
| HG1012781 | CLN00016650 | LIB00000017 | FP010N | Intestine, Pancreas, Pancreas pool, Stomach, Stomach pool, Trachea, Trachea pool |
| HG1012782 | CLN00017433 | LIB00000017 | FP010N | Intestine, Pancreas, Pancreas pool, Stomach, Stomach pool, Trachea, Trachea pool |
| HG1012785 | CLN00019493 | LIB00000017 | FP010N | Intestine, Pancreas, Pancreas pool, Stomach, Stomach pool, Trachea, Trachea pool |
| HG1012793 | CLN00024961 | LIB00000002 | FP003N | Bone Marrow, Bone Marrow pool, Liver |
| HG1012798 | CLN00039449 | LIB00000009 | FP006N | Adrenal Gland, Adrenal Gland pool |
| HG1012800 | CLN00040108 | LIB00000009 | FP006N | Adrenal Gland, Adrenal Gland pool |
| HG1012809 | CLN00060395 | LIB00000019 | FP011N | Kidney |
| HG1012814 | CLN00071567 | LIB00000010 | FP007C | Testis, Testis pool |
| HG1012827 | CLN00087149 | LIB00000019 | FP011N | Kidney |
| HG1012834 | CLN00110621 | LIB00000007 | FP005N | Liver |
| HG1012840 | CLN00116457 | LIB00000015 | FP009N | Bladder, Brain, Brain pool, Lung, Lung pool, Spleen, Spleen pool, Thymus, Thymus pool |
| HG1012842 | CLN00118287 | LIB00000015 | FP009N | Bladder, Brain, Brain pool, Lung, Lung pool, Spleen, Spleen pool, Thymus, Thymus pool |
| HG1012844 | CLN00120717 | LIB00000015 | FP009N | Bladder, Brain, Brain pool, Lung, Lung pool, Spleen, Spleen pool, Thymus, Thymus pool |
| HG1012858 | CLN00137844 | LIB00000014 | FP009C | Bladder, Brain, Brain pool, Lung, Lung pool, Spleen, Spleen pool, Thymus, Thymus pool |
| HG1012860 | CLN00141249 | LIB00000024 | FP014C | Lung, Lung pool |
| HG1012861 | CLN00141940 | LIB00000026 | FP015C | Prostate, Prostate pool |
| HG1012864 | CLN00144017 | LIB00000030 | FP017C | Kidney |
| HG1012875 | CLN00150953 | LIB00000055 | FP014X | Lung, Lung pool |
| HG1012876 | CLN00151148 | LIB00000055 | FP014X | Lung, Lung pool |
| HG1012882 | CLN00155728 | LIB00000011 | FP007HN | Testis, Testis pool |
| HG1012884 | CLN00155800 | LIB00000011 | FP007HN | Testis, Testis pool |
| HG1012887 | CLN00158047 | LIB00000021 | FP012HN | Placenta |
| HG1012888 | CLN00158725 | LIB00000021 | FP012HN | Placenta |
| HG1012894 | CLN00165897 | LIB00000031 | FP017S | Kidney |
| HG1012898 | CLN00167288 | LIB00000033 | FP018S | Skin, Skin pool |
| HG1012901 | CLN00169841 | LIB00000037 | FP020S | Tonsil, Tonsil pool |
| HG1012909 | CLN00192537 | LIB00000025 | FP014S | Lung, Lung pool |
| HG1012913 | CLN00196720 | LIB00000027 | FP015S | Prostate, Prostate pool |
| HG1012919 | CLN00204715 | LIB00000029 | FP016S | Colon |
| HG1012921 | CLN00212212 | LIB00000031 | FP017S | Kidney |
| HG1012933 | CLN00223392 | LIB00000033 | FP018S | Skin, Skin pool |
| HG1012935 | CLN00223851 | LIB00000033 | FP018S | Skin, Skin pool |
| HG1012956 | CLN00270184 | LIB00000039 | FP021S | PBMC, Spleen, Thymus, Thymus pool |
| HG1012957 | CLN00270227 | LIB00000039 | FP021S | PBMC, Spleen, Thymus, Thymus pool |
| HG1012981 | CLN00234852 | LIB00000035 | FP019S | Tonsil, Tonsil pool |
| HG1012982 | CLN00136882 | LIB00000014 | FP009C | Bladder, Brain, Brain pool, Lung, Lung pool, Spleen, Spleen pool, Thymus, Thymus pool |
| HG1012993 | CLN00188160 | LIB00000023 | FP013S | Breast |
| HG1013000 | CLN00111867 | LIB00000015 | FP009N | Bladder, Brain, Brain pool, Lung, Lung pool, Spleen, Spleen pool, Thymus, Thymus pool |
| HG1013001 | CLN00075810 | LIB00000010 | FP007C | Testis, Testis pool |
| HG1013003 | CLN00020198 | LIB00000017 | FP010N | Intestine, Pancreas, Pancreas pool, Stomach, Stomach pool, Trachea, Trachea pool |
| HG1013004 | CLN00018201 | LIB00000017 | FP010N | Pancreas, Trachea, Trachea pool |
| HG1013006 | CLN00169943 | LIB00000037 | FP020S | Tonsil, Tonsil pool |
| HG1013007 | CLN00187739 | LIB00000023 | FP013S | Breast |
| HG1013011 | CLN00139890 | LIB00000022 | FP013C | Breast |
| HG1013017 | CLN00088225 | LIB00000019 | FP011N | Kidney |
| HG1013018 | CLN00140475 | LIB00000024 | FP014C | Lung, Lung pool |
| HG1013023 | CLN00132470 | LIB00000001 | FP003C | Bone Marrow, Bone Marrow pool, Liver |
| HG1013025 | CLN00235393 | LIB00000035 | FP019S | Tonsil pool, Tonsil |
| HG1013033 | CLN00141615 | LIB00000026 | FP015C | Prostate, Prostate pool |
| HG1013048 | CLN00148376 | LIB00000036 | FP020C | Cord Blood, Cord Blood pool, Placenta, Placenta pool |
| HG1013049 | CLN00153052 | | | No library information available |
| HG1013052 | CLN00064053 | LIB00000019 | FP011N | Kidney |
| HG1013069 | CLN00022964 | LIB00000002 | FP003N | Bone Marrow, Bone Marrow pool, Liver |
| HG1013080 | CLN00024767 | LIB00000002 | FP003N | Bone Marrow, Bone Marrow pool, Liver |

TABLE 8
Tissue Localization and Predicted Function of Novel cDNA Clones

| FP ID | Source ID | Classification | Tissues | Predicted Function |
|---|---|---|---|---|
| HG1012840 | CLN00116457 | SEC | B-cell, CD8 cells, | immune system, |
| | | | lymph node, NK | immune system |
| | | | cells, skin, soft | activation, Grave's |
| | | | tissue, spleen, | disease, Hashimoto's |
| | | | thyroid | disease, |
| | | | | immunoregulation, |
| | | | | autoimmunity, immune |
| | | | | response, immune |
| | | | | potentiation, infectious |
| | | | | disease |
| HG1012858 | CLN00137844 | STM | CD8 cells, NK cells, | immune system, |
| | | | spleen, thyroid, | autoimmune thyroiditis, |
| | | | white blood cells | cancer, infectious |
| | | | | disease (viral), immune |
| | | | | regulation |
| HG1012909 | CLN00192537 | SEC | skeletal muscle, | fertility, type II diabetes |
| | | | fallopian tube, liver | |
| HG1012913 | CLN00196720 | SEC | CD4 cells, colon, | asthma, breast cancer, |
| | | | fallopian tube, | diabetes, fertility, |
| | | | jejunum, kidney, | immune regulation, |
| | | | lung, lymph node, | chronic obstructive |
| | | | ovary, pancreas, | pulmonary disease |
| | | | parotid, pituitary, | |
| | | | placenta, prostate, | |
| | | | rectum, skeletal | |
| | | | muscle, soft tissue, | |
| | | | spleen, subcutaneous | |
| | | | adipose tissue, testis, | |
| | | | thyroid, uterus | |
| HG1012919 | CLN00204715 | SEC | adrenal, colon, B- | immune function, |
| | | | cell, bladder, bone | Addison's disease, |
| | | | marrow, breast, CD4 | ulcerative colitis, |
| | | | cells, CD8 cells, | Crohn's disease, |
| | | | duodenum, fallopian | inflammatory bowel |
| | | | tube, gallbladder, | disease, psoriasis, |
| | | | heart, jejunum, | fertility, Grave's |
| | | | kidney, lung, lymph | disease, Hashimoto's |
| | | | node, monocytes, | disease, asthma, chronic |
| | | | NK cells, omentum, | obstructive pulmonary |
| | | | pituitary, placenta, | disease, immune |
| | | | protstate, rectum, | response, infectious |
| | | | skeletal muscle, | disease, T-cell |
| | | | skin, small intestine, | autoimmunity, B-cell |
| | | | soft tissue, spleen, | autoimmunity, |
| | | | stem cell, testis, | inflammation, immune |
| | | | thymus, thyroid, | regulation, |
| | | | uterus, white blood | lymphopoeisis, |
| | | | cells, B-cell, bone | monopoeisis, lymphoid |
| | | | marrow, CD4 cells, | differentiation, |
| | | | CD8 cells, lung, | monocyte |
| | | | monocytes, NK cells | differentiation |
| HG1012957 | CLN00270227 | STM | B-cell, CD8 cells, | B-cell function, immune |
| | | | rectum, soft tissue, | response, B-cell |
| | | | spleen | activation, B-cell |
| | | | | homing, B-cell |
| | | | | development, B-cell |
| | | | | maturation, B-cell |
| | | | | autoimmunity, |
| | | | | infectious disease |
| HG1013033 | CLN00141615 | SEC | colon, duodenum, | gastrointestinal |
| | | | gallbladder, | function, appetite |
| | | | jejunum, prostate, | modulation, celiac |
| | | | rectum, small | disease, colon cancer, |
| | | | intestine | obesity, type II diabetes, |
| | | | | ulcerative colitis, |
| | | | | inflammatory bowel |
| | | | | disease, Crohn's disease |

TABLE 9
Tissue Localization of Novel cDNA Clones

| FP ID | Source ID | Classification | Tissues |
|---|---|---|---|
| HG1012782 | CLN00017433 | SEC | CD4 cells, placenta |
| HG1012793 | CLN00024961 | STM | not detected* |
| HG1012798 | CLN00039449 | SEC | colon, CD8 cells, heart, jejunum, kidney, lung, myometrium, |
| | | | parotid, placenta, rectum, skeletal muscle, soft tissue, testis, |
| | | | thyroid |
| HG1012800 | CLN00040108 | SEC | not detected* |
| HG1012827 | CLN00087149 | STM | white blood cells |
| HG1012842 | CLN00118287 | STM | colon, B-cells, bone marrow, breast, CD8 cells, fallopian |
| | | | tubes, jejunum, kidney, lung, lymph node, monocytes, |
| | | | myometrium, NK cells, omentum, ovary, pituitary, placenta, |
| | | | prostate, rectum, skeletal muscle, skin, small intestine, soft |
| | | | tissue, spleen, stem cell, subcutaneous adipose tissue, testis, |
| | | | thymus, thyroid, uterus, white blood cell |
| HG1012844 | CLN00120717 | SEC | colon, liver, B-cells |
| HG1012860 | CLN00141249 | STM | adrenal, colon, B-cells, bladder, bone marrow, breast, CD4 |
| | | | cells, gallbladder, jejunum, kidney, lung, lymph node, |
| | | | myometrium, NK cells, omentum, pituitary, placenta, |
| | | | prostate, rectum, small intestine, soft tissue, spleen, |
| | | | subcutaneous adipose tissue, testis, thymus, thyroid, uterus, |
| | | | white blood cell |
| HG1012864 | CLN00144017 | SEC | adrenal, colon, B-cells, bladder, bone marrow, breast, CD4 |
| | | | cells, CD8 cells, fallopian tubes, gallbladder, heart, jejunum, |
| | | | kidney, liver, lung, lymph node, myometrium, NK cells, |
| | | | omentum, pancreas, parotid, prostate, rectum, small intestine, |
| | | | soft tissue, spleen, stem cell, subcutaneous adipose tissue, |
| | | | testis, thymus, thyroid, uterus |
| HG1012875 | CLN00150953 | SEC | spleen, lung |
| HG1012876 | CLN00151148 | SEC | not detected* |
| HG1012882 | CLN00155728 | MTM | adrenal, colon, B-cells, bladder, bone marrow, breast, CD4 |
| | | | cells, duodenum, fallopian tubes, galbladder, heart, jejunum, |
| | | | kidney, liver, lung, lymph node, monocytes, myometrium, NK |
| | | | cells, omentum, ovary, pituitary, protstate, rectum, skeletal |
| | | | muscle, skin, small intestine, spleen, stem cell, subcutaneous |
| | | | adipose tissue, testis, thyroid, uterus |
| HG1012884 | CLN00155800 | STM | testis, lung |
| HG1012887 | CLN00158047 | SEC | not detected* |
| HG1012888 | CLN00158725 | SEC | not detected* |
| HG1012898 | CLN00167288 | MTM | adrenal, colon, B-cells, bladder, bone marrow, breast, |
| | | | duodenum, fallopian tubes, jejunum, kidney, lung, lymph |
| | | | node, monocytes, NK cells, omentum, ovary, pituitary, |
| | | | prostate, rectum, skin, small intestine, soft tissue, spleen, stem |
| | | | cell, subcutaneous adipose tissue, testis, thymus, thyroid, |
| | | | white blood cell |
| HG1012909 | CLN00192537 | SEC | skeletal muscle, fallopian tubes, liver |
| HG1012933 | CLN00223392 | SEC | not detected* |
| HG1012935 | CLN00223851 | SEC | not detected* |
| HG1012956 | CLN00270184 | SEC | not detected* |
| HG1013007 | CLN00187739 | STM | not detected* |

*Not Detected: The following tissues were probed, and the novel cDNA clone was not detected: Normal adrenal, ascending colon, B-cells, bladder, bone marrow, breast, CD4 cells, CD8 cells, colon, duodenum, fallopian tubes, gall bladder, heart, jejunum, kidney, lung, liver, lymph node, monocytes, myometrium, NK cells, omentum, ovary, pancreas, parotid, pituitary, placenta, prostate, rectum, skeletal muscle, skin, small intestine, soft tissue, spleen, stem cell, subcutaneous adipose tissue, testis, thymus, thyroid, uterus, and white blood cells; Malignant breast, colon, lung adenocarcinoma, lung squamous cell carcinoma, and prostate.

1-68. (canceled)

69. A polypeptide comprising an amino acid sequence selected from the amino acid sequences of SEQ ID NOs: 55 to 108, and amino acid sequences that are 95% identical to the amino acid sequences of SEQ ID NOs: 55 to 108.

70. The polypeptide of claim 69, further comprising a fusion partner.

71. The polypeptide of claim 70, wherein the fusion partner comprises a polymer, human serum albumin, fetuin A, fetuin B, an Fc domain, or a fragment thereof.

72. The polypeptide of claim 71, wherein the fusion partner comprises an Fc domain.

73. The polypeptide of claim 70, wherein the polypeptide comprises a pegylated polypeptide.
